(19) United States
(12) Patent Application Publication
(10) Pub. No.: US 2020/0399689 A1
(43) Pub. Date: Dec. 24, 2020

(54) METHODS OF DETECTING NUCLEIC ACID SEQUENCES WITH HIGH SPECIFICITY

(71) Applicant: ADVANCED CELL DIAGNOSTICS, INC., Newark, CA (US)

(72) Inventors: Yuling LUO, San Ramon, CA (US); John James FLANAGAN, Walnut Creek, CA (US); Nan SU, San Ramon, CA (US); Huei-Yu Fay WANG, San Francisco, CA (US)

(73) Assignee: ADVANCED CELL DIAGNOSTICS, INC., Newark, CA (US)

(21) Appl. No.: 16/892,826

(22) Filed: Jun. 4, 2020

Related U.S. Application Data
(63) Continuation of application No. 13/498,249, filed on Oct. 4, 2012, now abandoned.
(60) Provisional application No. 61/355,246, filed on Jun. 16, 2010.
(60) Provisional application No. 61/355,244, filed on Jun. 16, 2010.
(60) Provisional application No. 61/283,503, filed on Dec. 7, 2009.
(60) Provisional application No. 61/277,563, filed on Sep. 28, 2009.

(51) Int. Cl. C12Q 1/6841

(52) U.S. Cl. CPC C12Q 1/6841

(57) ABSTRACT

The invention relates to methods of detecting nucleic acids, including methods of detecting one or more target nucleic acid sequences in multiplex branched-chain DNA assays, are provided. Nucleic acids captured on a solid support or suspending cells are detected, for example, through cooperative hybridization events that result in specific association of a label with the nucleic acids. The invention further relates to methods to improve probe hybridization specificity and their application in genotyping. The invention also relates to in situ detection of mis-joined nucleic acid sequences. The invention relates to reducing false positive signals and improve signal-to-background ratio in hybridization-based nucleic acid detection assay. The invention further relates to method to improve specificity in hybridization based nucleic acid using co-location probes. Compositions, tissue slides, sample of suspended cells, kits, and systems related to the methods are also described.

### CROSS-REFERENCE TO RELATED APPLICATIONS

This application is a continuation-in-part of U.S. patent application Ser. No. 12/284,163, filed Sep. 17, 2008, entitled “METHODS TO REDUCE FALSE POSITIVE SIGNALS AND IMPROVE SIGNAL-TO-BACKGROUND RATIO IN HYBRIDIZATION-BASED NUCLEIC ACID DETECTION ASSAY”, which is a continuation-in-part of U.S. patent application Ser. No. 11/471,278, filed Jun. 19, 2006, which claims priority to and benefit of U.S. Provisional Application No. 60/691,834, filed Jun. 20, 2005.

This application is also a continuation-in-part of U.S. patent application Ser. No. 12/660,524, filed Feb. 26, 2010, and a continuation-in-part of U.S. patent application Ser. No. 12/660,516, filed Feb. 26, 2010, which are a continuation and divisional, respectively, of U.S. patent application Ser. No. 11/471,025 filed Jun. 19, 2006, now U.S. Pat. No. 7,709,198, which claims priority to and benefit of U.S. Provisional Application No. 60/691,834, filed Jun. 20, 2005.

This application also claims priority to and benefit of U.S. Provisional Application No. 61/277,563 filed Sep. 28, 2009, entitled “METHODS TO IMPROVE PROBE HYBRIDIZATION SPECIFICITY AND THEIR APPLICATION IN GENOTYPING”; U.S. Provisional Application No. 61/355,244 filed Jun. 16, 2010, entitled “IN SITU DETECTION OF MIS-JOINED NUCLEIC ACID SEQUENCES”; and U.S. Provisional Application No. 61/355,246 filed Jun. 16, 2010, entitled “METHODS TO REDUCE FALSE POSITIVE SIGNALS AND IMPROVE SIGNAL-TO-BACKGROUND RATIO IN HYBRIDIZATION-BASED NUCLEIC ACID DETECTION ASSAY”

Each of the aforementioned applications and patents is incorporated herein by reference in its entirety for all purposes.

### FIELD OF THE INVENTION

The invention relates generally to nucleic acid chemistry and biochemical assays. More particularly, the invention relates to methods to detect one or more nucleic acids in multiplex branched-chain DNA assay. The invention also relates to methods to improve probe hybridization specificity and their application in genotyping. The invention also relates to in situ detection of mis-joined nucleic acid sequences. The invention further relates to reduce false positive signals and improve signal-to-background ratio in hybridization-based nucleic acid detection assay. Compositions, tissue slides, sample of suspended cells, kits, and systems relate to the methods are also described.

### BACKGROUND OF THE INVENTION

Many assays designed to detect target nucleic acid molecules of specific sequences involve associating one or multiple signal generating molecules (e.g. a fluorescent label) to the target nucleic acid specifically. The simplest approach is so called “direct labeling” method, in which a label probe is produced by chemically linking the fluorescent label to a section of nucleic acid with complementary sequence and then hybridize the label probe to the target. A more sophisticated method is called “indirect labeling”, in which a generic “signal generating probe” (SGP) is captured to the target molecule through one or multiple capture probes (CPs), which has one segment complementary to the target and another segment complementary to one component of the label. One of the advantages of this indirect labeling approach is that the LPS can be a relatively large scaffold allowing many more label molecules to be linked to the target thus creating a signal amplification effect.

Normally, the capture probe (CP) is designed so that the melting temperatures of the binding between CP and SGP and between CP and target are both above the assay hybridization temperature. In this way, the label molecules remain stably hybridized to the target through out the assay. However, there is a possibility that the CP could hybridizes to a non-specific sequence that does not belong to the intended target. This non-specific sequence could share the same sequence as the target or it could carry small number of mis-matches that are insufficient to be prevented from binding to CP nonspecifically. This will result in a false positive signal because the label is mistakenly captured to non-targets. One way to reduce the non-specific hybridization is to intentionally institute a capture probe set configuration between the label and the target as described in the U.S. Pat. No. 7,709,198. An example is shown in FIG. 3, where the CP is replaced by a set of capture probes. The melting temperatures of the hybridization between each capture probe in the set and SGP, or between each capture probe in the set and the target, or both are lower than the assay hybridization temperature. So each capture probe does not have sufficient binding strength to capture the SGP stably. But when all the capture probes in the set are present together, enough hybridization strength is created to maintain the stable link between the SGP and the target. Therefore, if one of the capture probes hybridizes non-specifically to a non-target sequence, it does not have sufficient binding strength to capture the SGP to the target through out the assay, thus preventing the generation of false positive signals and reducing the background signal.

The SGP may comprise a relatively large structure in order to attaching many label molecules on to it. This introduces a number of drawbacks. It may get “stuck” or trapped non-specifically in a void in solid surface in a solution-based assay or within cellular matrix in an in situ detection assay, which will also result in false positive signals and reduce signal-to-background ratio. If the SGP structure is large enough to contain many label molecules, the false positive or background signals can be significant, making it hard to be distinguished from the real signal. In addition, in in situ detection applications, the large structure may have difficulty to gain access to the target molecule inside cellular matrix, which may result in reduction in signal level.

Detecting events in which specific sections of nucleic acid sequences have aberrantly connected together is very important because such events often have biological and clinical implications. The unintended juxtaposition of two nucleic acid sequences can occur in multiple ways and have an impact both at the DNA and RNA levels. For example, the rearrangement of DNA through a translocation can lead to the fusion of two genes, potentially disrupting importing protein coding regions. Also, a gene fusion event can lead to the creation of a chimeric RNA sequence that has transformative properties. Finally, a point mutation in a splice acceptor site at an intron/exon boundary could cause the inclusion or exclusion of unintended sequences in the final mRNA due to aberrant splicing.

Of the various point mutations, chromosomal rearrangements, and epigenetic changes that can cause mis-joined nucleic acid sequences, chromosomal rearrangements resulting in gene fusions are the most prevalent somatic mutation in cancer development, accounting for 20% of deaths due to cancer. One result of this abnormal juxtaposition of genetic material is the creation of a chimeric mRNA transcript from the fusion of two different coding regions. The resulting protein is considered a driving cause of the underlying disease and a potential therapeutic target since its expression is limited to cancer cells. In addition, the restricted expression pattern of the fusion mRNA and protein make them ideal candidates for use as biomarkers in cancer diagnostics.

The best studied example of a gene fusion event is the creation of the Philadelphia chromosome from the reciprocal chromosomal translocation t(9;22), which joins the break point cluster region (BCR) with the Abelson kinase gene (ABL). It was the first example of a causal link between genetic alterations and the development of cancer, being present in 100% of chronic myeloid leukemia (CML) cases. Because of the direct association between the creation of the fusion protein and the disease, inhibition of ABL kinase signaling is a prime target for drug inhibition. In fact, the tyrosine kinase inhibitor imatinib (Gleevec) was developed and patients treated with the drug in a major clinical study showed an overall survival rate of >85% at 5 years regardless of the severity of the disease at diagnosis.

The early key finding that gene fusions have a causative role in carcinogenesis and the more recent evidence that the protein products can be selectively targeted by drug therapies has lead to an increased interest in identifying novel genomic rearrangements. Screening methods at both the DNA and transcript levels have brought the total number of known gene fusions in malignant cancers to over 300 including the previously identified ones. Of these, 75% are found in haematological disorders such as CML, ALL, and Burkitt's lymphoma, and the rest are present in solid tumors, mainly prostate, thyroid, breast, and lung. It has also been discovered that a single oncogene can have multiple fusion partners, though the specific disease outcome is always the same. Though a positive correlation with disease for most of the newly discovered gene fusions has yet to be determined, this large number of potential clinical biomarkers and therapeutic targets will require a new set of reagents for detection as research into disease association moves forward.

Methods for confirming the presence of a known gene fusion have been developed both at the DNA and RNA levels. For DNA, detection can be done using fluorescent in situ hybridization (FISH) with probes complimentary to specific DNA sequences. This method allows for the direct visualization of genomic rearrangements including translocations and inversions. In addition, amplification by PCR of genomic sequence surrounding potential DNA breakpoints, followed by sequencing of the product, can also be employed to detect sequence level alterations. For the detection of known gene fusions at the RNA level, RT-PCR can be used with a primer pair containing one primer homologous to either of the genes to be detected. A positive RT-PCR product confirms that two different genes are part of the same transcript. To the best knowledge of the inventors, there has been no prior art in detecting mis-joint of nucleic acid sequences in situ at RNA level. Methods have also been created for fusion gene discovery. These include transcriptome sequencing, genome-wide massively parallel paired-end sequencing, and paired-end diTags (PET).

In addition to gene fusion events leading to chimeric transcripts, mutations affecting RNA splicing can also create mis-joined RNA sequences that lead to disease. The causal mutations can occur directly on cis-acting elements within a gene, or can occur in trans-acting elements such as regulators of splicing. Either way, nucleic acid sequences that are normally present in the mRNA can be excluded, or new sequence can be introduced, both of which lead to a novel transcript.

One of the best studied examples of alternative splicing alterations leading to disease is the case of the transcription factor KLF6 in prostate cancer. A point mutation in the KLF6 gene causes to the use of a cryptic splice site, leading to a partial deletion of RNA sequences. Though some normal protein is still produced, it is believed that the new truncated protein product acts as a dominate-negative mutant, inhibiting the function of wild-type protein products. The end result is an increased susceptibility to prostate cancer.

Most nucleic acid based assays (e.g. PCR, microarray, bDNA, etc.) involve the use of specially designed nucleic acid probes binding to specific target sequences. It is highly desirable that such binding is highly specific, i.e. the designed probe binds only to the intended target sequence, not to identical or similar sequences else where. For nucleic acid detection assays, low specificity not only may produce false positive results, but also increases background noise, leading to reduced detection sensitivity.

In most conventional approaches, the probe comprises a section of nucleic acid sequences complimentary to the target sequence, as shown in FIG. 1A. The binding between the probe and its target has to be sufficiently strong so that the binding can remain stable under the assay condition (i.e. the melting temperature, Tₘ, of the probe-target pair is above the assay temperature). This requirement dictates that the probe sequence has to be of sufficient length, which typically ranging from 20 to 100 bases depending assay types and conditions. However, when the probe becomes long, its binding stability becomes not very sensitive to mis-matches in a small number of bases, which leads directly to increased possibility on non-specific binding. The problem is particularly severe in assays designed to detect single nucleotide polymorphisms (SNPs), where the target sequence is different from other genotypes by only a single base.

SNPs are the most frequently occurring genetic variation in the human genome. A SNP is a single nucleotide variation at a specific location in the genome. The average SNP frequency is approximately one per 1,000 base pair but much less frequent in the coding regions of the genome. SNPs can serve as disease markers because they may cause changes in biological processes inducing disease states. SNPs can also serve as markers in pharmacogenomic studies, where genetic polymorphisms underlie drug response. Despite the recent development of a variety of genotyping technologies (Kim S, Misra A. (2007) SNP genotyping: technologies and biomedical applications. Annu Rev Biomed Eng. 9:289-320), there is still significant unmet need in genotyping assays such as higher throughput, higher accuracy, and lower cost.

Genotyping typically involves the generation of allele-specific products for SNPs of interest followed by their detection for genotype determination. There are four major types of genotyping methods: single base extension-based (Sokolov B P. (1990) Primer extension technique for the detection of single nucleotide in genomic DNA. Nucleic Acids Res. 18(12):3671), hybridization-based (Kennedy G C, Matsuzaki H, Dong S, Liu W M, Huang J, Liu G, Su X, Cao M, Chen W, Zhang J, Liu W, Yang G, Di X, Ryder T, He Z, Surti U, Phillips M S, Boyce-Jacino M T, Fodor S P, Jones K W. (2003) Large-scale genotyping of complex DNA. Nat Biotechnol. 21(10):1233-7; and Livak K J. (1999) Allelic discrimination using fluorogenic probes and the 5′ nuclease assay. Genet Anal. 14(5-6):143-9), ligation-based (Landegren U, Kaiser R, Sanders J, Hood L. (1998) A ligase-mediated gene detection technique. Science. 241(4869):1077-80), enzymatic cleavage based (Lyamichev V, Mast A L, Hall J G, Prudent J R, Kaiser M W, Takova T, Kwiatkowski R W, Sander T J, de Arruda M, Arco D A, Neri B P, Brow M A. (1999) Polymorphism identification and quantitative detection of genomic DNA by invasive cleavage of oligonucleotide probes. Nat Biotechnol. 17(3):292-6), plus other methods (Oliphant A, Barker D L, Stuelpnagel J R, Chee M S. (2002) BeadArray technology: enabling an accurate, cost-effective approach to high-throughput genotyping. Biotechniques. Suppl:56-8, 60-1) that use the combination of two or more above four approaches. There are two issues in the current genotyping technologies related to probes used in genotyping. First, because the probes could bind non-specific locations of the genome, all current genotyping technologies with only a few exceptions require the PCR amplification step. The PCR amplification of a desired SNP-containing region is performed initially to introduce specificity and increase the number of molecules for detection following allelic discrimination. However, PCR amplification presents a major obstacle in genotyping throughput. Secondly, the genotyping probe that hybridizes to a SNP region may not offer sufficient difference in thermal stability between perfect match and mismatch sequences, thus they may not be able to achieve reliable allelic discrimination. Thus, there is a need to develop genotyping probes with increased specificity for SNP sites and increased allelic discrimination between match and mismatch sequences.

There are also single nucleotide variations that are caused by somatic mutation such as those occurring in cancer cells and in cells developing drug resistance. Because these SNPs are present in the genome of a small fraction of cells during early stage of disease and drug resistance, there is a need to determine SNPs at single cell level through in situ genotyping for the purpose of early therapeutic intervention. Furthermore, there is a need to determine whether the mutation occurs in one or both alleles of the genome in a cell. Current in situ genotyping technologies such as allele specific hybridization (O'Keefe C L, Matera A G. (2000) Alpha satellite DNA variant-specific oligoprobes differing by a single base can distinguish chromosome 15 homologs. Genome Res. 10(9):1342-50), RINS (Koch J E, Kolvraa S, Petersen K B, Gregersen N, Bolund L. (1989) Oligonucleotide-priming methods for the chromosome-specific labelling of alpha satellite DNA in situ. Chromosoma. 98(4):259-65.), and in situ PCR (Tokusashi Y, Nishikawa Y, Ogawa K. (1995) Differentiation of the normal and mutant rat albumin genes on hepatic tissue sections by in situ PCR. Nucleic Acids Res. 23(18):3790-1), padlock probe (Larsson C, Koch J, Nygren A, Janssen G, Raap A K, Landegren U, Nilsson M. (2004) In situ genotyping individual DNA molecules by target-primed rolling-circle amplification of padlock probes. Nat Methods. 1(3):227-32) may not be adequate for this purpose.

The present invention attempts to address above unmet needs and it can also benefit many other applications where highly specific hybridization and/or better discrimination between match and mismatch sequences are required. There are two applications in particular worth mentioning here. The first is for multiplexed detection of many nucleic acid targets in one assay, in which low specificity can generate cross-reactivity between different targets and significantly impact assay performance. The second is for in situ detection of nucleic acids, where low specificity will produce a high level of background noise, severely restricting detecting sensitivity.

### SUMMARY OF THE INVENTION

Methods of detecting nucleic acid targets in single cells, including methods of detecting multiple targets in a single cell, are provided. Methods of detecting individual cells, particularly rare cells from large heterogeneous cell populations, through detection of nucleic acids are described. Methods to improve probe hybridization specificity and their application in genotyping are described. In situ detection of mis-joined nucleic acid sequences, and methods to reduce false positive signals and improve signal-to-background ratio in hybridization-based nucleic acid detection assay are also described. Related compositions, tissue slides, sample of suspended cells, kits, and systems relate to the methods are also described.

A first general class of embodiments includes methods of detecting two or more nucleic acid targets in an individual cell. In the methods, a sample comprising the cell is provided. The cell comprises, or is suspected of comprising, a first nucleic acid target and a second nucleic acid target. A first label probe comprising a first label and a second label probe comprising a second label, wherein a first signal from the first label is distinguishable from a second signal from the second label, are provided. At least a first capture probe and at least a second capture probe are also provided.

The first capture probe is hybridized, in the cell, to the first nucleic acid target (when the first nucleic acid target is present in the cell), and the second capture probe is hybridized, in the cell, to the second nucleic acid target (when the second nucleic acid target is present in the cell). The first label probe is captured to the first capture probe and the second label probe is captured to the second capture probe, thereby capturing the first label probe to the first nucleic acid target and the second label probe to the second nucleic acid target. The first signal from the first label and the second signal from the second label are then detected. Since the first and second labels are associated with their respective nucleic acid targets through the capture probes, presence of the label(s) in the cell indicates the presence of the corresponding nucleic acid target(s) in the cell. The methods are optionally quantitative. Thus, an intensity of the first signal and an intensity of the second signal can be measured, and the intensity of the first signal can be correlated with a quantity of the first nucleic acid target in the cell while the intensity of the second signal is correlated with a quantity of the second nucleic acid target in the cell. As another example, a signal spot can be counted for each copy of the first and second nucleic acid targets to quantitate them.

In one aspect, the label probes bind directly to the capture probes. For example, in one class of embodiments, a single first capture probe and a single second capture probe are provided, the first label probe is hybridized to the first capture probe, and the second label probe is hybridized to the second capture probe. In a related class of embodiments, two or more first capture probes and two or more second capture probes are provided, as are a plurality of the first label probes (e.g., two or more identical first label probes) and a plurality of the second label probes (e.g., two or more identical second label probes). The two or more first capture probes are hybridized to the first nucleic acid target, and the two or more second capture probes are hybridized to the second nucleic acid target. A single first label probe is hybridized to each of the first capture probes, and a single second label probe is hybridized to each of the second capture probes.

In another aspect, the label probes are captured to the capture probes indirectly, for example, through binding of preamplifiers and/or amplifiers. In one class of embodiments in which amplifiers are employed, a single first capture probe, a single second capture probe, a plurality of the first label probes, and a plurality of the second label probes are provided. A first amplifier is hybridized to the first capture probe and to the plurality of first label probes, and a second amplifier is hybridized to the second capture probe and to the plurality of second label probes. In another class of embodiments, two or more first capture probes, two or more second capture probes, a plurality of the first label probes, and a plurality of the second label probes are provided. The two or more first capture probes are hybridized to the first nucleic acid target, and the two or more second capture probes are hybridized to the second nucleic acid target. A first amplifier is hybridized to each of the first capture probes, and the plurality of first label probes is hybridized to the first amplifiers. A second amplifier is hybridized to each of the second capture probes, and the plurality of second label probes is hybridized to the second amplifiers.

In one class of embodiments in which preamplifiers are employed, a single first capture probe, a single second capture probe, a plurality of the first label probes, and a plurality of the second label probes are provided. A first preamplifier is hybridized to the first capture probe, a plurality of first amplifiers is hybridized to the first preamplifier, and the plurality of first label probes is hybridized to the first amplifiers. A second preamplifier is hybridized to the second capture probe, a plurality of second amplifiers is hybridized to the second preamplifier, and the plurality of second label probes is hybridized to the second amplifiers. In another class of embodiments, two or more first capture probes, two or more second capture probes, a plurality of the first label probes, and a plurality of the second label probes are provided. The two or more first capture probes are hybridized to the first nucleic acid target, and the two or more second capture probes are hybridized to the second nucleic acid target. A first preamplifier is hybridized to each of the first capture probes, a plurality of first amplifiers is hybridized to each of the first preamplifiers, and the plurality of first label probes is hybridized to the first amplifiers. A second preamplifier is hybridized to each of the second capture probes, a plurality of second amplifiers is hybridized to each of the second preamplifiers, and the plurality of second label probes is hybridized to the second amplifiers.

In embodiments in which two or more first capture probes and/or two or more second capture probes are employed, the capture probes preferably hybridize to nonoverlapping polynucleotide sequences in their respective nucleic acid target.

In one class of embodiments, a plurality of the first label probes and a plurality of the second label probes are provided. A first amplified polynucleotide is produced by rolling circle amplification of a first circular polynucleotide hybridized to the first capture probe. The first circular polynucleotide comprises at least one copy of a polynucleotide sequence identical to a polynucleotide sequence in the first label probe, and the first amplified polynucleotide thus comprises a plurality of copies of a polynucleotide sequence complementary to the polynucleotide sequence in the first label probe. The plurality of first label probes is then hybridized to the first amplified polynucleotide. Similarly, a second amplified polynucleotide is produced by rolling circle amplification of a second circular polynucleotide hybridized to the second capture probe. The second circular polynucleotide comprises at least one copy of a polynucleotide sequence identical to a polynucleotide sequence in the second label probe, and the second amplified polynucleotide thus comprises a plurality of copies of a polynucleotide sequence complementary to the polynucleotide sequence in the second label probe. The plurality of second label probes is then hybridized to the second amplified polynucleotide. The amplified polynucleotides remain associated with the capture probe(s), and the label probes are thus captured to the nucleic acid targets.

The methods are useful for multiplex detection of nucleic acids, including simultaneous detection of more than two nucleic acid targets. Thus, the cell optionally comprises or is suspected of comprising a third nucleic acid target, and the methods optionally include: providing a third label probe comprising a third label, wherein a third signal from the third label is distinguishable from the first and second signals, providing at least a third capture probe, hybridizing in the cell the third capture probe to the third nucleic acid target (when present in the cell), capturing the third label probe to the third capture probe, and detecting the third signal from the third label. Fourth, fifth, sixth, etc. nucleic acid targets are similarly simultaneously detected in the cell if desired. Each hybridization or capture step is preferably accomplished for all of the nucleic acid targets at the same time.

A nucleic acid target can be essentially any nucleic acid that is desirably detected in the cell. For example, a nucleic acid target can be a DNA, a chromosomal DNA, an RNA, an mRNA, a microRNA, a ribosomal RNA, or the like. The nucleic acid target can be a nucleic acid endogenous to the cell. As another example, the target can be a nucleic acid introduced to or expressed in the cell by infection of the cell with a pathogen, for example, a viral or bacterial genomic RNA or DNA, a plasmid, a viral or bacterial mRNA, or the like.

The first and second (and/or optional third, fourth, etc.) nucleic acid targets can be part of a single nucleic acid molecule, or they can be separate molecules. In one class of embodiments, the first nucleic acid target is a first mRNA and the second nucleic acid target is a second mRNA. In another class of embodiments, the first nucleic acid target comprises a first region of an mRNA and the second nucleic acid target comprises a second region of the same mRNA. In another class of embodiments, the first nucleic acid target comprises a first chromosomal DNA polynucleotide sequence and the second nucleic acid target comprises a second chromosomal DNA polynucleotide sequence. The first and second chromosomal DNA polynucleotide sequences are optionally located on the same chromosome, e.g., within the same gene, or on different chromosomes. Optionally, the first nucleic acid target and/or the second nucleic acid target is a cytoplasmic RNA.

In one aspect, the signal(s) from nucleic acid target(s) are normalized. In one class of embodiments, the second nucleic acid target comprises a reference nucleic acid, and the method includes normalizing the first signal to the second signal. The label (first, second, third, etc.) can be essentially any convenient label that directly or indirectly provides a detectable signal. In one aspect, the first label is a first fluorescent label and the second label is a second fluorescent label.

The methods can be used to detect the presence of the nucleic acid targets in cells from essentially any type of sample. For example, the sample can be derived from a bodily fluid such as blood. The methods for detecting nucleic acid targets in cells can be used to identify the cells. For example, a cell can be identified as being of a desired type based on which nucleic acids, and in what levels, it contains. Thus, in one class of embodiments, the methods include identifying the cell as a desired target cell based on detection of the first and second signals (and optional third, fourth, etc. signals) from within the cell. As just a few examples, the cell can be a circulating tumor cell, a virally infected cell, a fetal cell in maternal blood, a bacterial cell or other microorganism in a biological sample, or an endothelial cell, precursor endothelial cell, or myocardial cell in blood. In one class of embodiments, the sample comprises a tissue section or other solid tissue sample (e.g., an FFPE section).

The cell is typically fixed and permeabilized before hybridization of the capture probes, to retain the nucleic acid targets in the cell and to permit the capture probes, label probes, etc. to enter the cell. The cell is optionally washed to remove materials not captured to one of the nucleic acid targets. The cell can be washed after any of various steps, for example, after hybridization of the capture probes to the nucleic acid targets to remove unbound capture probes, after hybridization of the preamplifiers, amplifiers, and/or label probes to the capture probes, and/or the like. It will be evident that double-stranded nucleic acid target(s) are preferably denatured, e.g., by heat, prior to hybridization of the corresponding capture probe(s) to the target(s).

Optionally, the cell is in suspension for all or most of the steps of the method. Thus, in one class of embodiments, the cell is in suspension in the sample comprising the cell, and/or the cell is in suspension during the hybridizing, capturing, and/or detecting steps. In other embodiments, the cell is in suspension in the sample comprising the cell, and the cell is fixed on a substrate during the hybridizing, capturing, and/or detecting steps. For example, the cell can be in suspension during the hybridization, capturing, and optional washing steps and immobilized on a substrate during the detection step. In embodiments in which the cell is in suspension, the first and second (and optional third, etc.) signals can be conveniently detected by flow cytometry. Signals from the labels are typically detected in a single operation.

The methods permit detection of even low or single copy number targets. Thus, in one class of embodiments, about 1000 copies or less of the first nucleic acid target and/or about 1000 copies or less of the second nucleic acid target are present in the cell (e.g., about 100 copies or less, about 50 copies or less, about 10 copies or less, about 5 copies or less, or even a single copy).

One general class of embodiments provides methods of assaying a relative level of one or more target nucleic acids in an individual cell. In the methods, a sample comprising the cell is provided. The cell comprises or is suspected of comprising a first, target nucleic acid, and it comprises a second, reference nucleic acid. A first label probe comprising a first label and a second label probe comprising a second label, wherein a first signal from the first label is distinguishable from a second signal from the second label, are also provided. In the cell, the first label probe is captured to the first, target nucleic acid (when present in the cell) and the second label probe is captured to the second, reference nucleic acid. The first signal from the first label and the second signal from the second label are then detected in the individual cell, and the intensity of each signal is measured. The intensity of the first signal is normalized to the intensity of the second (reference) signal. The level of the first, target nucleic acid relative to the level of the second, reference nucleic acid in the cell is thereby assayed, since the first and second labels are associated with their respective nucleic acids. The methods are optionally quantitative, permitting measurement of the amount of the first, target nucleic acid relative to the amount of the second, reference nucleic acid in the cell. Thus, the intensity of the first signal normalized to that of the second signal can be correlated with a quantity of the first, target nucleic acid present in the cell.

The label probes can bind directly to the nucleic acids. For example, the first label probe can hybridize to the first, target nucleic acid and/or the second label probe can hybridize to the second, reference nucleic acid. Alternatively, the label probes can be bound indirectly to the nucleic acids, e.g., via capture probes. In one class of embodiments, at least a first capture probe and at least a second capture probe are provided. In the cell, the first capture probe is hybridized to the first, target nucleic acid and the second capture probe is hybridized to the second, reference nucleic acid. The first label probe is captured to the first capture probe and the second label probe is captured to the second capture probe, thereby capturing the first label probe to the first, target nucleic acid and the second label probe to the second, reference nucleic acid. The features described for the methods above apply to these embodiments as well, with respect to configuration and number of the label and capture probes, optional use of preamplifiers and/or amplifiers, rolling circle amplification of circular polynucleotides, and the like.

The methods can be used for multiplex detection of nucleic acids, including simultaneous detection of two or more target nucleic acids. Thus, the cell optionally comprises or is suspected of comprising a third, target nucleic acid, and the methods optionally include: providing a third label probe comprising a third label, wherein a third signal from the third label is distinguishable from the first and second signals; capturing, in the cell, the third label probe to the third, target nucleic acid (when present in the cell); detecting the third signal from the third label, which detecting comprises measuring an intensity of the third signal; and normalizing the intensity of the third signal to the intensity of the second signal. Fourth, fifth, sixth, etc. nucleic acids are similarly simultaneously detected in the cell if desired.

The methods for assaying relative levels of target nucleic acids in cells can be used to identify the cells. For example, a cell can be identified as being of a desired type based on which nucleic acids, and in what levels, it contains. Thus, in one class of embodiments, the methods include identifying the cell as a desired target cell based on the normalized first signal (and optional normalized third, fourth, etc. signals).

Essentially all of the features noted for the methods above apply to these embodiments as well, as relevant; for example, with respect to type of target and reference nucleic acids, cell type, source of sample, fixation and permeabilization of the cell, washing the cell, denaturation of double-stranded target and reference nucleic acids, type of labels, use of optional blocking probes, detection of signals, detection (and intensity measurement) by flow cytometry or microscopy, presence of the cell in suspension, immobilized on a substrate, or in a tissue section, and/or the like.

Another general class of embodiments provides methods of performing comparative gene expression analysis in single cells. In the methods, a first mixed cell population comprising one or more cells of a specified type is provided. An expression level of one or more target nucleic acids relative to a reference nucleic acid is measured in the cells of the specified type of the first population, to provide a first expression profile. A second mixed cell population comprising one or more cells of the specified type is also provided, and an expression level of the one or more target nucleic acids relative to the reference nucleic acid is measured in the cells of the specified type of the second population, to provide a second expression profile. The first and second expression profiles are then compared.

Essentially all of the features noted for the methods above apply to these embodiments as well, as relevant; for example, with respect to type of target and reference nucleic acids, cell type, source of sample, fixation and permeabilization of the cell, washing the cell, denaturation of double-stranded target and reference nucleic acids, type of labels, use and configuration of label probes, capture probes, preamplifiers and/or amplifiers, use of optional blocking probes, detection of signals, detection (and intensity measurement) by flow cytometry or microscopy, presence of the cell in suspension, immobilized on a substrate, or in a tissue section, and/or the like.

In one aspect, the invention provides methods that facilitate association of a high density of labels to target nucleic acids in cells. One general class of embodiments provides methods of detecting two or more nucleic acid targets in an individual cell. In the methods, a sample comprising the cell is provided. The cell comprises or is suspected of comprising a first nucleic acid target and a second nucleic acid target. In the cell, a first label is captured to the first nucleic acid target (when present in the cell) and a second label is captured to the second nucleic acid target (when present in the cell). A first signal from the first label is distinguishable from a second signal from the second label. As noted, the labels are captured at high density. Thus, an average of at least one copy of the first label per nucleotide of the first nucleic acid target is captured to the first nucleic acid target over a region that spans at least 20 contiguous nucleotides of the first nucleic acid target, and an average of at least one copy of the second label per nucleotide of the second nucleic acid target is captured to the second nucleic acid target over a region that spans at least 20 contiguous nucleotides of the second nucleic acid target. The first signal from the first label and the second signal from the second label are detected.

In one class of embodiments, an average of at least four, eight, or twelve copies of the first label per nucleotide of the first nucleic acid target are captured to the first nucleic acid target over a region that spans at least 20 contiguous nucleotides of the first nucleic acid target, and an average of at least four, eight, or twelve copies of the second label per nucleotide of the second nucleic acid target are captured to the second nucleic acid target over a region that spans at least 20 contiguous nucleotides of the second nucleic acid target. In one embodiment, an average of at least sixteen copies of the first label per nucleotide of the first nucleic acid target are captured to the first nucleic acid target over a region that spans at least 20 contiguous nucleotides of the first nucleic acid target, and an average of at least sixteen copies of the second label per nucleotide of the second nucleic acid target are captured to the second nucleic acid target over a region that spans at least 20 contiguous nucleotides of the second nucleic acid target.

Essentially all of the features noted for the methods above apply to these embodiments as well, as relevant, for example, with respect to type of labels, detection of signals, type, treatment, presence in a tissue sample, and suspension of the cell, and/or the like. A like density of third, fourth, fifth, sixth, etc. labels is optionally captured to third, fourth, fifth, sixth, etc. nucleic acid targets.

Another general class of embodiments provides methods of detecting an individual cell of a specified type. In the methods, a sample comprising a mixture of cell types including at least one cell of the specified type is provided. A first label probe comprising a first label and a second label probe comprising a second label, wherein a first signal from the first label is distinguishable from a second signal from the second label, are provided. In the cell, the first label probe is captured to a first nucleic acid target (when the first nucleic acid target is present in the cell) and the second label probe is captured to a second nucleic acid target (when the second nucleic acid target is present in the cell). The first signal from the first label and the second signal from the second label are detected and correlated with the presence, absence, or amount of the corresponding, first and second nucleic acid targets in the cell. The cell is identified as being of the specified type based on detection of the presence, absence, or amount of both the first and second nucleic acid targets within the cell, where the specified type of cell is distinguishable from the other cell type(s) in the mixture on the basis of either the presence, absence, or amount of the first nucleic acid target or the presence, absence, or amount of the second nucleic acid target in the cell (that is, the nucleic acid targets are redundant markers for the specified cell type). An intensity of the first signal and an intensity of the second signal are optionally measured and correlated with a quantity of the corresponding nucleic acid present in the cell. In one class of embodiments, the cell comprises a first nucleic acid target and a second nucleic acid target, and the cell is identified as being of the specified type based on detection of the presence or amount of both the first and second nucleic acid targets within the cell, where the specified type of cell is distinguishable from the other cell type(s) in the mixture on the basis of either the presence or amount of the first nucleic acid target or the presence or amount of the second nucleic acid target in the cell.

The label probes can bind directly to the nucleic acid targets. For example, the first label probe can hybridize to the first nucleic acid target and/or the second label probe can hybridize to the second nucleic acid target. The label probes are optionally captured to the nucleic acid targets via capture probes. In one class of embodiments, at least a first capture probe and at least a second capture probe are provided. In the cell, the first capture probe is hybridized to the first nucleic acid target and the second capture probe is hybridized to the second nucleic acid target. The first label probe is captured to the first capture probe and the second label probe is captured to the second capture probe, thereby capturing the first label probe to the first nucleic acid target and the second label probe to the second nucleic acid target. The features described for the methods above apply to these embodiments as well, with respect to configuration and number of the label and capture probes, optional use of preamplifiers and/or amplifiers, rolling circle amplification of circular polynucleotides, and the like.

Third, fourth, fifth, etc. nucleic acid targets are optionally detected in the cell. For example, the method optionally includes: providing a third label probe comprising a third label, wherein a third signal from the third label is distinguishable from the first and second signals, capturing in the cell the third label probe to a third nucleic acid target (when the third target is present in the cell), and detecting the third signal from the third label. The third, fourth, fifth, etc. label probes are optionally hybridized directly to their corresponding nucleic acid, or they can be captured indirectly via capture probes as described for the first and second label probes.

The first and/or second signal can be normalized to the third signal. Thus, in some embodiments, the cell comprises the third nucleic acid target, and the methods include identifying the cell as being of the specified type based on the normalized first and/or second signal, e.g., in embodiments in which the target cell type is distinguishable from the other cell type(s) in the mixture based on the copy number of the first and/or second nucleic acid targets, rather than purely on their presence in the target cell type and not in the other cell type(s).

As another example, the third nucleic acid target can serve as a third redundant marker for the target cell type, e.g., to improve specificity of the assay for the desired cell type. Thus, in one class of embodiments, the methods include correlating the third signal detected from the cell with the presence, absence, or amount of the third nucleic acid target in the cell, and identifying the cell as being of the specified type based on detection of the presence, absence, or amount of the first, second, and third nucleic acid targets within the cell, wherein the specified type of cell is distinguishable from the other cell type(s) in the mixture on the basis of either presence, absence, or amount of the first nucleic acid target, presence, absence, or amount of the second nucleic acid target, or presence, absence, or amount of the third nucleic acid target in the cell.

The methods can be applied to detection and identification of even rare cell types. For example, the ratio of cells of the specified type to cells of all other type(s) in the mixture is optionally less than 1:1×10⁴, less than 1:1×10⁵, less than 1:1×10⁶, less than 1:1×10⁷, less than 1:1×10⁸, or even less than 1:1×10⁹.

Essentially all of the features noted for the methods above apply to these embodiments as well, as relevant; for example, with respect to type of nucleic acid targets, copy number, cell type, source of sample, fixation and permeabilization of the cell, washing the cell, denaturation of double-stranded nucleic acids, type of labels, use of optional blocking probes, detection of signals, detection (and intensity measurement) of signals from the individual cell by flow cytometry or microscopy, presence of the cell in suspension, immobilized on a substrate, or in a tissue section, and/or the like.

The invention also provides compositions useful in practicing or produced by the methods. One exemplary class of embodiments provides a composition that includes a fixed and permeabilized cell, which cell comprises or is suspected of comprising a first nucleic acid target and a second nucleic acid target, at least a first capture probe capable of hybridizing to the first nucleic acid target, at least a second capture probe capable of hybridizing to the second nucleic acid target, a first label probe comprising a first label, and a second label probe comprising a second label. A first signal from the first label is distinguishable from a second signal from the second label. The cell optionally comprises the first and second capture probes and label probes. The first and second capture probes are optionally hybridized to their respective nucleic acid targets in the cell.

The features described for the methods above for indirect capture of the label probes to the nucleic acid targets apply to these embodiments as well, for example, with respect to configuration and number of the label and capture probes, optional use of preamplifiers and/or amplifiers, and the like.

In one class of embodiments, the composition comprises a plurality of the first label probes, a plurality of the second label probes, a first amplified polynucleotide produced by rolling circle amplification of a first circular polynucleotide hybridized to the first capture probe, and a second amplified polynucleotide produced by rolling circle amplification of a second circular polynucleotide hybridized to the second capture probe. The first circular polynucleotide comprises at least one copy of a polynucleotide sequence identical to a polynucleotide sequence in the first label probe, and the first amplified polynucleotide comprises a plurality of copies of a polynucleotide sequence complementary to the polynucleotide sequence in the first label probe. The second circular polynucleotide comprises at least one copy of a polynucleotide sequence identical to a polynucleotide sequence in the second label probe, and the second amplified polynucleotide comprises a plurality of copies of a polynucleotide sequence complementary to the polynucleotide sequence in the second label probe. The composition can also include reagents necessary for producing the amplified polynucleotides, for example, an exogenously supplied nucleic acid polymerase, an exogenously supplied nucleic acid ligase, and/or exogenously supplied nucleoside triphosphates (e.g., dNTPs).

The cell optionally includes additional nucleic acid targets, and the composition (and cell) can include reagents for detecting these targets. For example, the cell can comprise or be suspected of comprising a third nucleic acid target, and the composition can include at least a third capture probe capable of hybridizing to the third nucleic acid target and a third label probe comprising a third label. A third signal from the third label is distinguishable from the first and second signals. The cell optionally includes fourth, fifth, sixth, etc. nucleic acid targets, and the composition optionally includes fourth, fifth, sixth, etc. label probes and capture probes.

The cell can be present in a mixture of cells, for example, a complex heterogeneous mixture. In one class of embodiments, the cell is of a specified type, and the composition comprises one or more other types of cells. These other cells can be present in excess, even large excess, of the cell. For example, the ratio of cells of the specified type to cells of all other type(s) in the composition is optionally less than 1:1×10⁴, less than 1:1×10⁵, less than 1:1×10⁶, less than 1:1×10⁷, less than 1:1×10⁸, or even less than 1:1×10⁹.

Essentially all of the features noted for the methods above apply to these embodiments as well, as relevant; for example, with respect to type of nucleic acid target, type and source of cell, location of various targets on a single molecule or on different molecules, type of labels, inclusion of optional blocking probes, and/or the like. The cell is optionally in suspension in the composition or in a tissue section or other solid tissue sample.

One general class of embodiments provides a composition comprising a cell, which cell includes a first nucleic acid target, a second nucleic acid target, a first label whose presence in the cell is indicative of the presence of the first nucleic acid target in the cell, and a second label whose presence in the cell is indicative of the presence of the second nucleic acid target in the cell, wherein a first signal from the first label is distinguishable from a second signal from the second label. An average of at least one copy of the first label is present in the cell per nucleotide of the first nucleic acid target over a region that spans at least 20 contiguous nucleotides of the first nucleic acid target, and an average of at least one copy of the second label is present in the cell per nucleotide of the second nucleic acid target over a region that spans at least 20 contiguous nucleotides of the second nucleic acid target.

In one class of embodiments, the copies of the first label are physically associated with the first nucleic acid target, and the copies of the second label are physically associated with the second nucleic acid target. For example, the first label can be part of a first label probe and the second label part of a second label probe, where the label probes are captured to the target nucleic acids.

Essentially all of the features noted for the embodiments above apply to these embodiments as well, as relevant, for example, with respect to type and number of labels, suspension of the cell, and/or the like. A like density of labels is optionally present for third, fourth, fifth, sixth, etc. nucleic acid targets.

Another aspect of the invention provides kits useful for practicing the methods. One general class of embodiments provides a kit for detecting a first nucleic acid target and a second nucleic acid target in an individual cell. The kit includes at least one reagent for fixing and/or permeabilizing the cell, at least a first capture probe capable of hybridizing to the first nucleic acid target, at least a second capture probe capable of hybridizing to the second nucleic acid target, a first label probe comprising a first label, and a second label probe comprising a second label, wherein a first signal from the first label is distinguishable from a second signal from the second label, packaged in one or more containers.

Essentially all of the features noted for the embodiments above apply to these embodiments as well, as relevant; for example, with respect to number of nucleic acid targets, configuration and number of the label and capture probes, inclusion of preamplifiers and/or amplifiers, inclusion of blocking probes, inclusion of amplification reagents, type of nucleic acid target, location of various targets on a single molecule or on different molecules, type of labels, inclusion of optional blocking probes, and/or the like.

Another general class of embodiments provides a kit for detecting an individual cell of a specified type from a mixture of cell types by detecting a first nucleic acid target and a second nucleic acid target. The kit includes at least one reagent for fixing and/or permeabilizing the cell, a first label probe comprising a first label, and a second label probe comprising a second label, wherein a first signal from the first label is distinguishable from a second signal from the second label, packaged in one or more containers. The specified type of cell is distinguishable from the other cell type(s) in the mixture by presence, absence, or amount of the first nucleic acid target in the cell or by presence, absence, or amount of the second nucleic acid target in the cell.

Essentially all of the features noted for the embodiments above apply to these embodiments as well, as relevant; for example, with respect to number of nucleic acid targets, inclusion of capture probes, configuration and number of the label and/or capture probes, inclusion of preamplifiers and/or amplifiers, inclusion of blocking probes, inclusion of amplification reagents, type of nucleic acid target, location of various targets on a single molecule or on different molecules, type of labels, inclusion of optional blocking probes, and/or the like.

Another aspect of the invention provides methods for detection of nucleic acids in cells in suspension, for example, rapid detection by flow cytometry. Accordingly, one general class of embodiments provides methods of detecting one or more nucleic acid targets in an individual cell that include: providing a sample comprising the cell, which cell comprises or is suspected of comprising a first nucleic acid target; providing a first label probe comprising a first label; providing at least a first capture probe; hybridizing, in the cell, the first capture probe to the first nucleic acid target, when present in the cell; capturing the first label probe to the first capture probe, thereby capturing the first label probe to the first nucleic acid target; and detecting, while the cell is in suspension, a first signal from the first label. For example, the signal can be conveniently detected by performing flow cytometry.

The methods are useful for multiplex detection of nucleic acids, including simultaneous detection of two or more nucleic acid targets. Thus, the cell optionally comprises or is suspected of comprising a second nucleic acid target, and the methods optionally include: providing a second label probe comprising a second label, wherein a second signal from the second label is distinguishable from the first signal, providing at least a second capture probe, hybridizing in the cell the second capture probe to the second nucleic acid target, when present in the cell, capturing the second label probe to the second capture probe, and detecting the second signal from the second label. Third, fourth, fifth, sixth, etc. nucleic acid targets are similarly simultaneously detected in the cell if desired. Each hybridization or capture step is preferably accomplished for all of the nucleic acid targets at the same time.

The methods permit detection of even low or single copy number targets. Thus, in one class of embodiments, about 1000 copies or less of the first nucleic acid target are present in the cell (e.g., about 100 copies or less, about 50 copies or less, about 10 copies or less, about 5 copies or less, or even a single copy).

Essentially all of the features noted for the methods above apply to these embodiments as well, as relevant; for example, with respect to type of nucleic acid targets, cell type, source of sample, fixation and permeabilization of the cell, washing the cell, denaturation of double-stranded nucleic acids, type of labels, use and configuration of label probes, capture probes, preamplifiers and/or amplifiers (including, e.g., hybridization of two capture probes to a single label probe, preamplifier, or amplifier molecule), use of optional blocking probes, detection of signals, detection (and intensity measurement) of signals from the individual cell by flow cytometry or microscopy, presence of the cell in suspension or immobilized on a substrate, and/or the like.

If the target is short, conventional FISH (or other direct label in situ methods) can not attain sufficient signal to achieve detection of the target. The methods described herein, however, enable in situ, high sensitivity detection of even short targets (e.g., a short nucleic acid molecule or a short region of polynucleotide sequence within a longer nucleic acid molecule), including, e.g., target sections of longer sequences and target molecules less than 1 kb. Accordingly, one general class of embodiments provides methods of detecting one or more nucleic acid targets in an individual cell that include: providing a sample comprising the cell, which cell comprises or is suspected of comprising a first nucleic acid target; providing a first label probe comprising a first label; providing a set of one or more first capture probes; hybridizing, in the cell, the first capture probes to the first nucleic acid target, when present in the cell, wherein the set of first capture probes hybridizes to a region of the first nucleic acid target (including, e.g., the entire target molecule or a portion thereof) that is 1000 nucleotides or less in length (e.g., 500 nucleotides or less in length); capturing the first label probe to the first capture probes, thereby capturing the first label probe to the first nucleic acid target; and detecting a first signal from the first label. For example, the set of first capture probes can hybridize to a region of the first nucleic acid target that is 200 nucleotides or less in length, 100 nucleotides or less in length, 50 nucleotides or less in length, or even 25 nucleotides or less in length, thus permitting detection of target nucleic acids as small as microRNAs, for example. Other exemplary targets include, but are not limited to, short or short regions of DNAs, chromosomal DNAs, RNAs, mRNAs, and ribosomal RNAs.

As for the embodiments above, the methods are useful for multiplex detection of nucleic acids, including simultaneous detection of two or more nucleic acid targets (e.g., short targets, or a combination of short and longer targets). Thus, the cell optionally comprises or is suspected of comprising a second nucleic acid target, and the methods optionally include: providing a second label probe comprising a second label, wherein a second signal from the second label is distinguishable from the first signal, providing a set of one or more second capture probes, hybridizing in the cell the second capture probes to the second nucleic acid target, when present in the cell, capturing the second label probe to the second capture probes, and detecting the second signal from the second label. Third, fourth, fifth, sixth, etc. nucleic acid targets are similarly simultaneously detected in the cell if desired. Each hybridization or capture step is preferably accomplished for all of the nucleic acid targets at the same time.

Essentially all of the features noted for the methods above apply to these embodiments as well, as relevant; for example, with respect to type of nucleic acid targets, copy number, cell type, source of sample, fixation and permeabilization of the cell, washing the cell, denaturation of double-stranded nucleic acids, type of labels, use and configuration of label probes, capture probes, preamplifiers and/or amplifiers (including, e.g., hybridization of two capture probes to a single label probe, preamplifier, or amplifier molecule), use of optional blocking probes, detection of signals, detection (and intensity measurement) of signals from the individual cell by flow cytometry or microscopy, presence of the cell in suspension or immobilized on a substrate, and/or the like.

As noted for the multiplex embodiments above, label probes can be captured indirectly to target nucleic acids through binding of capture probes and optionally also amplifiers and preamplifiers. Such indirect capture is also applicable to detection of single nucleic acids, e.g., in cells. Accordingly, one general class of embodiments provides methods of detecting a nucleic acid target in an individual cell. In the methods, a sample comprising the cell, a label probe comprising a label, and two or more capture probes are provided. The cell comprises (or is suspected of comprising) the nucleic acid target. In the cell, the two or more capture probes are hybridized to the nucleic acid target, and the label probe is captured to the two or more capture probes, thereby capturing the label probe to the nucleic acid target, by hybridizing the two or more capture probes to a copy of the label probe, by hybridizing the two or more capture probes to a copy of an amplifier and hybridizing the label probe to the amplifier, or by hybridizing the two or more capture probes to a copy of a preamplifier and hybridizing an amplifier to the preamplifier and the label probe to the amplifier. A signal from the label is detected.

Optionally, binding of only one (or of fewer than all) of the capture probes is not sufficient to capture the label probe to the target. In one class of embodiments, hybridizing the capture probes to the copy of the label probe, amplifier, or preamplifier is performed at a hybridization temperature that is greater than a melting temperature Tₘ of a complex between each individual capture probe and the label probe, amplifier, or preamplifier. Binding of a single capture probe to the label probe, amplifier, or preamplifier is thus unstable.

A number of capture probe configurations can be employed. For example, in one class of embodiments, each of the two or more capture probes comprises a section T complementary to a section on the nucleic acid target and a section L complementary to a section on the label probe, amplifier, or preamplifier, and each of the two or more capture probes has T 5′ of L or each of the two or more capture probes has T 3′ of L. Typically, the capture probes hybridize to unique and adjacent sections on the nucleic acid target.

The methods are applicable to cells in suspension, immobilized on solid supports, etc. Thus, in one class of embodiments, the sample comprises a tissue section. In another class of embodiments, the cell is in suspension in the sample comprising the cell, and/or the cell is in suspension during the hybridizing, capturing, and/or detecting steps.

The methods can be used for multiplex detection of nucleic acids, including simultaneous detection of two or more target nucleic acids. The cell optionally comprises or is suspected of comprising a second target nucleic acid, and the methods optionally include providing (a) a second label probe comprising a second label whose signal is distinguishable from that of the first label and (b) two or more second capture probes, hybridizing in the cell the two or more second capture probes to the second nucleic acid target, and capturing the second label probe to the two or more second capture probes by hybridizing the two or more second capture probes to a copy of the second label probe, by hybridizing the two or more second capture probes to a copy of a second amplifier and hybridizing the second label probe to the second amplifier, or by hybridizing the two or more second capture probes to a copy of a second preamplifier and hybridizing a second amplifier to the second preamplifier and the second label probe to the second amplifier. Signals from the label and second label are detected. Third, fourth, fifth, etc. nucleic acids are similarly simultaneously detected in the cell if desired, e.g., using third, fourth, fifth, etc. label probes, capture probes, amplifiers, and/or preamplifiers.

Essentially all of the features noted for the methods above apply to these embodiments as well, as relevant; for example, with respect to type of nucleic acid targets, copy number, cell type, source of sample, fixation and permeabilization of the cell, washing the cell, denaturation of double-stranded nucleic acids, type of labels, configuration of label probes, capture probes, preamplifiers and/or amplifiers, use of optional blocking probes, detection of signals, e.g., by flow cytometry or microscopy, and/or the like.

Compositions related to the methods are also a feature of the invention. Thus, one general class of embodiments provides a composition that includes a cell comprising a nucleic acid target, a label probe comprising a label, and two or more capture probes. The capture probes are capable of hybridizing (configured to hybridize) to the nucleic acid target. In one class of embodiments, one copy of the label probe is capable of hybridizing to the two or more capture probes. In another class of embodiments, one copy of an amplifier is capable of hybridizing to the two or more capture probes and to the label probe. In yet another class of embodiments, one copy of a preamplifier is capable of hybridizing to the two or more capture probes and to an amplifier which is capable of hybridizing to the label probe.

Essentially all of the features noted for the methods and compositions above apply to these embodiments as well, as relevant; for example, with respect to type of nucleic acid targets, copy number, cell type, source of sample, fixation and permeabilization of the cell, washing the cell, denaturation of double-stranded nucleic acids, type of labels, configuration of label probes, capture probes, preamplifiers and/or amplifiers, use of optional blocking probes, and/or the like. For example, optionally each of the two or more capture probes comprises a section T complementary to a section on the nucleic acid target and a section L complementary to a section on the label probe, amplifier, or preamplifier, and each of the two or more capture probes has T 5′ of L or each of the two or more capture probes has T 3′ of L. Typically, the capture probes hybridize to unique and adjacent sections on the nucleic acid target. In one class of embodiments, the two or more capture probes are hybridized to the target nucleic acid and to the copy of the label probe, amplifier, or preamplifier, and the composition is maintained at a hybridization temperature that is greater than a melting temperature Tₘ of a complex between each individual capture probe and the label probe, amplifier, or preamplifier. The cell can be, e.g., in a tissue section or in suspension. Optionally, a the cell comprises the label probe and/or capture probes.

Capture of multiple label probes, e.g., via amplifiers and preamplifiers, to each copy of the target nucleic acid according to the methods described herein can result in association of a large number of labels with each individual target nucleic acid molecule. This permits each individual copy of the nucleic acid target to be visualized, e.g., as a fluorescent spot when a fluorescent label is employed. Counting such spots provides a simple and convenient way to quantitate the target nucleic acid.

Accordingly, one general class of embodiments provides methods of quantitating a target nucleic acid (e.g., an RNA). In the methods, a sample comprising one or more copies of the target nucleic acid is provided. Typically, the target nucleic acid is endogenous to a cell. A plurality of copies of an optically detectable label are captured to each of the one or more copies of the target nucleic acid. The copies of the label are optically detected. An optical signal focus (or, equivalently, punctum, spot, or dot) is observable for each of the one or more copies of the target nucleic acid, and the one or more resulting foci are counted, thereby quantitating the target nucleic acid.

As noted, the target nucleic acid can be an RNA, e.g., an mRNA, a microRNA, a ribosomal RNA, or the like. The methods can be applied, e.g., to RNA in situ in a cell or free of any cell. Thus, in one class of embodiments, the sample comprises a cell lysate or other solution comprising the RNA. In another class of embodiments, the sample comprises the cell to which the target RNA is endogenous, and the capturing, detecting, and counting steps are performed in the cell. Optionally, the RNA is located in the cytoplasm of the cell.

The methods are particularly useful for quantitation of low abundance RNAs. Thus, in one embodiment, about 100 copies or less of the target RNA are present in the cell, cell lysate, etc., for example, about 10 copies or less, about 5 copies or less, or even a single copy. As noted, a large number of labels are captured to each molecule. For example, at least about 400 copies of the label can be captured to each of the one or more copies of the target RNA, e.g., at least about 1000 copies, at least about 2000 copies, at least about 4000 copies, or at least about 8000 copies. The label can be, e.g., a fluorescent label or an enzyme (e.g., an enzyme optically detectable using a fluorogenic or chromogenic substrate).

The label can be captured to the nucleic acid directly or indirectly. Optionally, the label is provided by providing one or more copies of a label probe, the label probe comprising one or more copies of the label. The label probe can be hybridized directly to the target nucleic acid. Preferably, however, the label probe is indirectly captured, e.g., by providing one or more capture probes, hybridizing a copy of each of the one or more capture probes to each of the one or more copies of the target nucleic acid, and capturing the one or more copies of the label probe to the one or more capture probes. As for the embodiments above, the label probe can bind directly to the capture probe, or more typically an amplifier or a preamplifier and amplifier serve as intermediates. Optionally, two or more capture probes bind each label probe, amplifier, or preamplifier.

Essentially all of the features noted for the methods above apply to these embodiments as well, as relevant; for example, with respect to cell type, type of target (including size), source of sample, fixation and permeabilization of the cell, washing the cell, denaturation of double-stranded nucleic acids, type of labels, configuration of label probes, capture probes, preamplifiers and/or amplifiers, label density, use of optional blocking probes, and/or the like.

A related general class of embodiments provides methods of quantitating a target RNA. In the methods, a sample comprising one or more copies of the target RNA is provided. The target RNA is generally endogenous to a cell. A plurality of copies of a fluorescent label are captured to each of the one or more copies of the target RNA. The copies of the label are exposed to excitation light (of an appropriate wavelength for the label), whereupon the copies of the label fluoresce, thereby providing a florescent focus (or, equivalently, punctum, spot, or dot) for each of the one or more copies of the target RNA. The one or more resulting fluorescent foci are counted, thereby quantitating the target RNA. The target RNA can be an mRNA, a microRNA, a ribosomal RNA, or the like.

The methods can be applied, e.g., to RNA in situ in a cell or free of any cell. Thus, in one class of embodiments, the sample comprises a cell lysate or other solution comprising the RNA. In another class of embodiments, the sample comprises the cell to which the target RNA is endogenous, and the capturing, exposing, and counting steps are performed in the cell.

The methods are particularly useful for quantitation of low abundance RNAs. Thus, in one embodiment, about 100 copies or less of the target RNA are present in the cell, cell lysate, etc., for example, about 10 copies or less, about 5 copies or less, or even a single copy. As noted, a large number of labels are captured to each molecule. For example, at least about 400 copies of the label can be captured to each of the one or more copies of the target RNA, e.g., at least about 1000 copies, at least about 2000 copies, at least about 4000 copies, or at least about 8000 copies. Optionally, the RNA is located in the cytoplasm of the cell.

The label can be captured to the RNA directly or indirectly. Optionally, the label is provided by providing one or more copies of a label probe, the label probe comprising one or more copies of the label. The label probe can be hybridized directly to the target RNA. Preferably, however, the label probe is indirectly captured, e.g., by providing one or more capture probes, hybridizing a copy of each of the one or more capture probes to each of the one or more copies of the target RNA, and capturing the one or more copies of the label probe to the one or more capture probes. As for the embodiments above, the label probe can bind directly to the capture probe, or more typically an amplifier or a preamplifier and amplifier serve as intermediates. Optionally, two or more capture probes bind each label probe, amplifier, or preamplifier.

Essentially all of the features noted for the methods above apply to these embodiments as well, as relevant; for example, with respect to cell type, type of target (including size), source of sample, fixation and permeabilization of the cell, washing the cell, denaturation of double-stranded nucleic acids, type of labels, configuration of label probes, capture probes, preamplifiers and/or amplifiers, label density, use of optional blocking probes, and/or the like.

The present invention also proposes the use of a probe pair to substitute a regular probe in an assay. As shown in FIG. 1A, a regular probe used in a nucleic acid-based assay (e.g. PCR, microarray, bDNA, etc.) will typically have sufficient hybridization strength to bind to a target sequence strongly and stably under the assay condition (i.e. the Tₘ of the probe or primer is higher than the assay temperature). For example, the oligonucleotide probes used in a microarray will normally have a Tₘ higher than the hybridization temperature. The two primers used in a PCR reaction also will have a Tₘ higher than the annealing temperature. However, because of this strong hybridization strength, such a probe could nonspecifically hybridize with sequences present in unintended regions, either because they matched perfectly or showed high degree of homology (FIG. 26B). Such non-specific hybridization problem is particularly severe in genotyping because the target allele differs from its wild type by only a single base (FIG. 26B). This invention describes a unique probe configuration to improve the hybridization specificity. In the invented configuration, the regular probe shown in FIGS. 26A and 26B is replaced by a probe pair, which is consisted of a first capture probe, also names as functional probe (FP) and a second capture probe, also named as location-anchoring probe (LP) as shown in FIGS. 26C and 26D. The FP contains at least a targeting region (region AB in FIG. 26), designed to bind to the intended target sequence and at least an anchoring region (BC in FIG. 26), designed to bind a corresponding region in LP. The LP contains also at least a targeting region (region DE in FIG. 26), designed to bind to its own target sequence right next or very close to the target sequence bond by FP on the target molecule and at least an anchoring region (EF in FIG. 26), designed to bind a corresponding region in FP. The targeting region of FP is designed as such that, if on its own, it will not bind to the target sequence or any other sequences strongly and stably (i.e. Tₘ is lower than the assay temperature). On the other hand, the targeting region of LP, if on its own, can either have strong or weak hybridization to the target sequence (i.e. Tₘ above or below the assay temperature). When F P and LP both exist and binds to their respective target sequences in the assay, a stable scaffold structure forms, as shown in FIG. 26C, which will exhibit a much stronger hybridization strength than FP or LP alone, thus enables FP to bind to its target sequence strongly and stably. Such an approach should have much higher assay specificity than the regular probe design because FP will not bind to the target or any other sequences on its own unless LP is present and nearby. If LP is hybridized nonspecifically to a sequence other than its intended target, a stable scaffold is unlikely to form because the anchoring regions do not have sufficient hybridization strength to hold FP and LP together. Although this design allows the binding between LP and the target to be strong, the assay specificity can be enhanced further if that binding is also weak (i.e. Tₘ below assay temperature). In this way, LP, on its own, will not able to hybridize to its target or any other sequence. When and only when both FP and LP are hybridized to their respective target sequences, the scaffold will become stable, enabling FP to bind strongly to its target under the assay condition.

With this invented design, FP has more power to discriminate between match and mismatch sequences because its targeting region (AB) is much shorter than a regular probe, typically in the range of 9-16 bases. The short targeting region makes the difference in thermal stability much bigger between match and mismatch sequences. The targeting region in LP (DE), on the other hand, can be as short as that in FP or slightly longer, for example, 15 to 30 bases. The anchoring regions (BC in FP and EF in LP) are designed to strengthen the hybridization interaction and should therefore at least partially complementary to each other. They each can be as short as 0 bases and as long as 15 bases. Typically, this complementary sequence of the anchoring regions of FP and LP is between 5 to 10 bases. Region EF may contain modified nucleotides such as LNA, PNA, ddNTP, etc. at the 3′ end to prevent it from serving as a probe or primer in an enzymatic reaction such as polymerization or ligation. As a result, the LP will only serve as a location-specific anchor for the binding of FP to target sequence. When the anchoring regions (BC in FP and EF in LP) are 0 base long, there is no direct binding between LP and FP. However, experimental data from the inventor showed that the base stacking between LP/FP can still provide sufficient improvement in binding strength, compared to FP or LP binding to the target alone, that enables the LP/FP to bind to the target stably throughout the assay.

An aspect of the invention is directed to a method of detecting at least one target nucleic acid, as described in claim 1 below.

Another aspect of the invention is directed to a method of capturing a label to at least one target nucleic acid, as described in claim 18 below.

Another aspect of the invention is directed to a method of detecting an individual cell of a specified type, as described in claim 35 below.

Another aspect of the invention is directed to a composition as described in claim 49 below.

Another aspect of the invention is directed to a tissue slide as described in claim 65 below.

Another aspect of the invention is directed to a sample of suspending cells as described in claim 75 below.

Another aspect of the invention is directed to a kit as described in claim 85 below.

Another aspect of the invention is directed to a method of detecting at least one target nucleic acid as described in claim 93 below.

Another aspect of the invention is directed to a method of detecting at least one target nucleic acid as described in claim 110 below.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 schematically illustrates QMAGEX technology workflow for an exemplary embodiment.

FIG. 2 schematically illustrates a direct labeling approach in which label probes are hybridized to the target nucleic acid.

FIG. 3 schematically illustrates an indirect labeling approach in which label probes are hybridized to capture probes hybridized to the target nucleic acid.

FIG. 4 schematically illustrates an indirect labeling capture probe design approach that utilizes a pair of independent capture probes to enhance the specificity of the label probe capture to the target nucleic acid.

FIG. 5 schematically illustrates an indirect labeling capture probe design approach that utilizes three or more independent capture probes to enhance the specificity of the label probe capture to the target nucleic acid.

FIGS. 6A-6B schematically illustrate probe design approaches to detect multiple target molecules in parallel using either direct labeling (FIG. 6A) or indirect labeling with two independent capture probes (FIG. 6B).

FIGS. 7A-7C schematically illustrate probe design approaches to reducing false positive rates in rare cell identification by attaching multiple types of signal-generating particles (labels) to the same target molecule. FIG. 7A shows multiple types of signal-generating particles (labels) on one target. FIG. 7B shows multiple types of signal-generating particles (labels) on more than one target, where the relative signal strengths of the particle set are maintained across all targets. FIG. 7C shows a set of signal-generating particles (labels) on a target molecule, where different targets have distinctively different sets.

FIGS. 8A-8D schematically illustrate different structures of exemplary amplifiers.

FIGS. 9A-9C schematically illustrate utilizing rolling circle amplification to amplify signal. As shown in FIG. 9A, a circular nucleotide molecule is attached to capture probe(s). As shown in FIG. 9B, a long chain molecule with many repeated sequences appears as a result of rolling circle amplification. As shown in FIG. 9C, many signal probes can be hybridized to the repeated sequences to achieve signal amplification.

FIG. 10 schematically illustrates one embodiment of the assay instrument configuration.

FIGS. 11A-11D schematically illustrate a multiplex assay for two nucleic acids in cells in suspension.

FIGS. 12A-12E illustrate detection of 18S RNA in HeLa cells using the 16×AMP2 system (FIG. 12A) versus controls using the 1×AMP3 system (FIG. 12B), capture probes complementary to the antisense strand (FIG. 12C), and half of the capture probe set (FIGS. 12D and 12E).

FIGS. 13A-13D illustrate multiplex detection of 18S RNA and Her-2 mRNA in HeLa cells (FIGS. 13A and 13C) and SKBR3 cells (FIGS. 13B and 13D). FIGS. 13C and 13D represent a control experiment, in which capture probes targeting the anti-sense strand of the Her-2 intron sequence were used.

FIG. 14 presents a graph comparing Alexa488 and Fast Red detection.

FIGS. 15A-15D illustrate detection of changes in expression of IL-6 and IL-8 in single cells. Resting HeLa cells are shown in FIGS. 15A and 15B and PMA-treated cells in FIGS. 15C and 15D. Expression of IL-6 is shown in FIG. 15A and FIG. 15C and expression of IL-8 is shown in FIG. 15B and FIG. 15D.

FIGS. 16A-16B illustrate detection of cancer cells in mixed cell populations. FIG. 16A illustrates detection of SKBR3 cells mixed with Jurkat cells. FIG. 16B illustrates detection of BT474 breast cancer cells mixed with blood cells.

FIGS. 17A-17D illustrate detection in suspended HeLa cells. FIG. 17A shows cells not hybridized with capture probes or signal amplifiers. FIG. 17B shows cells hybridized with 18S capture probes and a 1×AMP3 system. FIG. 17C shows cells hybridized with 18S capture probes and a 16×AMP2 system. FIG. 17D shows a corresponding flow cytometric histogram.

FIG. 18 presents a flow cytometric histogram illustrating detection of low copy mRNAs.

FIGS. 19A-191 schematically illustrate different capture probe configurations. The solid horizontal line represents the target nucleic acid, and the dashed horizontal line represents a label probe, amplifier, or preamplifier.

FIGS. 20A-20B illustrate specific detection of a splice variant. Binding of two capture probes to the splice variant results in its detection (FIG. 20A). Another variant, to which only one of the two capture probes binds, is not detected (FIG. 20B).

FIG. 21 illustrates specific detection of a splice variant through capture of two different labels to different regions of the variant.

FIGS. 22A-22D illustrate MAGEX detection of mRNAs in breast cancer FFPE tissue section: 18S in FIG. 22A, β-actin in FIG. 22B, Ck19 in FIG. 22C, and control 18S intron in FIG. 22D. Sections shown in FIGS. 22A-22D are also stained with DAPI.

FIGS. 23A-23F illustrate detection of a low copy mRNA in breast cancer FFPE tissue sections. Detection of Her-2 is shown in FIGS. 23A-23C; FIG. 23A shows Gill's Hematoxylin staining of cell nuclei, FIG. 23B shows detection of Her-2 mRNA using a MAGEX assay with a probe set for Her-2 and Fast Red substrate, and FIG. 23C shows a merged picture for Her-2 and Gill's Hematoxylin. A control in which no target probe was employed is shown in FIGS. 23D-23F; FIG. 23D shows Gill's Hematoxylin staining of cell nuclei, Panel E shows detection using Fast Red (but no target probe), and FIG. 23F shows a merged picture for Her-2 and Gill's Hematoxylin.

FIGS. 24A-24I illustrate detection of an mRNA in tissue microarray. FIGS. 24A-24C show Gill's Hematoxylin staining of cell nuclei in the tissue sections. FIGS. 24D-24F show the tissue sections labeled with a MAGEX assay using probes against CK19 (FIG. 24D), Her-2 (FIG. 24F), or a control with no probe (FIG. 24E). FIGS. 24G-24I show merged pictures for CK19 and Gill's Hematoxylin (FIG. 24G), Her-2 and Gill's Hematoxylin (FIG. 24I), and no probe control and Gill's Hematoxylin (FIG. 24H).

FIGS. 25A-25D schematically illustrate identification of CTCs in blood samples from four different breast cancer patients. Staining is Fast Red (for CK19) and DAPI.

FIGS. 26A-26D schematically depict paired probe configuration.

FIGS. 27A-27B schematically depict genotyping by single base extension using paired configuration.

FIGS. 28A-28B schematically depict multiplex genotyping using single base extension.

FIGS. 29A-29B schematically depict genotyping by hybridization.

FIG. 30 schematically depicts using paired probe in Taqman assay.

FIGS. 31A-31C schematically depict using paired probes in ligation assay.

FIGS. 32A-32B schematically depict signal amplification using paired probe configuration.

FIG. 33 schematically depicts different scaffold configurations.

FIGS. 34A-34B schematically depict scaffolds with additional support porbes.

FIG. 35 schematically depicts detection of target nucleic acid sequence by rolling circle amplification.

FIGS. 36A-36C schematically depict incorporating ligation into porbe scaffold to further improve specificity.

FIGS. 37A-37B schematically depict using ligation to improve specificity of rolling circle amplification.

FIG. 38 schematically depicts using cooperative hybridization in in situ genotyping.

FIG. 39 schematically depicts the concept for cooperative hybridization event not directly linked to the target.

FIG. 40 schematically depicts the concept for reduction of false positive or background signals using linkers which are directly hybridized to target nucleic acid sequence.

FIG. 41 schematically depicts the concept for reduction of false positive or background signals using linkers which are indirectly hybridized to target nucleic acid sequence, and indirectly hybridized to label probe system.

FIG. 42 depicts the concept for reduction of false positive or background signals using multiple linkers.

FIG. 43 schematically depicts the concept for reduction of false positive or background signals using linkers which are indirectly hybridized to target nucleic acid sequence, and indirectly hybridized to label probe system, where preamplifers are used as linkers.

FIG. 44 schematically depicts the concept for reduction of false positive or background signals using linkers which are indirectly hybridized to target nucleic acid sequence, and indirectly hybridized to label probe system, where the linkers (or preamplifiers) are directly bound to the target nucleic acid sequence without using capture probes.

FIG. 45 schematically depicts the concept that the pair of linker capture probes are integrated into one.

FIG. 46 schematically depicts the concept that the linker capture probe is integrated into the amplifier.

FIGS. 47A-47C schematically depict the use of capture probe set to detect SNP.

FIG. 48 schematically depicts the use of capture probe set to detect SNP with reduced false positive or background signals using linkers which are indirectly hybridized to target nucleic acid sequence.

FIG. 49 schematically depicts nucleic acid splicing detection using signal co-location approach.

FIG. 50 schematically depicts nucleic acid splicing detection using signal co-location approach with signal applification.

FIG. 51 schematically depicts nucleic acid splicing detection using signal co-location approach with RNAscope.

FIG. 52 schematically depicts combined detection of splice by different nucleic acid section.

FIG. 53 schematically depicts detection of specific slice junction.

FIG. 54 schematically depicts detection of a splice junction by deploying 3D oligo scaffold.

FIG. 55 schematically depicts detection of a splice junction by deploying 3D oligo scaffold without linker capture porbes.

FIGS. 56A-56B depicts assay result of detecting RNA fusion transcript. Jurkat (FIG. 56A) and K562 (FIG. 56B) cells were simultaneously hybridized with probe sets to BCR and ABL. BCR probe sets were detected with a red fluorescent dye, and ABL probe sets were detected with a green fluorescent dye. The presence of yellow dots (arrows) in the K562 cells indicates BCR-ABL fusion transcripts.

FIG. 57 schematically illustrates a typical standard bDNA assay.

FIGS. 58A-58E schematically depict a multiplex nucleic acid detection assay, in which the nucleic acids of interest are captured on distinguishable subsets of microspheres and then detected.

FIGS. 59A-59D schematically depict a multiplex nucleic acid detection assay, in which the nucleic acids of interest are captured at selected positions on a solid support and then detected. FIG. 59A shows a top view of the solid support, while FIGS. 59B-59D show the support in cross-section.

FIGS. 60A-60C schematically depict label extender configurations. FIG. 60A schematically depicts a double Z label extender configuration. FIG. 60B schematically depicts a cruciform label extender configuration. FIG. 60C depicts a bar graph comparing luminescence observed in bDNA assays using double Z configuration label extenders or cruciform label extenders.

FIG. 61 schematically depicts a number of clauses of nonspecific detection.

FIG. 62 schematically depicts nonspecific detection with an amplifier.

FIG. 63 schematically depicts the use of co-location probes.

FIG. 64 schematically depicts the use of co-location probes for in situ genotyping.

FIG. 65 schematically depicts co-location probes in multiplex in situ genotyping.

FIG. 66 schematically depicts the use of co-location probes and short capture probes for multiplex in situ genotyping.

Schematic figures are not necessarily to scale.

### Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

A “nucleic acid target” or “target nucleic acid” refers to a nucleic acid, or optionally a region thereof, that is to be detected.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

The term “gene” is used broadly to refer to any nucleic acid associated with a biological function. Genes typically include coding sequences and/or the regulatory sequences required for expression of such coding sequences. The term gene can apply to a specific genomic sequence, as well as to a cDNA or an mRNA encoded by that genomic sequence. Genes also include non-expressed nucleic acid segments that, for example, form recognition sequences for other proteins. Non-expressed regulatory sequences include promoters and enhancers, to which regulatory proteins such as transcription factors bind, resulting in transcription of adjacent or nearby sequences.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

A first polynucleotide “capable of hybridizing” to a second polynucleotide contains a first polynucleotide sequence that is complementary to a second polynucleotide sequence in the second polynucleotide. The first and second polynucleotides are able to form a stable duplex, e.g., under relevant assay conditions.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “capture probe” is a polynucleotide that is capable of hybridizing to a target nucleic acid and capturing a label probe to that target nucleic acid. The capture probe can hybridize directly to the label probe, or it can hybridize to one or more nucleic acids that in turn hybridize to the label probe; for example, the capture probe can hybridize to an amplifier or a preamplifier. The capture probe thus includes a first polynucleotide sequence that is complementary to a polynucleotide sequence of the target nucleic acid and a second polynucleotide sequence that is complementary to a polynucleotide sequence of the label probe, amplifier, preamplifier, or the like. The capture probe is preferably single-stranded.

An “amplifier” is a molecule, typically a polynucleotide, that is capable of hybridizing to multiple label probes. Typically, the amplifier hybridizes to multiple identical label probes. The amplifier also hybridizes to at least one capture probe or nucleic acid bound to a capture probe. For example, the amplifier can hybridize to at least one capture probe and to a plurality of label probes, or to a preamplifier and a plurality of label probes. The amplifier can be, e.g., a linear, forked, comb-like, or branched nucleic acid. As noted for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, and 5,849,481.

A “preamplifier” is a molecule, typically a polynucleotide, that serves as an intermediate between one or more capture probes and amplifiers. Typically, the preamplifier hybridizes simultaneously to one or more capture probes and to a plurality of amplifiers. Exemplary preamplifiers are described, for example, in U.S. Pat. Nos. 5,635,352 and 5,681,697.

The term “Signal Generating Probe” refers to an entity that binds to a target molecule, directly or indirectly, and enables the target to be detected, e.g., by a readout instrument. A signal generating probe (or “SGP”) is typically a single-stranded polynucleotide that comprises at least one label which directly or indirectly provides a detectable signal. The label can be covalently attached to the polynucleotide, or the polynucleotide can be configured to bind to the label (e.g., a biotinylated polynucleotide can bind a streptavidin-associated label). The label probe can, for example, hybridize directly to a target nucleic acid, or it can hybridize to a nucleic acid that is in turn hybridized to the target nucleic acid or to one or more other nucleic acids that are hybridized to the nucleic acid. Thus, SGP can comprise a polynucleotide sequence that is complementary to a polynucleotide sequence of the target nucleic acid, or it can comprise at least one polynucleotide sequence that is complementary to a polynucleotide sequence in a capture probe, amplifier, or the like. Or, SGP can comprise a label and an amplifier hybridized to the label and hybridized to said set of two or more capture probes. Further, SGP can comprise a label, an amplifier hybridized to the label probe, and a preamplifier hybridized to the amplifier and hybridized to said set of two or more capture probes.

The term “label probe” is identical in meaning with signal generating probe and thus can be used interchangeably.

The term “label probe system” (or “LPS”) is identical in meaning with signal generating probe thus can be used interchangeably.

The term “functional probe” (or “FP”) refers to a type of capture probe comprising at least a targeting region designed to bind to the intended target nucleic acid sequence, and an anchor region designed to bind to a corresponding region in location-anchoring probe.

The term “location-anchoring probe” (or “LP”) refers to a type of capture probe comprising at least a targeting region designed to bind to the intended target nucleic acid sequence, and an anchor region designed to bind to a corresponding region in functional probe.

The term “support probe” (or “SP”) refers to a type of capture probe comprising at least a targeting region designed to bind to a section of the target nucleic acid sequence adjacent to the section of target nucleic acid sequence which is complementary to the targeting region of function probe or location-anchoring probe. Support probe may optionally comprises a section which bind directly with functional probe or location-anchoring probe. Support probe may be placed on either side of the scaffold structure consisting of target nucleic acid sequence, LP, FP, and AMP, and to further increase the hybridization strength of the structure.

A “capture probe set” (or “CPS”) is a set of two or more capture probes.

The term “linker” refers to an entity that binds to a target nucleic acid sequence directly and indirectly, and also binds to signal generating probe directly and indirectly. Thus, a linker can comprise a polynucleotide sequence that is complementary to a polynucleotide sequence of the target nucleic acid, or it can comprise at least one polynucleotide sequence that is complementary to a polynucleotide sequence in a capture probe, an amplifier, or the like.

The term “linker capture probe” (or “LCP”) refers a type of capture probe that has one section capable of hybridizing to a linker and another section capable of hybridizing to a signal generating probe, an amplifier or a like.

A “capture extender” or “CE” is a polynucleotide that is capable of hybridizing to a nucleic acid of interest and to a capture probe. The capture extender typically has a first polynucleotide sequence C-1, which is complementary to the capture probe, and a second polynucleotide sequence C-3, which is complementary to a polynucleotide sequence of the target nucleic acid. Sequences C-1 and C-3 are typically not complementary to each other. The capture extender is preferably single-stranded.

A “capture pole” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

A “label extender” or “LE” is identical in meaning with capture probe thus can be used interchangeably.

An “amplification multimer” is identical in meaning with amplifier thus can be used interchangeably.

A “blocking probe” is a nucleic acids sequence which hybridize to regions of the target nucleic acids sequence not occupied by capture probes or label extenders. It is often used to reduce non-specific target probe binding.

A “pathogen” is a biological agent, typically a microorganism, that causes disease or illness to its host.

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A variety of additional terms are defined or otherwise characterized herein.

### DETAILED DESCRIPTION

Detection of nucleic acid analytes in biological samples can be broadly categorized into two types of methods: “whole-sample” and “in situ” detection. In the whole-sample detection method, the cells in the sample are lysed, which releases the molecules contained in the cells, including the nucleic acid analytes, into sample solution. Then the quantities of the nucleic acid analytes of the entire biological sample are measured in the solution. In the in situ detection method, the nucleic acid analytes are fixed within the host cells and their quantities are measured at an individual cell level. While the methods, compositions, and systems of the instant invention are primarily described herein with reference to in situ detection, many features of the invention can also be applied to whole-sample detection.

In situ detection of nucleic acid analytes is highly desirable for two major reasons. First, biological samples are usually heterogeneous, e.g., containing different types of cells where only a sub-population of the cells is disease relevant. Early in the onset of disease, the fraction of cells in the sample that are affected by the disease can be very small. Since many nucleic acid analytes that serve as disease markers exist not only in disease cells but also in normal cells, albeit at different levels, in such instances a whole-sample detection approach can distort measurement results. This problem is particularly acute if the disease cell population represents a tiny fraction of the cells in the sample. The second reason is that in situ detection maintains cell morphology and/or tissue structure intact. The fusion of information provided by molecular disease markers and cell morphology and/or tissue structure may yield additional scientific or clinical diagnostic value.

Fluorescent In Situ Hybridization (FISH) is a well established method of localizing and detecting DNA sequences in morphologically preserved tissue sections or cell preparations (Pinkel et al., 1986). The FISH assay typically employs specially constructed DNA probes, which are directly labeled with fluorescent dyes and collectively cover about 100,000 nucleotides per target. The methods described herein can also be adapted to detect and localize DNA sequences in situ, although they can employ signal amplification to add hundreds of fluorescent labels per probe pair that hybridizes to approximately 50 bases of target sequence. As a result, the base pair detection resolution is in the order of one thousand nucleotides or less, i.e. over one hundred times better than that of traditional FISH. In addition, unique features in the probe set design can significantly improve hybridization specificity, which facilitates easy multiplexing and improves signal-to-noise ratios. Use of synthetic oligos also brings the benefit of product scalability and quality consistency.

Similar in situ hybridization techniques, which are generally referred to as “ISH” technology, have been used to detect mRNA within individual cells (Hicks et al., 2004). There are four main types of probes that are typically used in performing ISH: oligonucleotide probes (usually 20-40 bases in length), single-stranded DNA probes (200-500 bases in length), double stranded DNA probes, or RNA probes (200-5000 bases in length). RNA probes are currently the most widely used probes for in situ hybridization as they have the advantage that RNA-RNA hybrids are very thermostable and are resistant to digestion by RNases. However, RNA probe is a direct labeling method that suffers a number of difficulties. First, separate labeled probes have to be prepared for detecting each mRNA of interest. Second, it is technically difficult to detect the expression of multiple mRNAs of interest in situ at the same time. As a result, only sequential detection of multiple mRNAs using different labeling methods has recently been reported (Schrock et al, 1996; Kosman et al, 2004). Furthermore, with direct labeling methods, there is no good way to control for potential cross-hybridization with non-specific sequences in cells. In short, the detection sensitivity of traditional ISH is limited to 10-20 mRNA copies per cell. In fact, there is currently no commercial ISH products available that can reliably detect mRNA below 50 copies per cell. This is a major handicap for the use of traditional ISH in diagnostics because more than 95% of human genes express at a level below 50 copies per cell (Zhang et al. 1997) and many of the detectable human genes that are high expressors are constitutively expressed housekeeping genes of less diagnostic interest.

A new type of in situ hybridization method employing Branched DNA (bDNA) has recently been developed for detecting mRNA in single cells (Player et al, 2001). This method uses a series of oligonucleotide probes that have one portion hybridizing to the specific mRNA of interest and another portion hybridizing to the bDNA for signal amplification and detection. bDNA ISH has the advantages that unlabeled oligonucleotide probes are used for detecting every mRNA of interest and that the signal amplification and detection reagents are generic components in the assay. However, the nonspecific hybridization of the oligonucleotide probes in bDNA ISH can become a serious problem when multiple of those probes have to be used for the detection of a low abundance mRNA. Some of the probes may hybridize to unintended sequences, leading to signal amplification of the background, thus reducing detection sensitivity. Similarly, although use of bDNA ISH to detect or quantitate multiple mRNAs is desirable, such nonspecific hybridization of the oligonucleotide probes is a potential problem.

Among other benefits, methods of the present invention overcome the above noted difficulties and provide unique mechanisms for background noise reduction and for improving detection sensitivity and specificity. As a result, they are capable of reliable detection of nucleic acid targets within individual cells at a sensitivity well below 50 copies per cell in a wide range of biological sample types, including, e.g., FFPE tissue sections. In addition, the methods of the present invention are particularly useful for identifying rare cells in a sample with mixed cell populations. Important exemplary applications include, but are not limited to, the detection of circulating tumor cells (CTC) in blood or other bodily fluids, detection of tumor cells in solid tissue sections, detection of cancer stem cells in solid tumor sections or in bodily fluids such as blood, and detection of fetal cells in maternal blood.

Among other aspects, the present invention provides multiplex assays that can be used for simultaneous detection, and optionally quantitation, of two or more nucleic acid targets in a single cell. A related aspect of the invention provides methods for detecting the level of one or more target nucleic acids, e.g., absolute or relative to that of a reference nucleic acid in an individual cell.

In general, in the assays of the invention, a label probe is captured to each target nucleic acid. The label probe can be captured to the target through direct binding of the label probe to the target. Preferably, however, the label probe is captured indirectly through binding to capture probes, amplifiers, and/or preamplifiers that bind to the target. Use of the optional amplifiers and preamplifiers facilitates capture of multiple copies of the label probe to the target, thus amplifying signal from the target without requiring enzymatic amplification of the target itself. Binding of the capture probes is optionally cooperative, reducing background caused by undesired cross hybridization of capture probes to non-target nucleic acids (a greater problem in multiplex assays than singleplex assays since more probes must be used in multiplex assays, increasing the likelihood of cross hybridization).

One aspect of the invention relates to detection of single cells, including detection of rare cells from a heterogeneous mixture of cells, e.g., in suspension or in solid tissue samples. Individual cells are detected through detection of nucleic acids whose presence, absence, copy number, or the like are characteristic of the cell.

Compositions, kits, and systems related to the methods are also provided.

### Methods of Detecting Nucleic Acids and Cells

Multiplex Detection of Nucleic Acids

As noted, one aspect of the invention provides multiplex nucleic acid assays in single cells. Thus, one general class of embodiments includes methods of detecting two or more nucleic acid targets in an individual cell. In the methods, a sample comprising the cell is provided. The cell comprises, or is suspected of comprising, a first nucleic acid target and a second nucleic acid target. A first label probe comprising a first label and a second label probe comprising a second label, wherein a first signal from the first label is distinguishable from a second signal from the second label, are provided. At least a first capture probe and at least a second capture probe are also provided.

The first capture probe is hybridized, in the cell, to the first nucleic acid target (when the first nucleic acid target is present in the cell), and the second capture probe is hybridized, in the cell, to the second nucleic acid target (when the second nucleic acid target is present in the cell). The first label probe is captured to the first capture probe and the second label probe is captured to the second capture probe, thereby capturing the first label probe to the first nucleic acid target and the second label probe to the second nucleic acid target. The first signal from the first label and the second signal from the second label are then detected. Since the first and second labels are associated with their respective nucleic acid targets through the capture probes, presence of the label(s) in the cell indicates the presence of the corresponding nucleic acid target(s) in the cell. The methods are optionally quantitative. Thus, an intensity of the first signal and an intensity of the second signal can be measured, and the intensity of the first signal can be correlated with a quantity of the first nucleic acid target in the cell while the intensity of the second signal is correlated with a quantity of the second nucleic acid target in the cell. As another example, a signal spot can be counted for each copy of the first and second nucleic acid targets to quantitate them, as described in greater detail below.

In one aspect, the label probes bind directly to the capture probes. For example, in one class of embodiments, a single first capture probe and a single second capture probe are provided, the first label probe is hybridized to the first capture probe, and the second label probe is hybridized to the second capture probe. In a related class of embodiments, two or more first capture probes and two or more second capture probes are provided, as are a plurality of the first label probes (e.g., two or more identical first label probes) and a plurality of the second label probes (e.g., two or more identical second label probes). The two or more first capture probes are hybridized to the first nucleic acid target, and the two or more second capture probes are hybridized to the second nucleic acid target. A single first label probe is hybridized to each of the first capture probes, and a single second label probe is hybridized to each of the second capture probes.

In another aspect, the label probes are captured to the capture probes indirectly, for example, through binding of preamplifiers and/or amplifiers. Use of amplifiers and preamplifiers can be advantageous in increasing signal strength, since they can facilitate binding of large numbers of label probes to each nucleic acid target.

In one class of embodiments in which amplifiers are employed, a single first capture probe, a single second capture probe, a plurality of the first label probes, and a plurality of the second label probes are provided. A first amplifier is hybridized to the first capture probe and to the plurality of first label probes, and a second amplifier is hybridized to the second capture probe and to the plurality of second label probes. In another class of embodiments, two or more first capture probes, two or more second capture probes, a plurality of the first label probes, and a plurality of the second label probes are provided. The two or more first capture probes are hybridized to the first nucleic acid target, and the two or more second capture probes are hybridized to the second nucleic acid target. A first amplifier is hybridized to each of the first capture probes, and the plurality of first label probes is hybridized to the first amplifiers. A second amplifier is hybridized to each of the second capture probes, and the plurality of second label probes is hybridized to the second amplifiers.

In one class of embodiments in which preamplifiers are employed, a single first capture probe, a single second capture probe, a plurality of the first label probes, and a plurality of the second label probes are provided. A first preamplifier is hybridized to the first capture probe, a plurality of first amplifiers is hybridized to the first preamplifier, and the plurality of first label probes is hybridized to the first amplifiers. A second preamplifier is hybridized to the second capture probe, a plurality of second amplifiers is hybridized to the second preamplifier, and the plurality of second label probes is hybridized to the second amplifiers. In another class of embodiments, two or more first capture probes, two or more second capture probes, a plurality of the first label probes, and a plurality of the second label probes are provided. The two or more first capture probes are hybridized to the first nucleic acid target, and the two or more second capture probes are hybridized to the second nucleic acid target. A first preamplifier is hybridized to each of the first capture probes, a plurality of first amplifiers is hybridized to each of the first preamplifiers, and the plurality of first label probes is hybridized to the first amplifiers. A second preamplifier is hybridized to each of the second capture probes, a plurality of second amplifiers is hybridized to each of the second preamplifiers, and the plurality of second label probes is hybridized to the second amplifiers. Optionally, additional preamplifiers can be used as intermediates between a preamplifier hybridized to the capture probe(s) and the amplifiers.

In the above classes of embodiments, one capture probe hybridizes to each label probe, amplifier, or preamplifier. In alternative classes of related embodiments, two or more capture probes hybridize to the label probe, amplifier, or preamplifier. See, e.g., the section below entitled “Implementation, applications, and advantages.”

In embodiments in which two or more first capture probes and/or two or more second capture probes are employed, the capture probes preferably hybridize to nonoverlapping polynucleotide sequences in their respective nucleic acid target. The capture probes can, but need not, cover a contiguous region of the nucleic acid target. Blocking probes, polynucleotides which hybridize to regions of the nucleic acid target not occupied by capture probes, are optionally provided and hybridized to the target. For a given nucleic acid target, the corresponding capture probes and blocking probes are preferably complementary to physically distinct, nonoverlapping sequences in the nucleic acid target, which nonoverlapping sequences are preferably, but not necessarily, contiguous. Having the capture probes and optional blocking probes be contiguous with each other can in some embodiments enhance hybridization strength, remove secondary structure, and ensure more consistent and reproducible signal.

In many embodiments, such as those above, enzymatic manipulation is not required to capture the label probes to the capture probes. In other embodiments, however, enzymatic manipulation, particularly amplification of nucleic acids intermediate between the capture probes and the label probes, facilitates detection of the nucleic acid targets. For example, in one class of embodiments, a plurality of the first label probes and a plurality of the second label probes are provided. A first amplified polynucleotide is produced by rolling circle amplification of a first circular polynucleotide hybridized to the first capture probe. The first circular polynucleotide comprises at least one copy of a polynucleotide sequence identical to a polynucleotide sequence in the first label probe, and the first amplified polynucleotide thus comprises a plurality of copies of a polynucleotide sequence complementary to the polynucleotide sequence in the first label probe. The plurality of first label probes is then hybridized to the first amplified polynucleotide. Similarly, a second amplified polynucleotide is produced by rolling circle amplification of a second circular polynucleotide hybridized to the second capture probe (preferably, at the same time the first amplified polynucleotide is produced). The second circular polynucleotide comprises at least one copy of a polynucleotide sequence identical to a polynucleotide sequence in the second label probe, and the second amplified polynucleotide thus comprises a plurality of copies of a polynucleotide sequence complementary to the polynucleotide sequence in the second label probe. The plurality of second label probes is then hybridized to the second amplified polynucleotide. The amplified polynucleotides remain associated (e.g., covalently) with the capture probe(s), and the label probes are thus captured to the nucleic acid targets. A circular polynucleotide can be provided and hybridized to the capture probe, or a linear polynucleotide that is circularized by ligation after it binds to the capture probe (e.g., a padlock probe) can be employed. Techniques for rolling circle amplification, including use of padlock probes, are well known in the art. See, e.g., Larsson et al. (2004) “In situ genotyping individual DNA molecules by target-primed rolling-circle amplification of padlock probes” Nat Methods. 1(3):227-32, Nilsson et al. (1994) Science 265:2085-2088, and Antson et al. (2000) “PCR-generated padlock probes detect single nucleotide variation in genomic DNA” Nucl Acids Res 28(12):E58.

Potential capture probe sequences are optionally examined for possible interactions with non-corresponding nucleic acid targets, the preamplifiers, the amplifiers, the label probes, and/or any relevant genomic sequences, for example. Sequences expected to cross-hybridize with undesired nucleic acids are typically not selected for use in the capture probes (but may be employed as blocking probes). Examination can be, e.g., visual (e.g., visual examination for complementarity), computational (e.g., a BLAST search of the relevant genomic database, or computation and comparison of binding free energies), and/or experimental (e.g., cross-hybridization experiments). Repetitive sequences are generally avoided. Label probe sequences are preferably similarly examined, to help minimize potential undesirable cross-hybridization.

A capture probe, preamplifier, amplifier, and/or label probe optionally comprises at least one non-natural nucleotide. For example, a capture probe and a preamplifier (or amplifier or label probe) that hybridizes to it optionally comprise, at complementary positions, at least one pair of non-natural nucleotides that base pair with each other but that do not Watson-Crick base pair with the bases typical to biological DNA or RNA (i.e., A, C, G, T, or U). Examples of nonnatural nucleotides include, but are not limited to, Locked NucleicAcid™ nucleotides (available from Exiqon A/S, www (dot) exiqon (dot) com; see, e.g., SantaLucia Jr. (1998) Proc Natl Acad Sci 95:1460-1465) and isoG, isoC, and other nucleotides used in the AEGIS system (Artificially Expanded Genetic Information System, available from EraGen Biosciences, www (dot) eragen (dot) com; see, e.g., U.S. Pat. Nos. 6,001,983, 6,037,120, and 6,140,496). Use of such non-natural base pairs (e.g., isoG-isoC base pairs) in the probes can, for example, reduce background and/or simplify probe design by decreasing cross hybridization, or it can permit use of shorter probes when the non-natural base pairs have higher binding affinities than do natural base pairs.

As noted, the methods are useful for multiplex detection of nucleic acids, including simultaneous detection of more than two nucleic acid targets. Thus, the cell optionally comprises or is suspected of comprising a third nucleic acid target, and the methods optionally include: providing a third label probe comprising a third label, wherein a third signal from the third label is distinguishable from the first and second signals, providing at least a third capture probe, hybridizing in the cell the third capture probe to the third nucleic acid target (when the third target is present in the cell), capturing the third label probe to the third capture probe, and detecting the third signal from the third label. Fourth, fifth, sixth, etc. nucleic acid targets are similarly simultaneously detected in the cell if desired.

A nucleic acid target can be essentially any nucleic acid that is desirably detected in the cell. For example, a nucleic acid target can be a DNA, a chromosomal DNA, an RNA (e.g., a cytoplasmic RNA), an mRNA, a microRNA, a ribosomal RNA, or the like. The nucleic acid target can be a nucleic acid endogenous to the cell. As another example, the target can be a nucleic acid introduced to or expressed in the cell by infection of the cell with a pathogen, for example, a viral or bacterial genomic RNA or DNA, a plasmid, a viral or bacterial mRNA, or the like.

The first and second (and/or optional third, fourth, etc.) nucleic acid targets can be part of a single nucleic acid molecule, or they can be separate molecules. Various advantages and applications of both approaches are discussed in greater detail below and in the section entitled “Implementation, applications, and advantages.” In one class of embodiments, the first nucleic acid target is a first mRNA and the second nucleic acid target is a second mRNA. In another class of embodiments, the first nucleic acid target comprises a first region of an mRNA and the second nucleic acid target comprises a second region of the same mRNA; this approach can increase specificity of detection of the mRNA. In another class of embodiments, the first nucleic acid target comprises a first chromosomal DNA polynucleotide sequence and the second nucleic acid target comprises a second chromosomal DNA polynucleotide sequence. The first and second chromosomal DNA polynucleotide sequences are optionally located on the same chromosome, e.g., within the same gene, or on different chromosomes.

The methods permit detection of even low or single copy number targets. Thus, in one class of embodiments, about 1000 copies or less of the first nucleic acid target and/or about 1000 copies or less of the second nucleic acid target are present in the cell (e.g., about 100 copies or less, about 50 copies or less, about 10 copies or less, about 5 copies or less, or even a single copy).

In one aspect, the signal(s) from nucleic acid target(s) are normalized. In one class of embodiments, the second nucleic acid target comprises a reference nucleic acid, and the method includes normalizing the first signal to the second signal. The reference nucleic acid is a nucleic acid selected as a standard of comparison. It will be evident that choice of the reference nucleic acid can depend on the desired application. For example, for gene expression analysis, where the first and optional third, fourth, etc. nucleic acid targets are mRNAs whose expression levels are to be determined, the reference nucleic acid can be an mRNA transcribed from a housekeeping gene. As another example, the first nucleic acid target can be an mRNA whose expression is altered in a pathological state, e.g., an mRNA expressed in a tumor cell and not a normal cell or expressed at a higher level in a tumor cell than in a normal cell, while the second nucleic acid target is an mRNA expressed from a housekeeping gene or similar gene whose expression is not altered in the pathological state. As yet another example, the first nucleic acid target can be a chromosomal DNA sequence that is amplified or deleted in a tumor cell, while the second nucleic acid target is another chromosomal DNA sequence that is maintained at its normal copy number in the tumor cell. Exemplary reference nucleic acids are described herein, and many more are well known in the art.

Optionally, results from the cell are compared with results from a reference cell. That is, the first and second targets are also detected in a reference cell, for example, a non-tumor, uninfected, or other healthy normal cell, chosen as a standard of comparison depending on the desired application. The signals can be normalized to a reference nucleic acid as noted above. As just one example, the first nucleic acid target can be the Her-2 gene, with the goal of measuring Her-2 gene amplification. Signal from Her-2 can be normalized to that from a reference gene, whose copy number is stably maintained in the genomic DNA. The normalized signal for the Her-2 gene from a target cell (e.g., a tumor cell or suspected tumor cell) can be compared to the normalized signal from a reference cell (e.g., a normal cell), to determine copy number in the cancer cell in comparison to normal cells.

The label (first, second, third, etc.) can be essentially any convenient label that directly or indirectly provides a detectable signal. In one aspect, the first label is a first fluorescent label and the second label is a second fluorescent label. Detecting the signal from the labels thus comprises detecting fluorescent signals from the labels. A variety of fluorescent labels whose signals can be distinguished from each other are known, including, e.g., fluorophores and quantum dots. As other examples, the label can be a luminescent label, a light-scattering label (e.g., colloidal gold particles), or an enzyme (e.g., alkaline phosphatase or horseradish peroxidase).

The methods can be used to detect the presence of the nucleic acid targets in cells from essentially any type of sample. For example, the sample can be derived from a bodily fluid, a bodily waste, blood, bone marrow, sputum, urine, lymph node, stool, vaginal secretions, cervical pap smear, oral swab or other swab or smear, spinal fluid, saliva, sputum, ejaculatory fluid, semen, lymph fluid, an intercellular fluid, a tissue (e.g., a tissue homogenate or tissue section), a biopsy, and/or a tumor. The sample and/or the cell can be derived from one or more of a human, an animal, a plant, and a cultured cell. Samples derived from even relatively large volumes of materials such as bodily fluid or bodily waste can be screened in the methods of the invention, and removal of such materials is relatively non-invasive. Samples are optionally taken from a patient, following standard laboratory methods after informed consent.

The methods for detecting nucleic acid targets in cells can be used to identify the cells. For example, a cell can be identified as being of a desired type based on which nucleic acids, and in what levels, it contains. Thus, in one class of embodiments, the methods include identifying the cell as a desired target cell based on detection of the first and second signals (and optional third, fourth, etc. signals) from within the cell. The cell can be identified on the basis of the presence or absence of one or more of the nucleic acid targets. Similarly, the cell can be identified on the basis of the relative signal strength from or expression level of one or more of the nucleic acid targets. Signals are optionally normalized as noted above and/or compared to those from a reference cell.

The methods can be applied to detection and identification of even rare cell types. Thus, the sample including the cell can be a mixture of desired target cells and other, nontarget cells, which can be present in excess of the target cells. For example, the ratio of target cells to cells of all other type(s) in the sample is optionally less than 1:1×10⁴, less than 1:1×10⁵, less than 1:1×10⁶, less than 1:1×10⁷, less than 1:1×10⁸, or even less than 1:1×10⁹.

Essentially any type of cell that can be differentiated based on its nucleic acid content (presence, absence, expression level or copy number of one or more nucleic acids) can be detected and identified using the methods and a suitable choice of nucleic acid targets. As just a few examples, the cell can be a circulating tumor cell or other tumor cell, a virally infected cell, a fetal cell in maternal blood, a bacterial cell or other microorganism in a biological sample (e.g., blood or other body fluid), an endothelial cell, precursor endothelial cell, or myocardial cell in blood, a stem cell, or a T-cell. Rare cell types can be enriched prior to performing the methods, if necessary, by methods known in the art (e.g., lysis of red blood cells, isolation of peripheral blood mononuclear cells, further enrichment of rare target cells through magnetic-activated cell separation (MACS), etc.). The methods are optionally combined with other techniques, such as DAPI staining for nuclear DNA or analysis of cellular morphology. It will be evident that a variety of different types of nucleic acid markers are optionally detected simultaneously by the methods and used to identify the cell. For example, a cell can be identified based on the presence or relative expression level of one nucleic acid target in the cell and the absence of another nucleic acid target from the cell; e.g., a circulating tumor cell can be identified by the presence or level of one or more markers found in the tumor cell and not found (or found at different levels) in blood cells, and its identity can be confirmed by the absence of one or more markers present in blood cells and not circulating tumor cells. The principle may be extended to using any other type of markers such as protein based markers in single cells.

The cell is typically fixed and permeabilized before hybridization of the capture probes, to retain the nucleic acid targets in the cell and to permit the capture probes, label probes, etc. to enter the cell. The cell is optionally washed to remove materials not captured to one of the nucleic acid targets. The cell can be washed after any of various steps, for example, after hybridization of the capture probes to the nucleic acid targets to remove unbound capture probes, after hybridization of the preamplifiers, amplifiers, and/or label probes to the capture probes, and/or the like.

The various capture and hybridization steps can be performed simultaneously or sequentially, in essentially any convenient order. Preferably, a given hybridization step is accomplished for all of the nucleic acid targets at the same time. For example, all the capture probes (first, second, etc.) can be added to the cell at once and permitted to hybridize to their corresponding targets, the cell can be washed, amplifiers (first, second, etc.) can be hybridized to the corresponding capture probes, the cell can be washed, the label probes (first, second, etc.) can be hybridized to the corresponding amplifiers, and the cell can then be washed again prior to detection of the labels. As another example, the capture probes can be hybridized to the targets, the cell can be washed, amplifiers and label probes can be added together and hybridized, and the cell can then be washed prior to detection. It will be evident that double-stranded nucleic acid target(s) are preferably denatured, e.g., by heat, prior to hybridization of the corresponding capture probe(s) to the target(s).

In some embodiments, the cell is in suspension for all or most of the steps of the method, for ease of handling. However, the methods are also applicable to cells in solid tissue samples (e.g., tissue sections) and/or cells immobilized on a substrate (e.g., a slide or other surface). Thus, in one class of embodiments, the cell is in suspension in the sample comprising the cell, and/or the cell is in suspension during the hybridizing, capturing, and/or detecting steps. For example, the cell can be in suspension in the sample and during the hybridization, capture, optional washing, and detection steps. In other embodiments, the cell is in suspension in the sample comprising the cell, and the cell is fixed on a substrate during the hybridizing, capturing, and/or detecting steps. For example, the cell can be in suspension during the hybridization, capture, and optional washing steps and immobilized on a substrate during the detection step. In other embodiments, the sample comprises a tissue section.

Signals from the labels can be detected, and their intensities optionally measured, by any of a variety of techniques well known in the art. For example, in embodiments in which the cell is in suspension, the first and second (and optional third, etc.) signals can be conveniently detected by flow cytometry. In embodiments in which cells are immobilized on a substrate, the first and second (and optional third etc.) signals can be detected, for example, by laser scanner or microscope, e.g., a fluorescent or automated scanning microscope. As noted, detection is at the level of individual, single cells. Signals from the labels are typically detected in a single operation (e.g., a single flow cytometry run or a single microscopy or scanning session), rather than sequentially in separate operations for each label. Such a single detection operation can, for example, involve changing optical filters between detection of the different labels, but it does not involve detection of the first label followed by capture of the second label and then detection of the second label. In some embodiments, the first and second (and optional third etc.) labels are captured to their respective targets simultaneously but are detected in separate detection steps or operations.

Additional features described herein, e.g., in the section below entitled “Implementation, applications, and advantages,” can be applied to the methods, as relevant. For example, as described in greater detail below, a label probe can include more than one label, identical or distinct. Signal strength is optionally adjusted between targets depending on their expected copy numbers, if desired; for example, the signal for an mRNA expressed at low levels can be amplified to a greater degree (e.g., by use of more labels per label probe and/or use of preamplifiers and amplifiers to capture more label probes per copy of the target) than the signal for a highly expressed mRNA.

In another aspect of the invention, two or more nucleic acids are detected by PCR amplification of the nucleic acids in situ in individual cells. To prevent leakage of the resulting amplicons out of the cells, a water-oil emulsion can be made as mentioned in Li et al. (2006) “BEAMing up for detection and quantification of rare sequence variants” Nature Methods 3(2):95-7 that separates single cells into different compartments.

Detection of Relative Levels by Normalization to Reference Nucleic Acids

As discussed briefly above, the signal detected for a nucleic acid of interest can be normalized to that of a standard, reference nucleic acid. One general class of embodiments thus provides methods of assaying a relative level of one or more target nucleic acids in an individual cell. In the methods, a sample comprising the cell is provided. The cell comprises or is suspected of comprising a first, target nucleic acid, and it comprises a second, reference nucleic acid. A first label probe comprising a first label and a second label probe comprising a second label, wherein a first signal from the first label is distinguishable from a second signal from the second label, are also provided. In the cell, the first label probe is captured to the first, target nucleic acid (when the first, target nucleic acid is present in the cell) and the second label probe is captured to the second, reference nucleic acid. The first signal from the first label and the second signal from the second label are then detected in the individual cell, and the intensity of each signal is measured. The intensity of the first signal is normalized to the intensity of the second (reference) signal. The level of the first, target nucleic acid relative to the level of the second, reference nucleic acid in the cell is thereby assayed, since the first and second labels are associated with their respective nucleic acids. The methods are optionally quantitative, permitting measurement of the amount of the first, target nucleic acid relative to the amount of the second, reference nucleic acid in the cell. Thus, the intensity of the first signal normalized to that of the second signal can be correlated with a quantity of the first, target nucleic acid present in the cell.

The label probes can bind directly to the nucleic acids. For example, the first label probe can hybridize to the first, target nucleic acid and/or the second label probe can hybridize to the second, reference nucleic acid. Alternatively, some or all of the label probes can be indirectly bound to their corresponding nucleic acids, e.g., through capture probes. For example, the first and second label probes can bind directly to the nucleic acids, or one can bind directly while the other binds indirectly, or both can bind indirectly.

The label probes are optionally captured to the nucleic acids via capture probes. In one class of embodiments, at least a first capture probe and at least a second capture probe are provided. In the cell, the first capture probe is hybridized to the first, target nucleic acid and the second capture probe is hybridized to the second, reference nucleic acid. The first label probe is captured to the first capture probe and the second label probe is captured to the second capture probe, thereby capturing the first label probe to the first, target nucleic acid and the second label probe to the second, reference nucleic acid. The features described for the methods above apply to these embodiments as well, with respect to configuration and number of the label and capture probes, optional use of preamplifiers and/or amplifiers, rolling circle amplification of circular polynucleotides, and the like.

The methods can be used for multiplex detection of nucleic acids, including simultaneous detection of two or more target nucleic acids. Thus, the cell optionally comprises or is suspected of comprising a third, target nucleic acid, and the methods optionally include: providing a third label probe comprising a third label, wherein a third signal from the third label is distinguishable from the first and second signals; capturing, in the cell, the third label probe to the third, target nucleic acid (when present in the cell); detecting the third signal from the third label, which detecting comprises measuring an intensity of the third signal; and normalizing the intensity of the third signal to the intensity of the second signal. Alternatively, the third signal can be normalized to that from a different reference nucleic acid. Fourth, fifth, sixth, etc. nucleic acids are similarly simultaneously detected in the cell if desired. The third, fourth, fifth, etc. label probes are optionally hybridized directly to their corresponding nucleic acid, or they can be captured indirectly via capture probes as described for the first and second label probes.

The methods can be used for gene expression analysis, detection of gene amplification or deletion, or detection or diagnosis of disease, as just a few examples. A target nucleic acid can be essentially any nucleic acid that is desirably detected in the cell. For example, a target nucleic acid can be a DNA, a chromosomal DNA, an RNA, an mRNA, a microRNA, a ribosomal RNA, or the like. The target nucleic acid can be a nucleic acid endogenous to the cell, or as another example, the target can be a nucleic acid introduced to or expressed in the cell by infection of the cell with a pathogen, for example, a viral or bacterial genomic RNA or DNA, a plasmid, a viral or bacterial mRNA, or the like. The reference nucleic acid can similarly be a DNA, an mRNA, a chromosomal DNA, an mRNA, an RNA endogenous to the cell, or the like.

As described above, choice of the reference nucleic acid can depend on the desired application. For example, for gene expression analysis, where the first and optional third, fourth, etc. target nucleic acids are mRNAs whose expression levels are to be determined, the reference nucleic acid can be an mRNA transcribed from a housekeeping gene. As another example, the first, target nucleic acid can be an mRNA whose expression is altered in a pathological state, e.g., an mRNA expressed in a tumor cell and not a normal cell or expressed at a higher level in a tumor cell than in a normal cell, while the reference nucleic acid is an mRNA expressed from a housekeeping gene or similar gene whose expression is not altered in the pathological state. In a similar example, the target nucleic acid can be a viral or bacterial nucleic acid while the reference nucleic acid is endogenous to the cell. As yet another example, the first, target nucleic acid can be a chromosomal DNA sequence that is amplified or deleted in a tumor cell, while the reference nucleic acid is another chromosomal DNA sequence that is maintained at its normal copy number in the tumor cell. Exemplary reference nucleic acids are described herein, and many more are well known in the art.

In one class of embodiments, the first, target nucleic acid is a first mRNA and the second, reference nucleic acid is a second mRNA. In another class of embodiments, the first, target nucleic acid comprises a first chromosomal DNA polynucleotide sequence and the second, reference nucleic acid comprises a second chromosomal DNA polynucleotide sequence. The first and second chromosomal DNA polynucleotide sequences are optionally located on the same chromosome or on different chromosomes.

Optionally, normalized results from the cell are compared with normalized results from a reference cell. That is, the target and reference nucleic acids are also detected in a reference cell, for example, a non-tumor, uninfected, or other healthy normal cell, chosen as a standard of comparison depending on the desired application. As just one example, the first, target nucleic acid can be the Her-2 gene, with the goal of measuring Her-2 gene amplification. Signal from Her-2 can be normalized to that from a reference gene whose copy number is stably maintained in the genomic DNA. The normalized signal for the Her-2 gene from a target cell (e.g., a tumor cell or suspected tumor cell) can be compared to the normalized signal from a reference cell (e.g., a normal cell), to determine copy number in the cancer cell in comparison to normal cells.

Signal strength is optionally adjusted between the target and reference nucleic acids depending on their expected copy numbers, if desired. For example, the signal for a target mRNA expressed at low levels can be amplified to a greater degree (e.g., by use of more labels per label probe and/or use of capture probes, preamplifiers and amplifiers to capture more label probes per copy of the target) than the signal for a highly expressed mRNA (which can, e.g., be detected by direct binding of the label probe to the reference nucleic acid, by use of capture probes and amplifier without a preamplifier, or the like).

The methods for assaying relative levels of target nucleic acids in cells can be used to identify the cells. For example, a cell can be identified as being of a desired type based on which nucleic acids, and in what levels, it contains. Thus, in one class of embodiments, the methods include identifying the cell as a desired target cell based on the normalized first signal (and optional normalized third, fourth, etc. signals). As described herein, the cell can be identified on the basis of the presence or absence of one or more of the target nucleic acids. Similarly, the cell can be identified on the basis of the relative signal strength from or expression level of one or more target nucleic acids. Signals are optionally compared to those from a reference cell.

The methods can be applied to detection and identification of even rare cell types. Thus, the sample including the cell can be a mixture of desired target cells and other, nontarget cells, which can be present in excess of the target cells. For example, the ratio of target cells to cells of all other type(s) in the sample is optionally less than 1:1×10⁴, less than 1:1×10⁵, less than 1:1×10⁶, less than 1:1×10⁷, less than 1:1×10⁸, or even less than 1:1×10⁹.

Essentially any type of cell that can be differentiated based on its nucleic acid content (presence, absence, or copy number of one or more nucleic acids) can be detected and identified using the methods and a suitable choice of target and reference nucleic acids. As just a few examples, the cell can be a circulating tumor cell or other tumor cell, a virally infected cell, a fetal cell in maternal blood, a bacterial cell or other microorganism in a biological sample (e.g., blood or other body fluid), or an endothelial cell, precursor endothelial cell, or myocardial cell in blood. Rare cell types can be enriched prior to performing the methods, if necessary, by methods known in the art (e.g., lysis of red blood cells, isolation of peripheral blood mononuclear cells, etc.). The methods are optionally combined with other techniques, such as DAPI staining for nuclear DNA. It will be evident that a variety of different types of nucleic acid markers are optionally detected simultaneously by the methods and used to identify the cell. For example, a cell can be identified based on the presence or relative expression level of one target nucleic acid in the cell and the absence of another target nucleic acid from the cell; e.g., a circulating tumor cell can be identified by the presence or level of one or more markers found in the tumor cell and not found (or found at different levels) in blood cells, and by the absence of one or more markers present in blood cells and not circulating tumor cells. The principle may be extended to using any other type of markers such as protein based markers in single cells.

Essentially all of the features noted for the methods above apply to these embodiments as well, as relevant; for example, with respect to source of sample, fixation and permeabilization of the cell, washing the cell, denaturation of double-stranded target and reference nucleic acids, type of labels, use of optional blocking probes, detection of signals, detection (and intensity measurement or spot counting) by flow cytometry or microscopy, presence of the cell in suspension, immobilized on a substrate, or in a tissue, and/or the like. Also, additional features described herein, e.g., in the section entitled “Implementation, applications, and advantages,” can be applied to the methods, as relevant.

The methods of the invention can be used for gene expression analysis in single cells. Currently, gene expression analysis deals with heterogeneous cell populations such as blood or tumor specimens. Blood contains various subtypes of leukocytes, and when changes in gene expression of whole blood or RNA isolated from blood are measured, it is not known what subtype of blood cells actually changed their gene expression. It is possible that gene expression of only a certain subtype of blood cells is affected in a disease state or by drug treatment, for example. Technology that can measure gene expression in single cells, so changes of gene expression in single cells can be examined, is thus desirable. Similarly, a tumor specimen contains a heterogeneous cell population including tumor cells, normal cells, stromal cells, immune cells, etc. Current technology looks at the sum of the expression of all those cells through total RNA or cell lysate. However, the overall expression change may not be representative of that in target tumor cells. So again, it would be useful to look at the expression changes in single cells so that the target tumor cells can be examined specifically, to see how the target cells change in gene expression and how they respond to drug treatment, for example.

In one aspect, the present invention provides methods for gene expression analysis in single cells. Single cell gene expression analysis can be accomplished by measuring expression of a target gene and normalizing against the expression of a housekeeping gene, as described above. As just a couple of examples, the normalized expression in a disease state can be compared to that in the normal state, or the expression in a drug treated state can be compared to that in the normal state. The change of expression level in single cells may have biological significance indicating disease progression, drug therapeutic efficacy and/or toxicity, tumor staging and classification, etc.

Accordingly, one general class of embodiments provides methods of performing comparative gene expression analysis in single cells. In the methods, a first mixed cell population comprising one or more cells of a specified type is provided. A second mixed cell population comprising one or more cells of the specified type is also provided. An expression level of one or more target nucleic acids relative to a reference nucleic acid is measured in the cells of the specified type of the first population, to provide a first expression profile. An expression level of the one or more target nucleic acids relative to the reference nucleic acid is measured in the cells of the specified type of the second population, to provide a second expression profile. The first and second expression profiles are compared.

In one class of embodiments, the one or more target nucleic acids are one or more mRNAs, e.g., two or more, three or more, four or more, etc. mRNAs. The expression level of each mRNA can be determined relative to that of a housekeeping gene whose mRNA serves as the reference nucleic acid.

The first and/or second mixed cell population contains at least one other type of cell in addition to the specified type, more typically at least two or more other types of cells, and optionally several to many other types of cells (e.g., as is found in whole blood, a tumor, or other complex biological sample). The ratio of cells of the specified type to cells of all other type(s) in the first or second mixed cell population is optionally less than 1:1×10⁴, less than 1:1×10⁵, less than 1:1×10⁶, less than 1:1×10⁷, less than 1:1×10⁸, or even less than 1:1×10⁹.

As will be evident, a change in gene expression profile between the two populations may indicate a disease state or progression, a drug response, a therapeutic efficacy, etc. Thus, for example, the first mixed cell population can be from a patient who has been diagnosed or who is to be diagnosed with a particular disease or disorder, while the second mixed population is from a healthy individual. Similarly, the first and second mixed populations can be from a single individual but taken at different time points, for example, to follow disease progression or to assess response to drug treatment. Accordingly, the first mixed cell population can be taken from an individual (e.g., a human) before treatment is initiated with a drug or other compound, while the second population is taken at a specified time after treatment is initiated. As another example, the first mixed population can be from a treated individual while the second mixed population is from an untreated individual.

Essentially all of the features noted for the methods above apply to these embodiments as well, as relevant; for example, with respect to type of target and reference nucleic acids, cell type, source of sample, fixation and permeabilization of the cell, washing the cell, denaturation of double-stranded target and reference nucleic acids, type of labels, use and configuration of label probes, capture probes, preamplifiers and/or amplifiers, use of optional blocking probes, detection of signals, detection (and intensity measurement or spot counting) by flow cytometry or microscopy, presence of the cell in suspension, immobilized on a substrate, or in a tissue, and/or the like. Exemplary target and reference nucleic acids are described herein.

In another aspect, the methods can be used to compare copy number in single cells from a first population (e.g., tumor cells) with copy number in single cells from a second population (e.g., normal cells used as a reference). The nucleic acid target(s) can be transcripts or genomic DNA, where, for example, the degree of amplification or deletion of genes such as her-2 can correlate with tumor progression. In another aspect, the methods can be applied to gene expression analysis in single cells in even a single population, including, for example, cells of the same type but at different stages of the cell cycle.

Label Density

The methods of the invention permit far more labels to be captured to small regions of target nucleic acids than do currently existing techniques. For example, standard FISH techniques typically use probes that cover 20 kb or more, and a probe typically has fluorophores chemically conjugated at a density of approximately one fluorescent molecule per seven nucleotides of the probe. When molecular beacon target detection is employed, one label pair is captured to the target in the region covered by the beacon, typically about 40 nucleotides. For additional discussion of exemplary current techniques, see, e.g., U.S. patent application publications 2004/0091880 and 2005/0181463, U.S. Pat. No. 6,645,731, and international patent application publications WO 95/09245 and 03/019141.

Methods described herein, in comparison, readily permit capture of hundreds of labels (e.g., 400 or more) to the region of the target covered by a single capture probe, e.g., 20-25 nucleotides or more. The theoretical degree of amplification achieved from a single capture probe is readily calculated for any given configuration of capture probes, amplifiers, etc; for example, the theoretical degree of amplification achieved from a single capture probe, and thus the number of labels per length in nucleotides of the capture probe, can be equal to the number of preamplifiers bound to the capture probe times the number of amplifiers that bind each preamplifier times the number of label probes that bind each preamplifier times the number of labels per label probe.

Thus, in one aspect, the invention provides methods that facilitate association of a high density of labels to target nucleic acids in cells. One general class of embodiments provides methods of detecting two or more nucleic acid targets in an individual cell. In the methods, a sample comprising the cell is provided. The cell comprises or is suspected of comprising a first nucleic acid target and a second nucleic acid target. In the cell, a first label is captured to the first nucleic acid target (when present in the cell) and a second label is captured to the second nucleic acid target (when present in the cell). A first signal from the first label is distinguishable from a second signal from the second label. As noted, the labels are captured at high density. Thus, an average of at least one copy of the first label per nucleotide of the first nucleic acid target is captured to the first nucleic acid target over a region that spans at least 20 contiguous nucleotides of the first nucleic acid target, and an average of at least one copy of the second label per nucleotide of the second nucleic acid target is captured to the second nucleic acid target over a region that spans at least 20 contiguous nucleotides of the second nucleic acid target. The first signal from the first label and the second signal from the second label are detected.

In one class of embodiments, an average of at least four, eight, or twelve copies of the first label per nucleotide of the first nucleic acid target are captured to the first nucleic acid target over a region that spans at least 20 contiguous nucleotides of the first nucleic acid target, and an average of at least four, eight, or twelve copies of the second label per nucleotide of the second nucleic acid target are captured to the second nucleic acid target over a region that spans at least 20 contiguous nucleotides of the second nucleic acid target. In one embodiment, an average of at least sixteen copies of the first label per nucleotide of the first nucleic acid target are captured to the first nucleic acid target over a region that spans at least 20 contiguous nucleotides of the first nucleic acid target, and an average of at least sixteen copies of the second label per nucleotide of the second nucleic acid target are captured to the second nucleic acid target over a region that spans at least 20 contiguous nucleotides of the second nucleic acid target.

Essentially all of the features noted for the methods above apply to these embodiments as well, as relevant, for example, with respect to type of labels, detection of signals, type, treatment, and suspension of the cell, and/or the like. The regions of the first and second nucleic acid targets optionally span at least 25, 50, 100, 200, or more contiguous nucleotides and/or at most 2000, 1000, 500, 200, 100, 50, or fewer nucleotides. A like density of third, fourth, fifth, sixth, etc. labels is optionally present for (e.g., captured to) third, fourth, fifth, sixth, etc. nucleic acid targets.

If the target is short, conventional FISH (or other direct label in situ methods) can not attain sufficient signal to achieve detection of the target. The methods described herein, however, enable in situ, high sensitivity detection of even short targets (e.g., a short nucleic acid molecule or a short region of polynucleotide sequence within a longer nucleic acid molecule), including, e.g., target sections of longer sequences and target molecules less than 1 kb. Accordingly, one general class of embodiments provides methods of detecting one or more nucleic acid targets in an individual cell that include: providing a sample comprising the cell, which cell comprises or is suspected of comprising a first nucleic acid target; providing a first label probe comprising a first label; providing a set of one or more first capture probes; hybridizing, in the cell, the first capture probes to the first nucleic acid target, when present in the cell, wherein the set of first capture probes hybridizes to a region of the first nucleic acid target (including, e.g., the entire target molecule or a portion thereof) that is 1000 nucleotides or less in length (e.g., 500 nucleotides or less in length); capturing the first label probe to the first capture probes, thereby capturing the first label probe to the first nucleic acid target; and detecting a first signal from the first label. For example, the set of first capture probes can hybridize to a region of the first nucleic acid target that is 200 nucleotides or less in length, 100 nucleotides or less in length, 50 nucleotides or less in length, or even 25 nucleotides or less in length, thus permitting detection of target nucleic acids as small as microRNAs, for example. Other exemplary targets include, but are not limited to, short or short regions of DNAs, chromosomal DNAs, RNAs, mRNAs, and ribosomal RNAs.

As for the embodiments above, the methods are useful for multiplex detection of nucleic acids, including simultaneous detection of two or more nucleic acid targets (e.g., short targets, or a combination of short and longer targets). Thus, the cell optionally comprises or is suspected of comprising a second nucleic acid target, and the methods optionally include: providing a second label probe comprising a second label, wherein a second signal from the second label is distinguishable from the first signal, providing a set of one or more second capture probes, hybridizing in the cell the second capture probes to the second nucleic acid target, when present in the cell, capturing the second label probe to the second capture probes, and detecting the second signal from the second label. Third, fourth, fifth, sixth, etc. nucleic acid targets are similarly simultaneously detected in the cell if desired. Each hybridization or capture step is preferably accomplished for all of the nucleic acid targets at the same time.

Essentially all of the features noted for the methods above apply to these embodiments as well, as relevant; for example, with respect to type of nucleic acid targets, copy number, cell type, source of sample, fixation and permeabilization of the cell, washing the cell, denaturation of double-stranded nucleic acids, type of labels, use and configuration of label probes, capture probes, preamplifiers and/or amplifiers (including, e.g., hybridization of two capture probes to a single label probe, preamplifier, or amplifier molecule), use of optional blocking probes, detection of signals, detection (and intensity measurement) of signals from the individual cell by flow cytometry or microscopy, presence of the cell in suspension, immobilized on a substrate, or in a tissue, and/or the like.

Detection of Target Cells

As described above, cells can be detected and identified by detecting their constituent nucleic acids. For certain applications, for example, detection of rare cells from large heterogeneous mixtures of cells, detection of multiple, redundant nucleic acid markers in order to detect the rare cell is advantageous. The following hypothetical example illustrates one advantage of detecting redundant markers.

Say that circulating tumor cells (CTC) are to be detected from a blood sample in which the CTC concentration is one in 10⁶ normal white blood cells. If a single nucleic acid marker for the CTC (e.g., a nucleic acid whose presence or copy number can uniquely and sufficiently distinguish the cell from the rest of the cell population) has a detection specificity of 1 in 10³, 1000 cells will be mistakenly identified as “CTC” when 10⁶ cells are counted. (Such false positives can result from random background signal generated by nonspecific binding of the relevant probe(s) or from similar factors.) If an additional independent marker is included which, on its own, also has a detection specificity of 1 in 10³, and if a cell is identified as a CTC only if both markers are positive, the combined detection specificity is now theoretically dramatically increased, to 1 in 10³×10³=10⁶. This specificity is sufficient for direct CTC detection in normal white blood cells under these assumptions. Similarly, if three independent redundant markers are used for identification of CTC, the detection specificity can be boosted to 1 in 10⁹. Use of two or more redundant markers thus reduces the number of false positives and facilitates detection of even rare cells from complex samples.

Accordingly, one general class of embodiments provides methods of detecting an individual cell of a specified type. In the methods, a sample comprising a mixture of cell types including at least one cell of the specified type is provided. A first label probe comprising a first label and a second label probe comprising a second label, wherein a first signal from the first label is distinguishable from a second signal from the second label, are provided. In the cell, the first label probe is captured to a first nucleic acid target (when the first nucleic acid target is present in the cell) and the second label probe is captured to a second nucleic acid target (when the second nucleic acid target is present in the cell). The first signal from the first label and the second signal from the second label are detected and correlated with the presence, absence, or amount of the corresponding, first and second nucleic acid targets in the cell. The cell is identified as being of the specified type based on detection of the presence, absence, or amount (e.g., a non-zero amount) of both the first and second nucleic acid targets within the cell, where the specified type of cell is distinguishable from the other cell type(s) in the mixture on the basis of either the presence, absence, or amount of the first nucleic acid target or the presence, absence, or amount of the second nucleic acid target in the cell (that is, the nucleic acid targets are redundant markers for the specified cell type). An intensity of the first signal and an intensity of the second signal are optionally measured and correlated with a quantity of the corresponding nucleic acid present in the cell. As another example, a signal spot can be counted for each copy of the first and second nucleic acid targets to quantitate them, as described in greater detail below.

Each nucleic acid target that serves as a marker for the specified cell type can distinguish the cell type by its presence in the cell, by its amount (copy number, e.g., its genomic copy number or its transcript expression level), or by its absence from the cell (a negative marker). A set of nucleic acid targets can include different types of such markers; that is, one nucleic acid target can serve as a positive marker, distinguishing the cell by its presence or non-zero amount in the cell, while another serves as a negative marker, distinguishing the cell by its absence from the cell. For example, in one class of embodiments, the cell comprises a first nucleic acid target and a second nucleic acid target, and the cell is identified as being of the specified type based on detection of the presence or amount of both the first and second nucleic acid targets within the cell, where the specified type of cell is distinguishable from the other cell type(s) in the mixture on the basis of either the presence or amount of the first nucleic acid target or the presence or amount of the second nucleic acid target in the cell.

The label probes can bind directly to the nucleic acid targets. For example, the first label probe can hybridize to the first nucleic acid target and/or the second label probe can hybridize to the second nucleic acid target. Alternatively, some or all of the label probes can be indirectly bound to their corresponding nucleic acid targets, e.g., through capture probes. For example, the first and second label probes can bind directly to the nucleic acid targets, or one can bind directly while the other binds indirectly, or both can bind indirectly.

The label probes are optionally captured to the nucleic acid targets via capture probes. In one class of embodiments, at least a first capture probe and at least a second capture probe are provided. In the cell, the first capture probe is hybridized to the first nucleic acid target and the second capture probe is hybridized to the second nucleic acid target. The first label probe is captured to the first capture probe and the second label probe is captured to the second capture probe, thereby capturing the first label probe to the first nucleic acid target and the second label probe to the second nucleic acid target. The features described for the methods above apply to these embodiments as well, with respect to configuration and number of the label and capture probes, optional use of preamplifiers and/or amplifiers, rolling circle amplification of circular polynucleotides, and the like.

Third, fourth, fifth, etc. nucleic acid targets are optionally detected in the cell. For example, the method optionally includes: providing a third label probe comprising a third label, wherein a third signal from the third label is distinguishable from the first and second signals, capturing in the cell the third label probe to a third nucleic acid target (when present in the cell), and detecting the third signal from the third label. The third, fourth, fifth, etc. label probes are optionally hybridized directly to their corresponding nucleic acid, or they can be captured indirectly via capture probes as described for the first and second label probes.

The additional markers can be used in any of a variety of ways. For example, the cell can comprise the third nucleic acid target, and the first and/or second signal can be normalized to the third signal. The methods can include identifying the cell as being of the specified type based on the normalized first and/or second signal, e.g., in embodiments in which the target cell type is distinguishable from the other cell type(s) in the mixture based on the copy number of the first and/or second nucleic acid targets, rather than purely on their presence in the target cell type and not in the other cell type(s). Examples include cells detectable based on a pattern of differential gene expression, CTC or other tumor cells detectable by overexpression of one or more specific mRNAs, and CTC or other tumor cells detectable by amplification or deletion of one or more specific chromosomal regions.

As another example, the third nucleic acid target can serve as a third redundant marker for the target cell type, e.g., to improve specificity of the assay for the desired cell type. Thus, in one class of embodiments, the methods include correlating the third signal detected from the cell with the presence, absence, or amount of the third nucleic acid target in the cell, and identifying the cell as being of the specified type based on detection of the presence, absence, or amount of the first, second, and third nucleic acid targets within the cell, wherein the specified type of cell is distinguishable from the other cell type(s) in the mixture on the basis of either presence, absence, or amount of the first nucleic acid target, presence, absence, or amount of the second nucleic acid target, or presence, absence, or amount of the third nucleic acid target in the cell.

As yet another example, the additional markers can assist in identifying the cell type. For example, the presence, absence, or amount of the first and third markers may suffice to identify the cell type, as could the presence, absence, or amount of the second and fourth markers; all four markers could be detected to provide two redundant sets of markers and therefore increased specificity of detection. As another example, one or more additional markers can be used in negative selection against undesired cell types; for example, identity of a cell as a CTC can be further verified by the absence from the cell of one or more markers present in blood cells and not circulating tumor cells.

Detection of additional nucleic acid targets can also provide further information useful in diagnosis, outcome prediction or the like, regardless of whether the targets serve as markers for the particular cell type. For example, additional nucleic acid targets can include markers for proliferating potential, apoptosis, or other metastatic, genetic, or epigenetic changes.

Signals from the additional targets are optionally normalized to a reference nucleic acid as described above. Signal strength is optionally adjusted between targets depending on their expected copy numbers, if desired. Signals from the target nucleic acids in the cell are optionally compared to those from a reference cell, as noted above.

A nucleic acid target can be essentially any nucleic acid that is desirably detected in the cell. For example, a nucleic acid target can be a DNA, a chromosomal DNA, an RNA, an mRNA, a microRNA, a ribosomal RNA, or the like. The nucleic acid target can be a nucleic acid endogenous to the cell. As another example, the target can be a nucleic acid introduced to or expressed in the cell by infection of the cell with a pathogen, for example, a viral or bacterial genomic RNA or DNA, a plasmid, a viral or bacterial mRNA, or the like.

The first and second (and/or optional third, fourth, etc.) nucleic acid targets can be part of a single nucleic acid molecule, or they can be separate molecules. Various advantages and applications of both approaches are discussed in greater detail below, e.g., in the section entitled “Implementation, applications, and advantages.” In one class of embodiments, the first nucleic acid target is a first mRNA and the second nucleic acid target is a second mRNA. In another class of embodiments, the first nucleic acid target comprises a first region of an mRNA and the second nucleic acid target comprises a second region of the same mRNA. In another class of embodiments, the first nucleic acid target comprises a first chromosomal DNA polynucleotide sequence and the second nucleic acid target comprises a second chromosomal DNA polynucleotide sequence. The first and second chromosomal DNA polynucleotide sequences are optionally located on the same chromosome, e.g., within the same gene, or on different chromosomes.

The methods can be applied to detection and identification of even rare cell types. For example, the ratio of cells of the specified type to cells of all other type(s) in the mixture is optionally less than 1:1×10⁴, less than 1:1×10⁵, less than 1:1×10⁶, less than 1:1×10⁷, less than 1:1×10⁸, or even less than 1:1×10⁹.

Essentially any type of cell that can be differentiated based on suitable markers (or redundant regions of a single marker, e.g., a single mRNA or amplified/deleted chromosomal region) can be detected and identified using the methods. As just a few examples, the cell can be a circulating tumor cell or other tumor cell, a virally infected cell, a fetal cell in maternal blood, a bacterial cell or other microorganism in a biological sample (e.g., blood or other body fluid), an endothelial cell, precursor endothelial cell, or myocardial cell in blood, stem cell, or T-cell. Rare cell types can be enriched prior to performing the methods, if necessary, by methods known in the art (e.g., lysis of red blood cells, isolation of peripheral blood mononuclear cells, etc.).

Essentially all of the features noted for the methods above apply to these embodiments as well, as relevant; for example, with respect to source of sample, fixation and permeabilization of the cell, washing the cell, denaturation of double-stranded nucleic acids, type of labels, use of optional blocking probes, detection of signals, detection (and intensity measurement or spot counting) of signals from the individual cell by flow cytometry or microscopy, presence of the cell in suspension, immobilized on a substrate, or in a tissue, and/or the like. Also, additional features described herein, e.g., in the section entitled “Implementation, applications, and advantages,” can be applied to the methods, as relevant.

In another aspect, detection of individual cells of a specified type is performed as described above, but the first and second nucleic acid targets need not be redundant markers for that cell type. The nucleic acid targets can be essentially any desired nucleic acids, including, for example, redundant and/or non-redundant markers for the cell type.

Detection of Nucleic Acids in Cells in Suspension

Another aspect of the invention provides methods for detection of nucleic acids in cells in suspension, for example, rapid detection by flow cytometry. Accordingly, one general class of embodiments provides methods of detecting one or more nucleic acid targets in an individual cell that include: providing a sample comprising the cell, which cell comprises or is suspected of comprising a first nucleic acid target; providing a first label probe comprising a first label; providing at least a first capture probe; hybridizing, in the cell, the first capture probe to the first nucleic acid target, when present in the cell; capturing the first label probe to the first capture probe, thereby capturing the first label probe to the first nucleic acid target; and detecting, while the cell is in suspension, a first signal from the first label. For example, the signal can be conveniently detected by performing flow cytometry.

The methods are useful for multiplex detection of nucleic acids, including simultaneous detection of two or more nucleic acid targets. Thus, the cell optionally comprises or is suspected of comprising a second nucleic acid target, and the methods optionally include: providing a second label probe comprising a second label, wherein a second signal from the second label is distinguishable from the first signal, providing at least a second capture probe, hybridizing in the cell the second capture probe to the second nucleic acid target, when present in the cell, capturing the second label probe to the second capture probe, and detecting the second signal from the second label. Third, fourth, fifth, sixth, etc. nucleic acid targets are similarly simultaneously detected in the cell if desired. Each hybridization or capture step is preferably accomplished for all of the nucleic acid targets at the same time.

The methods permit detection of even low or single copy number targets. Thus, in one class of embodiments, about 1000 copies or less of the first nucleic acid target are present in the cell (e.g., about 100 copies or less, about 50 copies or less, about 10 copies or less, about 5 copies or less, or even a single copy).

Essentially all of the features noted for the methods above apply to these embodiments as well, as relevant; for example, with respect to type of nucleic acid targets, cell type, source of sample, fixation and permeabilization of the cell, washing the cell, denaturation of double-stranded nucleic acids, type of labels, use and configuration of label probes, capture probes, preamplifiers and/or amplifiers (including, e.g., hybridization of two capture probes to a single label probe, preamplifier, or amplifier molecule), use of optional blocking probes, detection of signals, detection (and intensity measurement) of signals from the individual cell by flow cytometry or microscopy, presence of the cell in suspension or immobilized on a substrate, and/or the like.

Quantifying mRNA in Individual Cells Through Imaging and Spot Counting

In existing DNA FISH assays, the copy numbers of a target DNA sequence are usually visualized and counted on a “one spot per locus” basis either manually or using imaging processing software. However, it has been difficult to employ the same approach to quantify the copy number of mRNA transcripts in individual cells because mRNA, usually around 1000 nucleotides in length, is much shorter than the length of probes required to detect DNA (100,000 nucleotides). This leads to difficulty in the visualization of single RNA molecules. Most existing labeling methodologies cannot attach enough fluorescent label molecules onto an mRNA to generate sufficient signal intensity to visualize a single RNA molecule. Certain aspects of the invention described herein, however, employ a probe set system comprising preamplifiers and amplifiers, which significantly increases the number of label molecules that can be attached to a single RNA molecule and enables it to be observed using a normal microscope. Because an RNA molecule is so small in size, it produces a diffraction-limited spot, which is sharp and well-rounded and can be distinguished from background spots by its unique spatial features. In addition, some aspects of the invention employ a “cooperative hybridization” capture probe design that effectively reduces background noise caused by non-specific hybridization. The combination of these two factors means each copy of an RNA can be observed under an normal microscope as a sharp, bright spot clearly distinguishable from surrounding background. (See, e.g., Example 1 hereinbelow.) This enables truly reliable quantification of RNA copy number, of even endogenous RNAs, by spot counting either manually or automatically utilizing simple image processing software. Since capture probes can be designed against essentially any RNA, even endogenous RNAs can be quantitated, without need for creation of recombinant reporter constructs that include repetitive probe binding sites. For diagnostic applications in particular, since most human genes express less than 50 copies of their RNA per cell, spot counting is an effective and useful tool for the quantification of gene expression level. While the techniques are particularly useful for quantitating RNA in situ, as discussed in greater detail below they can also be applied to RNA that is not inside any cell.

One general class of embodiments provides methods of quantitating a target nucleic acid (e.g., an RNA). In the methods, a sample comprising one or more copies of the target nucleic acid is provided. Typically, the target nucleic acid is endogenous to a cell. A plurality of copies of an optically detectable label are captured to each of the one or more copies of the target nucleic acid (e.g., a fluorescent label or an enzyme that is optically detectable, e.g., with fast red substrate). The copies of the label are optically detected. An optical signal focus (or, equivalently, punctum, spot, or dot) is observable for each of the one or more copies of the target nucleic acid, and the one or more resulting foci are counted, thereby quantitating the target nucleic acid.

As noted, the target nucleic acid can be an RNA, e.g., an mRNA, a microRNA, a ribosomal RNA, or the like. The methods can be applied, e.g., to RNA in situ in a cell or free of any cell. Thus, in one class of embodiments, the sample comprises a cell lysate or other solution comprising the RNA. In another class of embodiments, the sample comprises the cell to which the target RNA is endogenous, and the capturing, detecting, and counting steps are performed in the cell. Optionally, the RNA is located in the cytoplasm of the cell.

The methods are particularly useful for quantitation of low abundance nucleic acids (e.g., RNAs). Thus, in one embodiment, about 100 copies or less of the target nucleic acid are present in the cell, cell lysate, etc., for example, about 10 copies or less, about 5 copies or less, or even a single copy. As noted, a large number of labels are captured to each molecule. For example, at least about 400 copies of the label can be captured to each of the one or more copies of the target nucleic acid, e.g., at least about 1000 copies, at least about 2000 copies, at least about 4000 copies, or at least about 8000 copies. The label can be, e.g., a fluorescent label or an enzyme (e.g., an enzyme optically detectable using a fluorogenic or chromogenic substrate, e.g., fast red).

The label can be captured to the nucleic acid directly or indirectly. Optionally, the label is provided by providing one or more copies of a label probe, the label probe comprising one or more copies of the label. The label probe can be hybridized directly to the target nucleic acid. Preferably, however, the label probe is indirectly captured, e.g., by providing one or more capture probes, hybridizing a copy of each of the one or more capture probes to each of the one or more copies of the target nucleic acid, and capturing the one or more copies of the label probe to the one or more capture probes. As for the embodiments above, the label probe can bind directly to the capture probe, or more typically an amplifier or a preamplifier and amplifier serve as intermediates. Optionally, two or more capture probes bind each label probe, amplifier, or preamplifier.

Essentially all of the features noted for the methods above apply to these embodiments as well, as relevant; for example, with respect to cell type, type of target (including size), source of sample, fixation and permeabilization of the cell, washing the cell, denaturation of double-stranded nucleic acids, type of labels, configuration of label probes, capture probes, preamplifiers and/or amplifiers, label density, use of optional blocking probes, and/or the like.

A related general class of embodiments provides methods of quantitating a target RNA. In the methods, a sample comprising one or more copies of the target RNA is provided. The target RNA is generally endogenous to a cell. (That is, the RNA is a naturally occurring RNA, as opposed to an RNA produced by human intervention, e.g., using recombinant DNA techniques to insert probe binding sites into an RNA to create a reporter RNA for the purpose of monitoring its presence, location, or quantity in the cell.) A plurality of copies of a fluorescent label are captured to each of the one or more copies of the target RNA. The copies of the label are exposed to excitation light (of an appropriate wavelength for the label), whereupon the copies of the label fluoresce, thereby providing a florescent focus (or, equivalently, punctum, spot, or dot) for each of the one or more copies of the target RNA. The one or more resulting fluorescent foci are counted, thereby quantitating the target RNA. The target RNA can be an mRNA, a microRNA, a ribosomal RNA, a nuclear RNA, a cytoplasmic RNA, or the like.

The methods can be applied, e.g., to RNA in situ in a cell or free of any cell. Thus, in one class of embodiments, the sample comprises a cell lysate or other solution comprising the RNA. The RNA is optionally bound to a solid support, e.g., before or after capture of the label to the RNA. The RNA can be directly bound to the support, or it can be bound to a moiety that is in turn directly or indirectly bound to the support, e.g., an oligonucleotide or oligonucleotides; see, e.g., the section entitled “Non-specific capture” hereinbelow and U.S. patent application publications 2006/0286583 and 2006/0263769. In another class of embodiments, the sample comprises the cell to which the target RNA is endogenous, and the capturing, exposing, and counting steps are performed in the cell.

The methods are particularly useful for quantitation of low abundance RNAs. Thus, in one embodiment, about 100 copies or less of the target RNA are present in the cell, cell lysate, etc., for example, about 10 copies or less, about 5 copies or less, or even a single copy. As noted, a large number of labels are captured to each molecule. For example, at least about 400 copies of the label can be captured to each of the one or more copies of the target RNA, e.g., at least about 1000 copies, at least about 2000 copies, at least about 4000 copies, or at least about 8000 copies.

The label can be captured to the RNA directly or indirectly. Optionally, the label is provided by providing one or more copies of a label probe, the label probe comprising one or more copies of the label. The label probe can be hybridized directly to the target RNA. Preferably, however, the label probe is indirectly captured, e.g., by providing one or more capture probes, hybridizing a copy of each of the one or more capture probes to each of the one or more copies of the target RNA, and capturing the one or more copies of the label probe to the one or more capture probes. As for the embodiments above, the label probe can bind directly to the capture probe, or more typically an amplifier or a preamplifier and amplifier serve as intermediates. Optionally, two or more capture probes bind each label probe, amplifier, or preamplifier. Counting of the foci can be manual (e.g., involving visual inspection through a microscope) or it can be automated; see, e.g., Raj et al. (2006) “Stochastic MRNA synthesis in mammalian cells” PLoS Biology 4(10) e309 1707-1719 and Vargas et al. (2005) “Mechanism of RNA transport in the nucleus” Proc Natl Acad Sci 102:17008-17013.

Essentially all of the features noted for the methods above apply to these embodiments as well, as relevant; for example, with respect to cell type, type of target (including size), source of sample, fixation and permeabilization of the cell, washing the cell, denaturation of double-stranded nucleic acids, type of labels, configuration of label probes, capture probes, preamplifiers and/or amplifiers, label density, use of optional blocking probes, and/or the like.

Detection of Nucleic Acid Splicing in Individual Cells

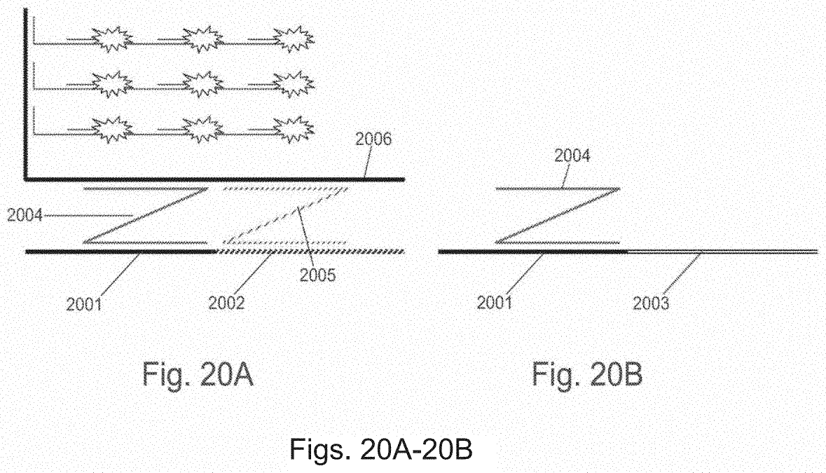

In one aspect, splicing of specific nucleic acid sequences can be detected using the instant technology. In one exemplary embodiment illustrated in FIG. 20 Panel A, capture probes 2004 and 2005 are designed to hybridize to a first splice variant. Capture probes 2004 and 2005 are complementary to sequences of the target nucleic acid (the first splice variant) on each side of the splice junction (sequences 2001 and 2002, respectively, e.g., a first exon and a second exon). If the splice has been formed (as in FIG. 20 Panel A), the two capture probes align side by side in the hybridization, which provides sufficient hybridization strength in the assay to maintain the attachment of preamplifier 2006, to which are hybridized multiple amplifiers and label probes. (It will be evident that the capture probes could instead hybridize, e.g., to an amplifier or label probe as described elsewhere herein.) Signal is then generated. If the splice is not formed or a different splice has been formed, the two capture probes will not be aligned side by side and there won't be sufficient hybridization strength to maintain the attachment of the preamplifier (or amplifier or label probe) and no signal will be generated. See FIG. 20 Panel B, which illustrates a second splice variant that includes sequences 2001 and 2003 (e.g., the first exon and a third exon). Capture probe 2004 but not 2005 can hybridize to the second splice variant. The hybridization of only capture probe 2004 is insufficient to capture preamplifier 2006, and thus the amplifier and label probe, to the second splice variant.

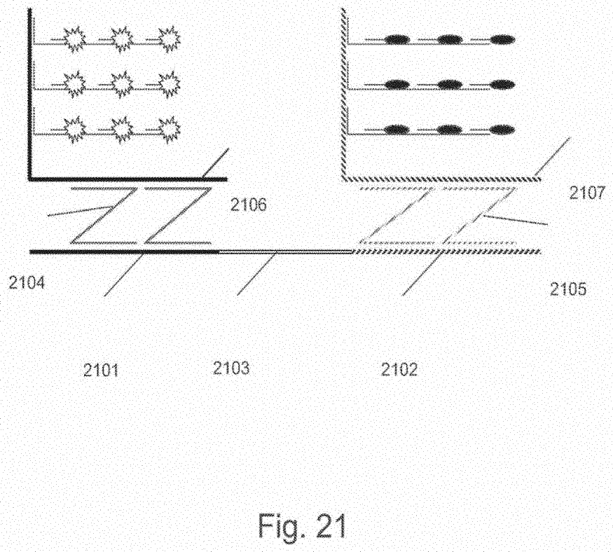

In another exemplary embodiment, different regions of the splice variant to be detected are tagged with different labels. This approach can be particularly useful for detection of a specific splice variant where the variant does not include a unique sequence (e.g., where other splice variants of the RNA include the same exons but in different combinations). In the embodiment shown in FIG. 21, the target splice variant includes sequences 2101 and 2102 (e.g., two exons present in the target splice variant but not present in combination in other splice variants of the mRNA) separated by sequence 2103. Capture probes 2104 capture preamplifier 2106, to which is hybridized a first amplifier and a first label probe. Capture probes 2105 capture preamplifier 2107, to which is hybridized a second preamplifier and a second label probe. The first and second labels emit different signals. If the splice is formed, the signals generated by the corresponding labels will spatially collocate at a single spot, yielding one new color; other variants that include either 2101 or 2102 but not both will bind only one of the two labels, therefore forming different spots of the two original colors.

In yet another example, one of the capture probes can be complementary to a region of the target splice variant that includes the splice junction, e.g., for variants in which the sequence at the splice junction is unique.

It will be evident that either exemplary configuration can be applied to singleplex or multiplex detection of splice variants.

Applications to “Whole-Sample” Analysis

All aspects of this invention are generally applicable to in situ detection of nucleic acids in individual cells. However, many features of this invention, including, but not limited to, probe set design, multiplexing, detection and quantification, can also be used in whole-sample nucleic acid detection applications. This section described several specific examples of such applications.

Non-Specific Capture

In existing hybridization-based assays, such as bDNA, only the target nucleic acid molecules are captured on a solid substrate while other nucleic acids are washed away. Such a measure reduces background noise and thus improves detection specificity. Techniques described herein, however, facilitate detection of a target nucleic acid (singleplex or multiplex) where essentially all nucleic acids in a given sample are immobilized non-specifically. Specific capture probes are designed to attach label molecules onto the target nucleic acid. As a result, only the target nucleic acid will produce signal. Any potential increase of background noise due to non-specific binding of nucleic acids can be more than compensated for by the noise reduction effect of the probe design, e.g., a double-Z design or other approach in which two or more capture probes are used to capture a preamplifier, amplifier, or label probe (see, e.g., the section entitled “Probe selection and design” hereinbelow). Such a probe set design scheme has the advantage of reduced probe set complexity, assay step simplification and cost reduction.

In in situ detection applications, nucleic acids are immobilized in cells through a cell fix step employing cross linking chemistry. In whole-sample detection applications, the nucleic acid molecules are released into solution from individual cells. They can be immobilized on solid substrates using any one of the existing nucleic acid immobilization methods, which include, but are not limited to, immobilization on nitrocellulose membranes or silica beads, attachment of poly-T oligo to a substrate surface, which in turn captures the poly-A section of RNA molecules to the substrate, and attachment of a long, random sequence nucleic acid on a substrate surface, which can provide affinity for RNA or DNA molecules.

Quantification of Gene Expression Level Through Imaging and Spot Counting

In existing whole-sample detection technologies, the expression level of a particular gene is quantified by measuring the intensity of the label attached to the target nucleic acid. The detection sensitivity is limited by the noise floor, which is produced by non-specific binding of label molecules or auto-fluorescence. When applying techniques described herein to whole-sample nucleic acid detection, the cells are lysed to release essentially all of the cellular nucleic acid molecules into a sample solution. Then the target nucleic acid molecules can be immobilized on solid substrate either specifically or non-specifically together with other nucleic acids. As described in previous sections, a large number of label probes can be attached to a single target nucleic acid molecule, which produces sufficient signal for each target nucleic acid molecule to be visualized as a spot under a normal microscope. Noise produced by non-specific label attachment or auto-fluorescence appears as larger patches with lower intensity, which are easily distinguishable from the real signal. As a result, the copy number of one or more target nucleic acid can be quantified by spot counting either manually or using simple image processing software. This quantification methodology is especially useful when the total number of target molecules in the sample is very small and the required detection accuracy is high.

Detection of Nucleic Acid Splicing in Whole Sample Solution

The splicing of nucleic acid molecules resulting in a either specific or non-specific sequence can be detected in similar ways to those described for detection in individual cells, except the nucleic acid molecules are released from cells into sample solutions and are typically immobilized on a substrate before detection.

### Compositions and Kits

The invention also provides compositions useful in practicing or produced by the methods. One exemplary class of embodiments provides a composition that includes a fixed and permeabilized cell, which cell comprises or is suspected of comprising a first nucleic acid target and a second nucleic acid target, at least a first capture probe capable of hybridizing to the first nucleic acid target, at least a second capture probe capable of hybridizing to the second nucleic acid target, a first label probe comprising a first label, and a second label probe comprising a second label. A first signal from the first label is distinguishable from a second signal from the second label. The cell optionally comprises the first and second capture probes and label probes. The first and second capture probes are optionally hybridized to their respective nucleic acid targets in the cell.

The features described for the methods above for indirect capture of the label probes to the nucleic acid targets apply to these embodiments as well. For example, the label probes can hybridize to the capture probes. In one class of embodiments, the composition includes a single first capture probe and a single second capture probe, where the first label probe is capable of hybridizing to the first capture probe and the second label probe is capable of hybridizing to the second capture probe. In another class of embodiments, the composition includes two or more first capture probes, two or more second capture probes, a plurality of the first label probes, and a plurality of the second label probes. A single first label probe is capable of hybridizing to each of the first capture probes, and a single second label probe is capable of hybridizing to each of the second capture probes.

In another aspect, amplifiers can be employed to increase the number of label probes captured to each target. For example, in one class of embodiments, the composition includes a single first capture probe, a single second capture probe, a plurality of the first label probes, a plurality of the second label probes, a first amplifier, and a second amplifier. The first amplifier is capable of hybridizing to the first capture probe and to the plurality of first label probes, and the second amplifier is capable of hybridizing to the second capture probe and to the plurality of second label probes. In another class of embodiments, the composition includes two or more first capture probes, two or more second capture probes, a multiplicity of the first label probes, a multiplicity of the second label probes, a first amplifier, and a second amplifier. The first amplifier is capable of hybridizing to one of the first capture probes and to a plurality of first label probes, and the second amplifier is capable of hybridizing to one of the second capture probes and to a plurality of second label probes.

In another aspect, preamplifiers and amplifiers are employed to capture the label probes to the targets. In one class of embodiments, the composition includes a single first capture probe, a single second capture probe, a multiplicity of the first label probes, a multiplicity of the second label probes, a plurality of first amplifiers, a plurality of second amplifiers, a first preamplifier, and a second preamplifier. The first preamplifier is capable of hybridizing to the first capture probe and to the plurality of first amplifiers, and the second preamplifier is capable of hybridizing to the second capture probe and to the plurality of second amplifiers. The first amplifier is capable of hybridizing to the first preamplifier and to a plurality of first label probes, and the second amplifier is capable of hybridizing to the second preamplifier and to a plurality of second label probes. In a related class of embodiments, the composition includes two or more first capture probes, two or more second capture probes, a multiplicity of the first label probes, a multiplicity of the second label probes, a multiplicity of first amplifiers, a multiplicity of second amplifiers, a plurality of first preamplifiers, and a plurality of second preamplifiers. The first preamplifier is capable of hybridizing to one of the first capture probes and to a plurality of first amplifiers, the second preamplifier is capable of hybridizing to one of the second capture probes and to a plurality of second amplifiers, the first amplifier is capable of hybridizing to the first preamplifier and to a plurality of first label probes, and the second amplifier is capable of hybridizing to the second preamplifier and to a plurality of second label probes. Optionally, additional preamplifiers can be used as intermediates between a preamplifier hybridized to the capture probe(s) and the amplifiers.

In the above classes of embodiments, one capture probe hybridizes to each label probe, amplifier, or preamplifier. In alternative classes of related embodiments, two or more capture probes hybridize to the label probe, amplifier, or preamplifier.

In one class of embodiments, the composition comprises a plurality of the first label probes, a plurality of the second label probes, a first amplified polynucleotide produced by rolling circle amplification of a first circular polynucleotide hybridized to the first capture probe, and a second amplified polynucleotide produced by rolling circle amplification of a second circular polynucleotide hybridized to the second capture probe. The first circular polynucleotide comprises at least one copy of a polynucleotide sequence identical to a polynucleotide sequence in the first label probe, and the first amplified polynucleotide comprises a plurality of copies of a polynucleotide sequence complementary to the polynucleotide sequence in the first label probe (and can thus hybridize to a plurality of the label probes). The second circular polynucleotide comprises at least one copy of a polynucleotide sequence identical to a polynucleotide sequence in the second label probe, and the second amplified polynucleotide comprises a plurality of copies of a polynucleotide sequence complementary to the polynucleotide sequence in the second label probe. The composition can also include reagents necessary for producing the amplified polynucleotides, for example, an exogenously supplied nucleic acid polymerase, an exogenously supplied nucleic acid ligase, and/or exogenously supplied nucleoside triphosphates (e.g., dNTPs).

The cell optionally includes additional nucleic acid targets, and the composition (and cell) can include reagents for detecting these targets. For example, the cell can comprise or be suspected of comprising a third nucleic acid target, and the composition can include at least a third capture probe capable of hybridizing to the third nucleic acid target and a third label probe comprising a third label. A third signal from the third label is distinguishable from the first and second signals. The cell optionally includes fourth, fifth, sixth, etc. nucleic acid targets, and the composition optionally includes fourth, fifth, sixth, etc. label probes and capture probes.

Essentially all of the features noted for the methods above apply to these embodiments as well, as relevant; for example, with respect to type of nucleic acid target, location of various targets on a single molecule or on different molecules, type of labels, inclusion of optional blocking probes, and/or the like. For example, it is worth noting that the second nucleic acid target optionally comprises a reference nucleic acid. In other embodiments, the first and second nucleic acid targets serve as markers for a specified cell type, e.g., redundant markers.

The cell can be essentially any type of cell from any source, particularly a cell that can be differentiated based on its nucleic acid content (presence, absence, or copy number of one or more nucleic acids). As just a few examples, the cell can be a circulating tumor cell or other tumor cell, a virally infected cell, a fetal cell in maternal blood, a bacterial cell or other microorganism in a biological sample (e.g., blood or other body fluid), or an endothelial cell, precursor endothelial cell, or myocardial cell in blood. For example, the cell can be derived from a bodily fluid, blood, bone marrow, sputum, urine, lymph node, stool, cervical pap smear, oral swab or other swab or smear, spinal fluid, saliva, sputum, semen, lymph fluid, an intercellular fluid, a tissue (e.g., a tissue homogenate), a biopsy, and/or a tumor. The cell is optionally in a tissue, e.g., a tissue section (e.g., an FFPE section) or other solid tissue sample. The cell can be derived from one or more of a human, an animal, a plant, and a cultured cell.

The cell can be present in a mixture of cells, for example, a complex heterogeneous mixture. In one class of embodiments, the cell is of a specified type, and the composition comprises one or more other types of cells. These other cells can be present in excess, even large excess, of the cell. For example, the ratio of cells of the specified type to cells of all other type(s) in the composition is optionally less than 1:1×10⁴, less than 1:1×10⁵, less than 1:1×10⁶, less than 1:1×10⁷, less than 1:1×10⁸, or even less than 1:1×10⁹.

The cell is optionally immobilized on a substrate, present in a tissue section, or the like. In certain embodiments, however, the cell is in suspension in the composition. The composition can be contained in a flow cytometer or similar instrument. Additional features described herein, e.g., in the section entitled “Implementation, applications, and advantages,” can be applied to the compositions, as relevant.

Another aspect of the invention provides compositions in which a large number of labels are correlated with each target nucleic acid. One general class of embodiments thus provides a composition comprising a cell, which cell includes a first nucleic acid target, a second nucleic acid target, a first label whose presence in the cell is indicative of the presence of the first nucleic acid target in the cell, and a second label whose presence in the cell is indicative of the presence of the second nucleic acid target in the cell, wherein a first signal from the first label is distinguishable from a second signal from the second label. An average of at least one copy of the first label is present in the cell per nucleotide of the first nucleic acid target over a region that spans at least 20 contiguous nucleotides of the first nucleic acid target, and an average of at least one copy of the second label is present in the cell per nucleotide of the second nucleic acid target over a region that spans at least 20 contiguous nucleotides of the second nucleic acid target.

In one class of embodiments, the copies of the first label are physically associated with the first nucleic acid target, and the copies of the second label are physically associated with the second nucleic acid target. For example, the first label can be part of a first label probe and the second label part of a second label probe, where the label probes are captured to the target nucleic acids.

In one class of embodiments, an average of at least four, eight, or twelve copies of the first label are present in the cell per nucleotide of the first nucleic acid target over a region that spans at least 20 contiguous nucleotides of the first nucleic acid target, and an average of at least four, eight, or twelve copies of the second label are present in the cell per nucleotide of the second nucleic acid target over a region that spans at least 20 contiguous nucleotides of the second nucleic acid target. In one embodiment, an average of at least sixteen copies of the first label are present in the cell per nucleotide of the first nucleic acid target over a region that spans at least 20 contiguous nucleotides of the first nucleic acid target, and an average of at least sixteen copies of the second label are present in the cell per nucleotide of the second nucleic acid target over a region that spans at least 20 contiguous nucleotides of the second nucleic acid target.

Essentially all of the features noted for the embodiments above apply to these embodiments as well, as relevant, for example, with respect to type of labels, suspension of the cell or presence of the cell in a tissue section, and/or the like. The regions of the first and second nucleic acid targets are typically regions covered by a probe, primer, or similar polynucleotide employed to detect the respective target. The regions of the first and second nucleic acid targets optionally span at least 25, 50, 100, 200, or more contiguous nucleotides and/or at most 2000, 1000, 500, 200, 100, 50, or fewer nucleotides. A like density of labels is optionally captured to third, fourth, fifth, sixth, etc. nucleic acid targets. The composition optionally includes PCR primers, a thermostable polymerase, and/or the like, in embodiments in which the targets are detected by multiplex in situ PCR.

Another aspect of the invention provides kits useful for practicing the methods. One general class of embodiments provides a kit for detecting a first nucleic acid target and a second nucleic acid target in an individual cell. The kit includes at least one reagent for fixing and/or permeabilizing the cell, at least a first capture probe capable of hybridizing to the first nucleic acid target, at least a second capture probe capable of hybridizing to the second nucleic acid target, a first label probe comprising a first label, and a second label probe comprising a second label, wherein a first signal from the first label is distinguishable from a second signal from the second label, packaged in one or more containers.

Essentially all of the features noted for the embodiments above apply to these embodiments as well, as relevant; for example, with respect to number of nucleic acid targets, configuration and number of the label and capture probes, inclusion of preamplifiers and/or amplifiers, inclusion of blocking probes, inclusion of amplification reagents, type of nucleic acid target, location of various targets on a single molecule or on different molecules, type of labels, inclusion of optional blocking probes, and/or the like. The kit optionally also includes instructions for detecting the nucleic acid targets in the cell and/or identifying the cell as being of a specified type, one or more buffered solutions (e.g., diluent, hybridization buffer, and/or wash buffer), reference cell(s) comprising one or more of the nucleic acid targets, and/or the like.

Another general class of embodiments provides a kit for detecting an individual cell of a specified type from a mixture of cell types by detecting a first nucleic acid target and a second nucleic acid target. The kit includes at least one reagent for fixing and/or permeabilizing the cell, a first label probe comprising a first label (for detection of the first nucleic acid target), and a second label probe comprising a second label (for detection of the second nucleic acid target), wherein a first signal from the first label is distinguishable from a second signal from the second label, packaged in one or more containers. The specified type of cell is distinguishable from the other cell type(s) in the mixture by presence, absence, or amount of the first nucleic acid target in the cell or by presence, absence, or amount of the second nucleic acid target in the cell (that is, the two targets are redundant markers for the specified cell type).

Essentially all of the features noted for the embodiments above apply to these embodiments as well, as relevant; for example, with respect to number of nucleic acid targets, inclusion of capture probes, configuration and number of the label and/or capture probes, inclusion of preamplifiers and/or amplifiers, inclusion of blocking probes, inclusion of amplification reagents, type of nucleic acid target, location of various targets on a single molecule or on different molecules, type of labels, inclusion of optional blocking probes, and/or the like. The kit optionally also includes instructions for identifying the cell as being of the specified type, one or more buffered solutions (e.g., diluent, hybridization buffer, and/or wash buffer), reference cell(s) comprising one or more of the nucleic acid targets, and/or the like.

### Implementation, Applications, and Advantages

Various aspects of the invention are described in additional detail below. Exemplary embodiments and applications are also described.

The new technology (methods, compositions, systems, and kits), QMAGEX (Quantitative Multiplex Analysis of Gene Expression in Single Cell), disclosed herein is capable of detection and quantification of multiple nucleic acids within individual cells. The technology is significantly different from existing ISH technology in several aspects, although they both can measure mRNA expression in individual cells. First, cells optionally remain in suspension status during all or at least most of the assay steps in the assays of the present invention, which greatly improves assay hybridization kinetics, resulting in better reproducibility and shorter assay time. Second, the instant technology has the capability for analyzing the expression of multiple mRNA transcripts within cells simultaneously and quantitatively. This is highly desirable, since, for example, detection of multiple tumor marker genes could greatly improve the accuracy of CTC identification (Mocellin et al., 2004) and greatly reduce the false positive rate. Quantitative analysis of gene expression level could not only further aid in discriminating the CTC from other types of cells but also could help in distinguishing the type and source of primary tumors as well as the stages of tumor progression. Third, the instant technology enables the use of a flow cytometer as the base for detection, which, compared with microscope-based detection instruments, offers higher throughput. In addition, the flow cytometer is capable of sorting out cells, e.g., tumor cells, for further study. Subsequent to the detection and quantification of mRNA expression, isolation of the CTC or other cells may be advantageous for further identity confirmation or for additional cytological and molecular analysis. Fourth, the instant technology has vastly improved detection sensitivity and reproducibility, and is capable of single copy gene detection and quantification. In addition, the instant technology uses a standard, generic set of probe labeling and detection technology (e.g., the same set of preamplifiers, amplifiers, and label probes can be used to detect multiple different sets of nucleic acid targets, requiring only synthesis of a new set of capture probes for each new set of nucleic acid targets), and optionally uses standardized procedures for cell fixation and permeation and for hybridization and washing. Furthermore, the technology can include built-in internal controls for assay specificity and efficiency.

The instant technology can be used not only for the detection and enumeration of rare CTC in blood samples or other body fluids, but also for any type of rare cell identification and enumeration events. Applications include, but are not limited to: detection of minimal residual disease in leukemia and lymphoma; recurrence monitoring after chemotherapy treatment (Hess et al.); detection of other pre-cancerous cells, such as the detection of HPV-containing cervical cells in body fluids; detection of viral or bacterial nucleic acid in an infected cell; detection of fetal cells in maternal blood; detection of micro-tumor lesions during early stage of tumor growth; or detection of residual tumor cells after surgery for margin management. In all of these cases, target cell specific gene expression is likely to be buried in the background of large numbers of heterogeneous cell populations. As a result, microarray or RT-PCR based expression analysis, which require the isolation of mRNA from a large population of cells, will have difficulty detecting the presence of those rare cell events accurately or reliably, whereas the invented technology can readily be applied.

It should also be noted that although single cell detection and quantification of multiple mRNA transcripts is illustrated here as the main application, such technology is equally applicable to detection of other rare cell events that include changes in chromosomal DNA or cellular nucleic acid content. Examples include, but are not limited to, detection of her-2/neu gene amplification, detection of Rb gene deletion, detection of somatic mutations, detection of chromosome translocation such as in chronic myelogenous leukemia (BCR-ABL), or detection of HPV insertion to chromosomal DNA of cervical cancer cells.

Finally, the probe design, multiplexing and amplification aspects of the instant technology can be applied in quantitative, multiplex gene expression analysis and in measuring chromosomal DNA changes at a single cell level in solid tissue sections, such as formalin-fixed, paraffin embedded (FFPE) tissue samples.

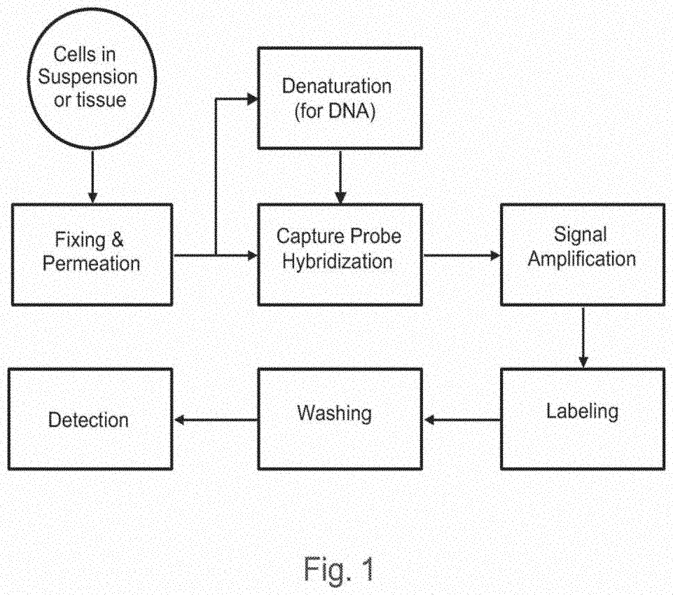

The QMAGEX technology comprises an assay and optional associated apparatus to implement the assay in an automated fashion. FIG. 1 illustrates major elements of the QMAGEX assay work flow, which, for one exemplary embodiment in which the cells are in suspension and amplifiers are employed, include:

Fixation and Permeation: Cells in the sample are fixed and permeated (permeabilized) in suspension. The fixation step immobilizes nucleic acids (e.g., mRNA or chromosomal DNA) and cross-links them to the cellular structure. Then the cell membrane is permeabilized so that target-specific nucleic acid probes and signal-generating particles, such as fluorescently labeled nucleic acid probes, can enter the cell and bind to the target.

Denaturation: If the detection target is double-stranded chromosomal DNA, a denaturation step is added to convert the double-stranded target into single-stranded DNA, ready to be bound with the target-specific probes.

Capture Probe Hybridization: Carefully selected target-specific capture probes or probe sets are hybridized to the target nucleic acids. The capture probes serve to link the target molecules specifically to signal-generating particles. The technology enables multiple target genes in the cell to be recognized by different probe sets simultaneously and with a high degree of specificity.

Signal Amplification: Signals from target molecules are amplified by binding a large scaffold molecule, an amplifier, to the capture probes or probe sets. Each scaffold has multiple locations to accept label probes and signal-generating particles. In a multiplex assay, multiple distinct amplifiers are used.

Labeling: Label probes, to which signal generating particles (labels) are attached, hybridize to the amplifier in this step. In a multiplex assay, multiple distinct label probes are used.

Washing: The excess probes or signal generating particles that are not bound or that are nonspecifically bound to the cells are removed through a washing step, which reduces background noise and improves the detection signal to noise ratio. Additional washing steps may be added during the capture probe hybridization or signal amplification steps to further enhance the assay performance.

Detection: The labeled suspension cells are detected using Fluorescent Activated Cell Sorting (FACS) or a flow cytometer, or are immobilized on a solid surface and detected using a microscope or scanner based instrument.

In the following section, major elements of the QMAGEX technology will be described in detail. In the following, the term label probe refers to an entity that binds to the target molecule, directly or indirectly, and enables the target to be detected by a readout instrument. The label probe, in general, comprises a nucleic acid or modified nucleic acid molecule that binds to the target, directly or indirectly, and one or more “signal generating particle” (i.e., label) that produces the signal recognizable by the readout instrument. In indirect mode, the label probe can either be attached to the target molecule through binding to a capture probe directly or through binding to an amplifier that is in turn linked to a capture probe. Exemplary signal-generating particles (labels) include, but are not limited to, fluorescent molecules, nano-particles, radioactive isotopes, chemiluminescent molecules (e.g., digoxigenin, dinitrophenyl). Fluorescent molecules include, but are not limited to, fluorescein (FITC), cy3, cy5, alexa dyes, phycoerythrin, etc. Nano-particles include, but are not limited to, fluorescent quantum dots, scattering particles, etc. The term capture probe refers to a nucleic acid or a modified nucleic acid that links the target to a specific type of label probe, directly or indirectly. The term “capture probe set” refers to multiple nucleic acids or modified nucleic acids that link a target to a specific type of label probe, directly or indirectly, for increased assay sensitivity. The term amplifier refers to a large scaffold molecule(s) that binds to one or more capture probes or to a preamplifier on one side and to multiple label probes on another side.

### Fixation

In this step, the nucleic acids are immobilized within cells by cross-linking them within the cellular structure. There are a variety of well known methods to fix cells in suspension with a fixative reagent and to block the endogenous RNase activities, which can be adapted for use in the present invention. Fixative reagents include formalin (formaldehyde), paraformaldehyde, gluteraldehyde, ethanol, methanol, etc. One common fixative solution for tissue sections includes 0.25% gluteraldehyde and 4% paraformaldehyde in phosphate buffer. Another common fixative solution for tissue sections includes 50% ethanol, 10% formalin (containing 37% formaldehyde), and 5% acetic acid. Different combinations of the fixative reagents at various concentrations are optionally tested to find the optimal composition for fixing cells in suspension, using techniques well known in the art. Duration of the fixing treatment can also be optimized. A number of different RNase inhibitors can be included in the fixative solution, such as RNAlater (Ambion), citric acid or LiCl, etc.

### Permeation

Fixation results in cross-linking of the target nucleic acids with proteins or other cellular components within cells, which may hinder or prevent infiltration of the capture probes into the cells and mask the target molecules for hybridization. The assays of the invention thus typically include a follow-on permeation step to enable in-cell hybridization. One technique involves the application of heat for varying lengths of time to break the cross-linking. This has been demonstrated to increase the accessibility of the mRNA in the cells for hybridization. Detergents (e.g., Triton X-100 or SDS) and Proteinase K can also be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the target mRNA. Again, optimal concentrations and duration of treatment can be experimentally determined as is well known in the art. A cell washing step can follow, to remove the dissolved materials produced in the permeation step.

Optionally, prior to fixation and permeation, cells in suspension are collected and treated to inactivate RNase and/or to reduce autofluorescence. DEPC treatment (e.g. Braissant and Wahli (1988) “A simplified in situ hybridization protocol using non-radioactively labeled probes to detect abundant and rare mRNAs on tissue sections” Biochemica 1:10-16) and RNAlater (Ambion, Inc.) have been demonstrated to be effective in stabilizing and protecting cellular RNA. Sodium borohydride and high heat have also been shown to preserve the integrity of RNA and to reduce autofluorescence, facilitating the detection of genes expressed at a low level (Capodieci et al. (2005) “Gene expression profiling in single cells within tissue” Nat Methods 2(9):663-5). Other methods of reducing cellular autofluorescence such as trypan blue (Mosiman et al. (1997) “Reducing cellular autofluorescence in flow cytometry: an in situ method” Cytometry 30(3):151-6) or singly labeled quencher oligonucleotide probe (Nolan et al. (2003) “A simple quenching method for fluorescence background reduction and its application to the direct, quantitative detection of specific mRNA” Anal Chem. 2003 75(22):6236-43) are optionally employed.

### Capture Probe Hybridization

In this assay step, the capture probe or capture probe set binds to the intended target molecule by hybridization. One indicator for a successful target hybridization is specificity, i.e. the capture probes or probe sets should substantially only link the label probes to the specific target molecule of interest, not to any other molecules. Probe selection and design are important in achieving specific hybridization.

Probe Selection and Design

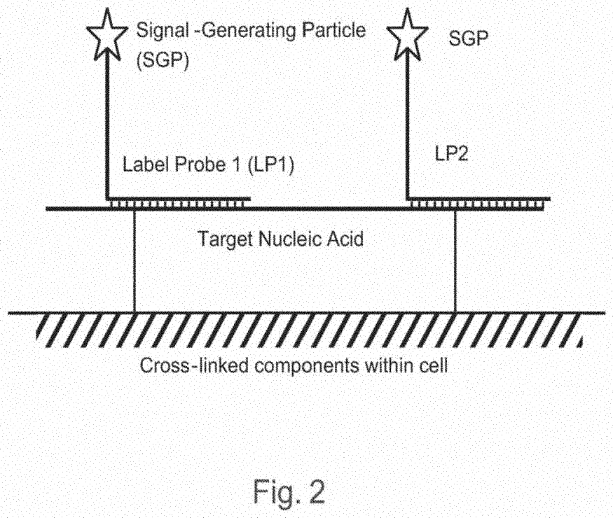

The assays of the invention employ two types of approaches in probe design to link the target nucleic acids in cells to signal generating particles: “direct labeling” and “indirect labeling”. In the direct labeling approach, the target molecule hybridizes to or captures one or more label probes (LP) directly. The LPs contain the signal-generating particles (SGP), as shown in FIG. 2. A different LP needs to be used to attach additional SGP at different positions on the target molecule. In order to ensure hybridization specificity, the label probe is preferably stringently selected to ensure that it does not cross-hybridize with nonspecific nucleic acid sequences.

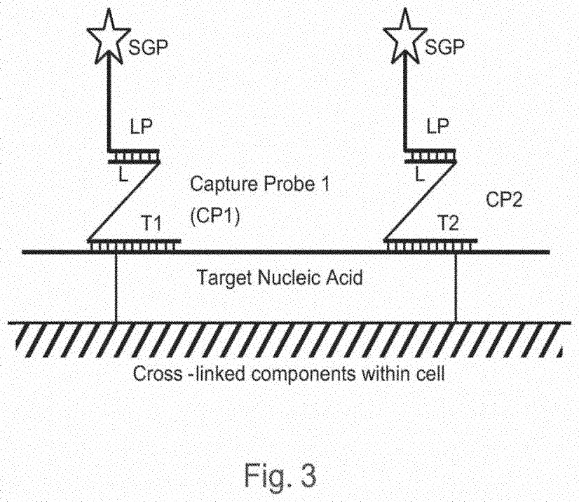

In the indirect labeling approach, an additional capture probe (CP) is employed. An example is shown in FIG. 3. The target molecule captures the label probe through the capture probe. In each capture probe, there is at least one section, T, complementary to a section on the target molecule, and another section, L, complementary to a section on the label probe. The T and L sections are connected by a section C. To attach more SGPs to different positions on the same target molecule, different capture probes are needed, but the label probe can remain the same. The sequence of L is carefully selected to ensure that it does not cross-hybridize substantially with any sequences in the nucleic acids in cells. In a further embodiment, the L portion of the capture probe and the label probe contain chemically modified or nonnatural nucleotides that do not hybridize with natural nucleotides in cells. In another embodiment, L and the label probe (or a portion thereof) are not even nucleic acid sequences. For example, L can be a weak affinity binding antibody that recognizes the signal-generating probe, which in this case is or includes an antigen; L can be covalently conjugated to an oligonucleotide that comprises the T section of the capture probe. Optionally, for two adjacent capture probes, the T sections hybridize to the target and two of the low affinity binding antibody binds to the antigen on the label probe at the same time, which results in strong affinity binding of the antigen. The capture and label probes are specific for a target gene of interest. Multiple capture probes (probe set) can be bound to the same target gene of interest in order to attach more signal-generating particles for higher detection sensitivity. In this situation, the probe set for the same target gene can share the same label probe.

Although both approaches can be used in the instant technology, the indirect capture approach is preferred because it enables the label probe to be target independent and further disclosure will show that it can offer better specificity and sensitivity.

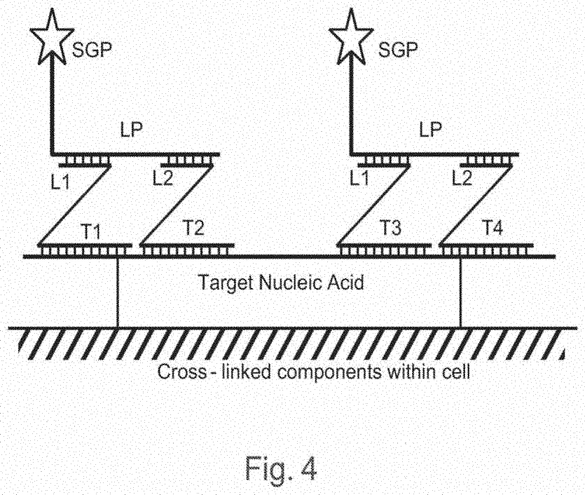

In a further indirect capture embodiment shown in FIG. 4, two adjacent capture probes are incorporated in a probe set targeting a gene of interest. T1 and T2 are designed to be complementary to two unique and adjacent sections on the target nucleic acid. L₁ and L₂, which can be different or the same, are complementary to two adjacent sections on the label probe. Their binding sections, T, L or both, are designed so that the linkage between the label probe and the target is unstable and tends to fall off at hybridization temperature when only one of the capture probes is in place. Such a design should enable exceptional specificity because the signal-generating label probe can only be attached to the target gene of interest when two independent capture probes both recognize the target and bind to the adjacent sequences or in very close proximity of the target gene. In one embodiment, the melting temperature, Tₘ, of the T sections of the two capture probes are designed to be significantly above the hybridization temperature while the Tₘ of the L sections is below the hybridization temperature. As a result, T sections bind to the target molecule strongly and stably during hybridization, while L sections bind to the label probe weakly and unstably if only one of the capture probes is present. However, if both capture probes are present, the combination of L₁ and L₂ holds the label probe strongly and stably during hybridization. For example, the T sections can be 20-30 nucleotides in length while the L sections are 13-15 nucleotides in length; C can be 0 to 10 nucleotides in length, e.g., 5 nucleotides. In another embodiment, Tₘ of the T sections is below hybridization temperature while Tₘ of the L sections is substantially above. In the same way, the linkage between the label probe and the target can only survive the hybridization when both capture probes are hybridized to the target in a cooperative fashion. See Example 1 hereinbelow and U.S. patent application publication 2007/0015188 entitled “Multiplex detection of nucleic acids” by Luo et al. for additional details on design of capture probes.

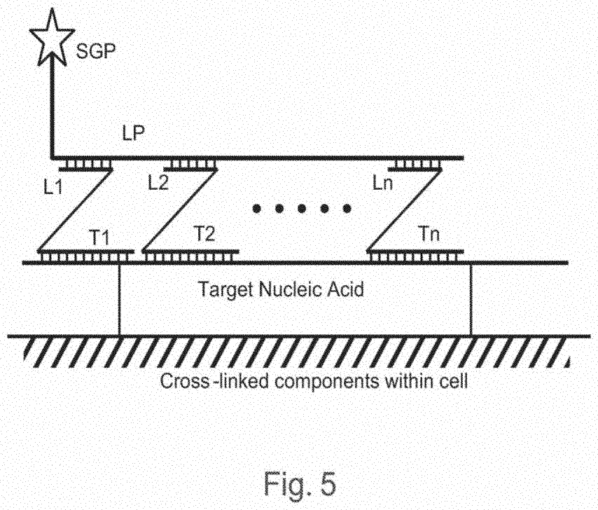

In another embodiment, three or more of the target nucleic acid specific, neighboring capture probes are used for the stable capture of one label probe within cells (FIG. 5). The basic design of the probes is the same as discussed above, but the capture of one signal-generating probe should have even higher specificity than when two neighboring probes are used since now three independent probes have to bind to the same target molecule of interest in neighboring positions in order to generate signal.

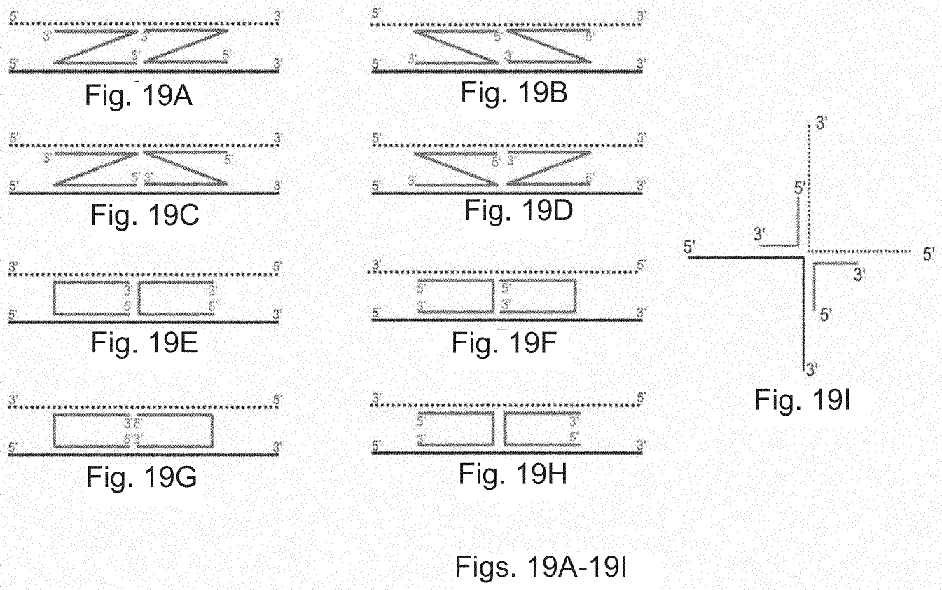

It will be evident that, while the embodiments above are described in terms of capture probe configurations such as those shown in FIGS. 3-5 and FIG. 19 Panels A-B, other capture probe configurations can readily be employed. Additional exemplary capture probe configurations that can be adapted to the practice of the present invention are illustrated in FIG. 19 Panels C-I. As for the embodiments above, two, three, or more such capture probes can bind to a single label probe, amplifier, or preamplifier. Also as described above, optionally sections T, L, or both are designed such that stable capture of the label probe, amplifier, or preamplifier requires binding of more than one of the capture probes. For example, the T sections can be 20-30 nucleotides in length while the L sections are 13-15 nucleotides in length; C can be 0-10 nucleotides in length, e.g., 5 nucleotides. It is worth noting that, in certain configurations, the ends of adjacent capture probes can optionally be ligated to each other when the capture probes are bound to the target nucleic acid and/or the label probe, amplifier, or preamplifier; see FIG. 19 Panels C, D and G.

Multiplexing

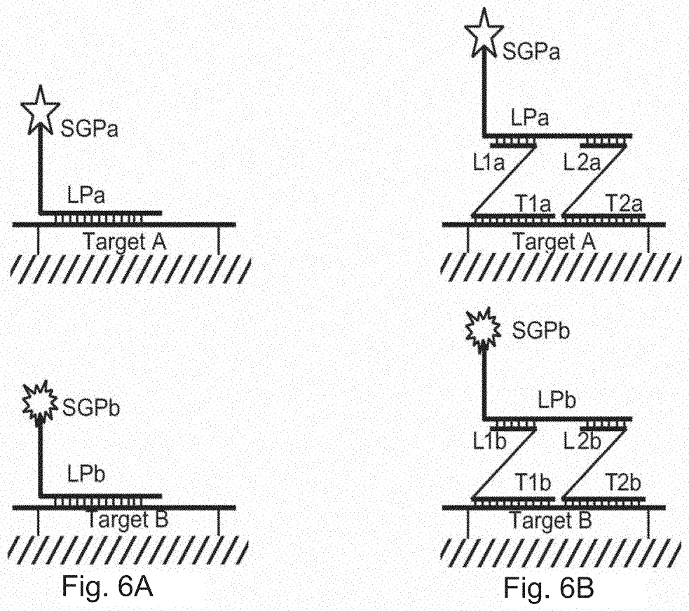

To perform multiplexed detection for more than one target gene, e.g., as shown in FIG. 6, each target gene has to be specifically bound by different capture and label probes. In addition, the signal generating particle (the label) attached to the label probe should provide distinctively different signals for each target that can be read by the detection instrument. In the direct labeling approach (e.g., FIG. 6 Panel A), suitable label probes with minimal cross-hybridization can be harder to find because each label probe has to be able to bind to the target strongly but not cross-hybridize to any other nucleic acid molecules in the system. For this approach to provide optimal results, the target binding portion of the label probe should be judiciously designed so that it does not substantially cross-hybridize with nonspecific sequences. In the indirect labeling approach (e.g., FIG. 6 Panel B), because of the unique multiple capture probe design approach, even when one capture probe binds to a nonspecific target, it will not result in the binding of the label probe to the nonspecific target. The assay specificity can be greatly improved. Thus the capture probe design illustrated in FIG. 4 and FIG. 5 is typically preferred in some multiplex assay applications. In one class of embodiments, the signal-generating particles attached to different target genes are different fluorescent molecules with distinctive emission spectra.

The capacity of the instant technology to measure more than one parameter simultaneously can enable detection of rare cells in a large heterogeneous cell population. As noted above, the concentration of CTC is estimated to be in the range of one tumor cell among every 10⁶-10⁷ normal blood cells. In existing FACS based immunoassays, on the other hand, random dye aggregation in cells may produce one false positive cell count in every ten thousand cells. Such an assay can thus not be used for CTC detection due to the unacceptably high false positive rates. This problem can be solved elegantly using the instant technology. In one particular embodiment, expression of more than one tumor genes are used as the targets for multiplex detection. Only cells that express all the target genes are counted as tumor cells. In this way, the false positive rate of the CTC detection can be dramatically reduced. For example, since dye aggregation in cells is a random event, if the false positive rate of a single color detection is 10⁻⁴, the false positive rate for two color or three color detection can be as low as 10⁻⁸ or 10⁻¹², respectively. In situations where the relative levels of expression of the target genes are known, these relative levels can be measured using the multiplex detection methods disclosed herein and the information can be used to further reduce the false positive rate of the detection.

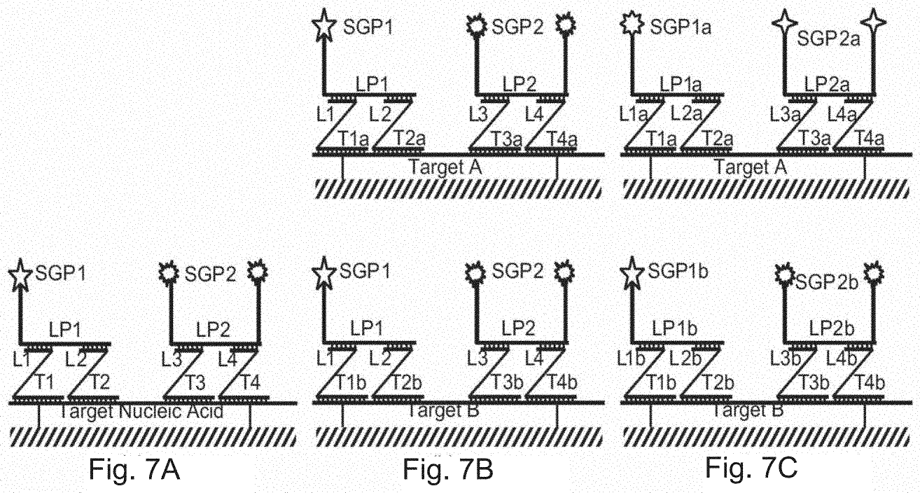

In another embodiment, schematically illustrated in FIG. 7 Panel A, more than one signal-generating particles are linked to the same target nucleic acid. These particles generate distinct signals in the detection instrument. The relative strengths of these signals can be pre-determined by designing the number of each type of particles attached to the target. The number of signal-generating particles on a target can be controlled in probe design by changing the number of probe sets or employing different signal amplification methods, e.g., as described in the following section. The rare cells are identified only when the relative signal strengths of these particles measured by the detection instrument equal the pre-determined values. This embodiment is useful when there are not enough suitable markers or when their expression levels are unknown in a particular type of rare cells. In yet another embodiment, shown in FIG. 7 Panel B, the same set of signal-generating particles are attached to more than one target. The relative signal strengths of the particle set are controlled to be the same on all selected targets. This embodiment is useful in situations in which the rare cell is identified when any of the target molecules are present. In yet another embodiment, depicted in FIG. 7 Panel C, each target molecule has a set of signal generating particles attached to it, but the particle sets are distinctively different from target to target.

The detection of multiple target nucleic acid species of interest can be applied to quantitative measurement of one target. Due to different sample and experimental conditions, the abundance of a particular target molecule in a cell normally may not be determined quantitatively through the detection of the signal level associated with the target alone in embodiments in which intensity levels are measured. More precise measurement can potentially be accomplished by normalizing the signal of a gene of interest to that of a reference/housekeeping gene. A reference/housekeeping gene is defined as a gene that is generally always present or expressed in cells. The expression of the reference/housekeeping gene is generally constitutive and tends not to change under different biological conditions. 18S, 28S, GAPD, ACTB, PPIB etc. have generally been considered as reference or housekeeping genes, and they have been used in normalizing gene expression data generated from different samples and/or under varying assay conditions.

In another embodiment, a special label probe set can be designed that does not bind to any capture probe or target specifically. The signal associated to this label probe can be used to establish the background of hybridization signal in individual cells. Thus the abundance of a particular target molecule can be quantitatively determined by first subtracting the background hybridization signal, then normalizing against the background subtracted reference/housekeeping gene hybridization signal.

In yet another embodiment, two or more chromosomal DNA sequences of interest can be detected simultaneously in cells. In the detection of multiple DNA sequences in cells, the label probes for the DNA sequences are distinct from each other and they do not cross-hybridize with each other. In embodiments in which cooperative indirect capture is employed, because of the design scheme, even when one probe binds to a nonspecific DNA sequence, it will not result in the capture of the signal-generating probe to the nonspecific DNA sequences.

In yet another embodiment, the detection of multiple target chromosomal DNA sequences of interest enables quantitative analysis of gene amplification, gene deletion, or gene translocations in single cells. This is accomplished by normalizing the signal of a gene of interest to that of a reference gene. The signal ratio of the gene of interest to the reference gene for a particular cell of interest is compared with the ratio in reference cells. A reference gene is defined as a gene that stably maintains its copy numbers in the genomic DNA. A reference cell is defined as a cell that contains the normal copy number of the gene of interest and the reference gene. If the signal ratio is higher in the cells of interest in comparison to the reference cells, gene amplification is detected. If the ratio is lower in the cells of interest in comparison to the reference cells, then gene deletion is detected.

### Signal Amplification & Labeling

The direct labeling approach depicted in FIG. 2 and FIG. 6 Panel A offers only limited sensitivity because only a relatively small number of signal-generating particles (labels) can be attached to each label probe. One way to increase sensitivity is to use in vitro transcribed RNA that incorporates signal-generating particles, but specificity will suffer as a result.

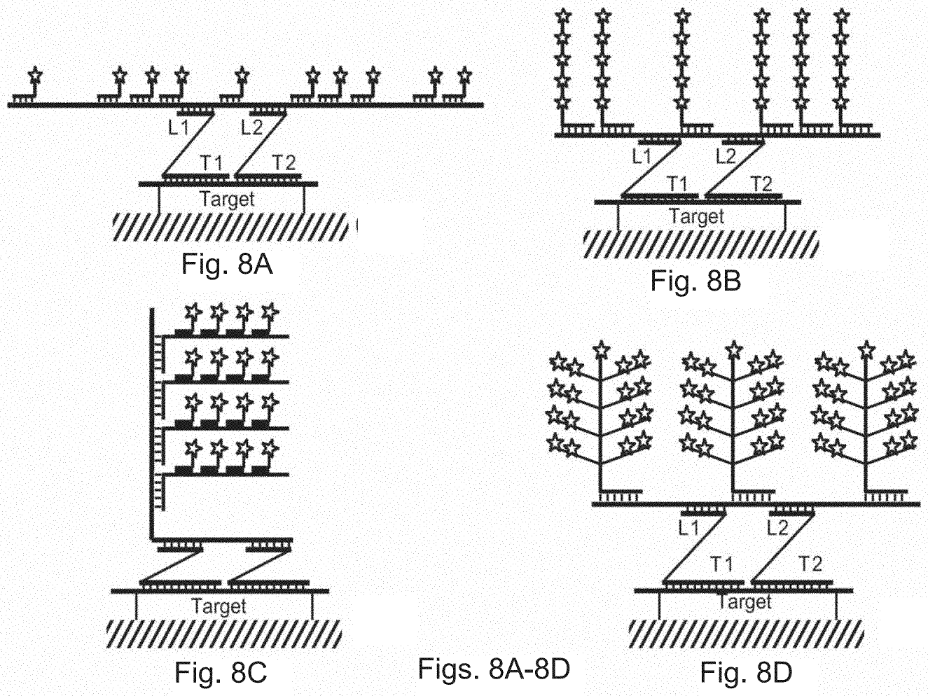

The “indirect labeling” approach not only can improve specificity as described above but also can be used to improve the detection sensitivity. In this approach, the label probe is hybridized or connected to an amplifier molecule, which provides many more attachment locations for label probes. The structure and attachment method of the amplifier can take many forms. FIG. 8 Panels A-D show a number of amplification schemes as illustrative examples. In Panel A, multiple singly-labeled label probes bind to the amplifier. In Panel B, multiple multiply-labeled label probes bind to the amplifier. In Panel C, multiple singly-labeled label probes bind to the amplifier, and multiple copies of the amplifier are bound to a preamplifier. In one particular embodiment, the amplifier is one or multiple branched DNA molecules (Panel D). The sequence of the label probe is preferably selected carefully so that it does not substantially cross-hybridize with any endogenous nucleic acids in the cell. In fact, the label probe does not have to be a natural polynucleotide molecule. Chemical modification of the molecule, for example, inclusion of nonnatural nucleotides, can ensure that the label probe only hybridizes to the amplifier and not to nucleic acid molecules naturally occurring in the cells. In multiplex assays, distinct amplifiers and label probes will be designed and used for the different targets.

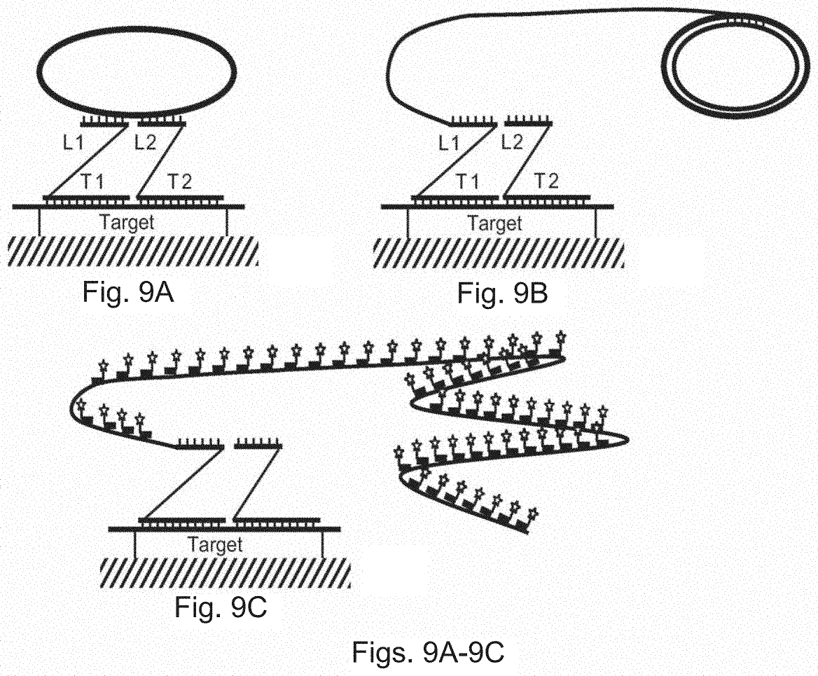

In one embodiment, as schematically illustrated in FIG. 9, a circular polynucleotide molecule is captured by the capture probe set. Along the circle, there can be one sequence or more than one repeat of the same sequence that binds to label probe (FIG. 9 Panel A). In the signal amplification step of the assay, a rolling circle amplification procedure (Larsson et al, 2004) is carried out. As the result of this procedure, a long chain polynucleotide molecule attached to the capture probes is produced (FIG. 9 Panel B). There are many repeating sequences along the chain, on which label probes can be attached by hybridization (FIG. 9 Panel C). In multiplex assays, distinct capture probes, rolling circles, and label probes will be designed and used.

In one embodiment, a portion of the signal-generating probe can be PCR-amplified. In another embodiment, each portion of multiple signal-generating probes can be PCR-amplified simultaneously.

Although a specific capture approach (indirect labeling with capture probe pairs) has been used to illustrate the labeling and amplification schemes in FIGS. 8 and 9, it is important to note that any other probe capture approaches, direct or indirect, described in previous sections can be used in combination with the labeling and amplification schemes described in these sections. The capture probe, labeling methods, and amplifier configurations described above are independent of each other and can be used in any combination in a particular assay design, e.g., in in situ or whole sample detection.

### Hybridization Conditions

The composition of the hybridization solution can affect efficiency of the hybridization process. Hybridization typically depends on the ability of the oligonucleotide to anneal to a complementary mRNA strand below its melting point (Tₘ). The value of the Tₘ is the temperature at which half of the oligonucleotide duplex is present in a single stranded form. The factors that influence the hybridization of the oligonucleotide probes to the target nucleic acids can include temperature, pH, monovalent cation concentration, presence of organic solvents, etc. A typical hybridization solution can contain some or all of the following reagents, e.g., dextran sulfate, formamide, DTT (dithiothreitol), SSC (NaCl plus sodium citrate), EDTA, etc. Other components can also be added to decrease the chance of nonspecific binding of the oligonucleotide probes, including, e.g., single-stranded DNA, tRNA acting as a carrier RNA, polyA, Denhardt's solution, etc. Exemplary hybridization conditions can be found in the art and/or determined empirically as well known in the art. See, e.g., U.S. patent application publication 2002/0172950, Player et al. (2001) J. Histochem. Cytochem. 49:603-611, and Kenny et al. (2002) J. Histochem. Cytochem. 50:1219-1227, which also describe fixation, permeabilization, and washing.

An additional prehybridization is optionally carried out to reduce background staining. Prehybridization involves incubating the fixed tissue or cells with a solution that is composed of all the elements of the hybridization solution, minus the probe.

Washing

Following the labeling step, the cells are preferably washed to remove unbound probes or probes which have loosely bound to imperfectly matched sequences. Washing is generally started with a low stringency wash buffer such as 2×SSC+1 mM EDTA (1×SSC is 0.15M NaCl, 0.015M Na-citrate), then followed by washing with higher stringency wash buffer such as 0.2×SSC+1 mM EDTA or 0.1×SSC+1 mM EDTA.

Washing is important in reducing background noise, improving signal to noise ratio of and quantification with the assay. Established washing procedures can be found, e.g., in Bauman and Bentvelzen (1988) “Flow cytometric detection of ribosomal RNA in suspended cells by fluorescent in situ hybridization” Cytometry 9(6):517-24 and Yu et al. (1992) “Sensitive detection of RNAs in single cells by flow cytometry” Nucleic Acids Res. 20(1):83-8.

Washing can be accomplished by executing a suitable number of washing cycles, i.e., one or more. Each cycle in general includes the following steps: mixing the cells with a suitable buffer solution, detaching non-specifically bound materials from the cells, and removing the buffer together with the waste. Each step is described in more detail below.

Mix the cells with wash buffer: In some assays, the cells are immobilized on the surface of a substrate before being washed. In such cases, the washing buffer is mixed together with the substrate surface. In many other embodiments, the cells to be washed are free-floating. The washing buffer is added to cell pellets or to the solution in which the cells are floating.

Detach non-specifically bound materials from cells: Any of a number of techniques can be employed here to reduce nonspecific binding after cell permeability treatment and probe hybridization to encourage non-specifically bound probes to detach from the cells and dissolve into the wash buffer. These include raising the temperature to somewhere just below the melting temperature of the specifically bound probes and employing agitation using a magnetic or mechanical stirrer or perturbation with sonic or ultrasonic waves. Agitation of the mixture can also be achieved by shaking the container with a rocking or vortex motion.

Remove buffer together with waste: Any convenient method can be employed to separate and remove the washing buffer and waste from the target cells in the sample. For example, the floating cells or substrates that the cells bound to are separated from the buffer and waste through centrifugation. After the spin, the cells or substrates form a pellet at the bottom of the container. The buffer and waste are decanted from the top.

As another example, the mixture is optionally transferred to (or formed in) a container the bottom of which is made of a porous membrane. The pore size of the membrane is chosen to be smaller than the target cells or the substrates that the cells are bound to but large enough to allow for debris and other waste materials to pass through. To remove the waste, the air or liquid pressure is optionally adjusted such that the pressure is higher inside the container than outside, thus driving the buffer and waste out of the container while the membrane retains the target cells inside. The waste can also be removed, e.g., by filtering the buffer and waste through the membrane driven by the force of gravity or by centrifugal force.

As yet another example, the cells can be immobilized on the surface of a large substrate, for example, a slide or the bottom of a container, through cell fixing or affinity attachment utilizing surface proteins. The buffer and waste can be removed directly by either using a vacuum to decant from the top or by turning the container upside down. As yet another example, the cells are optionally immobilized on magnetic beads, e.g., by either chemical fixing or surface protein affinity attachment. The beads can then be immobilized on the container by attaching a magnetic field on the container. The buffer and waste can then be removed directly without the loss of cells the same way as described in the previous example. As yet another example, the cells are optionally immobilized on beads that are larger than or comparable in size to the target cells, e.g., by either chemical fixing or surface protein affinity attachment. The buffer and waste can then be removed through a porous membrane with pore size smaller than the beads. Alternatively, beads together with cells can be separated from buffer and waste by gravity or centrifugal force with the latter being removed from the top layer. As yet another example, the nonspecifically bound probes within cells are induced to migrate out of the cells by electrophoretic methods while the specifically bound probes remain.

As stated before, a washing cycle is completed by conducting each of the three steps above, and the washing procedure is accomplished by executing one or more (e.g., several) such washing cycles. Different washing buffers, detachment, or waste removal techniques may be used in different washing cycles.

### Detection

In the instant technology, the target cells that have signal-generating particles (labels) specifically hybridized to nucleic acid targets in them can be identified out of a large heterogeneous population after non-specifically bound probes and other wastes are removed through washing. Essentially any convenient method for the detection and identification can be employed.

In one embodiment, the suspension cells are immobilized onto a solid substrate after the labeling or washing step described above. The detection can be achieved using microscope based instruments. Specifically, in cases where the signal generated by the probes is chemiluminescent light, an imaging microscope with a CCD camera or a scanning microscope can be used to convert the light signal into digital information. In cases where the probe carries a label emitting a fluorescent signal, a fluorescent imaging or scanning microscope based instrument can be used for detection. In addition, since the target cells are, in general, rare among a large cell population, automatic event finding algorithms can be used to automatically identify and count the number of target cells in the population. Cells in suspension can be immobilized onto solid surfaces by any of a number of techniques. In one embodiment, a container with large flat bottom surface is used to hold the solution with the suspended cells. The container is then centrifuged to force the floating cells to settle on the bottom. If the surface is sufficiently large in comparison to the concentration of cells in the solution, cells are not likely to overlap on the bottom surface. In most cases, even if the cells overlap, the target cells will not because they are relatively rare in a large population. In another embodiment, suspended cells are cytospun onto a flat surface. After removal of fluids, the cells are immobilized on the surface by surface tension.

In certain embodiments of this invention, cells are floating (in suspension) or are immobilized on floating substrates, such as beads, so that pre-detection procedures, such as hybridization and washing, can be carried out efficiently in solution. There are several methods to detect rare target cells out of a large floating cell population. The preferred method is to use a detection system based on the concept of flow cytometry, where the floating cells or substrates are streamlined and pass in front of excitation and detection optics one by one. The target cells are identified through the optical signal emitted by the probes specifically bound to the nucleic acid targets in the cells. The optical signal can, e.g., be luminescent light or fluorescent light of a specific wavelength.

### Advantages

In summary, the instant QMAGEX technology has a number of unique elements that enable multiplex nucleic acid detection in single cells and detection of target cells. These elements include the following.

Nucleic acid molecules immobilized inside cells are used as markers for the identification of CTC (or other cell types). Compared with protein based markers, nucleic acids are more stable, widely available, and provide better signal to noise ratio in detection. In addition, the detection technique can be readily applied to a wide range of tumors or even other applications related to cell identification or classification. As another advantage, nucleic acid molecules are quantifiably measured at an individual cell level, instead of in a mixed cell population. This feature ensures that the cell as a key functional unit in the biological system is preserved for study. In many applications involving a mixed population of cells, this feature can be very useful in extracting real, useful information out of the assay. (For example, a CTC can be identified based on detection of the presence or expression level(s) of a set of nucleic acid marker(s) in the cell; the presence or copy number of additional nucleic acids in the cell can then provide additional information useful in diagnosis, predicting outcome, or the like.)

Cells optionally remain in suspension or in pellets that can be re-suspended in all steps of the assay before final detection. This feature significantly improves assay kinetics, simplifies the process, enhances the reproducibility, and keeps the cell in its most functional relevant status. On the other hand, significant aspects of the invention, including probe selection and design, multiplexing, amplification and labeling, can be applied directly to in situ hybridization technique for the detection and enumeration of rare cells in tissue samples.

A unique indirect capture probe design approach is optionally employed to achieve exceptional target hybridization specificity, which results in better signal to noise ratio in detection.

The assays enable the detection of multiple target genes or multiple parameters on the same gene simultaneously. This feature benefits the detection of rare cells such as CTC in a number of ways. First, it can reduce the false positive rate, which is essential in cancer diagnostics. Second, it can provide additional, clinically important information related to the detected tumor cell, which may include the progression stage and/or original type and source of the primary tumor.

The invented technology incorporates a signal amplification scheme, which boosts the detection sensitivity and enables the detection of rare cells among a large number of normal cells with high confidence.

Detection can be implemented on FACS or flow cytometer based instruments or on microscope based platforms. The former can be fully automated and provides fast detection and the additional benefit of sorting out identified cells for further study, if desired. The latter platform is more widely available and has the benefit of allowing final manual identification through morphology.

### Systems

In one aspect, the invention provides systems and apparatus configured to carry out the procedures of the novel assays. The apparatus or system comprises one or more (and preferably all) of at least the following elements.

Fluid handling: The apparatus optionally includes a subsystem that can add reagents, and if required by the assay, decant fluids from the sample container (e.g., a removable or fixed, disposable or reusable container, for example a sample tube, multiwell plate, or the like). The subsystem can be based on a pipette style fluid transfer system where different fluids are handled by one pump head with disposable tips. As an alternative example, each reagent may have its own dedicated fluid channel.

Mixing and agitation: The apparatus optionally includes a device to mix different reagents in the sample solution and encourage any non-specifically bound material to detach from the cells. The device may have a mechanism to introduce a vortex or rocking motion to the holder of the sample container or to couple sound or ultrasound to the container. Alternatively, a magnetic stirrer can be put into the sample container and be driven by rotating magnetic field produced by an element installed in a holder for the container.

Temperature control: The temperature of the sample can be controlled to a level above the room temperature by installing a heater and a temperature probe to the chamber that holds the sample container. A peltier device can be used to control the temperature to a level above or below ambient. Temperature control is important, e.g., for performance of the hybridization and washing procedures in the assays.

Cell and waste fluid separation: The apparatus optionally includes a device that can remove waste fluid from the sample mixture while retaining cells for further analysis. The device may comprise a sample container that has a porous membrane as its bottom. The pore size of the membrane is smaller than the cells (or beads on which the cells are immobilized) but larger than the waste material in the mixed solution. The space below the membrane can be sealed and connected to a vacuum pump. As an alternative example, the space above the membrane can be sealed and connected to a positive pressure source. In a different embodiment, the device can comprise a centrifuge. The container with the membrane bottom is loaded into the centrifuge, which spins to force the waste solution to filter out through the membrane. In another configuration of this device, the sample container has a solid bottom. Cells deposit at the bottom after centrifugation, and the waste solution is decanted from the top by the fluid handling subsystem described above.

This device can also perform a function that prepares the sample for final readout. In embodiments where the readout is by microscopy, the cells are typically deposited and attached to a flat surface. A centrifuge in the device can achieve this if the bottom of the container is flat. In another approach, a flat plate can spin within its plane, and the system can employ the fluid handling device to drop the solution containing the cells at the center of the spin. The cells will be evenly spun on the plate surface.

Detection: The detection element of the invented apparatus can be integrated with the rest of the system, or alternatively it can be separate from the rest of the subsystems described above. (For example, for FFPE sections assay steps can be performed in an automated ISH station such as those commercially available from Ventana Medical Systems Inc. or Leica Microsystems, then detection can be performed on a separate microscope.) In one embodiment, the readout device is based on a microscope, which may be an imaging or scanning microscope. In another embodiment, the device is based on a fluorescent imaging or scanning microscope with multiple excitation and readout wavelengths for different probes. In a preferred embodiment when the cells are in suspension, the readout device is based on flow cytometry. The cytometry approach is preferred because it can read floating cells directly out of fluid at multiple wavelengths thus greatly improving the efficiency of the assay.

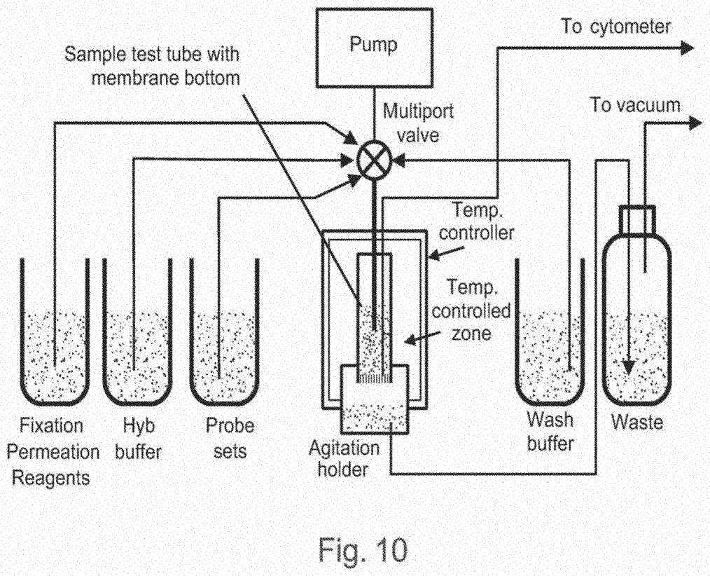

All of the above elements can be integrated into one instrument. Alternatively, these elements may be included in a number of instruments, which work together as a system to perform the assay. FIG. 10 illustrates one particular exemplary embodiment of the instrument configuration. In this particular configuration, the sample is held in a container (sample test tube) with a membrane bottom. Reagents are added from the top of the tube using a pump through a multiport valve. Waste is removed from bottom by vacuum. The holder for the sample container is fixed on an agitation table and the space around the sample is temperature controlled (temp controlled zone) by the temperature controller. The fluid handling element can introduce reagents (fixation and permeation reagents, hybridization buffer, probes sets, and wash buffer) into the sample tube, remove waste into a waste container, and feed cells to a flow cytometer for detection.

One class of embodiments provides a system comprising a holder configured to accept a sample container; a temperature controller configured to maintain the sample container at a selected temperature (e.g., a temperature selected by a user of the system or a preset temperature, different temperatures are optionally selected for different steps in an assay procedure); a fluid handling element fluidly connected to the sample container and configured to add fluid to and/or remove fluid from the sample container; a mixing element configured to mix (e.g., stir or agitate) contents of the sample container; and a detector for detecting one or more signals from within individual cells, wherein the detector is optionally fluidly connected to the sample container. One of more fluid reservoirs (e.g., for fixation or permeabilization reagents, wash buffer, probe sets, and/or waste) are optionally fluidly connected to the sample container.

A system of the invention optionally includes a computer. The computer can include appropriate software for receiving user instructions, either in the form of user input into a set of parameter fields, e.g., in a GUI, or in the form of preprogrammed instructions, e.g., preprogrammed for a variety of different specific operations. As just one example, the software can be preprogrammed for one or more operation such as sample handling, slide handling, de-paraffinization, de-crosslinking, hybridization, washing, etc. as described herein. The software optionally converts these instructions to appropriate language for controlling the operation of components of the system (e.g., for controlling a fluid handling element and/or laser). The computer can also receive data from other components of the system, e.g., from a detector, and can interpret the data, provide it to a user in a human readable format, or use that data to initiate further operations, in accordance with any programming by the user.

### Nucleic Acid Targets

As noted, a nucleic acid target can be essentially any nucleic acid that is desirably detected in a cell. Choice of targets will obviously depend on the desired application, e.g., expression analysis, disease diagnosis, staging, or prognosis, target identification or validation, pathway analysis, drug screening, drug efficacy studies, or any of many other applications. Large numbers of suitable targets have been described in the art, and many more can be identified using standard techniques.

For detection of CTC, as just one example, a variety of suitable nucleic acid targets are known. For example, a multiplex panel of markers for CTC detection could include one or more of the following markers: epithelial cell-specific (e.g. CK19, Muc1, EpCAM), blood cell-specific as negative selection (e.g. CD45), tumor origin-specific (e.g. PSA, PSMA, HPN for prostate cancer and mam, mamB, her-2 for breast cancer), proliferating potential-specific (e.g. Ki-67, CEA, CA15-3), apoptosis markers (e.g. BCL-2, BCL-XL), and other markers for metastatic, genetic and epigenetic changes. As another example, targets can include HOXB13 and IL17BR mRNAs, whose ratio in primary tumor has been shown to predict clinical outcome of breast cancer patients treated with tamoxifen (Ma et al. (2004) “A two-gene expression ratio predicts clinical outcome in breast cancer patients treated with tamoxifen” Cancer Cell 5(6):607-16 and Goetz et al. (2006) “A Two-Gene Expression Ratio of Homeobox 13 and Interleukin-17B Receptor for Prediction of Recurrence and Survival in Women Receiving Adjuvant Tamoxifen” Clin Cancer Res 12:2080-2087). See also, e.g., Gewanter, R. M., A. E. Katz, et al. (2003) “RT-PCR for PSA as a prognostic factor for patients with clinically localized prostate cancer treated with radiotherapy” Urology 61(5):967-71; Giatromanolaki et al. (2004) “Assessment of highly angiogenic and disseminated in the peripheral blood disease in breast cancer patients predicts for resistance to adjuvant chemotherapy and early relapse” Int J Cancer 108(4):620-7; Halabi et al. (2003) “Prognostic significance of reverse transcriptase polymerase chain reaction for prostate-specific antigen in metastatic prostate cancer: a nested study within CALGB 9583” J Clin Oncol 21(3):490-5; Hardingham et al. (2000) “Molecular detection of blood-borne epithelial cells in colorectal cancer patients and in patients with benign bowel disease” Int J Cancer 89(1):8-13; Hayes et al. (2002) “Monitoring expression of HER-2 on circulating epithelial cells in patients with advanced breast cancer” Int J Oncol 21(5):1111-7; Jotsuka, et al. (2004) “Persistent evidence of circulating tumor cells detected by means of RT-PCR for CEA mRNA predicts early relapse: a prospective study in node-negative breast cancer” Surgery 135(4):419-26; Allen-Mersh T et al. (2003) “Colorectal cancer recurrence is predicted by RT-PCR detection of circulating cancer cells at 24 hours after primary excision” ASCO meeting, Chicago, May 2003; Shariat et al. (2003) “Early postoperative peripheral blood reverse transcription PCR assay for prostate-specific antigen is associated with prostate cancer progression in patients undergoing radical prostatectomy” Cancer Res 63(18):5874-8; Smith et al. (2000) “Response of circulating tumor cells to systemic therapy in patients with metastatic breast cancer: comparison of quantitative polymerase chain reaction and immunocytochemical techniques” J Clin Oncol 18(7):1432-9; Stathopoulou et al. (2002) “Molecular detection of cytokeratin-19-positive cells in the peripheral blood of patients with operable breast cancer: evaluation of their prognostic significance” J Clin Oncol 20(16):3404-12; and Xenidis et al. (2003) “Peripheral blood circulating cytokeratin-19 mRNA-positive cells after the completion of adjuvant chemotherapy in patients with operable breast cancer” Ann Oncol 14(6):849-55.

One preferred class of nucleic acid targets to be detected in the methods herein are those involved in cancer. Any nucleic acid that is associated with cancer can be detected in the methods of the invention, e.g., those that encode over expressed or mutated polypeptide growth factors (e.g., sis), overexpressed or mutated growth factor receptors (e.g., erb-B1), over expressed or mutated signal transduction proteins such as G-proteins (e.g., Ras) or non-receptor tyrosine kinases (e.g., abl), over expressed or mutated regulatory proteins (e.g., myc, myb, jun, fos, etc.) and/or the like. In general, cancer can often be linked to signal transduction molecules and corresponding oncogene products, e.g., nucleic acids encoding Mos, Ras, Raf, and Met; and transcriptional activators and suppressors, e.g., p53, Tat, Fos, Myc, Jun, Myb, Rel, and/or nuclear receptors. p53, colloquially referred to as the “molecular policeman” of the cell, is of particular relevance, as about 50% of all known cancers can be traced to one or more genetic lesion in p53. Additional exemplary markers useful for detection of breast cancer cells include, but are not limited to, uPA (urokinase-type plasminogen activator), PAI-1 (plasminogen activator inhibitor-1), PAI-2, and/or uPAR (urokinase-type plasminogen activator receptor). Other additional exemplary markers include, but are not limited to, CK18, CK20, C-met, EGFR, and ERCC1 (a marker for resistance to cisplatin; patients with completely resected NSCLC and ERCC1-negative tumors are helped by cisplatin-based chemotherapy, while in contrast, patients with ERCC1-positive tumors may endure the toxicities of therapy with little benefit).

Taking one class of genes that are relevant to cancer as an example for discussion, many nuclear hormone receptors have been described in detail and the mechanisms by which these receptors can be modified to confer oncogenic activity have been worked out. For example, the physiological and molecular basis of thyroid hormone action is reviewed in Yen (2001) “Physiological and Molecular Basis of Thyroid Hormone Action” Physiological Reviews 81(3):1097-1142, and the references cited therein. Known and well characterized nuclear receptors include those for glucocorticoids (GRs), androgens (ARs), mineralocorticoids (MRs), progestins (PRs), estrogens (ERs), thyroid hormones (TRs), vitamin D (VDRs), retinoids (RARs and RXRs), and the peroxisome proliferator activated receptors (PPARs) that bind eicosanoids. The so called “orphan nuclear receptors” are also part of the nuclear receptor superfamily, and are structurally homologous to classic nuclear receptors, such as steroid and thyroid receptors. Nucleic acids that encode any of these receptors, or oncogenic forms thereof, can be detected in the methods of the invention. About 40% of all pharmaceutical treatments currently available are agonists or antagonists of nuclear receptors and/or oncogenic forms thereof, underscoring the relative importance of these receptors (and their coding nucleic acids) as targets for analysis by the methods of the invention.

One exemplary class of target nucleic acids are those that are diagnostic of colon cancer, e.g., in samples derived from stool. Colon cancer is a common disease that can be sporadic or inherited. The molecular basis of various patterns of colon cancer is known in some detail. In general, germline mutations are the basis of inherited colon cancer syndromes, while an accumulation of somatic mutations is the basis of sporadic colon cancer. In Ashkenazi Jews, a mutation that was previously thought to be a polymorphism may cause familial colon cancer. Mutations of at least three different classes of genes have been described in colon cancer etiology: oncogenes, suppressor genes, and mismatch repair genes. One example nucleic acid encodes DCC (deleted in colon cancer), a cell adhesion molecule with homology to fibronectin. An additional form of colon cancer is an autosomal dominant gene, hMSH2, that comprises a lesion. Familial adenomatous polyposis is another form of colon cancer with a lesion in the MCC locus on chromosome number 5. For additional details on colon cancer, see, Calvert et al. (2002) “The Genetics of Colorectal Cancer” Annals of Internal Medicine 137 (7): 603-612 and the references cited therein. For a variety of colon cancers and colon cancer markers that can be detected in stool, see, e.g., Boland (2002) “Advances in Colorectal Cancer Screening: Molecular Basis for Stool-Based DNA Tests for Colorectal Cancer: A Primer for Clinicans” Reviews In Gastroenterological Disorders Volume 2, Supp. 1 and the references cited therein. As with other cancers, mutations in a variety of other genes that correlate with cancer, such as Ras and p53, are useful diagnostic indicators for cancer.

Cervical cancer is another exemplary target for detection, e.g., by detection of nucleic acids that are diagnostic of such cancer in samples obtained from vaginal secretions. Cervical cancer can be caused by the papova virus (e.g., human papilloma virus) and has two oncogenes, E6 and E7. E6 binds to and removes p53 and E7 binds to and removes PRB. The loss of p53 and uncontrolled action of E2F/DP growth factors without the regulation of pRB is one mechanism that leads to cervical cancer. E6 and/or E7 (e.g., from specific HPV strains, particularly high risk strains such as HPV16 and HPV18) can thus be used as markers for detection of cervical cancer. Other useful markers include, but are not limited to, factors involved in cell cycle control and/or DNA replication that are aberrantly expressed in cervical cancer such as p16^{INK4a}, topoisomerase II alpha (TOP IIA), and mini-chromosome maintenance 2 (Mdm2).

Another exemplary target for detection by the methods of the invention is retinoblastoma, e.g., in samples derived from tears. Retinoblastoma is a tumor of the eyes which results from inactivation of the pRB gene. It has been found to transmit heritably when a parent has a mutated pRB gene (and, of course, somatic mutation can cause non-heritable forms of the cancer).

Neurofibromatosis Type 1 can be detected in the methods of the invention. The NF1 gene is inactivated, which activates the GTPase activity of the ras oncogene. If NF1 is missing, ras is overactive and causes neural tumors. The methods of the invention can be used to detect Neurofibromatosis Type 1 in CSF or via tissue sampling.

Many other forms of cancer are known and can be found by detecting associated genetic lesions using the methods of the invention. Cancers that can be detected by detecting appropriate lesions include cancers of the lymph, blood, stomach, gut, colon, testicles, pancreas, bladder, cervix, uterus, skin, and essentially all others for which a known genetic lesion exists. For a review of the topic, see, e.g., The Molecular Basis of Human Cancer Coleman and Tsongalis (Eds) Humana Press; ISBN: 0896036340; 1st edition (August 2001).

Similarly, nucleic acids from pathogenic or infectious organisms can be detected by the methods of the invention, e.g., for infectious fungi, e.g., Aspergillus, or Candida species; bacteria, particularly E. coli, which serves a model for pathogenic bacteria (and, of course certain strains of which are pathogenic), as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), ( ) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

As noted previously, gene amplification or deletion events can be detected at a chromosomal level using the methods of the invention, as can altered or abnormal expression levels. One preferred class of nucleic acid targets to be detected in the methods herein include oncogenes or tumor suppressor genes subject to such amplification or deletion. Exemplary nucleic acid targets include, but are not limited to, integrin (e.g., deletion), receptor tyrosine kinases (RTKs; e.g., amplification, point mutation, translocation, or increased expression), NF1 (e.g., deletion or point mutation), Akt (e.g., amplification, point mutation, or increased expression), PTEN (e.g., deletion or point mutation), EGFR (amplification), c-met (amplification), MDM2 (e.g., amplification), SOX (e.g., amplification), RAR (e.g., amplification), CDK2 (e.g., amplification or increased expression), Cyclin D (e.g., amplification or translocation), Cyclin E (e.g., amplification), Aurora A (e.g., amplification or increased expression), P53 (e.g., deletion or point mutation), NBS1 (e.g., deletion or point mutation), Gli (e.g., amplification or translocation), Myc (e.g., amplification or point mutation), HPV-E7 (e.g., viral infection), and HPV-E6 (e.g., viral infection).

For embodiments in which a nucleic acid target is used as a reference, suitable reference nucleic acids have similarly been described in the art or can be determined. For example, a variety of genes whose copy number is stably maintained in various tumor cells is known in the art. Housekeeping genes whose transcripts can serve as references in gene expression analyses include, for example, 18S rRNA, 28S rRNA, GAPD, ACTB, and PPIB. Additional similar nucleic acids have been described in the art and can be adapted to the practice of the present invention.

### Labels

A wide variety of labels are well known in the art and can be adapted to the practice of the present invention. For example, luminescent labels and light-scattering labels (e.g., colloidal gold particles) have been described. See, e.g., Csaki et al. (2002) “Gold nanoparticles as novel label for DNA diagnostics” Expert Rev Mol Diagn 2:187-93.

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. See e.g., The Handbook: A Guide to Fluorescent Probes and Labeling Technologies, Tenth Edition or Web Edition (2006) from Invitrogen (available on the world wide web at probes (dot) invitrogen (dot) com/handbook), for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science 298:1759; Nature Biotechnology (2003) 21:41-46; and Nature Biotechnology (2003) 21:47-51.

Labels can be introduced to molecules, e.g. polynucleotides, during synthesis or by postsynthetic reactions by techniques established in the art. For example, kits for fluorescently labeling polynucleotides with various fluorophores are available from Molecular Probes, Inc. (www (dot) molecularprobes (dot) com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection and the like are well known in the art. Instruments for detection of labels are likewise well known and widely available, e.g., scanners, microscopes, flow cytometers, etc. For example, flow cytometers are widely available, e.g., from Becton-Dickinson (www (dot) bd (dot) com) and Beckman Coulter (www (dot) beckman (dot) com).

### Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2008). Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

Making Polynucleotides

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. Nos. 5,635,352, 5,124,246, 5,710,264, and 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company (www (dot) mcrc (dot) com), The Great American Gene Company (www (dot) genco (dot) com), ExpressGen Inc. (www (dot) expressgen (dot) com), Qiagen (oligos (dot) qiagen (dot) com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology (www (dot) piercenet (dot) com). Similarly, any nucleic acid can be fluorescently labeled, for example, by using commercially available kits such as those from Molecular Probes, Inc. (www (dot) molecularprobes (dot) com) or Pierce Biotechnology (www (dot) piercenet (dot) com) or by incorporating a fluorescently labeled phosphoramidite during chemical synthesis of a polynucleotide.

### Application of Cooperative Hybridization in Genotyping

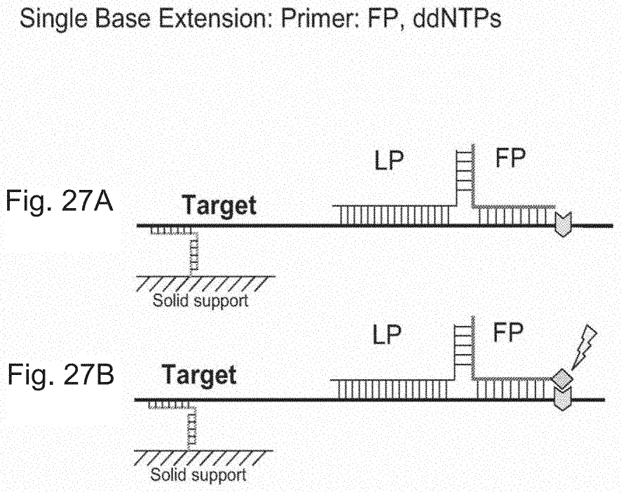

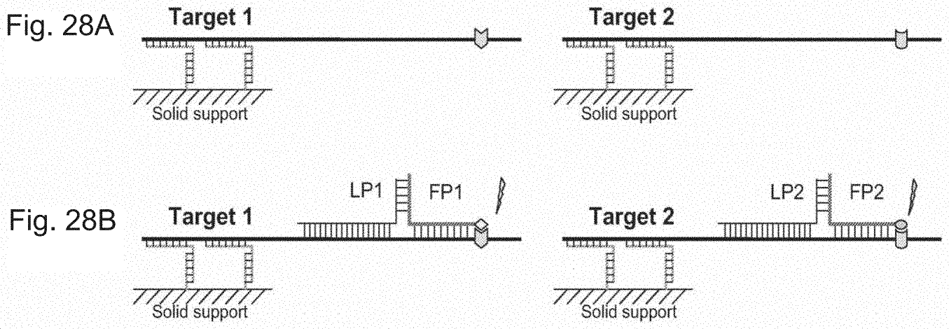

Another application of the described invention is genotyping using primer extension-based method. As shown in FIG. 27A, the FP serves as a primer that anneals to the target sequence with its 3′ end adjacent to a SNP site only when the LP is present. The FP will extend with modified nucleotides through the SNP site by polymerase enzyme and the identity of the extended base is determined either by fluorescence or mass to reveal SNP genotype (FIG. 27B). Because of the high level of specificity imparted by the probe pair, no PCR amplification step of the target sequence is required. Whole genome DNA or amplified whole genome DNA can be used directly as target sequence in the genotyping assay. Furthermore, because primer selection and assay design are simplified, multiple SNPs can be detected simultaneously. For example, many different target sequences can be first captured to different solid supports or a solid support of different locations through capture probes shown in FIG. 28A. Then their individual genotypes can be determined using different paired probes and single base extension (FIG. 28B). Of course, it is also possible to reverse the assay step sequence, i.e. first forming paired probe scaffold then capture the target nucleic acid to different solid supports.

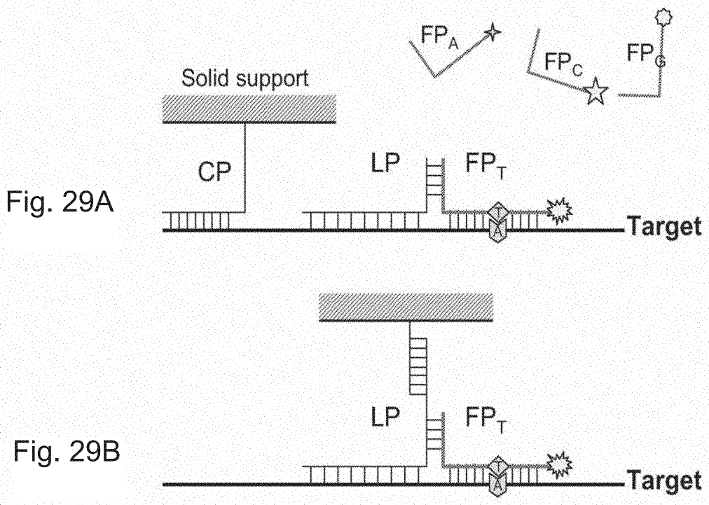

Another application is in hybridization-based genotyping method. Hybridization approaches use differences in thermal stability to distinguish between perfectly matched and mismatched target probe pairs for achieving allelic discrimination. Unlike the primary extension approach shown in FIG. 27, the functional probe used in hybridization approach locates the particular base that compliment to the target SNP within its targeting region, usually near center of the region. A unique fluorescent or other type of signaling label is incorporated to the FP. Four different functional probes corresponding to four allele types: FPA, FPC, FPG and FPT, each with different label, are added into the assay. Only the right FP that perfectly matches the target SNP sequence can form stable probe pair scaffold under the given assay condition. The genotype is detected by the unique label carried by the incorporated FP. The rest, “wild-type” functional probes will be washed away (FIG. 29A). The target nucleic acid can be captured to a solid support using dedicated capture probes, as shown in FIG. 29A. Alternatively, the LP can also be utilized to capture the target to solid support, as shown in FIG. 29B. Because of shorter matching sequence with the target, the FP will offer better discrimination between match and mismatch sequences. Similar to the scheme described in FIG. 28 of primary extension method, different genotypes can be interrogated at the same time by capturing different target nucleic acids to different supports using different capture probes (or LPs). This method offers the potential for highly multiplexed genotyping capability because of the high specificity offered by the invented paired probe approach.

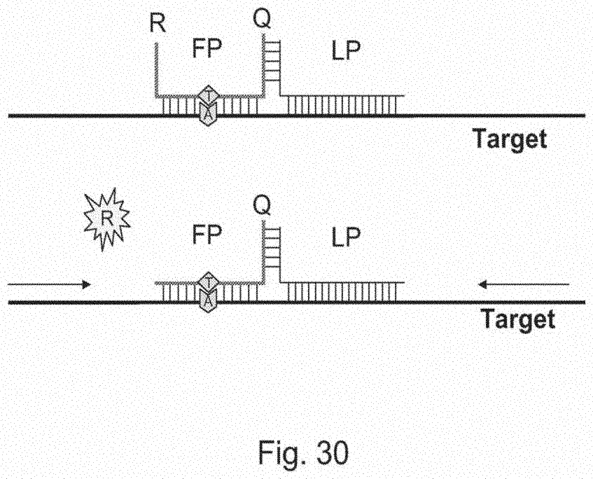

Another example of hybridization-based genotyping is Taqman assay. As shown in FIG. 30, Reporter (R) and Quencher (Q) are incorporated at 5′ and 3′ end of the functional probe, respectively. The paired probe design could enhance discrimination between match and mismatch sequences. Only a perfect match will form a stable scaffold, which enables the FP to bind to the target stably. The R can then be cleaved off and starts to emit signal the same as common Taqman assay.

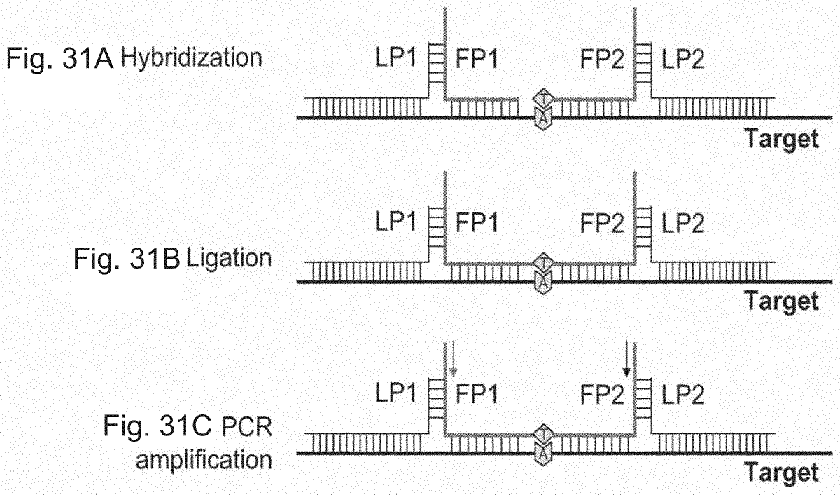

Still another application is in ligation-based genotyping method. Ligation approaches employ the specificity of ligase enzymes to achieve allelic discrimination. When two oligonucleotides hybridize to single-stranded template with perfect complementarity, adjacent to each other, ligase enzymes join them to form a single nucleotide. As shown in FIG. 31A, one or both of the oligonucleotide probes in the ligation pair can be replaced with the invented FP/LP probe pair. The specificity of the assay is enhanced through two levels of specific reaction, one is the more specific hybridization to the target sequence enabled by the paired probes, and another is the co-localization of two FP probes to allow the ligation (FIG. 31B). The particular genotype is identified by amplifying and detecting the product of the ligation using, for example, PCR methods (FIG. 31C). In one specific embodiment, Taqman type of assay can be designed to allow fluorescent detection.

Signal Amplification

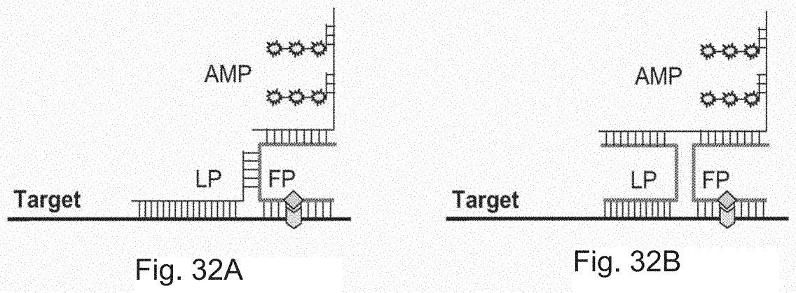

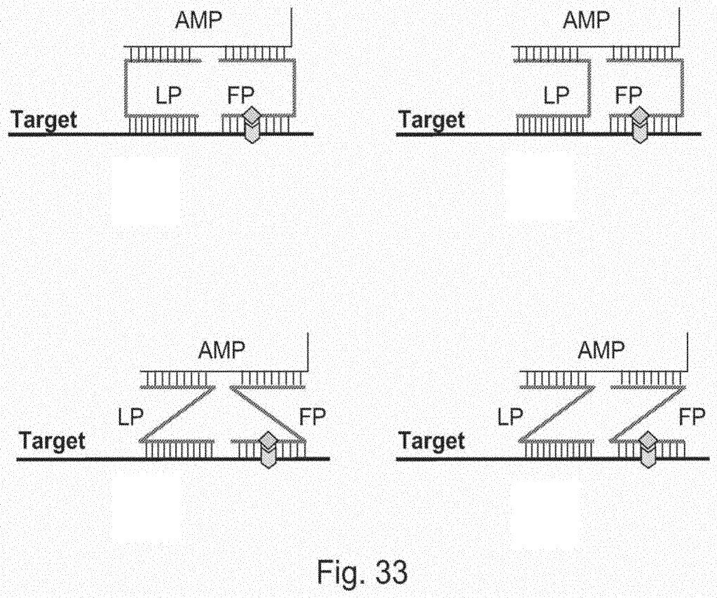

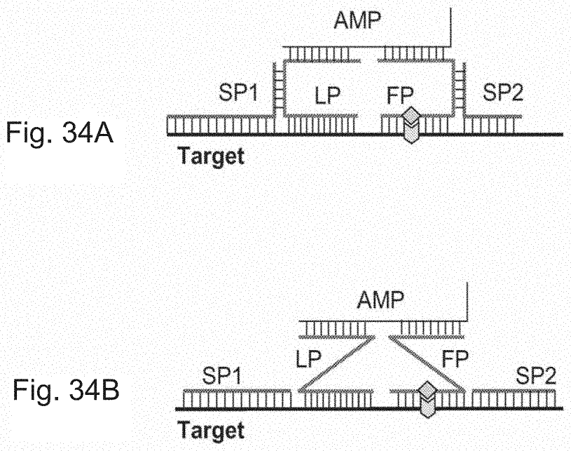

The invented paired probe can be adapted to many exiting amplification techniques to improve detection sensitivity of the assay. The proceeding section described the use of PCR to amplify the product of a ligation as a segregate for the target nucleic acid. FIG. 32 shows an example of different signal amplification approaches, where a large amplifier is hybridized onto the probe pair. Fluorescent or other types of signaling labels are incorporated on to the amplifier. Because the amplifier can be much larger than the target sequence, many more label molecules can be associated to a target thus providing many fold signal amplification. The amplifier can be a large molecule, such as the Branched DNA (U.S. patent Ser. No. 05/635,352), or a large scaffold assembled from multiple molecules through hybridization (U.S. patent Ser. No. 07/033,758). In FIG. 32A, the amplifier (AMP) is hybridized to either FP or LP alone or one each. In FIG. 32B, however, the AMP is hybridized to both FP and LP. There may or may not be any direct hybridization bond between FP and LP. In this configuration, the scaffold interconnects the target, LP, FP and AMP. The hybridization strength between any of the two components of the scaffold is weak and unstable under the assay condition. But due to the interconnections among the four components, the scaffold has much greatly thermal stability, which enables the amplifier to strongly and specifically attach to the target. The scaffold interconnecting these four parts can take many different forms. FIG. 33 shows several additional examples. Additional support probes (SP) may be placed on either side of the scaffold, as shown in FIG. 34, to further increase the hybridization strength of the structure. These support probes may have regions that bind directly with LP or FP, as shown in FIG. 34A, or simply hybridize to the target or the amplifier immediately adjacent to LP or FP (FIG. 34B).

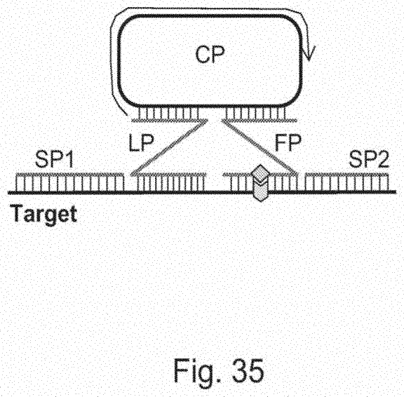

The fact that the LP/FP/AMP scaffold can only be formed under a highly specific condition can be utilized in other signal amplification approaches. In FIG. 35, the AMP is replaced by a circular probe (CP). A rolling circle amplification is commenced using the section of LP or FP that binds to the CP as the primer. The product of the amplification (copies of CP) is detected, which indicates the presence or quantity of the target. Detection specificity of the assay is assured by the highly specific condition under which CP binds to LP and FP.

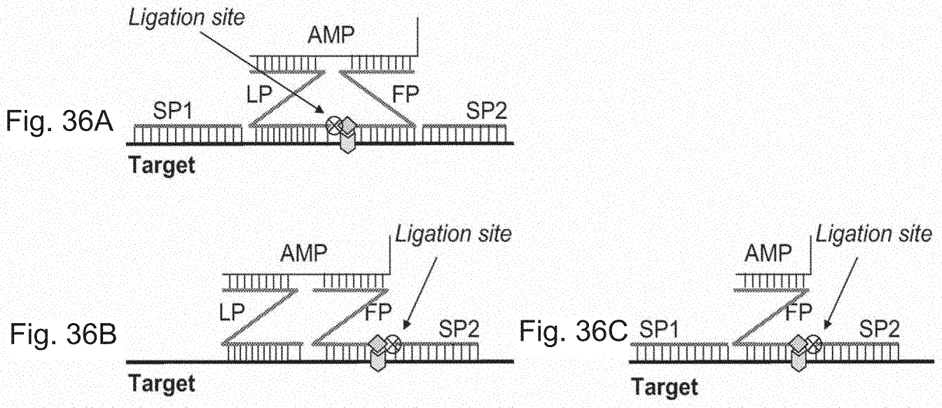

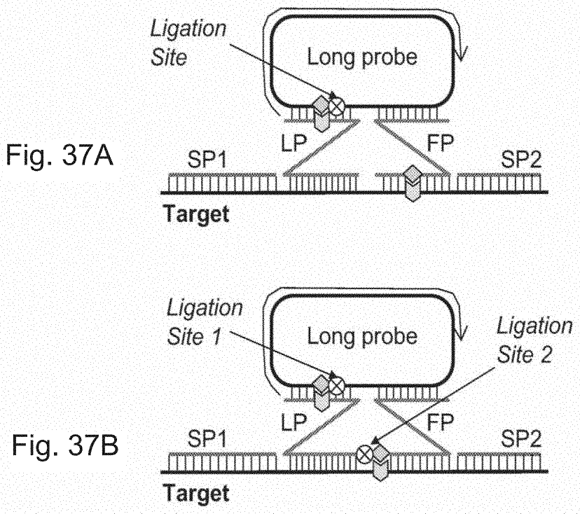

The probe scaffold configurations, genotyping methods and amplification approaches described above can be used in combination to further enhance specificity and sensitivity of the assay. FIG. 36 shows several examples of such combinations. In FIG. 36A, targeting regions of LP and/or FP are designed to be so short that stable scaffold can be maintained when and only when the targeting regions of these two probes are ligated together. Highly specific genotyping is achieved because such ligation is only possible if the end base of the FP is complimentary to the SNP. In FIGS. 36B and 36C, the ligation occurs between the FP and one of the SPs. The support probe SP2 in FIG. 36C can also be recognized as a LP with a zero base anchoring region. Ligation can also be utilized to further boost the specificity of the rolling circle amplification assay described in FIG. 35. As shown in FIG. 37, the circular probe is replaced with a long probe that can be fold into a circle when it is hybridized to the LP and FP. The circle is completed by ligation allowing rolling circle amplification to occur. High specificity is achieved because the ligation can only occur when the long probe is folded into a circle and binds to LP and FP at exact locations. In this assay, it is also possible to add another ligation to targeting region of FP as shown in FIG. 37B.

In Situ Genotyping:

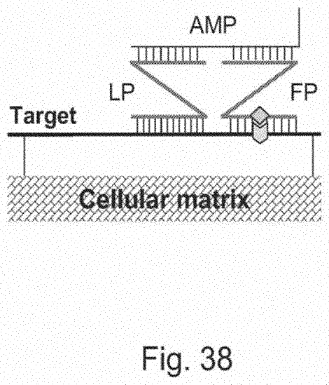

All of above described methods could potentially be used in in situ genotyping within individual cells. The only difference is that the target nucleic acid molecule is anchored to cellular matrix within cells (FIG. 38) instead of to a solid support using dedicated capture probes (FIGS. 27, 28 and 29A) or location probe (FIG. 29B). One challenge for in situ genotyping is the presence of great excess of nonspecific nucleic acid sequences in cells. The LP/FP probe pair design should greatly increase the specificity to enable highly specific genotyping of intended target sequence in the presence of excess amount of nonspecific sequences. Another challenge for in situ genotyping is the limited number of target sequences in single cells available for genotyping. Thus the sensitivity of in situ genotyping detection needs to be as high as being able to detect the genotype of a single nucleic acid molecule.

To achieve the level of detection sensitivity for single copy in situ genotyping, a number of approaches can be used. One approach involves the deployment of signal amplification schemes, such as the ones described above and depicted in FIGS. 32, 33 and 34. Another approach involves the use of PCR amplification as shown in FIG. 31. Since the PCR reaction is on the ligated oligo sequences that do not experience chemical modifications such as formalin fixation in the target sequence, the in situ PCR reaction should have much higher efficiency. To prevent the PCR reaction product leaking out of the cells, strategies used by RainDance Technologies or BEAMing (Li M, Diehl F, Dressman D, Vogelstein B, Kinzler K W. (2006) BEAMing up for detection and quantification of rare sequence variants. Nat Methods. 3(2):95-7) to use water-in-oil emulsion to wrap around cells can be used. A variety of PCR reactions can be used for genotyping. One example is the use of TaqMan probes.

Yet another approach involves using amplification of a surrogate using rolling circle amplification approach as shown in FIGS. 35 and 37. In general, all amplification methods, probe configurations and specificity enhancement approaches described in this invention can be adapted for in situ genotyping.

### Reducing False Positive Signals and Improving Signal to Background Ratio by Reducing the Size of Signal Generating Probe

This invention also describes new approaches to reduce false positive signals and improve signal-to-background ratio. As described in prior sections, Signal Generating Probe comprises one or more labels and is capable of hybridizing a set of two or more capture probes (also called “Capture Probe Set” (CPS)). SGP is also called “Label Probe System” (LPS). A set of capture probes is also called “Capture Probe Set” (CPS). The LPS may comprise a relatively large structure in order to attaching many label molecules on to it. In prior arts, the “cooperative hybridization” event between LPS and target is one-to-one association through one CPS and the CPS is typically directly associated with the target.

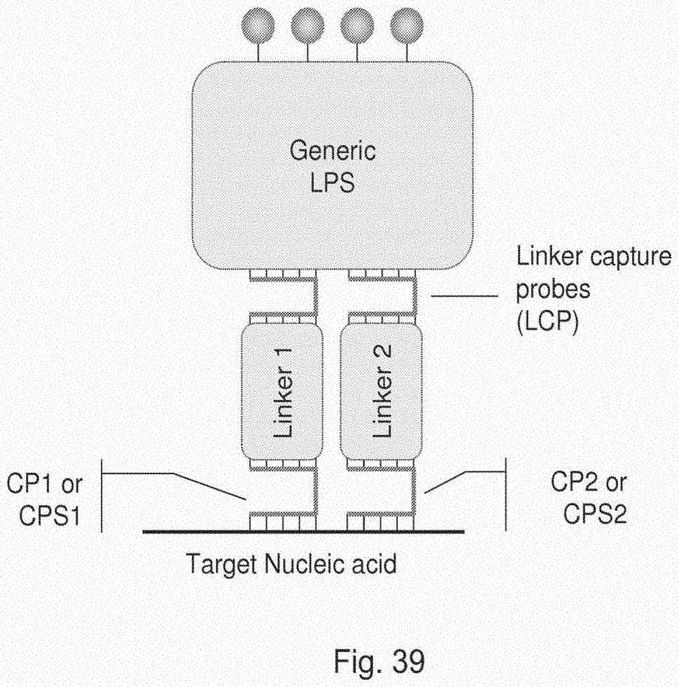

In one embodiment of the current invention, the “cooperative hybridization” event doesn't have to be directly associated with the target probe. As illustrated in FIG. 39, the “cooperative hybridization” can happen via “Linker Capture Probes (LCP)” between the Linkers and LPS. Thus it is only indirectly associated with the target. The only condition that needs to be satisfied here is that the two or more LCPs are indirectly associated with two or more independent regions of the target. Each LCP does not have the sufficient binding strength to capture or bind the LPS stably alone, but the combined binding strength of two or more LCPs can capture or bind LPS stably.

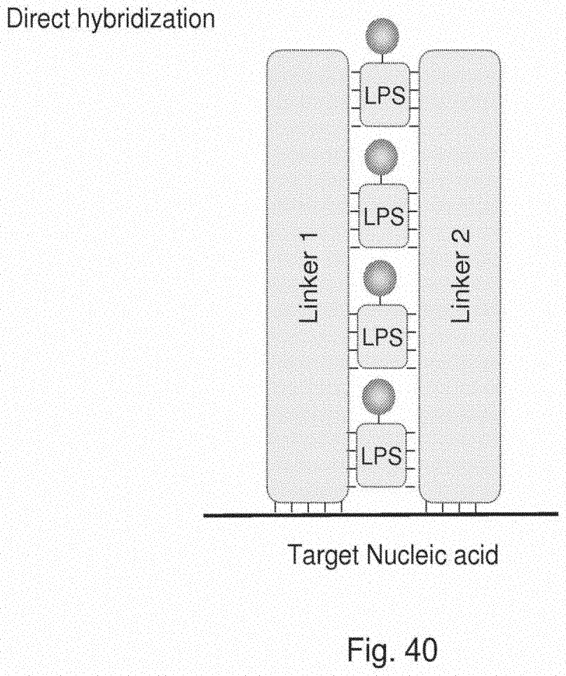

In another embodiment of the current invention, multiple LPS can be associated with one target sequence through multiple LCP anywhere between the target and the label. Because multiple LPS are now associated with one target sequence by multiple LCP, each LPS can be much smaller in size, but together they can still achieve the same level of signal amplification as one big LPS. Due to the smaller number of labels in the smaller-sized LPS, the false positive or background signals due to trapping or nonspecific hybridization are now greatly reduced. FIG. 40 illustrates one example of such design concept.

In general, the current invention of incorporating multiple smaller LPS in the place of one large LPS to be associated with a target satisfies following conditions. To ensure binding specificity, the multiple LPS will only be associated with the target through two or more independent linkers. Each linker will bind to one independent region of the target. Each linker alone doesn't have sufficient binding strength to bind or capture the multiple LPS stably to the target. But two or more linkers together can stably bind or capture the multiple LPS. An example of the above concept is illustrated in FIG. 40, the linker is associated with target on one end and the multiple LPS on the other end. The binding between Linker 1 and each LPS and between Linker 2 and each LPS is weak, but the combined binding of Linker 1 and LPS and Linker 2 and LPS is now strong enough to hold each of the multiple LPS stably.

It is understood that the target can be either nucleic acids or proteins. Each linker can be consisted of one or multiple sequences or entities so long as they are linked with both the target and multiple LPS. The linker can be associated with the target or multiple LPS directly or indirectly. The target region each linker binds to can be small. If the target is a protein, the target region can be a single epitope. If the target is a nucleic acid, it can typically be less than 100 base, preferably less than 50 base, more preferably less than 35 base, more preferably less than 30 base. It is further understood that the binding strength each linker contribute to the capture of each LPS will be weak, insufficient to capture or bind each LPS stably alone. The linker that captures each of the multiple LPS can be one or more antibodies that bind weakly to each of the multiple LPS. Alternatively, the linker that captures each of the multiple LPS can be one or more nucleic acids. The sequence that hybridizes between the linker and each of the multiple LPS will preferably be below the hybridization temperature, by 3 degree, 5 degree, 10 degree or more. The length of the sequence involved in the hybridization between the linker and each LPS can be 20 base or less, more preferably 16 base or less, 15, 14, 13, or 12 base or less. The two or more linkers binding to the independent region of the target can preferably close together in the target within 100 base, more preferably within 50, or 20, or 10, or 5 base, or right next to each other with no base gap.

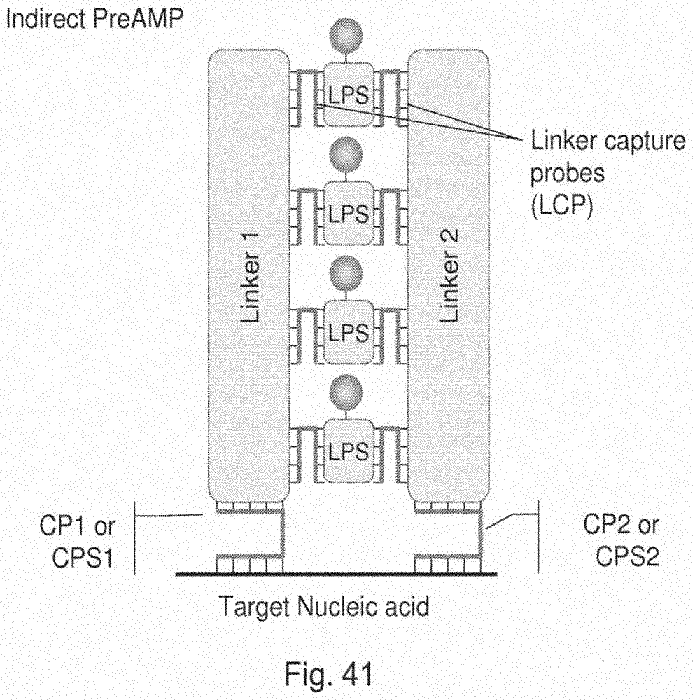

FIG. 41 shows one embodiment where the Linkers are not directly linked to either target or LPS. The target is associated with the Linkers through a CP or a CPS. Each of the multiple LPS is associated to the Linkers via multiple LCP. Each LCP has one portion that binds to the Linker and another portion that binds to the LPS. Each of the multiple LPS is held by one or more LCPs from each Linker. Again the binding strength between each LCP and the Linker, or between each LCP and the LPS, or both are intentionally designed to be weak so that a single LCP can not hold the LPS stably to the linker, but the combined binding strength of multiple LCPs from different linkers will stably capture the LPS. Therefore, two or more linkers each containing one or more LCPs binding to each LPS are designed to amplify the signal whereas a single linker will not generate signal. In another word, when and only when multiple linkers are present, a signal will be generated.

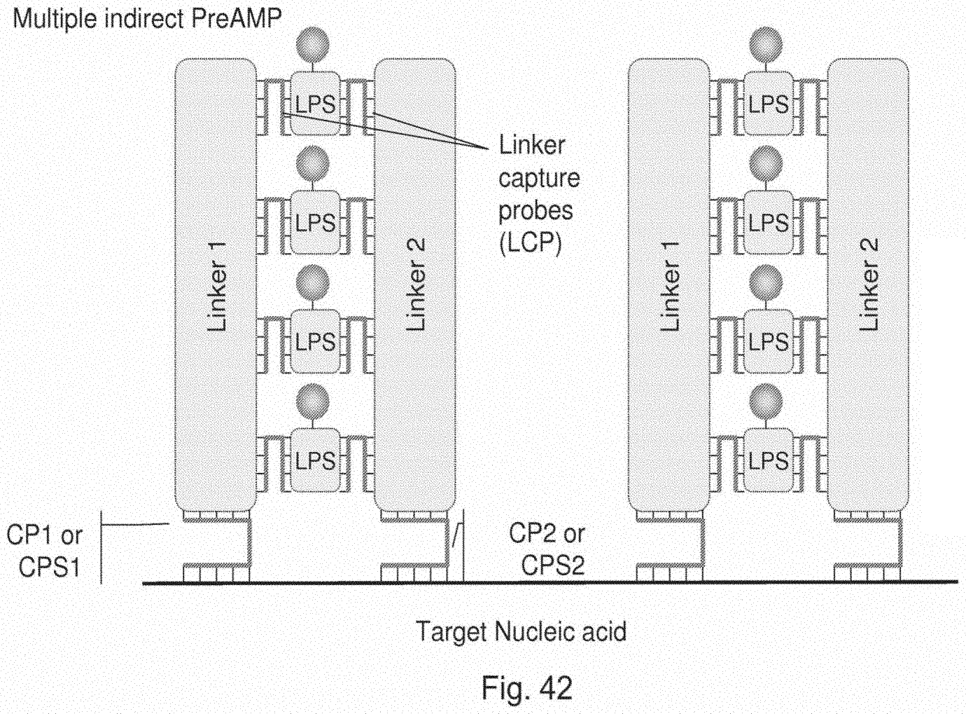

Although FIG. 40 and FIG. 41 illustrate the capture of multiple LPS to one target region, it is understood that there can be multiple target regions within each region multiple LPS can be captured as illustrated in FIG. 42.

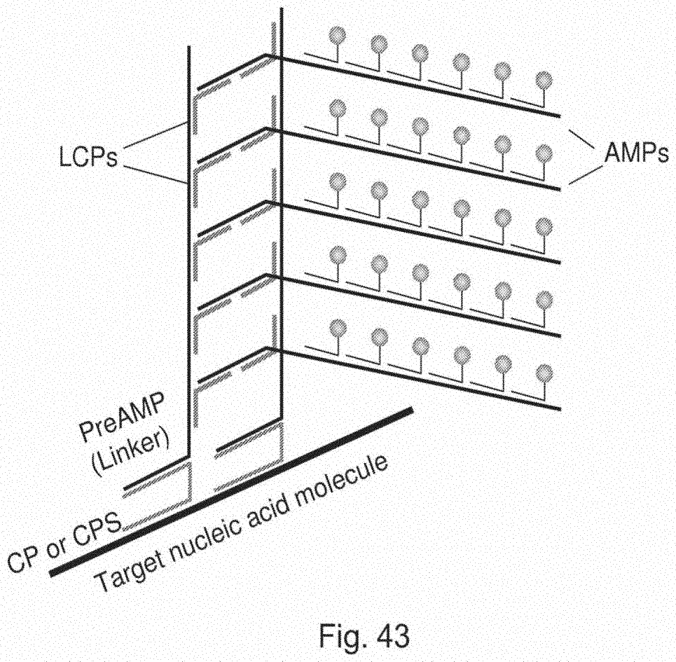

FIG. 43 shows one implementation example of the approach illustrated as a concept in FIG. 41. Two PreAMPs are captured to the two independent regions of the target by their respective capture probe (CP) or capture probe set (CPS). The PreAMP here serves as the Linker in FIG. 41. The AMPs are not coupled to the PreAMPs directly. Instead, a section of AMP is designed to bind simultaneously to two linker capture probes (LCP), one of which binds to one PreAMP and the other binds to the other adjacent PreAMP. Again, the melting temperature of the hybridization between the LCP and the AMP is designed to be lower than the hybridization temperature of the assay, so that a single LCP can not hold an AMP to the PreAMPs stably through the assay. But the binding of two LCPs produces sufficient binding strength to hold the AMP to the PreAMPs through the assay. In this way, one non-specifically bound or trapped PreAMP will not produce any false positive signal. A non-specifically located AMP, on the other hand, may still bind multiple label molecules. However, the level of such false positive or background signal becomes insignificant compared to the real signal.

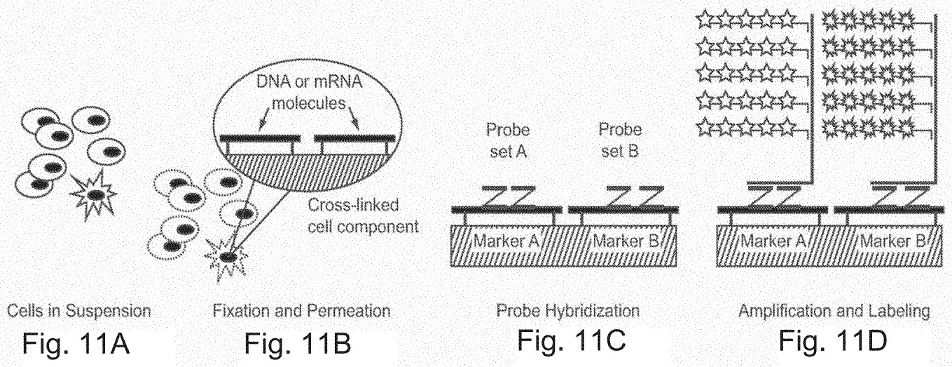

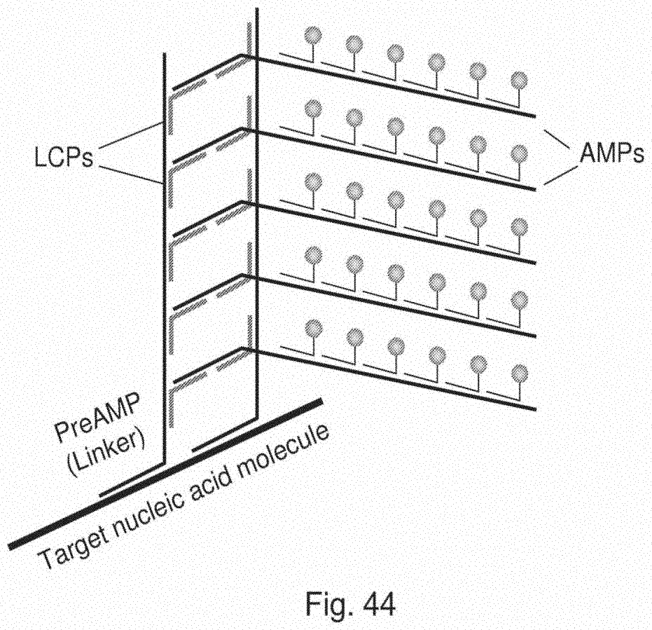

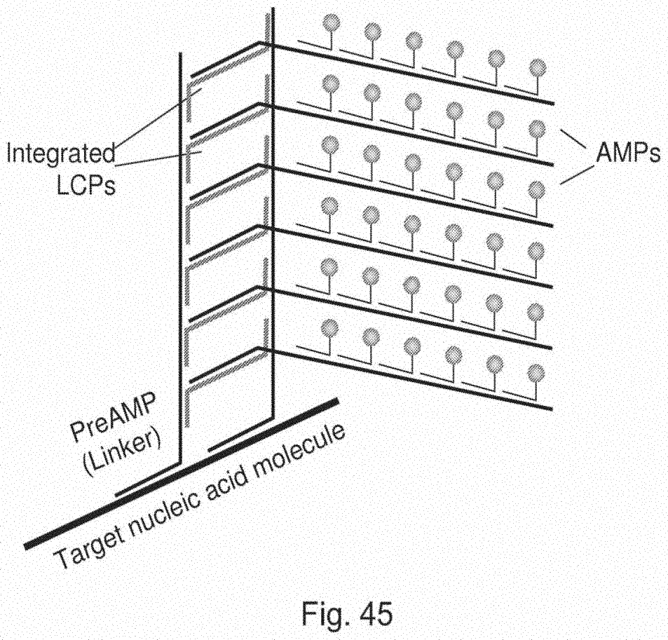

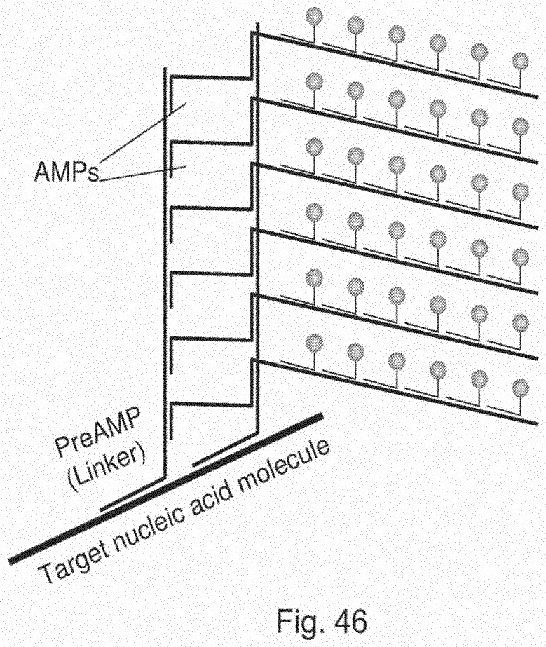

FIG. 44 shows another specific implementation example of the approach illustrated as a concept in FIG. 40. Compared with the configuration in FIG. 43, the Linkers (or PreAmps) are now directly bound to the target nucleic acid without using capture probes or capture probe sets. FIG. 45 shows yet another implementation example where the pair of linker capture probes (LCP) in FIGS. 10 and 11 is integrated into a single one. The binding between the LCP and the Linker can be designed to be intentionally weak so that the binding between LCP and a single linker is not sufficiently strong to hold the integrated LCP through the assay hybridization. When and only when both linkers are in position and LCP binds to both linkers simultaneously that sufficient binding strength is generated that holds the LCP to linkers, thus capturing the label probes to the target through the hybridization. FIG. 46 shows a further simplification of the probe configuration with the linker capture probes (LCP) is integrated into the amplifier (Amp). Again the binding between Amp and individual Linker can be intentionally weak so that when and only when both Linkers are in position and the Amp binds to them simultaneously that sufficient binding strength is generated to hold the Amp to the Linker thus capturing the label probes to the target. FIGS. 45 to 46 have shown probe set configurations without capture probe or capture probe sets capturing the linkers to the target. It is obvious that the same schemes will also work with the capture probes or capture probe sets in place as illustrated in FIG. 43.

### Application of Cooperative Hybridization to Detect Abnormal Juxtapositon and Other Genetic Mutations

The methods described in this invention can be used to reduce background and false positive signal in all applications related to detection of nucleic acid targets. They are particularly useful in the detection of nucleic acid targets inside individual cells, where cellular matrix usually produces higher level of background noise. These methods are even more useful in applications involving the detection of single point mutation (SNP) and the detection of abnormal juxtaposition of genetic material because background noise in these applications can directly lead to false positive test result.

Detecting events in which specific sections of nucleic acid sequences have aberrantly connected together is very important because such events often have biological and clinical implications. The unintended juxtaposition of two nucleic acid sequences can occur in multiple ways and have an impact both at the DNA and RNA levels. For example, the rearrangement of DNA through a translocation can lead to the fusion of two genes, potentially disrupting importing protein coding regions. Also, a gene fusion event can lead to the creation of a chimeric RNA sequence that has transformative properties. Finally, a point mutation in a splice acceptor site at an intron/exon boundary could cause the inclusion or exclusion of unintended sequences in the final mRNA due to aberrant splicing.

Of the various point mutations, chromosomal rearrangements, and epigenetic changes that can cause mis-joined nucleic acid sequences, chromosomal rearrangements resulting in gene fusions are the most prevalent somatic mutation in cancer development, accounting for 20% of deaths due to cancer. One result of this abnormal juxtaposition of genetic material is the creation of a chimeric mRNA transcript from the fusion of two different coding regions. The resulting protein is considered a driving cause of the underlying disease and a potential therapeutic target since its expression is limited to cancer cells. In addition, the restricted expression pattern of the fusion mRNA and protein make them ideal candidates for use as biomarkers in cancer diagnostics.

The best studied example of a gene fusion event is the creation of the Philadelphia chromosome from the reciprocal chromosomal translocation t(9;22), which joins the break point cluster region (BCR) with the Abelson kinase gene (ABL). It was the first example of a causal link between genetic alterations and the development of cancer, being present in 100% of chronic myeloid leukemia (CML) cases. Because of the direct association between the creation of the fusion protein and the disease, inhibition of ABL kinase signaling is a prime target for drug inhibition. In fact, the tyrosine kinase inhibitor imatinib (Gleevec) was developed and patients treated with the drug in a major clinical study showed an overall survival rate of >85% at 5 years regardless of the severity of the disease at diagnosis.

The early key finding that gene fusions have a causative role in carcinogenesis and the more recent evidence that the protein products can be selectively targeted by drug therapies has lead to an increased interest in identifying novel genomic rearrangements. Screening methods at both the DNA and transcript levels have brought the total number of known gene fusions in malignant cancers to over 300 including the previously identified ones. Of these, 75% are found in haematological disorders such as CML, ALL, and Burkitt's lymphoma, and the rest are present in solid tumors, mainly prostate, thyroid, breast, and lung. It has also been discovered that a single oncogene can have multiple fusion partners, though the specific disease outcome is always the same. Though a positive correlation with disease for most of the newly discovered gene fusions has yet to be determined, this large number of potential clinical biomarkers and therapeutic targets will require a new set of reagents for detection as research into disease association moves forward.

Methods for confirming the presence of a known gene fusion have been developed both at the DNA and RNA levels. For DNA, detection can be done using fluorescent in situ hybridization (FISH) with probes complimentary to specific DNA sequences. This method allows for the direct visualization of genomic rearrangements including translocations and inversions. In addition, amplification by PCR of genomic sequence surrounding potential DNA breakpoints, followed by sequencing of the product, can also be employed to detect sequence level alterations. For the detection of known gene fusions at the RNA level, RT-PCR can be used with a primer pair containing one primer homologous to either of the genes to be detected. A positive RT-PCR product confirms that two different genes are part of the same transcript. To the best knowledge of the inventors, there has been no prior art in detecting mis-joint of nucleic acid sequences in situ at RNA level. Methods have also been created for fusion gene discovery. These include transcriptome sequencing, genome-wide massively parallel paired-end sequencing, and paired-end diTags (PET).

In addition to gene fusion events leading to chimeric transcripts, mutations affecting RNA splicing can also create mis-joined RNA sequences that lead to disease. The causal mutations can occur directly on cis-acting elements within a gene, or can occur in trans-acting elements such as regulators of splicing. Either way, nucleic acid sequences that are normally present in the mRNA can be excluded, or new sequence can be introduced, both of which lead to a novel transcript.

One of the best studied examples of alternative splicing alterations leading to disease is the case of the transcription factor KLF6 in prostate cancer. A point mutation in the KLF6 gene causes to the use of a cryptic splice site, leading to a partial deletion of RNA sequences. Though some normal protein is still produced, it is believed that the new truncated protein product acts as a dominate-negative mutant, inhibiting the function of wild-type protein products. The end result is an increased susceptibility to prostate cancer.

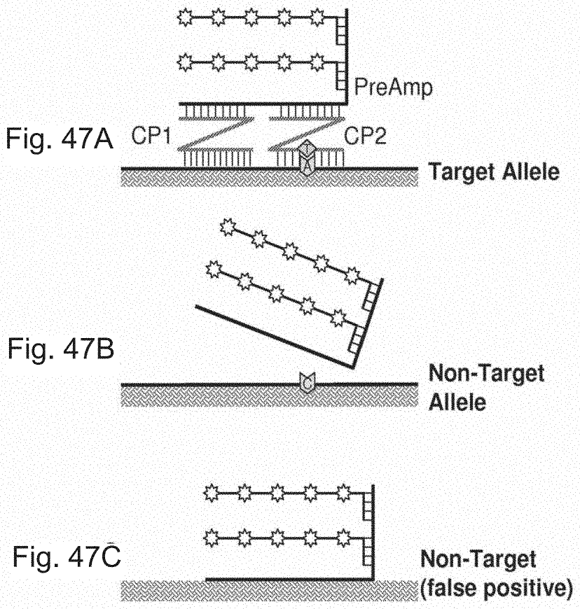

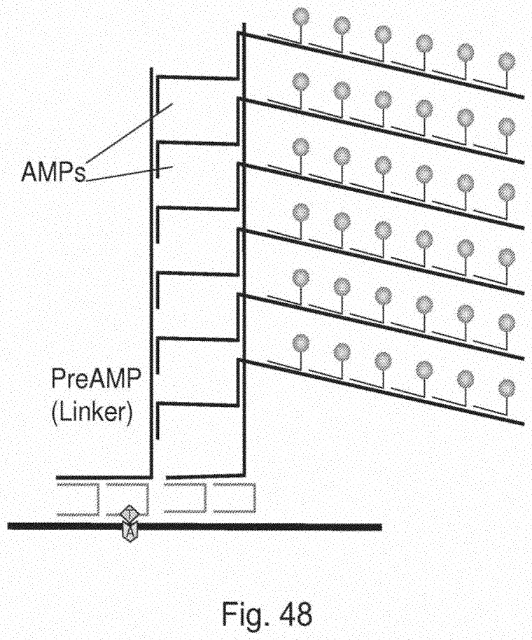

FIG. 47 shows an example method of detecting a particular type of allele. It utilizes a capture probe set to capture label probes to the target (FIG. 47A). The binding between target and each capture probe is intentionally designed to be weak. The label probes can only be hold stable to the target allele through the hybridization step when and only when both capture probes are present. Therefore, if there is a base mismatch at one of the capture probe, this capture probe can no longer hold on the target. With one capture probe absent, the remaining capture probe does not have sufficient binding strength to hold the label probe system to the target and will be washed away (FIG. 47B). One problem with this method of detection is that if the PreAmp molecule binds nonspecifically to other nucleic acid or stuck in cellular matrix, a false positive signal will be generated (FIG. 47C). All false positive signal reduction methods described in this invention can be applied here. FIG. 48 shows a particular way to use the method illustrated in FIG. 46 to reduce false positive signal. Multiple amplifiers (Amp) are captured to the target when and only when both PreAmps (Linkers) are captured to the target by their respective the capture probe sets. If one amplifier is bound non-specifically, it can only produce a much lower level of signal than that of a real target. The chance of false positive results is therefore reduced.

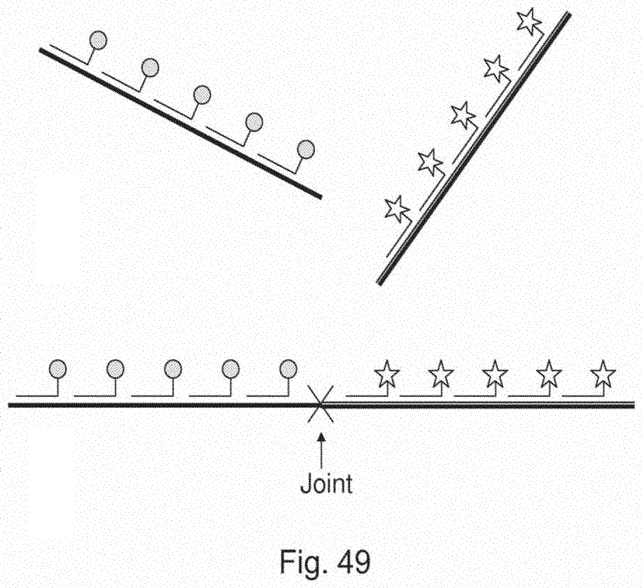

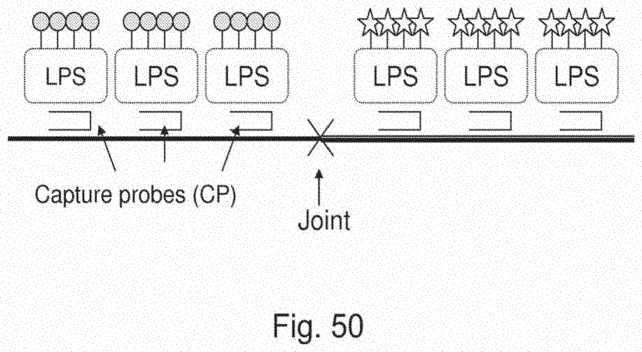

FIG. 49 illustrates a method of detecting the unintended juxtaposition of two nucleic acid sequences. Fundamentally, this method binds in situ different labels that produce distinguishable signals to the two different sections of nucleic acid that are suspected of being mis-joined. If these two sections are indeed connected together, directly or indirectly, these two associated signals can be detected to be spatially co-located or have fixed spatial associations. For example, if one section is bound to green fluorescent dye and the other red dye, the spliced sections can be observed under fluorescent microscope as yellow dots in combined color image or the green and red dots appear at the same location in separate color images. Instead of binding labels directly to the nucleic acid as illustrated in FIG. 49, labels can be bound to a Label Probe System, which is in turn captured to the nucleic acid through capture probes. In such “indirect labeling” scheme, shown in FIG. 50, more label molecules can be attached to the same nucleic acid section creating a signal amplification effect.

We have developed an in situ hybridization method (U.S. Pat. No. 7,709,198) called RNAscope, that allows for the direct visualization of RNA in situ. This method utilizes the oligonucleotide probe sets and novel signal amplification systems previously described. Our assay can be used on a variety of sample types including cultured cells, peripheral blood mononuclear cells (PBMCs), frozen tissue, and formalin-fixed paraffin embedded (FFPE) tissue. In addition, the assay can utilize both chromogenic and fluorescent detection reagents.

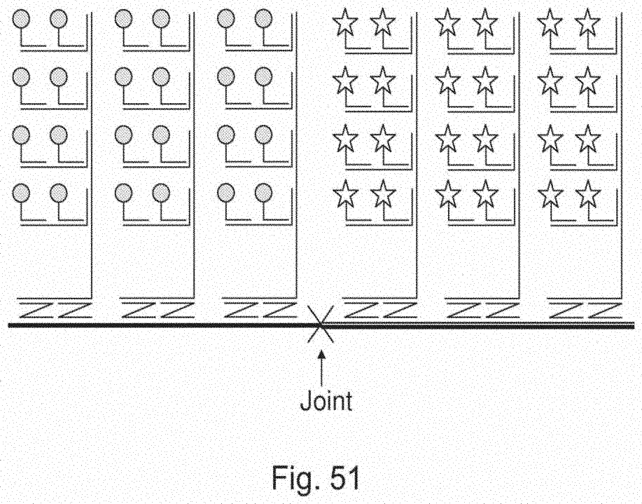

This invention concerns the in situ visualization of mis-joint nucleic acid sequences, in particular, RNA transcripts derived from gene fusions and aberrant splicing. This invention further concerns the adaption of RNAscope assay technology to detect, in situ, mis-joint nucleic acid sequences at RNA or DNA level. FIG. 51 illustrates a specific approach of this invention. Two oligonucleotide probe sets are designed and synthesized: one set is complimentary to the 5′ portion of the RNA transcript containing sequences from one gene in the fusion, and the other set is complementary to the 3′ portion of the RNA transcript containing sequences from the second gene in the fusion. Both probe sets are then hybridized simultaneously to the sample. Following probe set hybridization, two different PreAmplifiers (PreAmp), each of which recognizes a specific probe set, are simultaneously hybridized to the target probes. Following PreAmp hybridization, two different Amplifiers (Amp), each of which recognizes a specific PreAmp, are simultaneously hybridized to the PreAmps. Finally, label probe molecules, each of which recognizes a specific Amp, are simultaneously hybridized to the Amps. For example, if detection of the fusion RNA transcript using colorimetric reagents is desired, then one of the label probes used is conjugated to horseradish peroxidase (HRP) and the other to alkaline phosphatase (AP). After addition of AP and HRP specific substrates, the HRP molecule will deposit a color precipitate at the site of the target probe hybridization, and the AP molecule will deposit a precipitate of different color. Overlapping precipitates of different colors indicates the presence of the gene fusion transcript. If detection of the fusion transcripts using fluorescence is desired, then each of the label probes is conjugated a different fluorescent dye. After label probe hybridization, presence of the fusion transcript can be visualized under a fluorescent microscope and is identified by the overlap of the two fluorescent signals.

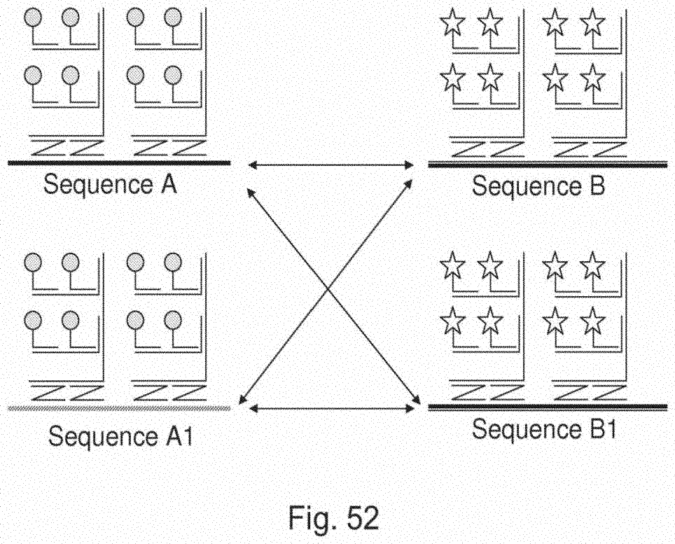

Sometimes the joining of a one nucleic acid sequence to multiple partners produces the same clinical or biological outcome. For example, the gene MLL can be fused with over 60 different partner genes; however, regardless of the fusion partner, the outcome is still acute leukemia, and monitoring of the various fusion transcripts after treatment must still be done. As shown in FIG. 52, for example, jointed sequences produced by two different 3′ side sequences A and A1, and two 5′ side sequences B and B1 can produce up to four difference combinations (AB, A1B, AB1, A1B1). If all these combinations share the same clinical outcome, such an outcome can be identified by associating all possible nucleic acid sections on one side of the splice to one label and all possible nucleic acid sections on the other side of the splice to another, distinguishable label. The outcome is always indicated by the co-location of these two labels no matter which combination of splices have occurred.

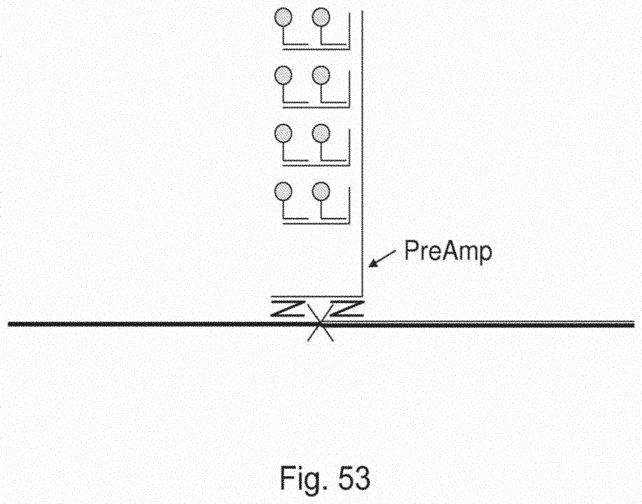

Some other times, it is useful to detect exactly the specific junction of the splicing event. FIG. 53 illustrates one possible approach to detect such a junction. The label probe system, which, in this example, includes pre-amplifier (PreAmp), amplifiers and label probes, is capture to the spliced nucleic acid at the junction by a pair of capture probes (CP), which are designed to bind to each side of the splicing junction, respectively. Similar to the capture probe design in RNAscope assay, the binding strengths between the CPs and the spliced nucleic acid or between the CPs and the PreAmp, or both, are designed as such that a single CP can not hold the PreAmp stably through the assay hybridization process. When and only when both CPs present at their designed position that the PreAmp will be captured securely to the nucleic acid. Therefore, if the splicing event does not occur, the PreAmp will not be attached to either side of the nucleic acid. No signal will be generated. When and only when a splice event occurs, both CPs in the pair will present and PreAmp will then be captured to the spliced nucleic acid. A signal can then be detected.

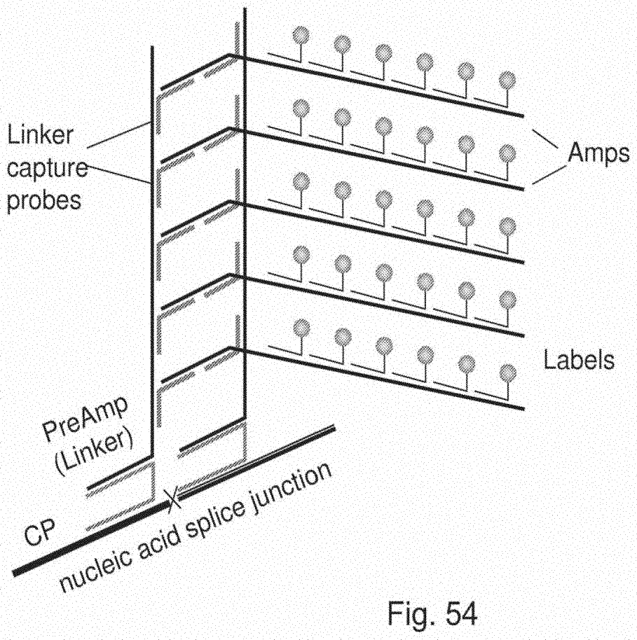

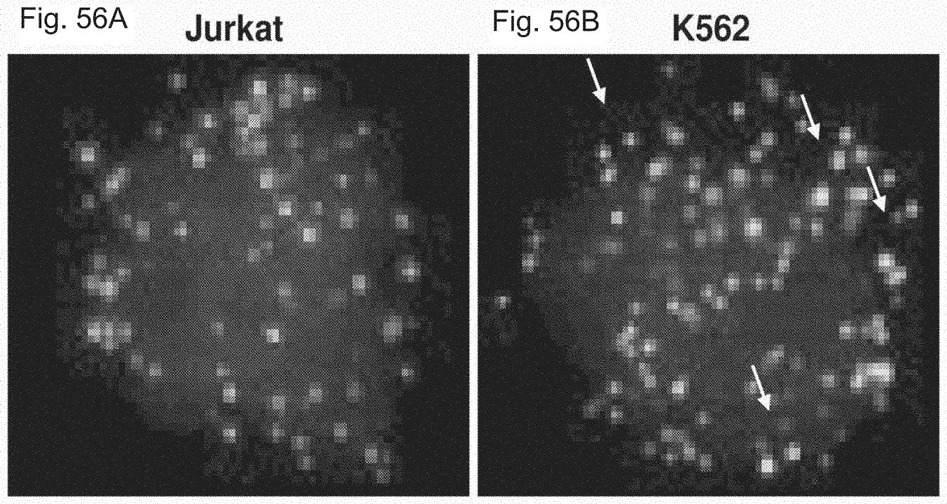

The approach in FIG. 53 may encounter the problem of false positive signal of a PreAmp is stuck or trapped unspecifically. An improved probe system design as shown in FIG. 54 can be used to reduce the false positive signal. Two separate PreAmps are captured to each side of the junction by two separate CPs or CP pairs. Each amplifier (Amp) is captured to the PreAmp pair by a pair of Linker Capture Probes (LCP), which has one section complimentary to a section of the PreAmp and another section to a section of the Amp. Again, the binding strengths between the LCP and PreAmp or between LCP and Amp or both are designed to be intentionally “weak” so that a single LCP can not hold the Amp securely to PreAmp through the assay hybridization step. When and only when both LCPs in the pair present, the pair produces sufficient binding strength to capture the Amp securely to PreAmp. In this new design, a single PreAmp can not produce signal. A unspecifically attached Amp, on the other hand, carries much smaller number of label probes thus produces much lower level of signal compared with that produced by a real junction. The chance of false positives will be significantly reduced. FIG. 56 shows a slightly modified scaffold configuration, where the LCP is eliminated and the Amp binds directly to the Linkers. Again, the binding sections between the Amp and the Linkers are designed as such that a single Linker can not hold the Amp securely, but when and only when both Linkers present, the combined strength of two binding regions hold the Amp securely at hybridization temperature.

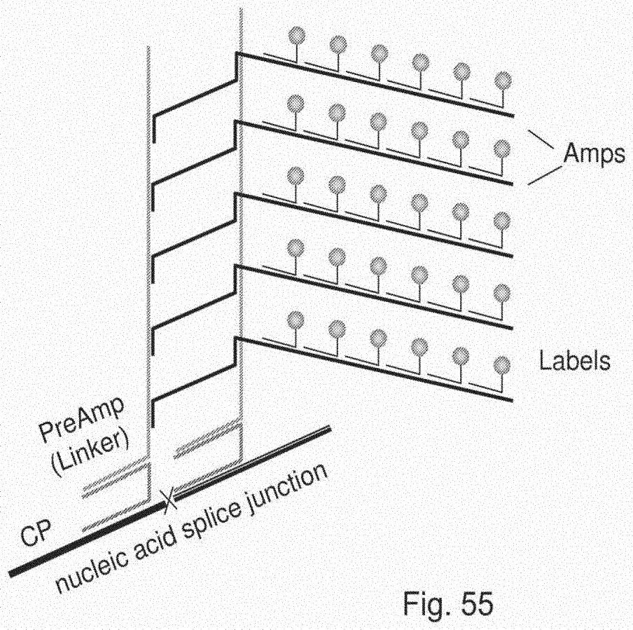

Of course, the approaches described in FIGS. 55 and 56 can be used broadly for the detection of mis-joint nucleic acid sequences without limiting to detecting specific junctions. Multiple Linker pairs can be deployed on each side of the junction to boost signal. In fact, the application of these approaches can be expanded further to the detection of any nucleic acid.

Of course, all the methods and approaches described above can be used to detect a reverse condition in situ, where normally joint sequences become separated.

Although a method for the detection of transcripts from known gene fusions and mis-splicing events currently exists, namely RT-PCR, our new method offers several novel features. First, RT-PCR assays require the destruction of the sample in order to purify RNA for the assay, thereby removing histological data such tumor size and type. Our assay allows for the preservation of tissue morphology since the detection of the fusion transcript is done in situ. Next, RT-PCR requires difficult multiplexing for the detection of fusion transcripts based on oncogenes that carry multiple fusion partners, since a unique primer pair is required for each possible fusion transcript. In that case, multiple PCR products are generated and need to be analyzed. Because our probe sets are based on oligonucleotides that hybridize in a reproducible fashion to the amplifiers, and multiple probe sets can be linking to a single amplification system, we can overcome this type of multiplexing problem by generating a probe pool of all the potential fusion partners for an oncogene and label it in a single color, while the oncogene fusion partner is labeled in a second color. This allows for the routine detection of multiple fusion transcripts in a single sample while still using a two color system. Finally, RT-PCR also requires long intact stretches of RNA in order to generate the amplification product, which can be difficult to come by in older FFPE tissue. Our assay, however, is not prone to failure due to degradation of the target RNA over time. In addition to these unique characteristics that have advantages over RT-PCR, our assay is the first that is capable of visualizing individual RNA molecules from gene fusions. Though other RNA in situ technologies exist, their lack of single molecule sensitivity and difficulty in multiplexing make them unsuited for this type of analysis.

Our method for detection of fusion transcripts and mis-splicing can also be applied to direct visualization of genomic rearrangements such as translocations and inversions. In a similar fashion to conventional DNA FISH probes, we can design and synthesize probes to two genes that are being scored for a fusion event. After fluorescent or chromogenic labeling as described above, an alteration as compared to wild type chromosomes can be seen if present. In the case of known inversions that cause gene fusions (e.g. EML4-ALK), a separation of the probe signals would occur indicating that DNA sequences normally side-by-side have now moved. In the case of a translocation event leading to the generation of a known fusion gene (e.g. BCR-ABL), two signals normally appearing separate would now be seen merged indicating that a fragment of DNA has moved to the new location.

Currently there are many DNA FISH technologies that are widely in use for the detection of gene fusions, and though our method is similar in nature, our use of a small number of oligonucleotides allows for high resolution mapping of small changes in DNA structure. In contrast, traditional DNA FISH probes are derived from large (>100 kb) BAC sequences and can not distinguish the alteration of small DNA fragments due to the long length of sequence necessary for the hybridization of these probes. Since we have already demonstrated that as little as 1 kb of sequence is required for our assay to give a positive signal, we are in a position to detect gene fusions caused by microdeletions, small inversions, and the translocation of small fragments of DNA.

### Methods of Detecting Nucleic Acid Sequence Captured on a Solid Support

The present invention also provides methods, compositions, tissue slides, and kits for detecting target nucleic acid sequences, particularly multiplex detection. Target nucleic acid sequences are captured to a solid support and then detected, preferably in a branched-chain DNA assay.

Branched-chain DNA (bDNA) signal amplification technology has been used, e.g., to detect and quantify mRNA transcripts in cell lines and to determine viral loads in blood. The bDNA assay is a sandwich nucleic acid hybridization procedure that enables direct measurement of mRNA expression, e.g., from crude cell lysate. It provides direct quantification of nucleic acid molecules at physiological levels. Several advantages of the technology distinguish it from other DNA/RNA amplification technologies, including linear amplification, good sensitivity and dynamic range, great precision and accuracy, simple sample preparation procedure, and reduced sample-to-sample variation.

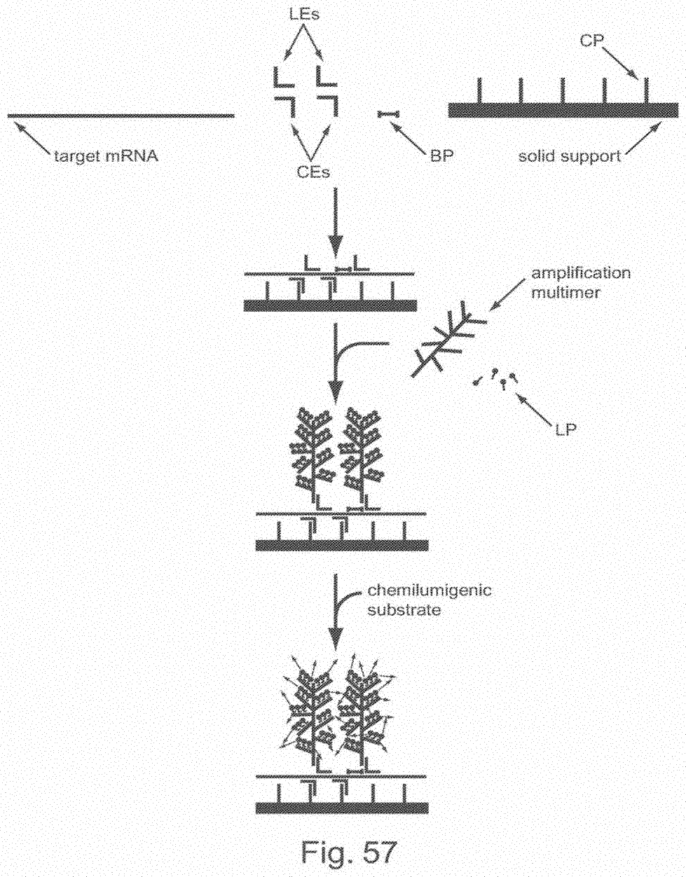

In brief, in a typical bDNA assay for gene expression analysis (FIG. 57), a target mRNA whose expression is to be detected is released from cells and captured by a Capture Pole (CP) on a solid surface (e.g., a well of a microtiter plate) through synthetic oligonucleotide probes called Capture Extenders (CEs). Each capture extender has a first polynucleotide sequence that can hybridize to the target mRNA and a second polynucleotide sequence that can hybridize to the capture probe. Typically, two or more capture extenders are used. Probes of another type, called Label Extenders (LEs), hybridize to different sequences on the target mRNA and to sequences on an amplification multimer. Label Extender is also called Capture Probe in this application. Amplification multimer is also called Amplifer in this application. Additionally, Blocking Probes (BPs), which hybridize to regions of the target mRNA not occupied by CEs or LEs, are often used to reduce non-specific target probe binding. A probe set for a given mRNA thus consists of CEs, LEs, and optionally BPs for the target mRNA. The CEs, LEs, and BPs are complementary to nonoverlapping sequences in the target mRNA, and are typically, but not necessarily, contiguous.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. Nos. 5,635,352 and 5,681,697, which further amplifies the signal from a single target mRNA. In yet another example, the label extenders hybridize directly to the label probes and no amplification multimer or preamplifier is used, so the signal from a single target mRNA molecule is only amplified by the number of distinct label extenders that hybridize to that mRNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. Nos. 5,359,100; 5,571,670; 5,614,362; 6,235,465; 5,712,383; 5,747,244; 6,232,462; 5,681,702; 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QuantiGene™ kits, comprising instructions and reagents such as amplification multimers, alkaline phosphatase labeled label probes, chemilumigenic substrate, capture poles immobilized on a solid support, and the like) are commercially available, e.g., from Panomics, Inc. (on the world wide web at (www.) panomics.com). Software for designing probe sets for a given mRNA target (i.e., for designing the regions of the CEs, LEs, and optionally BPs that are complementary to the target) is also commercially available (e.g., ProbeDesigner™ from Panomics, Inc.; see also Bushnell et al. (1999) “ProbeDesigner: for the design of probe sets for branched DNA (bDNA) signal amplification assays Bioinformatics 15:348-55). The basic bDNA assay, however, permits detection of only a single target nucleic acid per assay, while, as described above, detection of multiple nucleic acids is frequently desirable.

Among other aspects, the present invention provides multiplex bDNA assays that can be used for simultaneous detection of two or more target nucleic acids. Similarly, one aspect of the present invention provides bDNA assays, singleplex or multiplex, that have reduced background from nonspecific hybridization events.

In general, in the assays of the invention, two or more label extenders are used to capture a single component of the label probe system (e.g., a preamplifier or amplification multimer). The assay temperature and the stability of the complex between a single LE and the component of the label probe system (e.g., the preamplifier or amplification multimer) can be controlled such that binding of a single LE to the component is not sufficient to stably associate the component with a nucleic acid to which the LE is bound, whereas simultaneous binding of two or more LEs to the component can capture it to the nucleic acid. Requiring such cooperative hybridization of multiple LEs for association of the label probe system with the nucleic acid(s) of interest results in high specificity and low background from cross-hybridization of the LEs with other, non-target nucleic acids.

For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous LE-label probe system component interactions for the capture of the label probe system to a target nucleic acid minimizes the chance that nonspecific capture will occur, even when some nonspecific CE-LE or LE-CP interactions, for example, do occur. This reduction in background through minimization of undesirable cross-hybridization events thus facilitates multiplex detection of the nucleic acids of interest.

The methods of the invention can be used, for example, for multiplex detection of two or more nucleic acids simultaneously, from even complex samples, without requiring prior purification of the nucleic acids. In one aspect, the methods involve capture of the nucleic acids to particles (e.g., distinguishable subsets of microspheres), while in another aspect, the nucleic acids are captured to a spatially addressable solid support. Compositions, kits, and systems related to the methods are also provided.

Methods

As noted, one aspect of the invention provides multiplex nucleic acid assays. Thus, one general class of embodiments includes methods of detecting two or more nucleic acids of interest. In the methods, a sample comprising or suspected of comprising the nucleic acids of interest, two or more subsets of m label extenders, wherein m is at least two, and a label probe system are provided. Each subset of m label extenders is capable of hybridizing to one of the nucleic acids of interest. The label probe system comprises a label, and a component of the label probe system is capable of hybridizing simultaneously to at least two of the m label extenders in a subset.

Those nucleic acids of interest present in the sample are captured on a solid support. Each nucleic acid of interest captured on the solid support is hybridized to its corresponding subset of m label extenders, and the label probe system is hybridized to the m label extenders. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

Essentially any suitable solid support can be employed in the methods. For example, the solid support can comprise particles such as microspheres, or it can comprise a substantially planar and/or spatially addressable support. Different nucleic acids are optionally captured on different distinguishable subsets of particles or at different positions on a spatially addressable solid support. The nucleic acids of interest can be captured to the solid support by any of a variety of techniques, for example, by binding directly to the solid support or by binding to a moiety bound to the support, or through hybridization to another nucleic acid bound to the solid support. Preferably, the nucleic acids are captured to the solid support through hybridization with capture extenders and capture poles.

In one class of embodiments, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture pole.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture poles, thereby associating each subset of n capture extenders with a selected subset of the particles. Each of the nucleic acids of interest present in the sample is hybridized to its corresponding subset of n capture extenders and the subset of n capture extenders is hybridized to its corresponding capture pole, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

Essentially any suitable particles, e.g., particles having distinguishable characteristics and to which capture poles can be attached, can be used. For example, in one preferred class of embodiments, the particles are microspheres. The microspheres of each subset can be distinguishable from those of the other subsets, e.g., on the basis of their fluorescent emission spectrum, their diameter, or a combination thereof. For example, the microspheres of each subset can be labeled with a unique fluorescent dye or mixture of such dyes, quantum dots with distinguishable emission spectra, and/or the like. As another example, the particles of each subset can be identified by an optical barcode, unique to that subset, present on the particles.

The particles optionally have additional desirable characteristics. For example, the particles can be magnetic or paramagnetic, which provides a convenient means for separating the particles from solution, e.g., to simplify separation of the particles from any materials not bound to the particles.

In other embodiments, the nucleic acids are captured at different positions on a non-particulate, spatially addressable solid support. Thus, in one class of embodiments, the solid support comprises two or more capture poles, wherein each capture pole is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture poles, thereby associating each subset of n capture extenders with a selected position on the solid support. Each of the nucleic acids of interest present in the sample is hybridized to its corresponding subset of n capture extenders and the subset of n capture extenders is hybridized to its corresponding capture pole, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

The solid support typically has a planar surface and is typically rigid, but essentially any spatially addressable solid support can be adapted to the practice of the present invention. Exemplary materials for the solid support include, but are not limited to, glass, silicon, silica, quartz, plastic, polystyrene, nylon, and nitrocellulose. As just one example, an array of capture poles can be formed at selected positions on a glass slide as the solid support.

In any of the embodiments described herein in which capture extenders are utilized to capture the nucleic acids to the solid support, n, the number of capture extenders in a subset, is at least one, preferably at least two, and more preferably at least three. n can be at least four or at least five or more. Typically, but not necessarily, n is at most ten. For example, n can be between three and ten, e.g., between five and ten or between five and seven, inclusive. Use of fewer capture extenders can be advantageous, for example, in embodiments in which nucleic acids of interest are to be specifically detected from samples including other nucleic acids with sequences very similar to that of the nucleic acids of interest. In other embodiments (e.g., embodiments in which capture of as much of the nucleic acid as possible is desired), however, n can be more than 10, e.g., between 20 and 50. n can be the same for all of the subsets of capture extenders, but it need not be; for example, one subset can include three capture extenders while another subset includes five capture extenders. The n capture extenders in a subset preferably hybridize to nonoverlapping polynucleotide sequences in the corresponding nucleic acid of interest. The nonoverlapping polynucleotide sequences can, but need not be, consecutive within the nucleic acid of interest.

Each capture extender is capable of hybridizing to its corresponding capture pole. The capture extender typically includes a polynucleotide sequence C-1 that is complementary to a polynucleotide sequence C-2 in its corresponding capture pole. Capture of the nucleic acids of interest via hybridization to the capture extenders and capture poles optionally involves cooperative hybridization. In one aspect, the capture extenders and capture poles are configured as described in U.S. patent application Ser. No. 11/471,025 filed Jun. 19, 2006 by Luo et al., entitled “Multiplex branched-chain DNA assays.”

The capture pole can include polynucleotide sequence in addition to C-2, or C-2 can comprise the entire polynucleotide sequence of the capture pole. For example, each capture pole optionally includes a linker sequence between the site of attachment of the capture pole to the particles and sequence C-2 (e.g., a linker sequence containing 8 Ts, as just one possible example).

The methods are useful for multiplex detection of nucleic acids, optionally highly multiplex detection. Thus, the two or more nucleic acids of interest (i.e., the nucleic acids to be detected) optionally comprise five or more, 10 or more, 20 or more, 30 or more, 40 or more, 50 or more, or even 100 or more nucleic acids of interest, while the two or more subsets of m label extenders comprise five or more, 10 or more, 20 or more, 30 or more, 40 or more, 50 or more, or even 100 or more subsets of m label extenders. In embodiments in which capture extenders, particulate solid supports, and/or spatially addressable solid support are used, a like number of subsets of capture extenders, subsets of particles, and/or selected positions on the solid support are provided.

The label probe system optionally includes an amplification multimer and a plurality of label probes, wherein the amplification multimer is capable of hybridizing to the label extenders and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes. As another example, the label probe system can include only label probes, which hybridize directly to the label extenders. In one class of embodiments, the label probe comprises the label, e.g., a covalently attached label. In other embodiments, the label probe is configured to bind a label; for example, a biotinylated label probe can bind to a streptavidin-associated label.

The label can be essentially any convenient label that directly or indirectly provides a detectable signal. In one aspect, the label is a fluorescent label (e.g., a fluorophore or quantum dot). Detecting the presence of the label on the particles thus comprises detecting a fluorescent signal from the label. In embodiments in which the solid support comprises particles, fluorescent emission by the label is typically distinguishable from any fluorescent emission by the particles, e.g., microspheres, and many suitable fluorescent label-fluorescent microsphere combinations are possible. As other examples, the label can be a luminescent label, a light-scattering label (e.g., colloidal gold particles), or an enzyme (e.g., HRP).

As noted above, a component of the label probe system is capable of hybridizing simultaneously to at least two of the m label extenders in a subset. Typically, the component of the label probe system that hybridizes to the two or more label extenders is an amplification multimer or preamplifier. Preferably, binding of a single label extender to the component of the label probe system (e.g., the amplification multimer or preamplifier) is insufficient to capture the label probe system to the nucleic acid of interest to which the label extender binds. Thus, in one aspect, the label probe system comprises an amplification multimer or preamplifier, which amplification multimer or preamplifier is capable of hybridizing to the at least two label extenders, and the label probe system (or the component thereof) is hybridized to the m label extenders at a hybridization temperature, which hybridization temperature is greater than a melting temperature Tₘ of a complex between each individual label extender and the amplification multimer or preamplifier. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ. It is worth noting that the hybridization temperature can be the same or different than the temperature at which the label extenders and optional capture extenders are hybridized to the nucleic acids of interest.

Each label extender typically includes a polynucleotide sequence L-1 that is complementary to a polynucleotide sequence in the corresponding nucleic acid of interest and a polynucleotide sequence L-2 that is complementary to a polynucleotide sequence in the component of the label probe system (e.g., the preamplifier or amplification multimer). It will be evident that the amount of overlap between each individual label extender and the component of the label probe system (i.e., the length of L-2 and M-1) affects the Tₘ of the complex between the label extender and the component, as does, e.g., the GC base content of sequences L-2 and M-1. Optionally, all the label extenders have the same length sequence L-2 and/or identical polynucleotide sequences L-2. Alternatively, different label extenders can have different length and/or sequence polynucleotide sequences L-2. It will also be evident that the number of label extenders required for stable capture of the component to the nucleic acid of interest depends, in part, on the amount of overlap between the label extenders and the component (i.e., the length of L-2 and M-1).

Stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized.

Appropriate combinations of the amount of complementarity between the label extenders and the component of the label probe system, number of label extenders binding to the component, and stringency of hybridization can, for example, be determined experimentally by one of skill in the art. For example, a particular number of label extenders and a particular set of hybridization conditions can be selected, while the number of nucleotides of complementarity between the label extenders and the component is varied until hybridization of the label extenders to a nucleic acid captures the component to the nucleic acid while hybridization of a single label extender does not efficiently capture the component. Stringency can be controlled, for example, by controlling the formamide concentration, chaotropic salt concentration, salt concentration, pH, organic solvent content, and/or hybridization temperature.

As noted, the Tₘ of any nucleic acid duplex can be directly measured, using techniques well known in the art. For example, a thermal denaturation curve can be obtained for the duplex, the midpoint of which corresponds to the Tₘ. It will be evident that such denaturation curves can be obtained under conditions having essentially any relevant pH, salt concentration, solvent content, and/or the like.

The Tₘ for a particular duplex (e.g., an approximate Tₘ) can also be calculated. For example, the Tₘ for an oligonucleotide-target duplex can be estimated using the following algorithm, which incorporates nearest neighbor thermodynamic parameters: Tₘ (Kelvin)=ΔH°/(ΔS°+R ln Ct), where the changes in standard enthalpy (ΔH°) and entropy (ΔS°) are calculated from nearest neighbor thermodynamic parameters (see, e.g., SantaLucia (1998) “A unified view of polymer, dumbbell, and oligonucleotide DNA nearest-neighbor thermodynamics” Proc. Natl. Acad. Sci. USA 95:1460-1465, Sugimoto et al. (1996) “Improved thermodynamic parameters and helix initiation factor to predict stability of DNA duplexes” Nucleic Acids Research 24: 4501-4505, Sugimoto et al. (1995) “Thermodynamic parameters to predict stability of RNA/DNA hybrid duplexes” Biochemistry 34:11211-11216, and et al. (1998) “Thermodynamic parameters for an expanded nearest-neighbor model for formation of RNA duplexes with Watson-Crick base pairs” Biochemistry 37: 14719-14735), R is the ideal gas constant (1.987 cal·K-1mole-1), and Ct is the molar concentration of the oligonucleotide. The calculated Tₘ is optionally corrected for salt concentration, e.g., Na+ concentration, using the formula 1/Tₘ(Na+)=1/Tₘ(1M)+(4.29f (G·C)−3.95)×10⁻⁵ ln [Na⁺]+9.40×10⁻⁶ ln 2[Na⁺]. See, e.g., Owczarzy et al. (2004) “Effects of Sodium Ions on DNA Duplex Oligomers: Improved Predictions of Melting Temperatures” Biochemistry 43:3537-3554 for further details. A Web calculator for estimating Tₘ using the above algorithms is available on the Internet at scitools.idtdna.com/analyzer/oligocalc.asp. Other algorithms for calculating Tₘ are known in the art and are optionally applied to the present invention.

Typically, the component of the label probe system (e.g., the amplification multimer or preamplifier) is capable of hybridizing simultaneously to two of the m label extenders in a subset, although it optionally hybridizes to three, four, or more of the label extenders. In one class of embodiments, e.g., embodiments in which two (or more) label extenders bind to the component of the label probe system, sequence L-2 is 20 nucleotides or less in length. For example, L-2 can be between 9 and 17 nucleotides in length, e.g., between 12 and 15 nucleotides in length, between 13 and 15 nucleotides in length, or between 13 and 14 nucleotides in length. As noted, m is at least two, and can be at least three, at least five, at least 10, or more. m can be the same or different from subset to subset of label extenders.

The label extenders can be configured in any of a variety ways. For example, the two label extenders that hybridize to the component of the label probe system can assume a cruciform arrangement, with one label extender having L-1 5′ of L-2 and the other label extender having L-1 3′ of L-2. Unexpectedly, however, a configuration in which either the 5′ or the 3′ end of both label extenders hybridizes to the nucleic acid while the other end binds to the component yields stronger binding of the component to the nucleic acid than does a cruciform arrangement of the label extenders. Thus, in one class of embodiments, the at least two label extenders (e.g., the m label extenders in a subset) each have L-1 5′ of L-2 or each have L-1 3′ of L-2. For example, L-1, which hybridizes to the nucleic acid of interest, can be at the 5′ end of each label extender, while L-2, which hybridizes to the component of the label probe system, is at the 3′ end of each label extender (or vice versa). L-1 and L-2 are optionally separated by additional sequence. In one exemplary embodiment, L-1 is located at the 5′ end of the label extender and is about 20-30 nucleotides in length, L-2 is located at the 3′ end of the label extender and is about 13-14 nucleotides in length, and L-1 and L-2 are separated by a spacer (e.g., 5 Ts).

A label extender, preamplifier, amplification multimer, label probe, capture pole and/or capture extender optionally comprises at least one non-natural nucleotide. For example, a label extender and the component of the label probe system (e.g., the amplification multimer or preamplifier) optionally comprise, at complementary positions, at least one pair of non-natural nucleotides that base pair with each other but that do not Watson-Crick base pair with the bases typical to biological DNA or RNA (i.e., A, C, G, T, or U). Examples of nonnatural nucleotides include, but are not limited to, Locked NucleicAcid™ nucleotides (available from Exiqon A/S, (www.) exiqon.com; see, e.g., SantaLucia Jr. (1998) Proc Natl Acad Sci 95:1460-1465) and isoG, isoC, and other nucleotides used in the AEGIS system (Artificially Expanded Genetic Information System, available from EraGen Biosciences, (www.) eragen.com; see, e.g., U.S. Pat. Nos. 6,001,983, 6,037,120, and 6,140,496). Use of such non-natural base pairs (e.g., isoG-isoC base pairs) in the probes can, for example, reduce background and/or simplify probe design by decreasing cross hybridization, or it can permit use of shorter probes (e.g., shorter sequences L-2 and M-1) when the non-natural base pairs have higher binding affinities than d\

The methods can optionally be used to quantitate the amounts of the nucleic acids of interest present in the sample. For example, in one class of embodiments, an intensity of a signal from the label is measured, e.g., for each subset of particles or selected position on the solid support, and correlated with a quantity of the corresponding nucleic acid of interest present.

As noted, blocking probes are optionally also hybridized to the nucleic acids of interest, which can reduce background in the assay. For a given nucleic acid of interest, the corresponding label extenders, optional capture extenders, and optional blocking probes are preferably complementary to physically distinct, nonoverlapping sequences in the nucleic acid of interest, which are preferably, but not necessarily, contiguous. The Tₘs of the capture extender-nucleic acid, label extender-nucleic acid, and blocking probe-nucleic acid complexes are preferably greater than the temperature at which the capture extenders, label extenders, and/or blocking probes are hybridized to the nucleic acid, e.g., by 5° C. or 10° C. or preferably by 15° C. or more, such that these complexes are stable at that temperature. Potential C E and LE sequences (e.g., potential sequences C-3 and L-1) are optionally examined for possible interactions with non-corresponding nucleic acids of interest, LEs or CEs, the preamplifier, the amplification multimer, the label probe, and/or any relevant genomic sequences, for example; sequences expected to cross-hybridize with undesired nucleic acids are typically not selected for use in the CEs or LEs. See, e.g., Player et al. (2001) “Single-copy gene detection using branched DNA (bDNA) in situ hybridization” J Histochem Cytochem 49:603-611 and U.S. patent application 60/680,976. Examination can be, e.g., visual (e.g., visual examination for complementarity), computational (e.g., computation and comparison of binding free energies), and/or experimental (e.g., cross-hybridization experiments). Capture pole sequences are preferably similarly examined, to ensure that the polynucleotide sequence C-1 complementary to a particular capture pole's sequence C-2 is not expected to cross-hybridize with any of the other capture poles that are to be associated with other subsets of particles or selected positions on the support.

At any of various steps, materials not captured on the solid support are optionally separated from the support. For example, after the capture extenders, nucleic acids, label extenders, blocking probes, and support-bound capture poles are hybridized, the support is optionally washed to remove unbound nucleic acids and probes; after the label extenders and amplification multimer are hybridized, the support is optionally washed to remove unbound amplification multimer; and/or after the label probes are hybridized to the amplification multimer, the support is optionally washed to remove unbound label probe prior to detection of the label.

In embodiments in which different nucleic acids are captured to different subsets of particles, one or more of the subsets of particles is optionally isolated, whereby the associated nucleic acid of interest is isolated. Similarly, nucleic acids can be isolated from selected positions on a spatially addressable solid support. The isolated nucleic acid can optionally be removed from the particles and/or subjected to further manipulation, if desired (e.g., amplification by PCR or the like).

The methods can be used to detect the presence of the nucleic acids of interest in essentially any type of sample. For example, the sample can be derived from an animal, a human, a plant, a cultured cell, a virus, a bacterium, a pathogen, and/or a microorganism. The sample optionally includes a cell lysate, an intercellular fluid, a bodily fluid (including, but not limited to, blood, serum, saliva, urine, sputum, or spinal fluid), and/or a conditioned culture medium, and is optionally derived from a tissue (e.g., a tissue homogenate), a biopsy, and/or a tumor. Similarly, the nucleic acids can be essentially any desired nucleic acids (e.g., DNA, RNA, mRNA, rRNA, miRNA, etc.). As just a few examples, the nucleic acids of interest can be derived from one or more of an animal, a human, a plant, a cultured cell, a microorganism, a virus, a bacterium, or a pathogen.

As noted, the methods can be used for gene expression analysis. Accordingly, in one class of embodiments, the two or more nucleic acids of interest comprise two or more mRNAs. The methods can also be used for clinical diagnosis and/or detection of microorganisms, e.g., pathogens. Thus, in certain embodiments, the nucleic acids include bacterial and/or viral genomic RNA and/or DNA (double-stranded or single-stranded), plasmid or other extra-genomic DNA, or other nucleic acids derived from microorganisms (pathogenic or otherwise). It will be evident that double-stranded nucleic acids of interest will typically be denatured before hybridization with capture extenders, label extenders, and the like.

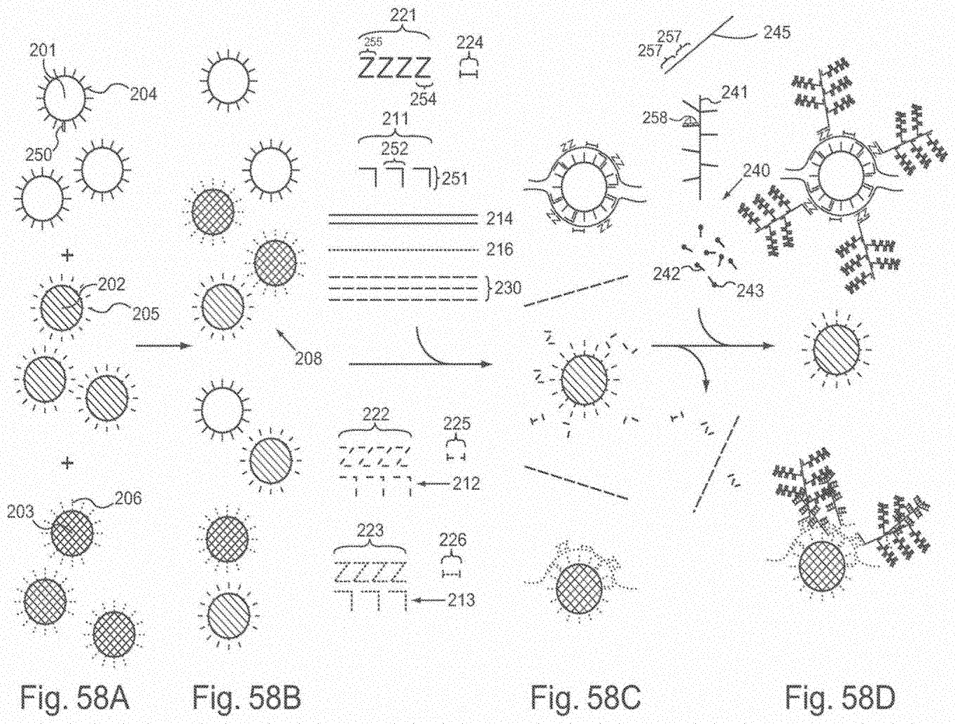

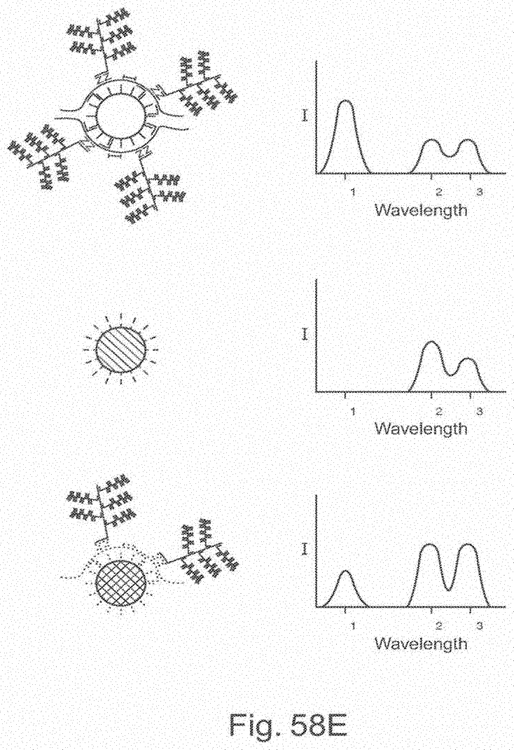

An exemplary embodiment is schematically illustrated in FIG. 58. Panel A illustrates three distinguishable subsets of microspheres 201, 202, and 203, which have associated therewith capture poles 204, 205, and 206, respectively. Each capture pole includes a sequence C-2 (250), which is different from subset to subset of microspheres. The three subsets of microspheres are combined to form pooled population 208 (Panel B). A subset of capture extenders is provided for each nucleic acid of interest; subset 211 for nucleic acid 214, subset 212 for nucleic acid 215 which is not present, and subset 213 for nucleic acid 216. Each capture extender includes sequences C-1 (251, complementary to the respective capture pole's sequence C-2) and C-3 (252, complementary to a sequence in the corresponding nucleic acid of interest). Three subsets of label extenders (221, 222, and 223 for nucleic acids 214, 215, and 216, respectively) and three subsets of blocking probes (224, 225, and 226 for nucleic acids 214, 215, and 216, respectively) are also provided. Each label extender includes sequences L-1 (254, complementary to a sequence in the corresponding nucleic acid of interest) and L-2 (255, complementary to M-1). Non-target nucleic acids 230 are also present in the sample of nucleic acids.

Subsets of label extenders 221 and 223 are hybridized to nucleic acids 214 and 216, respectively. In addition, nucleic acids 214 and 216 are hybridized to their corresponding subset of capture extenders (211 and 213, respectively), and the capture extenders are hybridized to the corresponding capture poles (204 and 206, respectively), capturing nucleic acids 214 and 216 on microspheres 201 and 203, respectively (Panel C). Materials not bound to the microspheres (e.g., capture extenders 212, nucleic acids 230, etc.) are separated from the microspheres by washing. Label probe system 240 including preamplifier 245 (which includes two sequences M-1 257), amplification multimer 241 (which includes sequences M-2 258), and label probe 242 (which contains label 243) is provided. Each preamplifier 245 is hybridized to two label extenders, amplification multimers 241 are hybridized to the preamplifier, and label probes 242 are hybridized to the amplification multimers (Panel D). Materials not captured on the microspheres are optionally removed by washing the microspheres. Microspheres from each subset are identified, e.g., by their fluorescent emission spectrum (λ2 and λ3, Panel E), and the presence or absence of the label on each subset of microspheres is detected (λ1, Panel E). Since each nucleic acid of interest is associated with a distinct subset of microspheres, the presence of the label on a given subset of microspheres correlates with the presence of the corresponding nucleic acid in the original sample.

As depicted in FIG. 58, all of the label extenders in all of the subsets typically include an identical sequence L-2. Optionally, however, different label extenders (e.g., label extenders in different subsets) can include different sequences L-2. Also as depicted in FIG. 58, each capture pole typically includes a single sequence C-2 and thus hybridizes to a single capture extender. Optionally, however, a capture pole can include two or more sequences C-2 and hybridize to two or more capture extenders. Similarly, as depicted, each of the capture extenders in a particular subset typically includes an identical sequence C-1, and thus only a single capture pole is needed for each subset of particles; however, different capture extenders within a subset optionally include different sequences C-1 (and thus hybridize to different sequences C-2, within a single capture pole or different capture poles on the surface of the corresponding subset of particles).

In the embodiment depicted in FIG. 58, the label probe system includes the preamplifier, amplification multimer, and label probe. It will be evident that similar considerations apply to embodiments in which the label probe system includes only an amplification multimer and label probe or only a label probe.

The various hybridization and capture steps can be performed simultaneously or sequentially, in any convenient order. For example, in embodiments in which capture extenders are employed, each nucleic acid of interest can be hybridized simultaneously with its corresponding subset of m label extenders and its corresponding subset of n capture extenders, and then the capture extenders can be hybridized with capture poles associated with the solid support. Materials not captured on the support are preferably removed, e.g., by washing the support, and then the label probe system is hybridized to the label extenders.

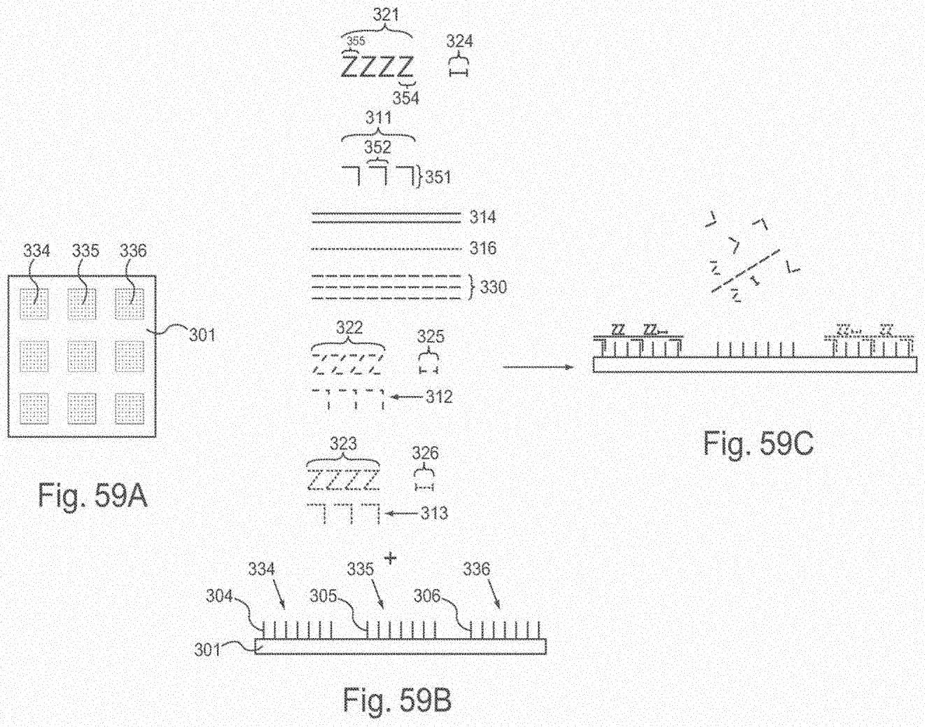

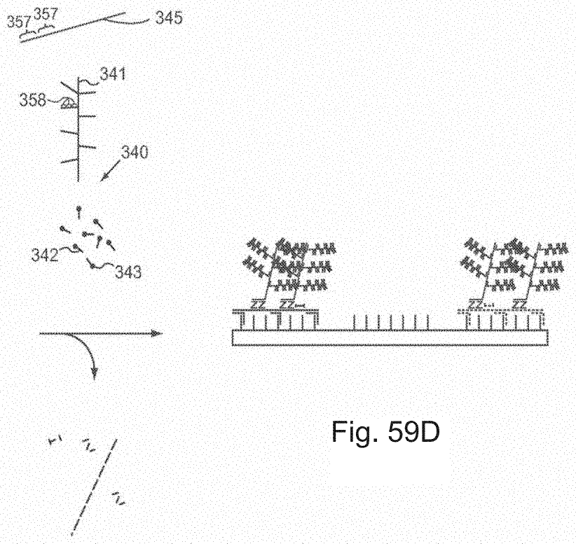

Another exemplary embodiment is schematically illustrated in FIG. 59. Panel A depicts solid support 301 having nine capture poles provided on it at nine selected positions (e.g., 334-336). Panel B depicts a cross section of solid support 301, with distinct capture poles 304, 305, and 306 at different selected positions on the support (334, 335, and 336, respectively). A subset of capture extenders is provided for each nucleic acid of interest. Only three subsets are depicted; subset 311 for nucleic acid 314, subset 312 for nucleic acid 315 which is not present, and subset 313 for nucleic acid 316. Each capture extender includes sequences C-1 (351, complementary to the respective capture pole's sequence C-2) and C-3 (352, complementary to a sequence in the corresponding nucleic acid of interest). Three subsets of label extenders (321, 322, and 323 for nucleic acids 314, 315, and 316, respectively) and three subsets of blocking probes (324, 325, and 326 for nucleic acids 314, 315, and 316, respectively) are also depicted (although nine would be provided, one for each nucleic acid of interest). Each label extender includes sequences L-1 (354, complementary to a sequence in the corresponding nucleic acid of interest) and L-2 (355, complementary to M-1). Non-target nucleic acids 330 are also present in the sample of nucleic acids.

Subsets of label extenders 321 and 323 are hybridized to nucleic acids 314 and 316, respectively. Nucleic acids 314 and 316 are hybridized to their corresponding subset of capture extenders (311 and 313, respectively), and the capture extenders are hybridized to the corresponding capture poles (304 and 306, respectively), capturing nucleic acids 314 and 316 at selected positions 334 and 336, respectively (Panel C). Materials not bound to the solid support (e.g., capture extenders 312, nucleic acids 330, etc.) are separated from the support by washing. Label probe system 340 including preamplifier 345 (which includes two sequences M-1 357), amplification multimer 341 (which includes sequences M-2 358) and label probe 342 (which contains label 343) is provided. Each preamplifier 345 is hybridized to two label extenders, amplification multimers 341 are hybridized to the preamplifier, and label probes 342 are hybridized to the amplification multimers (Panel D). Materials not captured on the solid support are optionally removed by washing the support, and the presence or absence of the label at each position on the solid support is detected. Since each nucleic acid of interest is associated with a distinct position on the support, the presence of the label at a given position on the support correlates with the presence of the corresponding nucleic acid in the original sample.

Another general class of embodiments provides methods of detecting one or more nucleic acids, using the novel label extender configuration described above. In the methods, a sample comprising or suspected of comprising the nucleic acids of interest, one or more subsets of m label extenders, wherein m is at least two, and a label probe system are provided. Each subset of m label extenders is capable of hybridizing to one of the nucleic acids of interest. The label probe system comprises a label, and a component of the label probe system (e.g., a preamplifier or an amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

Those nucleic acids of interest present in the sample are captured on a solid support. Each nucleic acid of interest captured on the solid support is hybridized to its corresponding subset of m label extenders, and the label probe system (or the component thereof) is hybridized to the m label extenders at a hybridization temperature. The hybridization temperature is greater than a melting temperature Tₘ of a complex between each individual label extender and the component of the label probe system. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

Typically, the one or more nucleic acids of interest comprise two or more nucleic acids of interest, and the one or more subsets of m label extenders comprise two or more subsets of m label extenders.

The various hybridization and capture steps can be performed simultaneously or sequentially, in any convenient order. For example, in embodiments in which capture extenders are employed, each nucleic acid of interest can be hybridized simultaneously with its corresponding subset of m label extenders and its corresponding subset of n capture extenders, and then the capture extenders can be hybridized with capture poles associated with the solid support. Materials not captured on the support are preferably removed, e.g., by washing the support, and then the label probe system is hybridized to the label extenders.

Another exemplary embodiment is schematically illustrated in FIG. 59. Panel A depicts solid support 301 having nine capture poles provided on it at nine selected positions (e.g., 334-336). Panel B depicts a cross section of solid support 301, with distinct capture poles 304, 305, and 306 at different selected positions on the support (334, 335, and 336, respectively). A subset of capture extenders is provided for each nucleic acid of interest. Only three subsets are depicted; subset 311 for nucleic acid 314, subset 312 for nucleic acid 315 which is not present, and subset 313 for nucleic acid 316. Each capture extender includes sequences C-1 (351, complementary to the respective capture pole's sequence C-2) and C-3 (352, complementary to a sequence in the corresponding nucleic acid of interest). Three subsets of label extenders (321, 322, and 323 for nucleic acids 314, 315, and 316, respectively) and three subsets of blocking probes (324, 325, and 326 for nucleic acids 314, 315, and 316, respectively) are also depicted (although nine would be provided, one for each nucleic acid of interest). Each label extender includes sequences L-1 (354, complementary to a sequence in the corresponding nucleic acid of interest) and L-2 (355, complementary to M-1). Non-target nucleic acids 330 are also present in the sample of nucleic acids.

Subsets of label extenders 321 and 323 are hybridized to nucleic acids 314 and 316, respectively. Nucleic acids 314 and 316 are hybridized to their corresponding subset of capture extenders (311 and 313, respectively), and the capture extenders are hybridized to the corresponding capture poles (304 and 306, respectively), capturing nucleic acids 314 and 316 at selected positions 334 and 336, respectively (Panel C). Materials not bound to the solid support (e.g., capture extenders 312, nucleic acids 330, etc.) are separated from the support by washing. Label probe system 340 including preamplifier 345 (which includes two sequences M-1 357), amplification multimer 341 (which includes sequences M-2 358) and label probe 342 (which contains label 343) is provided. Each preamplifier 345 is hybridized to two label extenders, amplification multimers 341 are hybridized to the preamplifier, and label probes 342 are hybridized to the amplification multimers (Panel D). Materials not captured on the solid support are optionally removed by washing the support, and the presence or absence of the label at each position on the solid support is detected. Since each nucleic acid of interest is associated with a distinct position on the support, the presence of the label at a given position on the support correlates with the presence of the corresponding nucleic acid in the original sample.

Another general class of embodiments provides methods of detecting one or more nucleic acids, using the novel label extender configuration described above. In the methods, a sample comprising or suspected of comprising the nucleic acids of interest, one or more subsets of m label extenders, wherein m is at least two, and a label probe system are provided. Each subset of m label extenders is capable of hybridizing to one of the nucleic acids of interest. The label probe system comprises a label, and a component of the label probe system (e.g., a preamplifier or an amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

Those nucleic acids of interest present in the sample are captured on a solid support. Each nucleic acid of interest captured on the solid support is hybridized to its corresponding subset of m label extenders, and the label probe system (or the component thereof) is hybridized to the m label extenders at a hybridization temperature. The hybridization temperature is greater than a melting temperature Tₘ of a complex between each individual label extender and the component of the label probe system. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

Typically, the one or more nucleic acids of interest comprise two or more nucleic acids of interest, and the one or more subsets of m label extenders comprise two or more subsets of m label extenders.

The various hybridization and capture steps can be performed simultaneously or sequentially, in any convenient order. For example, in embodiments in which capture extenders are employed, each nucleic acid of interest can be hybridized simultaneously with its corresponding subset of m label extenders and its corresponding subset of n capture extenders, and then the capture extenders can be hybridized with capture poles associated with the solid support. Materials not captured on the support are preferably removed, e.g., by washing the support, and then the label probe system is hybridized to the label extenders.

As for the methods described above, essentially any suitable solid support can be employed. For example, the solid support can comprise particles such as microspheres, or it can comprise a substantially planar and/or spatially addressable support. Different nucleic acids are optionally captured on different distinguishable subsets of particles or at different positions on a spatially addressable solid support. The nucleic acids of interest can be captured to the solid support by any of a variety of techniques, for example, by binding directly to the solid support or by binding to a moiety bound to the support, or through hybridization to another nucleic acid bound to the solid support. Preferably, the nucleic acids are captured to the solid support through hybridization with capture extenders and capture poles.

In one class of embodiments in which the one or more nucleic acids of interest comprise two or more nucleic acids of interest and the one or more subsets of m label extenders comprise two or more subsets of m label extenders, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture pole.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture poles, thereby associating each subset of n capture extenders with a selected subset of the particles. Each of the nucleic acids of interest present in the sample is hybridized to its corresponding subset of n capture extenders and the subset of n capture extenders is hybridized to its corresponding capture pole, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

In other embodiments in which the one or more nucleic acids of interest comprise two or more nucleic acids of interest and the one or more subsets of m label extenders comprise two or more subsets of m label extenders, the nucleic acids are captured at different positions on a non-particulate, spatially addressable solid support. Thus, in one class of embodiments, the solid support comprises two or more capture poles, wherein each capture pole is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture poles, thereby associating each subset of n capture extenders with a selected position on the solid support. Each of the nucleic acids of interest present in the sample is hybridized to its corresponding subset of n capture extenders and the subset of n capture extenders is hybridized to its corresponding capture pole, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

Essentially all of the features noted for the methods above apply to these embodiments as well, as relevant; for example, with respect to composition of the label probe system; type of label; type of solid support; inclusion of blocking probes; configuration of the capture extenders, capture poles, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

In one aspect, the invention provides methods for capturing a labeled probe to a target nucleic acid, through hybridization of the labeled probe directly to label extenders hybridized to the nucleic acid or through hybridization of the labeled probe to one or more nucleic acids that are in turn hybridized to the label extenders.

Accordingly, one general class of embodiments provides methods of capturing a label to a first nucleic acid of interest in a multiplex assay in which two or more nucleic acids of interest are to be detected. In the methods, a sample comprising the first nucleic acid of interest and also comprising or suspected of comprising one or more other nucleic acids of interest is provided. A first subset of m label extenders, wherein m is at least two, and a label probe system comprising the label are also provided. The first subset of m label extenders is capable of hybridizing to the first nucleic acid of interest, and a component of the label probe system is capable of hybridizing simultaneously to at least two of the m label extenders in the first subset. The first nucleic acid of interest is hybridized to the first subset of m label extenders, and the label probe system is hybridized to the m label extenders, thereby capturing the label to the first nucleic acid of interest.

Essentially all of the features noted for the embodiments above apply to these methods as well, as relevant; for example, with respect to configuration of the label extenders, number of label extenders per subset, composition of the label probe system, type of label, number of nucleic acids of interest, source of the sample and/or nucleic acids, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

Another general class of embodiments provides methods of capturing a label to a nucleic acid of interest. In the methods, m label extenders, wherein m is at least two, are provided. The m label extenders are capable of hybridizing to the nucleic acid of interest. A label probe system comprising the label is also provided. A component of the label probe system is capable of hybridizing simultaneously to at least two of the m label extenders. Each label extender comprises a polynucleotide sequence L-1 that is complementary to a polynucleotide sequence in the nucleic acid of interest and a polynucleotide sequence L-2 that is complementary to a polynucleotide sequence in the component of the label probe system, and the m label extenders each have L-1 5′ of L-2 or wherein the m label extenders each have L-1 3′ of L-2. The nucleic acid of interest is hybridized to the m label extenders, and the label probe system is hybridized to the m label extenders at a hybridization temperature, thereby capturing the label to the nucleic acid of interest. Preferably, the hybridization temperature is greater than a melting temperature Tₘ of a complex between each individual label extender and the component of the label probe system.

Essentially all of the features noted for the embodiments above apply to these methods as well, as relevant; for example, with respect to configuration of the label extenders, number of label extenders per subset, composition of the label probe system, type of label, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

Compositions

Compositions related to the methods are another feature of the invention. Thus, one general class of embodiments provides a composition for detecting two or more nucleic acids of interest. In one aspect, the composition includes a pooled population of particles. The population comprises two or more subsets of particles, with a plurality of the particles in each subset being distinguishable from a plurality of the particles in every other subset. The particles in each subset have associated therewith a different capture pole. In another aspect, the composition includes a solid support comprising two or more capture poles, wherein each capture pole is provided at a selected position on the solid support.

The composition also includes two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture poles and thereby associating each subset of n capture extenders with a selected subset of the particles or with a selected position on the solid support. Similarly, each subset of m label extenders is capable of hybridizing to one of the nucleic acids of interest.

The composition optionally includes a sample comprising or suspected of comprising at least one of the nucleic acids of interest, e.g., two or more, three or more, etc. nucleic acids. Optionally, the composition comprises one or more of the nucleic acids of interest. In one class of embodiments, each nucleic acid of interest present in the composition is hybridized to its corresponding subset of n capture extenders, and the corresponding subset of n capture extenders is hybridized to its corresponding capture pole. Each nucleic acid of interest is thus associated with an identifiable subset of the particles. In this class of embodiments, each nucleic acid of interest present in the composition is also hybridized to its corresponding subset of m label extenders. The component of the label probe system (e.g., the amplification multimer or preamplifier) is hybridized to the m label extenders. The composition is maintained at a hybridization temperature that is greater than a melting temperature Tₘ of a complex between each individual label extender and the component of the label probe system (e.g., the amplification multimer or preamplifier). The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ.

Essentially all of the features noted for the methods above apply to these embodiments as well, as relevant; for example, with respect to composition of the label probe system; type of label; inclusion of blocking probes; configuration of the capture extenders, capture poles, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a composition for detecting one or more nucleic acids of interest. The composition includes a solid support comprising one or more capture poles, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture poles and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

In one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture pole. The capture extenders in each subset are capable of hybridizing to one of the capture poles and thereby associating each subset of n capture extenders with a selected subset of the particles.

In another class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises two or more capture poles, wherein each capture pole is provided at a selected position on the solid support. The capture extenders in each subset are capable of hybridizing to one of the capture poles and thereby associating each subset of n capture extenders with a selected position on the solid support.

Essentially all of the features noted for the embodiments above apply to these embodiments as well, as relevant; for example, with respect to composition of the label probe system; type of label; inclusion of blocking probes; configuration of the capture extenders, capture poles, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, the label probe system can include an amplification multimer or preamplifier, which amplification multimer or preamplifier is capable of hybridizing to the at least two label extenders. The composition optionally includes one or more of the nucleic acids of interest, wherein each nucleic acid of interest is hybridized to its corresponding subset of m label extenders and to its corresponding subset of n capture extenders, which in turn is hybridized to its corresponding capture pole. The amplification multimer or preamplifier is hybridized to the m label extenders. The composition is maintained at a hybridization temperature that is greater than a melting temperature Tₘ of a complex between each individual label extender and the amplification multimer or preamplifier (e.g., about 5° C. or more, about 7° C. or more, about 10° C. or more, about 12° C. or more, about 15° C. or more, about 17° C. or more, or about 20° C. or more greater than the Tₘ).

Kits

Yet another general class of embodiments provides a kit for detecting two or more nucleic acids of interest. In one aspect, the kit includes a pooled population of particles. The population comprises two or more subsets of particles, with a plurality of the particles in each subset being distinguishable from a plurality of the particles in every other subset. The particles in each subset have associated therewith a different capture pole. In another aspect, the kit includes a solid support comprising two or more capture poles, wherein each capture pole is provided at a selected position on the solid support.

The kit also includes two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture poles and thereby associating each subset of n capture extenders with a selected subset of the particles or with a selected position on the solid support. Similarly, each subset of m label extenders is capable of hybridizing to one of the nucleic acids of interest. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture poles, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a kit for detecting one or more nucleic acids of interest. The kit includes a solid support comprising one or more capture poles, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture poles and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture poles, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, in one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture pole. The capture extenders in each subset are capable of hybridizing to one of the capture poles and thereby associating each subset of n capture extenders with a selected subset of the particles.

In another class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises two or more capture poles, wherein each capture pole is provided at a selected position on the solid support. The capture extenders in each subset are capable of hybridizing to one of the capture poles and thereby associating each subset of n capture extenders with a selected position on the solid support.

Systems

In one aspect, the invention includes systems, e.g., systems used to practice the methods herein and/or comprising the compositions described herein. The system can include, e.g., a fluid and/or microsphere handling element, a fluid and/or microsphere containing element, a laser for exciting a fluorescent label and/or fluorescent microspheres, a detector for detecting light emissions from a chemiluminescent reaction or fluorescent emissions from a fluorescent label and/or fluorescent microspheres, and/or a robotic element that moves other components of the system from place to place as needed (e.g., a multiwell plate handling element). For example, in one class of embodiments, a composition of the invention is contained in a flow cytometer, a Luminex 100™ or HTS™ instrument, a microplate reader, a microarray reader, a luminometer, a colorimeter, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

Labels

A wide variety of labels are well known in the art and can be adapted to the practice of the present invention. For example, luminescent labels and light-scattering labels (e.g., colloidal gold particles) have been described. See, e.g., Csaki et al. (2002) “Gold nanoparticles as novel label for DNA diagnostics” Expert Rev Mol Diagn 2:187-93.

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. See e.g., The Handbook: A Guide to Fluorescent Probes and Labeling Technologies, Tenth Edition or Web Edition (2006) from Invitrogen (available on the world wide web at probes.invitrogen.com/handbook), for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science 298:1759; Nature Biotechnology (2003) 21:41-46; and Nature Biotechnology (2003) 21:47-51.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. ((www.) molecularprobes.com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art.

Microspheres

Microspheres are preferred particles in certain embodiments described herein since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra, for example.

Luminex Corporation ((www.) luminexcorp.com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions ((www.) radixbiosolutions.com) and Upstate Biotechnology ((www.) upstatebiotech.com). Alternatively, BD Biosciences ((www.) bd.com) and Bangs Laboratories, Inc. ((www.) bangslabs.com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and Iannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., polynucleotide capture poles with free amine, carboxyl, aldehyde, sulfhydryl or other reactive groups) to the microspheres. As another example, microspheres with surface avidin or streptavidin are available and can bind biotinylated capture poles; similarly, microspheres coated with biotin are available for binding capture poles conjugated to avidin or streptavidin. In addition, services that couple a capture reagent of the customer's choice to microspheres are commercially available, e.g., from Radix Biosolutions ((www.) radixbiosolutions.com).

Protocols for using such commercially available microspheres (e.g., methods of covalently coupling polynucleotides to carboxylated microspheres for use as capture poles, methods of blocking reactive sites on the microsphere surface that are not occupied by the polynucleotides, methods of binding biotinylated polynucleotides to avidin-functionalized microspheres, and the like) are typically supplied with the microspheres and are readily utilized and/or adapted by one of skill. In addition, coupling of reagents to microspheres is well described in the literature. For example, see Yang et al. (2001) “BADGE, Beads Array for the Detection of Gene Expression, a high-throughput diagnostic bioassay” Genome Res. 11:1888-98; Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of analyzing microsphere populations (e.g. methods of identifying microsphere subsets by their size and/or fluorescence characteristics, methods of using size to distinguish microsphere aggregates from single uniformly sized microspheres and eliminate aggregates from the analysis, methods of detecting the presence or absence of a fluorescent label on the microsphere subset, and the like) are also well described in the literature. See, e.g., the above references.

Suitable instruments, software, and the like for analyzing microsphere populations to distinguish subsets of microspheres and to detect the presence or absence of a label (e.g., a fluorescently labeled label probe) on each subset are commercially available. For example, flow cytometers are widely available, e.g., from Becton-Dickinson ((www.) bd.com) and Beckman Coulter ((www.) beckman.com). Luminex 100™ and Luminex HTS™ systems (which use microfluidics to align the microspheres and two lasers to excite the microspheres and the label) are available from Luminex Corporation ((www.) luminexcorp.com); the similar Bio-Plex™ Protein Array System is available from Bio-Rad Laboratories, Inc. ((www.) bio-rad.com). A confocal microplate reader suitable for microsphere analysis, the FMAT™ System 8100, is available from Applied Biosystems ((www.) appliedbiosystems.com).

As another example of particles that can be adapted for use in the present invention, sets of microbeads that include optical barcodes are available from CyVera Corporation ((www.) cyvera.com). The optical barcodes are holographically inscribed digital codes that diffract a laser beam incident on the particles, producing an optical signature unique for each set of microbeads.

### Arrays

In an array of capture poles on a solid support (e.g., a membrane, a glass or plastic slide, a silicon or quartz chip, a plate, or other spatially addressable solid support), each capture pole is typically bound (e.g., electrostatically or covalently bound, directly or via a linker) to the support at a unique selected location. Methods of making, using, and analyzing such arrays (e.g., microarrays) are well known in the art. See, e.g., Baldi et al. (2002) DNA Microarrays and Gene Expression: From Experiments to Data Analysis and Modeling, Cambridge University Press; Beaucage (2001) “Strategies in the preparation of DNA oligonucleotide arrays for diagnostic applications” Curr Med Chem 8:1213-1244; Schena, ed. (2000) Microarray Biochip Technology, pp. 19-38, Eaton Publishing; technical note “Agilent SurePrint Technology: Content centered microarray design enabling speed and flexibility” available on the web at chem.agilent.com/temp/rad01539/00039489.pdf; and references therein. Arrays of pre-synthesized polynucleotides can be formed (e.g., printed), for example, using commercially available instruments such as a GMS 417 Arrayer (Affymetrix, Santa Clara, Calif.). Alternatively, the polynucleotides can be synthesized at the selected positions on the solid support; see, e.g., U.S. Pat. Nos. 6,852,490 and 6,306,643, each to Gentanlen and Chee entitled “Methods of using an array of pooled probes in genetic analysis.”

Suitable solid supports are commercially readily available. For example, a variety of membranes (e.g., nylon, PVDF, and nitrocellulose membranes) are commercially available, e.g., from Sigma-Aldrich, Inc. ((www.) sigmaaldrich.com). As another example, surface-modified and pre-coated slides with a variety of surface chemistries are commercially available, e.g., from TeleChem International ((www.) arrayit.com), Corning, Inc. (Corning, N.Y.), or Greiner Bio-One, Inc. ((www.) greinerbiooneinc.com). For example, silanated and silyated slides with free amino and aldehyde groups, respectively, are available and permit covalent coupling of molecules (e.g., polynucleotides with free aldehyde, amine, or other reactive groups) to the slides. As another example, slides with surface streptavidin are available and can bind biotinylated capture poles. In addition, services that produce arrays of polynucleotides of the customer's choice are commercially available, e.g., from TeleChem International ((www.) arrayit.com) and Agilent Technologies (Palo Alto, Calif.).

Suitable instruments, software, and the like for analyzing arrays to distinguish selected positions on the solid support and to detect the presence or absence of a label (e.g., a fluorescently labeled label probe) at each position are commercially available. For example, microarray readers are available, e.g., from Agilent Technologies (Palo Alto, Calif.), Affymetrix (Santa Clara, Calif.), and Zeptosens (Switzerland).

### REFERENCES

Hess C J, et al. Gene expression profiling of minimal residual disease in acute myeloid leukaemia by novel multiplex-PCR-based method. Leukemia. 2004 December; 18(12):1981-8.
Vogel I et al. Detection and prognostic impact of disseminated tumor cells in pancreatic carcinoma. Pancreatology. 2002; 2(2):79-88.
Gilbey A M et al. The detection of circulating breast cancer cells in blood. J Clin Pathol. 2004 September; 57(9):903-11.
Molnar B et al. Molecular detection of circulating cancer cells. Role in diagnosis, prognosis and follow-up of colon cancer patients. Dig Dis. 2003; 21(4):320-5.
Vlems F A et al. Detection and clinical relevance of tumor cells in blood and bone marrow of patients with colorectal cancer. Anticancer Res. 2003 January-February; 23(1B):523-30.
Ma P C et al Circulating tumor cells and serum tumor biomarkers in small cell lung cancer. Anticancer Res. 2003 January-February; 23(1A):49-62.
Mocellin S et al (2004) Molecular detection of circulating tumor cells in an independent prognostic factor in patients with high-risk cutaneous melanoma. Int J Cancer 111:741-745
Cristofanilli M. et al., (2004) Circulating tumor cells, disease progression, and survival in metastatic breast cancer. N Engl J Med. 2004 Aug. 19; 351(8):781-91.
Ito S et al., (2002) Quantitative detection of CEA expressing free tumor cells in the peripheral blood of colorectal cancer patients during surgery with the real-time RT-PCR on a Light Cycler, Cancer Letters, 183:195-203.
Hicks D G et al., In situ hybridization in the pathology laboratory: General principles, automation, and emerging research applications for tissue-based studies of gene expression. J Mol Histol. 2004 August; 35(6):595-601.
Herzenberg L A et al. The history and future of the fluorescence activated cell sorter and flow cytometry: a view from Stanford. Clin Chem. 2002 October; 48(10):1819-27.
Timm E A Jr et al. Amplification and detection of a Y-chromosome DNA sequence by fluorescence in situ polymerase chain reaction and flow cytometry using cells in suspension. Cytometry. 1995 September 15; 22(3):250-5.
Bauman J G, Bentvelzen P. Flow cytometric detection of ribosomal RNA in suspended cells by fluorescent in situ hybridization. Cytometry. 1988 November; 9(6):517-24.
Timm E A Jr, Stewart C C. Fluorescent in situ hybridization en suspension (FISHES) using digoxigenin-labeled probes and flow cytometry. Biotechniques. 1992 March; 12(3): 362-7.
Bains M A Flow cytometric quantitation of sequence-specific mRNA in hemopoietic cell suspensions by primer-induced in situ (PRINS) fluorescent nucleotide labeling. Exp Cell Res. 1993 September; 208(1):321-6.
Patterson B K Detection of HIV-1 DNA and messenger RNA in individual cells by PCR-driven in situ hybridization and flow cytometry. Science. 1993 May 14; 260(5110):976-9.
Rufer N Telomere length dynamics in human lymphocyte subpopulations measured by flow cytometry. Nat Biotechnol. 1998 August; 16(8):743-7.
Hultdin M Telomere analysis by fluorescence in situ hybridization and flow cytometry. Nucleic Acids Res. 1998 Aug. 15; 26(16):3651-6.
Fava T A, et al Ectopic expression of guanylyl cyclase C in CD34+ progenitor cells in peripheral blood. J Clin Oncol. 2001 Oct. 1; 19(19):3951-9.
Kosman D, Mizutani C M, Lemons D, Cox W G, McGinnis W, Bier E. Multiplex detection of RNA expression in Drosophila embryos. Science. 2004 Aug. 6; 305(5685):846.
Player A N, Shen L P, Kenny D, Antao V P, Kolberg J A. Single-copy gene detection using branched DNA (bDNA) in situ hybridization. J Histochem Cytochem. 2001 May; 49(5): 603-12.
Schrock E, du Manoir S, Veldman T, Schoell B, Wienberg J, Ferguson-Smith M A, Ning Y, Ledbetter D H, Bar-Am I, Soenksen D, Garini Y, Ried T. Multicolor spectral karyotyping of human chromosomes. Science. 1996 Jul. 26; 273(5274):494-7.
Larsson C, Koch J, Nygren A, Janssen G, Raap A K, Landegren U, Nilsson M. In situ genotyping individual DNA molecules by target-primed rolling-circle amplification of padlock probes. Nat Methods. 2004 December; 1(3):227-32. Epub 2004 Nov. 18.
Zhang, L., Zhou, W., Velculescu, V. E., Kern, S. E., Hruban, R. H., Hamilton, S. R., Vogelstein, B., and Kinzler, K. W. (1997). Gene expression profiles in normal and cancer cells. Science (New York, N.Y. 276, 1268-1272.
Pinkel, D., Straume, T., Gray, J. W. Cytogenetic analysis using quantitative, high-sensitivity, fluorescence hybridization. Proc. Natl. Acad. Sci. USA 1986 83:2934-2938.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1: Detection of Nucleic Acids in Individual Cells

The following sets forth a series of experiments that demonstrate in-cell detection of nucleic acid. The results demonstrate, for example, that when staining cells on a glass substrate with QMAGEX, we can obtain a highly specific signal with a sensitivity of detecting a single mRNA molecule. Moreover, we can achieve staining of multiple mRNAs at the same time using a combination of different target probes and amplifiers. These results further demonstrate the feasibility of detecting cancer cells exhibiting transcriptional upregulation within a population of cells with normal gene expression. The results also demonstrate staining of cells in suspension and identification of them using flow cytometry, eliminating need for a solid support for the cells and allowing for rapid detection of stained cells. These results further demonstrate the ability to detect cells exhibiting transcriptional upregulation from those with low basal levels of mRNA expression in a rapid manner using flow cytometry.

Overview of Assay

We have developed an assay for detecting multiple RNA transcripts in situ in individual cells over a large cell population that we have named QMAGEX. The assay can be performed, e.g., on cells attached to a glass substrate and examined using a fluorescent microscope or on cells in suspension and analyzed using a flow cytometer. This assay is analogous in some respects to traditional RNA ISH/FISH but possesses the following unique features: 1) it has the sensitivity to detect a single mRNA transcript; 2) it is easy to conduct multiplex in situ for simultaneous detection of markers that can be correlated with cell morphology; 3) it can provide an internal control staining of a housekeeping gene through its multiplex capability to determine RNA integrity and assay quality (important for regulatory approval); and 4) the signals from QMAGEX are semi-quantitative and/or quantitative.

The basic assay procedure (FIG. 11 Panels A-D) can be done within a day and generally includes the following steps. After being fixed and permeablized, cells either on substrate or in suspension are hybridized to the following series of oligonucleotide probes. First, a set of capture probes is hybridized to the target RNA inside the cells. Next, preamplifier molecules (PreAMP) are hybridized to the capture probes, providing a bridge for the hybridization of amplifier molecules (AMP). Finally, amplification of the signal is accomplished by the binding of, e.g., up to 20 AMPs to each PreAMP, and 20 label probes (LPs) to each AMP, giving a total of 400 fluorescent labels or alkaline phosphatase (AP) labels to each target probe. (It is worth noting that signal intensity can be enhanced further by including more than one label in each LP; as just one example, by conjugating up to three fluorescent molecules per LP instead of one fluorescent molecule per LP.) In the case when AP-conjugated LPs are used in combination with Fast Red substrate, signal amplification is enhanced further due to deposition of red fluorescent precipitate in the vicinity of the target nucleic acid. Signals are detected, e.g., with either a regular fluorescent microscope with appropriate filters or with a multicolor flow cytometer.

Nonspecific hybridization can be prevented or minimized through the “cooperative hybridization” concept (for additional details, see Flagella et al. (2006) “A multiplex branched DNA assay for parallel quantitative gene expression profiling” Anal Biochem. 352(1):50-60 and U.S. patent application publication 2007/0015188 entitled “Multiplex detection of nucleic acids” by Luo et al.). Nonspecific hybridization can be prevented or minimized, for example, by designing probe sets targeting a specific mRNA sequence using a double “Z” probe design. Target double “Z” probes are prescreened against the GenBank database to ensure minimal cross-hybridization with unintended nucleic acid sequences. In the double “Z” design, two neighboring probes each contain a target-hybridizing sequence, e.g., 20 to 30 base in length with a Tₘ significantly above the assay temperature, and a PreAMP-hybridizing sequence, e.g., only 14 bases in length with a Tₘ well below the assay temperature (FIG. 11 Panels C-D). As a result, a single capture probe is able to bind to target RNA strongly and stably during hybridization, but will bind to the PreAMP weakly and unstably due to the 14 base pair region of homology having a Tₘ well below the assay temperature. However, when two capture probes are present in neighboring positions, the combined hybridization strength, e.g., of 28 complementary base pairs, holds the PreAMP strongly and stably at the assay temperature, enabling signal amplification to occur. Such a double “Z” design ensures high detection specificity and simplifies probe design for simultaneous detection of multiple targets.

Two signal amplifiers have been tested in the assay, one with 400-fold (400×AMP1) amplification and another with 16-fold (16×AMP2) amplification. The 400×AMP1 is composed of 20 AMP binding site per PreAMP and 20 AP or fluorescent conjugated-LP binding sites per AMP molecule to provide 400 labeling molecule per capture probe pair (20×20=400). The 16×AMP2 is composed of 4 AMP binding sites per PreAMP and 4 AP or fluorescent conjugated-LP binding sites per AMP to give rise to 16 labeling molecules per capture probe pair (4×4=16). The two amplifying systems have been shown experimentally to have no cross reactivity to each other.

In Cell Detection of 18S RNA

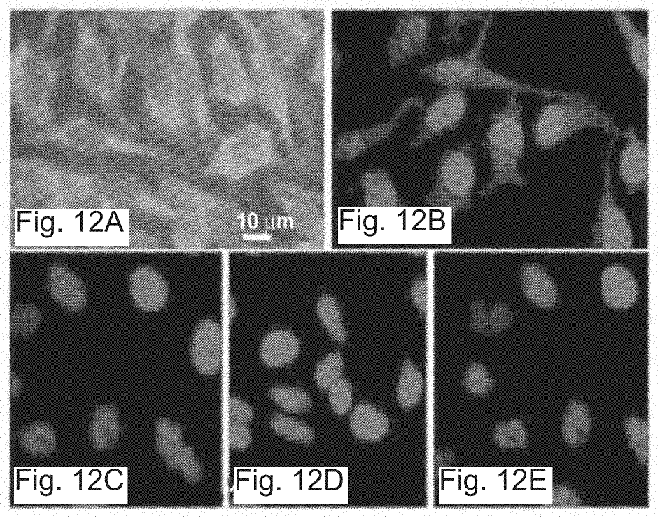

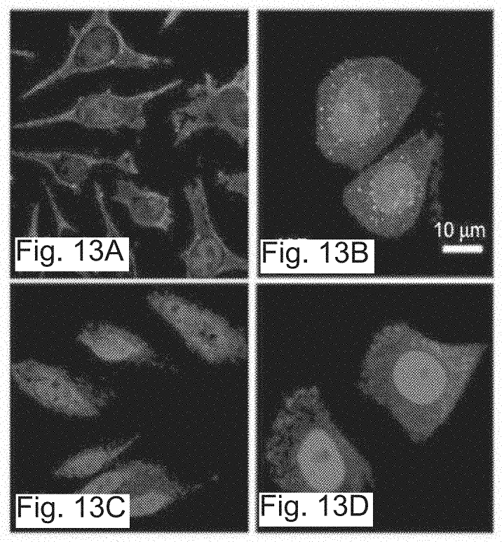

In an initial experiment, 18S capture probes (capture probes complementary to 18S RNA) in combination with 16×AMP2 were used on HeLa cells grown on coverslips. The goal of this initial effort was to identify an assay condition that produces maximal signal-to-background ratio. As will be discussed below, we have achieved a signal-to-background ratio sufficient for single copy mRNA detection. To understand the magnitude of signal enhancement by the amplifiers, we conducted parallel experiments in which the same set of 18S capture probes were used to probe 18S RNA in HeLa cells. One set of capture probes was amplified by 16×AMP2/Alexa 488-LP while the other set was probed with an amplifier designed to have only one PreAMP/AMP and one Alexa 488-LP binding site (1×AMP3). By setting the camera exposure time constant, we captured the 18S signal in cells labeled with 16×AMP2 (FIG. 12 Panel A) and 1×AMP3 (FIG. 12 Panel B). We reproducibly saw a higher 18S signal in cells labeled with 16×AMP2 than with 1×AMP1, suggesting that signal amplification is necessary to gain a greater signal-to-background ratio. To confirm the specificity of the capture probe design, we used a probe set targeting the anti-sense strand of the 18S intron sequence, and it showed a low to absent background signal (FIG. 12 Panel C). We have also found that the 18S signal is completely removed when the cells are pre-treated with RNase or when the cells are incubated with either no capture probe set or with only the tail sequence complementary to the PreAMP (data not shown). These results thus indicate that the fluorescent signal we observed is specific in labeling 18S RNA. The double “Z” capture probe design used in QMAGEX greatly improves the assay specificity. In experiments in which one half or the other of the double “Z” probe set was used, signal is greatly reduced as compared to that when the full probe set is used (FIG. 12 Panels D and E vs. Panel A). Based on the above results, we conclude that QMAGEX performs to our intended design principle and the assay is the first of its kind in simultaneous signal amplification (PreAMP/AMP) and background reduction (double Z design) to achieve high signal and great specificity.

Duplex QMAGEX Assay

To explore its potential for in situ detection of low copy RNA transcripts and its capability for multiplex detection, we developed a multiplex QMAGEX assay using 18S and Her-2 as the model genes. HeLa and SKBR3 are labeled with DAPI to facilitate the identification of nuclei (blue). Her-2 mRNA was labeled with the 400×AMP1/Alexa 488-LP (green) while 18S RNA was labeled with the 16×AMP2/Alexa 555-LP (red). High 18S expression in HeLa (FIG. 13 Panels A and C) and SKBR3 (FIG. 13 Panels B and D) resulted in a ubiquitous staining pattern around the entire cells. When labeling Her-2 mRNA (green), signals appeared to be punctate fluorescent dots with SKBR3 cells showing a higher number of dots per cell (FIG. 13 Panel B) than HeLa (FIG. 13 Panel A), consistent with the fact that SKBR3 is a breast cancer cell line with HER2 gene amplification whereas HeLa has no HER2 amplification. Since a control probe set targeting the anti-sense strand of the Her-2 intron sequence gave rise to no green fluorescent dots in any cells (FIG. 13 Panels C and D), we concluded that the capture probes designed for Her-2 mRNA are specific in detecting Her-2 mRNA transcripts. We also noticed the variation of RNA dots in individual HeLa cells. Considering the relative same level of 18S (a housekeeping gene) staining in all HeLa cells, we believe that the variation in dot number seen in HeLa is likely to be an intrinsic property of gene expression, rather than assay variability, and is consistent with previous observations on stochastic expression of mRNA transcripts (e.g. reviewed by Shav-Tal et al. (2004) “Imaging gene expression in single living cells” Nat Rev Mol Cell Biol. 5(10):855-61). Thus we have demonstrated using a Her-2/18S duplex that the QMAGEX assay can be used to detect two RNA transcripts simultaneously and the relative signals can be used to compare gene expression.

Single Copy mRNA Detection

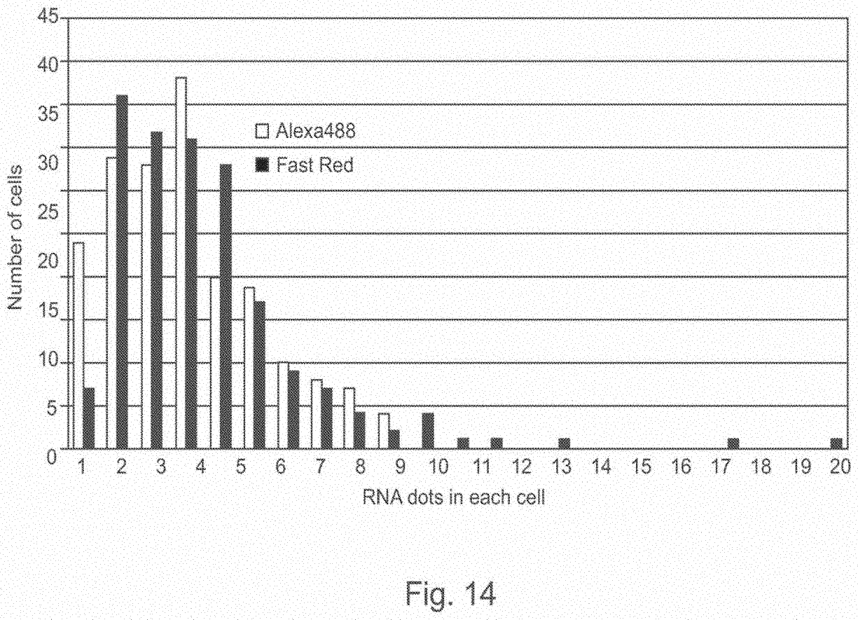

The punctate expression pattern of Her-2 in HeLa and SKBR3 cells detected using QMAGEX suggests that each fluorescent dot is one mRNA; however, we can not exclude the possibility that each puncta represents two or more mRNAs in close proximity to one another. We designed two experiments in order to distinguish between these two possibilities. The first experiment utilized QuantiGene 2.0, an established quantitative assay, to compare the average copy number of transcripts per cell to the number of fluorescent dots seen in QMAGEX. We labeled Her-2 mRNA in HeLa cells with capture probes designed for the Her-2 gene followed by 400×AMP1/Alexa488-LP or 400×AMP1/AP-LP and Fast Red substrate reaction to ensure sensitive and reproducible detection of all RNA dots. In both assays, 200 cells were randomly selected. The number of fluorescent dots in each cell was counted and the average dots per cell were calculated. The histogram of fluorescent dots per cell by both labeling schemes (FIG. 14) showed a similar stochastic distribution with a median value at 3 copies per cell and an average value of 3.2-3.4 copies per cell. The similar number of dots seen using both fluorescence and Fast Red indicated that the extra signal amplification created by the Fast Red substrate is not necessary to elucidate all of the RNAs present in the cells. Using the QuantiGene 2.0 assay, the same batch of HeLa cells were tested and showed an average of ˜5 Her-2 mRNA transcripts per cell, which is close to our results using the QMAGEX assay (Table 1). To further confirm these results, we designed a second experiment in which we measured the fluorescent intensity of each dot for Her-2 mRNA, and compared them with the fluorescent intensity of each dot in HER2 genomic DNA. In this experiment, RNA and DNA QMAGEX assays were run in parallel on the same batch of HeLa cells using the same capture probes. With a constant camera exposure time, pictures were taken from both DNA and RNA QMAGEX assays. The CellProfiler program (www (dot) cellprofiler (dot) org) was utilized to measure fluorescent intensity of each dot. Since we used the same probe set for both RNA and DNA FISH, a similar distribution of fluorescent intensity would be expected if RNA was being measured at a single copy resolution. This is because each fluorescent dot in DNA FISH represents a single gene copy. In our analysis of fluorescent intensity distribution (data not shown), the range of fluorescent intensity from the RNA dots does not exceed the fluorescent intensity from each DNA dot, confirming that each RNA dot is indeed representative of a single copy mRNA. In situ detection of single copy mRNA by routine fluorescent microscopy is a major achievement because this has not been done before. Traditional ISH/FISH assays only have a detection sensitivity around 50 copies per cell, which excludes 95% of the genes which are expressed at a level that is less than 50 transcripts per cell (Zhang et al. (1997) “Gene expression profiles in normal and cancer cells” Science 276(5316):1268-72).

TABLE 1
Average mRNA copies/cell determined by QG2.0.

| | HeLa | | |
|---|---|---|---|
| Genes | Control | Induced | SKBR3 |
| Her-2 | ~5 | NA | ~100 |
| IL-6 | ~2 | ~5 | NA |
| IL-8 | ~1 | ~275 | NA |

Determination of Gene Expression Changes in Single Cells

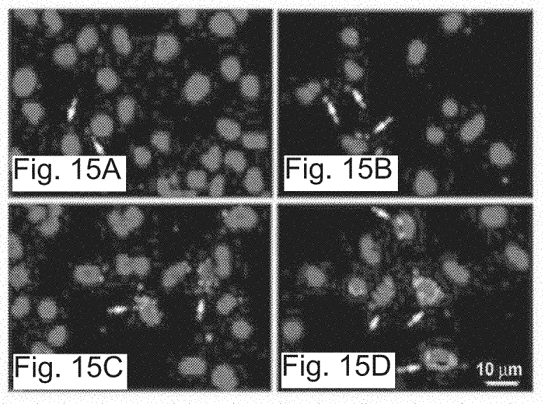

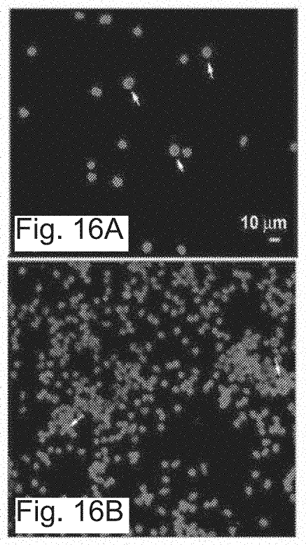

The induction of cytokine gene expression in HeLa cells upon PMA-treatment is a classic model for validation of expression profiling technologies. It has been shown that IL-6 and IL-8 mRNA are expressed at very low levels in resting HeLa cells, but they are induced significantly upon PMA treatment (e.g. Zhang et al. (2005) “Small interfering RNA and gene expression analysis using a multiplex branched DNA assay without RNA purification” J Biomol Screen. 10(6):549-56). Using QuantiGene 2.0, we have determined that, on average, there are only about 1 to 2 copies of IL-8 and IL-6 mRNA per cell in resting HeLa cells and upon PMA induction IL-8 and IL-6 increase to ˜275 copies and ˜5 copies per cell, respectively (Table 1). Since existing technologies (e.g. microarray, qRT-PCR, QuantiGene 2.0) measure gene expression in purified RNA or cell lysates, the measurement represents an average response of groups of cells in the sample. In contrast, QMAGEX offers a unique opportunity to determine mRNA expression in single cells in response to PMA treatment. Using 400×AMP1 in combination with Alexa 488-label probe, we have determined expression for IL-6 and IL-8 mRNA in resting (FIG. 15 Panels A and B) and PMA-treated (FIG. 15 Panels C and D) HeLa cells at the single cell level. While very low levels of IL-6 and IL-8 mRNA expression are observed in resting HeLa cells, significant induction of IL-6 and extremely high level of induction of IL-8 are observed in some, but not all of the PMA-treated HeLa cells. Thus, while IL-6 and IL-8 expression measured in single cell by QMAGEX assay are consistent with the average expression response obtained by QuantiGene 2.0, there is a dramatic variation in single cell response as some cells show extremely high levels of induction while other cells remain unchanged (FIG. 15 Panels C and D). The dramatic variation in single cell expression profile underscores the heterogeneity in individual cell's response to PMA treatment, even with a supposed homogenous cell line. To our knowledge this is the first study to look at the induction response of native gene expression at the single cell level. The observed heterogeneous expression response underlines the value of studying single cell biology for which QMAGEX can be a valuable tool.

Detection of Cancer Cells in Mixed Cell Populations

In order to determine the feasibility of QMAGEX in CTC detection, we mixed breast cancer cells into Jurkat cells (T cell origin) or WBCs, and evaluated the capability of QMAGEX to distinguish breast cancer cells from Jurkat cells or WBCs. For example, we mixed SKBR3 cells with Jurkat cells at 1:50 ratio, cultured them for a day, and detected the mRNA expression of the common cancer cell marker CK19 in the mixed cells by QMAGEX. Using capture probes targeting CK19 in combination with 400×AMP1/AP-LP and Fast Red substrate, SKBR3 cells were identified by their high expression of CK19 among CK19 negative Jurkat cells (FIG. 16 Panel A). We have also spiked BT474 breast cancer cells into Ficoll-purified blood cells at a 1:1,000 ratio, cytospun the cells onto a slide, and performed QMAGEX with capture probes targeting CK19 in combination with 400×AMP1/AP-LP and Fast Red substrate. Similar to the Jurkat/SKBR3 mix cells, 1 per 1000 cell was labeled with CK19 (FIG. 16 Panel B), suggesting that the QMAGEX assay could be used to discriminate cells based on differential gene expression level. In addition to CK19, we also showed that QMAGEX with Her-2 capture probe is as effective in identifying SKBR3 cells among HeLa, Jurkat and WBCs (data not shown). These results thus prove the feasibility of using the QMAGEX assay for CTC detection in patient blood samples.

Flow Cytometry Based QMAGEX Assay (FC-QMAGEX)

Currently, CTC detection in patient blood samples requires a CTC enrichment step (e.g. immunomagnetic separation) followed by staining and scanning a large population of cells on a glass substrate for identification of rare, positively stained CTCs. Enrichment, deposition of cells on a glass substrate, and scanning using an automated digital microscope are laborious and time consuming procedures. In order to circumvent these steps, we tested the capability of the QMAGEX assay to stain cells in suspension and for the positively stained cells to be identified by flow cytometry.

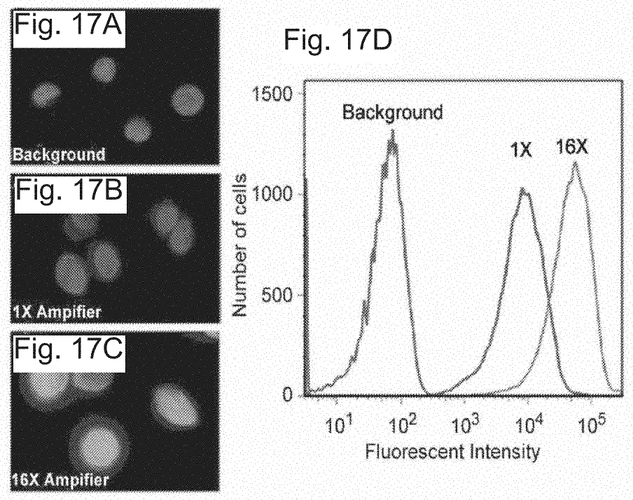

For the FC-QMAGEX assay, we first trypsinized HeLa cells grown on a substrate into suspension cells, and then hybridized the cells with 18S capture probes followed by signal amplification with either a 16×AMP2 or a 1×AMP3 and labeling using Alexa488. Positive staining was identified in the suspension HeLa cells by fluorescent microscopy and compared with control cells not hybridized with capture probes or signal amplifiers (FIG. 17 Panels A-C). The 16×AMP2 had a stronger fluorescent stain in rounded suspension HeLa cells than the 1×AMP3, consistent with the previous results on cells grown on substrate (FIG. 12 Panels A-B). We next determined the sensitivity of flow cytometry (LSR II, BD Biosciences) to detect and quantify 18S RNA expression in single cells with 50,000 cells counted per assay. The flow cytometric histogram (FIG. 17 Panel D) showed the detection of the 1×AMP3 having signals ˜100-fold above background, demonstrating a high level of detection sensitivity. Detection of cells with the 16×AMP2 lead to an approximately 10-fold increase in signal intensity over that seen with the 1×AMP3. Since the signal of 16×AMP2 is at the point of saturation in the detection scale, the 10-fold increase in signal over the 1×AMP3 is likely an underestimate of the true signal amplification achieved. To understand the contribution of background fluorescence in flow cytometry, we compared the background fluorescence from 1) cells hybridized with no capture probes and no signal amplifier or label probe (a measure of cellular autofluorescence); 2) cells hybridized with no capture probes but with 400×AMP1 and Alexa488 label probe; or 3) cells hybridized with 18S intron capture probes followed by 400×AMP1 and Alexa488 label probe. Little difference was seen in all the background fluorescence (data not shown) measured, suggesting that the background is mainly contributed by cellular autofluorescence. This result again demonstrates the value of the double “Z” design in reducing non-specific hybridization-related background, which had been several folds higher than cellular autofluorescence (e.g. Yu et al. (1991) “Sensitive detection of RNAs in single cells by flow cytometry” Nucleic Acids Res. 20(1):83-8). This study demonstrates that specific labeling and detection of 18S RNA can be achieved for HeLa cells in suspension and the 18S RNA level can be measured quantitatively by flow cytometry.

We tested a second marker, CK19, in the MCF7 cell line. We were also able to detect a strong positive signal over background by ˜400-fold (data not shown) These results demonstrate the feasibility of performing the QMAGEX assay in suspension, negating the need for a solid support and increasing the scanning speed to over 20,000 cells per second, far outpacing an automated digital microscope. Furthermore, the ability of a flow cytometer to detect a 1× amplification indicates that we can detect very low expressing transcripts and distinguish these from higher expressing mRNAs.

Detection of Low Copy mRNA Transcripts Using FC-QMAGEX

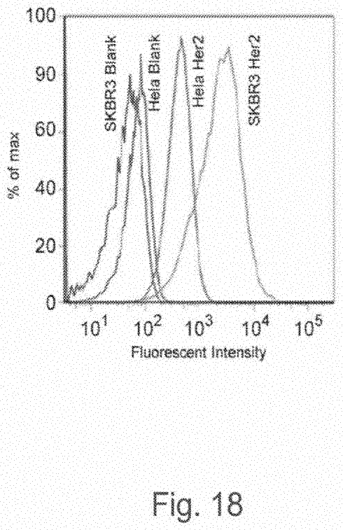

One of the hallmarks of cellular transformation is the upregulation of cancer specific genes. This increase in transcript number can be the result of genetic changes such as gene amplification, as is the case with a subset of breast cancers distinguished by an increase in HER2 gene copy number. To determine whether our flow cytometry based QMAGEX assay could distinguish these transformed cells from a general population that expresses only low basal levels of mRNA, we again used the SKBR3 cell line, which contains a HER2 gene amplification, and compared the Her-2 mRNA expression levels to those seen in the unamplified HeLa cell line. SKBR3 and HeLa cells were hybridized with Her2 capture probes, amplified with the 400×AMP1, and labeled with Alexa488. Unhybridized cells were used as a negative control for background fluorescence. The flow cytometric histogram showed an increase in signal intensity for both HeLa and SKBR3 cells over background (FIG. 18). Since HeLa cells showed an average expression level of 5 copies of mRNA per cell in QuantiGene 2.0 and an average of 3 copies per cell in QMAGEX, this results suggest that the FC-QMAGEX assay is already highly sensitive, having detection sensitivity below 5 copies per cell. This result is in sharp contrast with the previous reported detection limit of 1,800 RNA transcripts in flow cytometry (Yu et al. (1991) “Sensitive detection of RNAs in single cells by flow cytometry” Nucleic Acids Res. 20(1):83-8), suggesting that FC-QMAGEX assays are able to detect a much greater number of functionally relevant genes in cell. In FC-QMAGEX, the SKBR3 cells, which contain a Her-2 gene amplification, showed an approximately 10-fold higher level of Her-2 expression than HeLa cells, consistent with previous observation when examined on glass substrate (FIG. 13 Panels A-B). Interestingly, the SKBR3 cell line shows a wider range of fluorescent intensities than HeLa cells. This is likely due to different levels of gene amplification in different cells resulting in varying degrees of Her-2 expression, a phenomenon that would not occur in HeLa cells carrying a normal gene copy number. These results demonstrate the feasibility of detecting both basal and overexpressed mRNAs in a mixed cell population using FC-QMAGEX. More importantly, these experiments indicate that CTCs overexpressing cancer cell markers can be identified by QMAGEX separately from WBCs without enrichment due to the fast sampling rate of over 20,000 cells per second by flow cytometry.

Detection of mRNA Transcripts in FFPE Tissue Sections and Microarrays

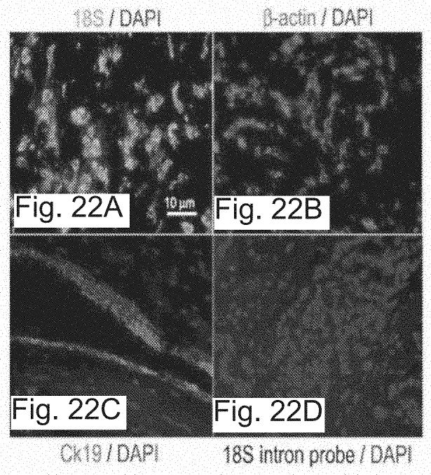

FFPE tissue section is a sample type widely used in pathology. FFPE tissue sections are generally considered to be more difficult to work with than cell lines and blood cells due to additional issues such as target access, RNA stability and autofluorescence. The techniques described herein, however, permit convenient detection of nucleic acids in FFPE tissue sections. The following experiments illustrate the potential and capability of QMAGEX for in situ detection of RNA transcripts in this particular sample type. FIG. 22 illustrates detection of various targets in breast cancer FFPE tissue section. FIG. 22 Panels A and B illustrate detection of genes with high levels of expression (>1,000 copies per cell), such as 18S (Alexa-488) and beta-actin (Fast Red) (FIG. 22 Panels A and B, respectively). Detection of mid-level expression genes (>100 and <1,000) such as CK19 (Fast Red) is illustrated in FIG. 22 Panel C. CK19 is a marker for epithelial cells and cancer epithelial cells. The fact that CK19 RNA is specifically detected in epithelial and cancer epithelial cells but not in neighboring stromal cells (FIG. 22 Panel C), and the fact the assay background is very low in FFPE tissue section (FIG. 22 Panel D), indicates that the FFPE-MAGEX assay is highly specific and is also applicable to very low copy RNA detection. Techniques are similar to those described for detection of RNA in situ in cell lines, although the FFPE tissue sections are also first subjected to de-paraffinization, de-crosslinking, and autofluorescence reduction using standard techniques.

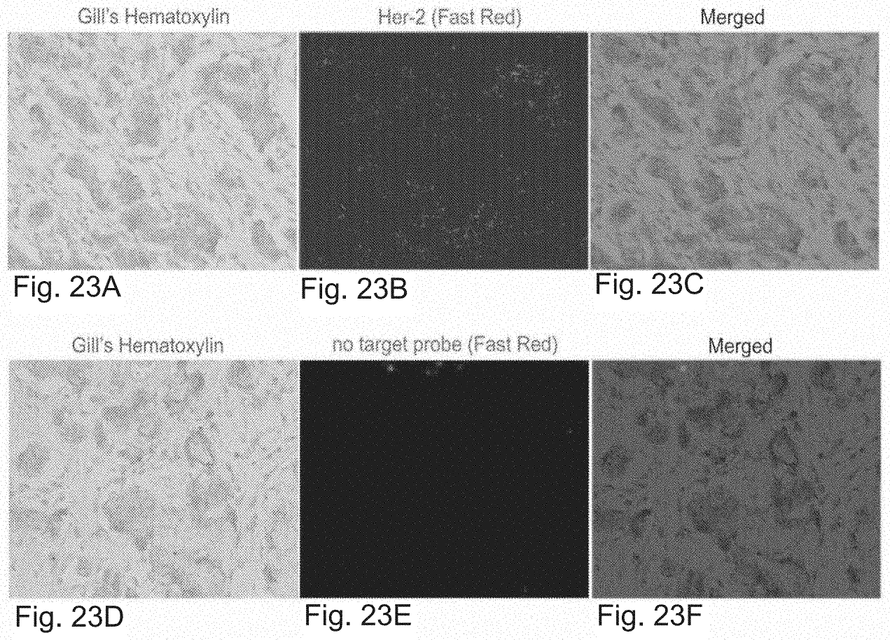

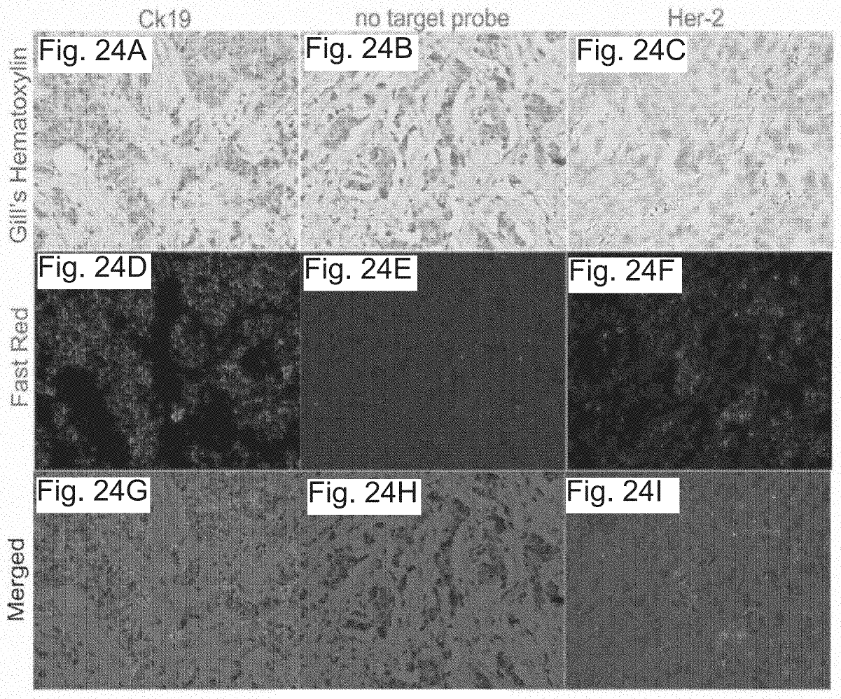

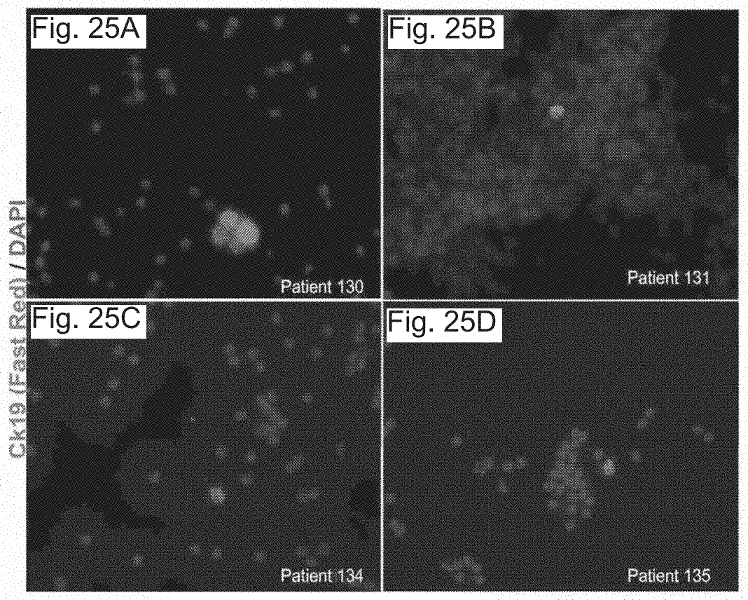

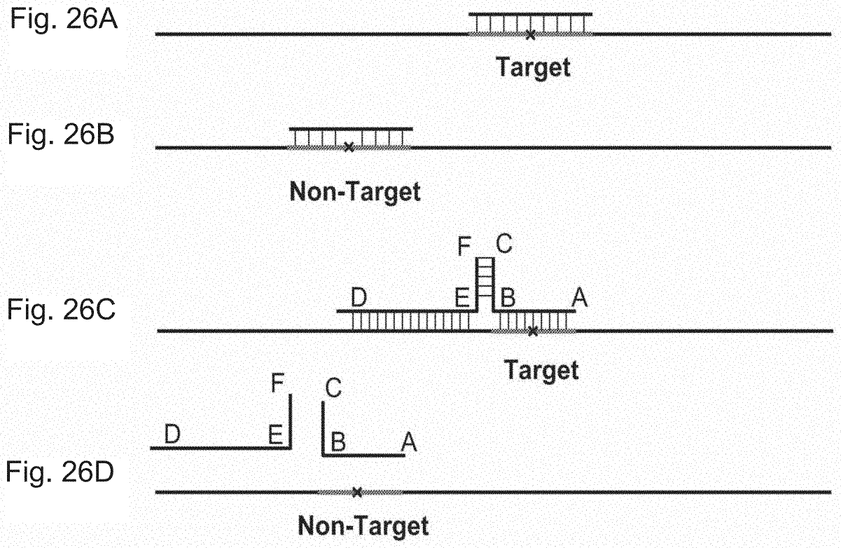

A further experiment showing that techniques described herein permit detection of low copy RNAs in FFPE tissue sections is illustrated in FIG. 23, which illustrates Her-2 mRNA detection in breast cancer FFPE samples. FFPE sections from breast cancer tissue were labeled using a MAGEX assay with either a probe set for the Her-2 marker (FIG. 23 Panels A-C) or no target probe (FIG. 23 Panels D-F). The left column (Panels A and D) shows Gill's Hematoxylin staining of the cell nuclei in the tissue section. The middle column (Panels B and E) shows the tissue section stained with a MAGEX assay using Her-2 probe (Panel B) or no target probe (Panel E) in combination with Fast Red substrate. The right column shows the merged pictures for Her-2/Gill's Hematoxylin (Panel C) and no target probe/Gill's Hematoxylin (Panel F). Low copy Her-2 is readily visualized and optionally quantitated in the FFPE samples.

FIG. 24 illustrates mRNA detection in breast cancer tissue microarray (TMA) FFPE samples. FFPE tissue microarray from breast cancer tissues were labeled using a MAGEX assay with Ck19 (FIG. 24 left column, Panels A, D and G), Her-2 (right column, Panels C, F, and I) or no target probe (middle column, Panels B, E, and H). The top row (Panels A-C) shows Gill's Hematoxylin staining of the cell nuclei in the tissue sections. The middle row (Panels D-F) shows the tissue sections labeled with MAGEX assay using Ck19 probe (Panel D), Her-2 probe (Panel F) or no target probe (Panel E) in combination with Fast Red as a substrate. The bottom row shows merged pictures for Ck19/Gill's Hematoxylin (Panel G), Her-2/Gill's Hematoxylin (Panel I) and no target probe/Gill's Hematoxylin (Panel H).

CTC Identification in Breast Cancer Patients

As noted, one exemplary application of techniques described herein is in identification of CTCs. FIG. 25 illustrates identification of CTCs in blood samples from breast cancer patients.

Nucleated cells were first purified from patient blood samples. Cells were then fixed onto glass slides and a MAGEX assay using Ck19 as the marker was used to identify the cancer cells. FIG. 25 Panels A-D show MAGEX Ck19 labeling of the cancer cells in four patient blood cell samples.

Exemplary Marker Panel

As noted above, a number of markers can be employed to identify various cell types, including, for example, CTCs. As just one example, a panel of markers including mRNA transcripts CK19, MamA (mammaglobin A), CD45, and/or Her-2 can be employed, e.g., in a 4-plex QMAGEX assay identifying and characterizing SKBR3 cells spiked into blood or CTCs in metastatic breast cancer patients. CK19 has proven to be a highly expressed generic marker for tumor cells of epithelial origin. We have demonstrated its sensitivity and specificity in distinguishing cancer cells from white blood cells. MamA is another established marker for distinguishing breast cancer cell from blood cells (reviewed by Lacroix (2006) “Significance, detection and markers of disseminated breast cancer cells” Endocr Relat Cancer. 13(4):1033-67). This marker is particularly useful in eliminating potential CK19 false positive skin epithelial cells which are introduced through needle aspiration of blood. CD45 can be used as a negative marker for cancer cell because it is a well known marker for blood cells and we have determined it to have no expression in cancer cells. Her-2 is used here to demonstrate the capability of QMAGEX for providing functional information on the CTCs. Several studies have shown that Her-2 gene amplification can be detected in CTCs not only in patients whose primary tumor is HER2+, but also in some patients whose primary tumor is HER2− (e.g., Hayes et al. (2002) “Monitoring expression of HER-2 on circulating epithelial cells in patients with advanced breast cancer” Int J Oncol. 21(5):1111-7, Meng et al. (2004) “HER-2 gene amplification can be acquired as breast cancer progresses” Proc. Nat. Acad. Sci. 101(25):9393-9398, and Wulfing et al. (2006) “HER2-positive circulating tumor cells indicate poor clinical outcome in stage I to III breast cancer patients” Clin Cancer Res. 12(6):1715-20). More interestingly, breast cancer patients whose primary tumor is HER2− but CTC HER2+ can respond to Herceptin treatment, suggesting that determining HER2 status in CTC could be an effective way of guiding targeted therapy (Meng et al. (2004) supra). At the 2007 ASCO meeting, there were a number of studies showing that some patients with primary tumor HER2− status can also benefit from Herceptin treatment (e.g. Paik et al. (2007) “Benefit from adjuvant trastuzumab may not be confined to patients with IHC 3+ and/or FISH-positive tumors: central testing results from NSABP B-31” Program and abstracts of the 43rd American Society of Clinical Oncology Annual Meeting; Jun. 1-5, 2007; Chicago, Ill. Abstract 511). Thus it would be valuable to investigate whether HER2 status in CTCs can serve as a surrogate marker for targeted therapy selection. We believe that Her-2 mRNA is potentially a more accurate marker than HER2 DNA gene amplification because it is more directly related to its protein expression. In summary, three of the four RNA markers (CK19, MamA and CD45) are used to detect and distinguish breast cancer cells in blood through “Boolean Conditioning” (use of more than one independent markers to increase specificity of detection and decrease false positives, as described hereinabove) and one marker (Her-2) is used to provide functional information about the CTCs. Additional RNA markers for breast cancer cell detection in blood can also be employed (e.g., see review by Lacroix (2006) supra).

### Materials and Methods

Cell Culture and PMA Induction

All cell lines were obtained from American Type Cell Culture Collection (ATCC; Manassas, Va.) and cultured in appropriate media. Cells were grown on glass coverslips coated with 1:10 dilution of poly-L-lysine solution (Sigma Diagnostics, Inc.; St. Louis, Mo.) using conditions provided by the ATCC. For PMA induction experiments, HeLa cells were cultured until 60%-70% confluency (18-20 hr at 37° C.) in Dulbecco's Modified Eagle's Medium (DMEM, Invitrogen, Carlsbad, Calif.) containing 10% serum followed by serum-free DMEM for 18 hr. Cells were then treated with 10 ng/ml PMA (CalBiochem, San Diego) in serum-free DMEM and collected at various time point for analysis.

Cell Fixation and Storage

Cells grown on coverslips were fixed with 4% formaldehyde in PBS (0.01 M phosphate buffer, pH7.5) at room temperature for 30 minutes. Fixed cells were washed in PBS, dehydrated through a graded ethanol series (50%, 70% and 100%) at room temperature and stored in 100% ethanol at −20° C. For in situ staining in suspension, cells were trypsinized and collected by centrifugation at 290 g for 10 min at room temperature. Pellets were re-suspended in 1×PBS and centrifuged at 290 g for 10 min at room temperature. Suspension cells were re-suspended in 4% formaldehyde in 1×PBS for 30 min at room temperature. Fixed cells were collected by centrifugation and dehydrated in the same way as for cells grown on coverslips.

Oligonucleotide Probes and Signal Amplification System

Target probes were designed using modified Probe Design Software (ProbeDesigner™ from Panomics, Inc.; see also Bushnell et al. (1999) “ProbeDesigner: for the design of probe sets for branched DNA (bDNA) signal amplification assays Bioinformatics 15:348-55). 13 pairs of DNA oligonucleotides containing sequence complementary to unique region of 18S rRNA were used to label 18S rRNAs. 52 pairs of DNA oligonucleotides complementary to region in ERBB2(Her-2) were used in detecting Her-2 mRNA. 23 pairs of DNA oligonucleotides complementary to region of Interlukin-6 (IL-6) were used in detecting IL-6 mRNA. 20 pairs of DNA oligonucleotides complementary to unique region of Interlukin-8 (IL-8) were used in detecting IL-8 mRNA. Signal amplification system including preAMP and AMP and fluorescent molecules or Alkaline phosphatase (AP)-conjugated label probes.

RNA In Situ Hybridization on Cells Grown on Coverslips

Fixed cells were re-hydrated through a graded ethanol series (100%, 70% and 50%) and washed 3 times in PBS. To access nuclear RNA, cells were washed in 1×PBS containing 0.1% Tween 20 for 3 min at room temperature. Cells were incubated in 2.5-5 μg/ml proteinase K in PBS for 10 min at room temperature and washed 3 times with PBS for 10 min total. After the proteinase K treatment, cells were incubated with 1 pmole of target probes in target buffer containing 6×SSC, 25% formamide, 0.2% Brij-35, 0.2% casein and 0.25% Blocking Reagent (Roche Diagnostics, Indianapolis, Ind.) at 40° C. in a humidifying chamber for 3 hrs. For detecting 18S rRNA, 0.2 pmole target probe and 1.5 hr incubation time at 45° C. in a humidifying chamber is sufficient. Cells were washed at room temperature with 2×SSC, 0.2×SSC and 0.1×SSC containing 0.0025% Brij-35 detergent for 2 min each. Cells were then incubated with 100 fmole preAMP in Hybridization buffer B (15% formamide, 5×SSC, 0.3% SDS, 10% Dextran Sulfate, 1 mM ZnCl₂, 10 mM MgCl₂, 0.025% Blocking Reagent (Roche Diagnostics, Indianapolis, Ind.), 0.1 mg/ml denatured ss DNA and 50 μg/ml yeast tRNA) in a humidifying chamber at 40° C. for 25 min. Coverslips were washed in 0.1×SSC containing 1 mM EDTA 2 times for 2 min and 5 min at room temperature. Cells were incubated with 100 fmole AMP in hybridization buffer B in a humidifying chamber at 40° C. for 15 min. Coverslips were washed in 0.1×SSC containing 1 mM EDTA 2 times for 2 min and 5 min at room temperature. Cells were incubated with 100 fmole AP-conjugated label probe or 5 pmole fluorescent molecules-conjugated label probe in hybridization buffer C (5×SSC, 0.3% SDS, 10% Dextran Sulfate, 1 mM ZnCl₂, 10 mM MgCl₂, 0.025% Blocking Reagent, 0.1 mg/ml denatured ss DNA and 50 μg/ml yeast tRNA) in a humidifying chamber at 40° C. for 15 min. Coverslips were washed in 0.1×SSC containing 1 mM EDTA 2 times for 2 min and 5 min at room temperature. If the AP-conjugated label probe was used, cells were incubated in Tris-HCl, pH8 containing 0.1% Brij-35, 1 mM ZnCl₂ and 10 mM MgCl₂ for 5 min followed by exposing the cells to Fast Red Substrate (Dako, Carpinteria, Calif.) for 10 min at room temperature. For using 16×AMP system, preAMP, AMP and label probes were used at 1 pmole, 1 pmole and 5 pmole concentrations. Coverslips were mounted onto slides using Vectashield containing DAPI (Vector Laboratories Inc., Burlingame, Calif.) or Prolong Gold anti-Fade Mounting medium (Invitrongen, Carlsbad, Calif.).

RNA In Situ Hybridization on Cells in Suspension

Fixed cells were collected by centrifuging at 290 g for 5 min at room temperature. Cells were re-hydrated through Ethanol series (100%, 70% and 50%) and washed with 100 μl 1×PBS containing 2% BSA for 2 times. Cells were re-suspended and incubated in 100 μl of 1×PBS containing 0.25-0.5 μg proteinase K for 8 min at room temperature. Immediately after 8 min incubation with proteinase K solution, 25 μl of 10% BSA was added and cells were centrifuged at 290 g for 2 min. Supernatant was removed and cells were re-suspended in 100 μl 1×PBS containing 2% BSA. Cells were centrifuged at 290 g for 5 min and re-suspended in 100 μl 1×PBS containing 2% BSA. After centrifuging at 290 g for 5 min, supernatant was removed and cells were re-suspended in 100 μl of target buffer containing 1 pmole of target probes to incubate at 40° C. water bath for 3 hrs. After hybridization, 25 μl of 10% BSA was added to each sample and centrifuged at 290 g for 5 min. Cells were washed at room temperature with 2×SSC, 0.2×SSC and 0.1×SSC containing 0.0025% Brij-35 and 2% BSA for 2 min each. Cells were then incubated with 300 fmole preAMP in Hybridization buffer B′ B (15% formamide, 5×SSC, 0.3% SDS, 5% Dextran Sulfate, 1 mM ZnCl₂, 10 mM MgCl₂, 0.025% Blocking Reagent (Roche Diagnostics, Indianapolis, Ind.), 0.1 mg/ml denatured ss DNA and 50 μg/ml yeast tRNA) in a 40° C. water bath for 25 min. After hybridization, 25 μl of 10% BSA was added to each sample and centrifuged at 290 g for 5 min to collect cell pellets. Pellets were re-suspended and washed in 0.1×SSC containing 1 mM EDTA and 2% BSA for 2 times for 2 min and 5 min at room temperature. Cells were incubated with 300 fmole AMP in hybridization buffer B′ at 40° C. water bath for 15 min. After hybridization, 25 μl of 10% BSA was added to each sample and centrifuged at 290 g for 5 min to collect cell pellets. Cells were washed in 0.1×SSC containing 1 mM EDTA and 2% BSA for 2 times for 2 min and 5 min at room temperature. Cells were incubated with 300 fmole AP-conjugated label probe or 15 pmole fluorescent molecules-conjugated label probe in hybridization buffer C′ (5×SSC, 0.3% SDS, 5% Dextran Sulfate, 1 mM ZnCl₂, 10 mM MgCl₂, 0.025% Blocking Reagent (Roche Diagnostics, Indianapolis, Ind.), 0.1 mg/ml denatured ss DNA and 50 μg/ml yeast tRNA) at 40° C. water bath for 15 min. After hybridization, 25 μl of 10% BSA was added to each sample and centrifuged at 290 g for 5 min to collect cell pellets. Cells were washed in 0.1×SSC containing 1 mM EDTA and 2% BSA for 2 times for 2 min and 5 min at room temperature. If the AP-conjugated label probe was used, cells were incubated in Tris-HCl, pH8 containing 0.1% Brij-35, 1 mM ZnCl₂ and 10 mM MgCl₂ for 5 min followed by exposing the cells to Fast Red Substrate (Dako, Carpinteria, Calif.) for 10 min at room temperature. For using 16×preAMP/AMP system, preAMP, AMP and label probes were used at 3 pmole, 3 pmole and 15 pmole concentrations. Fluorescent intensity of individual cells was analyzed using LSR flow cytometer (BD Biosciences, Franklin Lakes, N.J.).

Flow Cytometric Analysis

Labeled cells in suspension were analyzed using an LSR flow cytometer (BD Biosciences, Franklin Lakes, N.J.). Flow cytometric data were analyzed using FlowJo Software (Tree Star Inc., Ashland, Oreg.).

Microscope and Imaging

Slides were viewed under an Olympus IX71 fluorescent microscope and images were taken using Micro Suite B3 software. Fluorescent dot intensity was measured using CellProfiler (www (dot) cellprofiler (dot) org) and images were generated using Adobe Photoshop.

Cell Density and mRNA Copy Number Estimation

To estimate the cell number on each coverslip, 4 coverslips were transferred to a clean 24-well dish, washed with PBS and treated with trypsin (Gibco) for 5-10 min at room temperature until the cells were detached. Trypsin was inactivated by adding 2 volume of medium containing 10% serum and cells were centrifuged at 200 g at room temperature for 5 min. Cells were re-suspended in 100 μl medium and cell number was estimated using a hemocytometer or Z2 Coulter Particle Counter (Beckman Coulter, Fullerton, Calif.). To estimate the average number of mRNA transcripts within each cell, 4 coverslips were transferred to clean 24-well dish and wash with PBS. Cell lysates were prepared, stored and mRNA copy numbers per cell were assayed according to QuantiGene 2.0 kit protocol (Panomics, Fremont, Calif.). RNA copy number was estimated by comparing signals from in vitro transcribed RNAs.

### Example 2: In Situ Detection of Bcr-Abl Gene Fusion

To demonstrate the feasibility of detecting RNA fusion transcripts using our assay, we simultaneously hybridized cultured K562 and Jurkat cells with probe sets to BCR and ABL. K562 cells are known to carry the BCR-ABL gene fusion, while Jurkat cell do not. The BCR and ABL probe sets were simultaneously detected with a signal amplification system labeled with a green fluorescent dye and a signal amplification system with a red fluorescent dye, respectively. As shown in FIG. 56, Jurkat cells stained in this manner showed individual green or red dots, indicating the presence of wild type BCR and ABL transcripts. However, as expected K562 cells showed a large number of yellow dots due to the juxtaposition of the BCR and ABL probe sets on the same transcript, indicating that a fusion gene was present. To our knowledge this is the first demonstration of in situ visualization of a fusion transcript.

### Example 3: Capture Probe (Label Extender) Design

The following sets forth a series of experiments that illustrate label extender design and that demonstrate that a configuration in which the 5′ ends of the label extenders hybridize to a nucleic acid of interest while the 3′ ends of the label extenders hybridize to a preamplifier results in stronger binding of the preamplifier to the nucleic acid than does a cruciform arrangement of the label extenders.

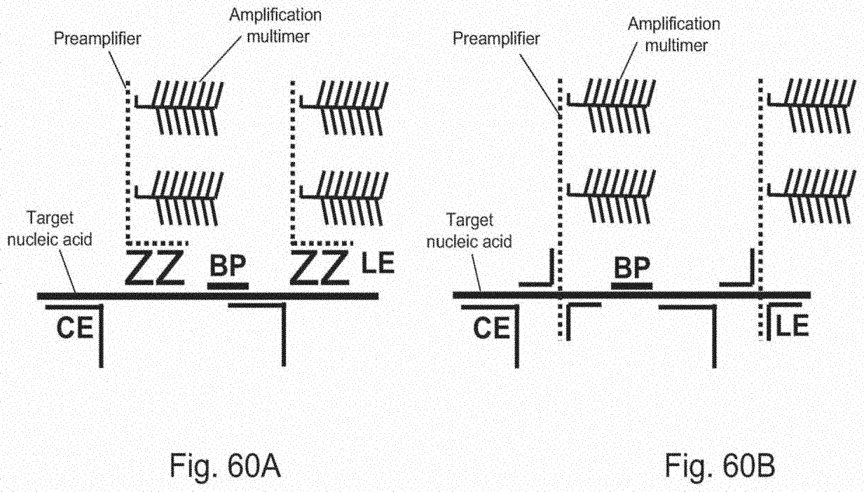

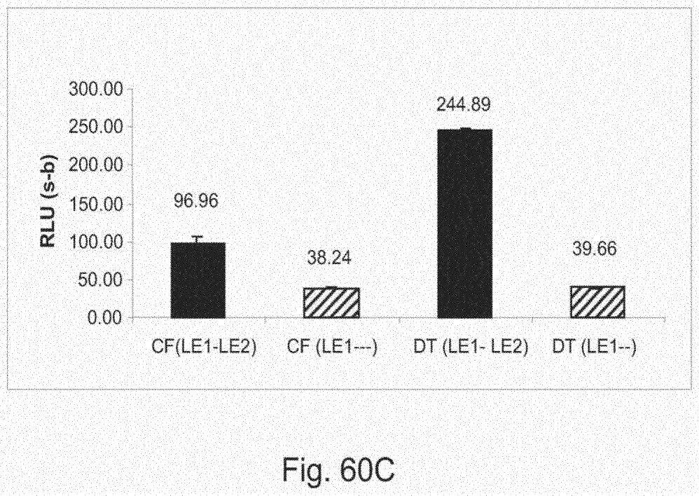

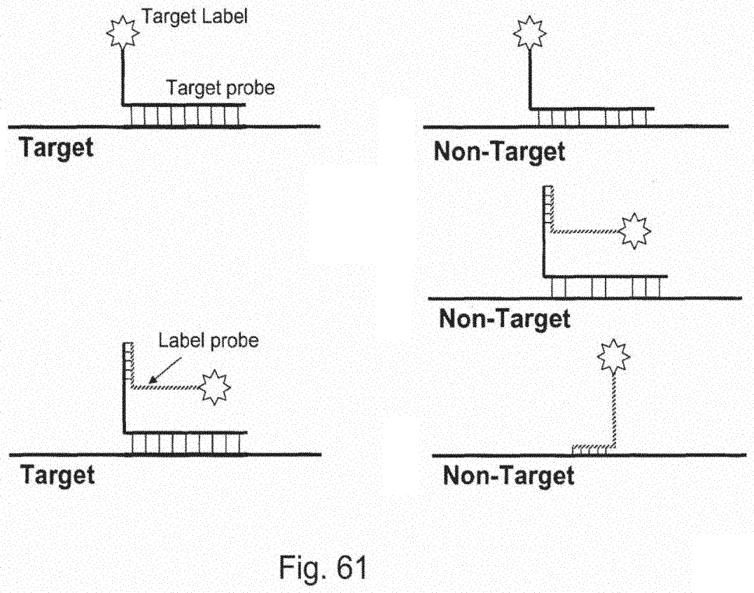

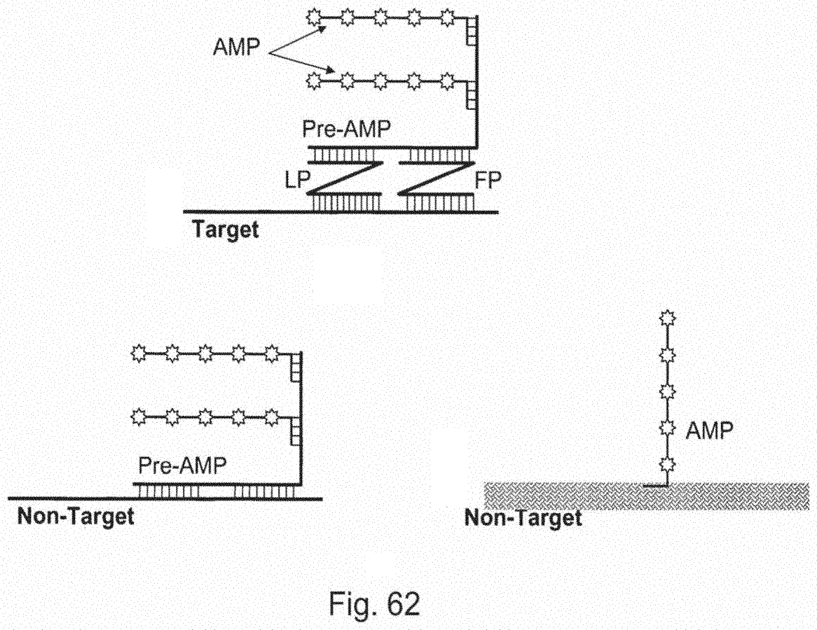

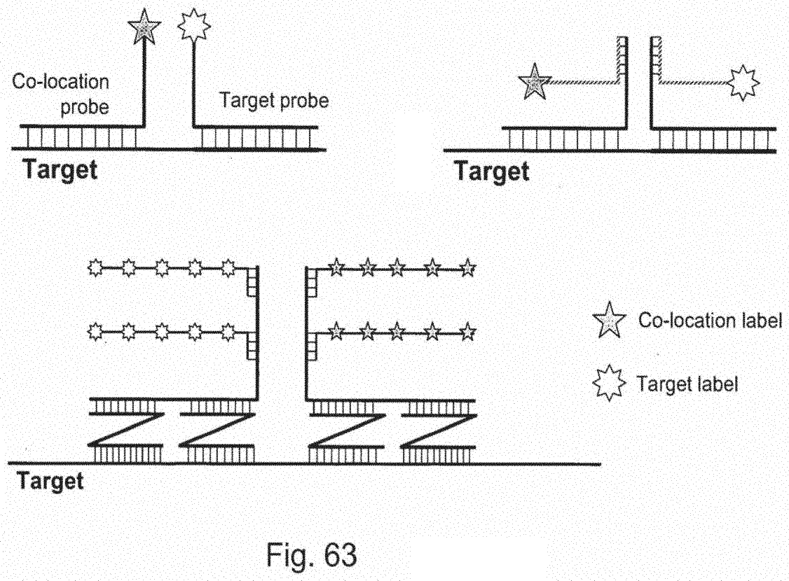

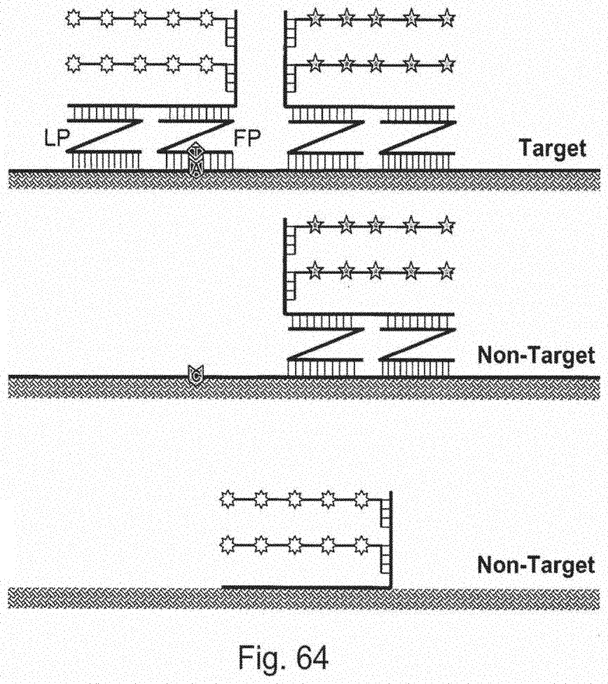

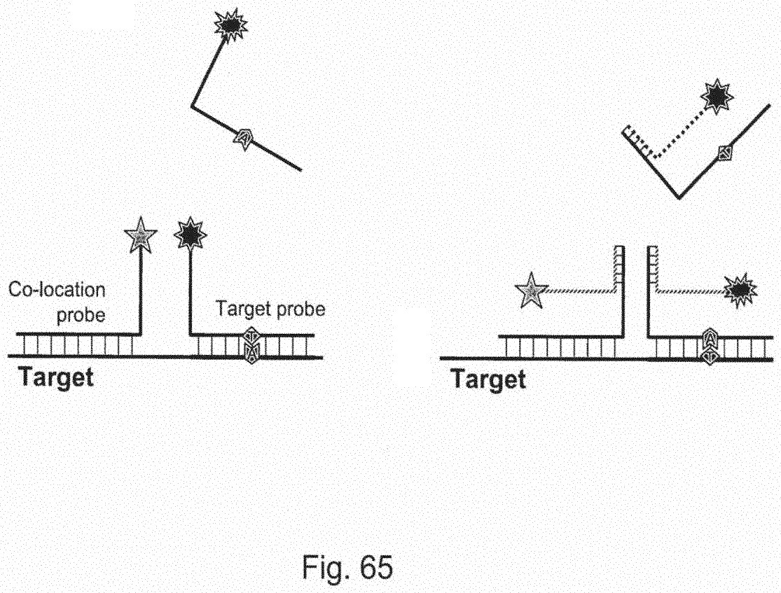

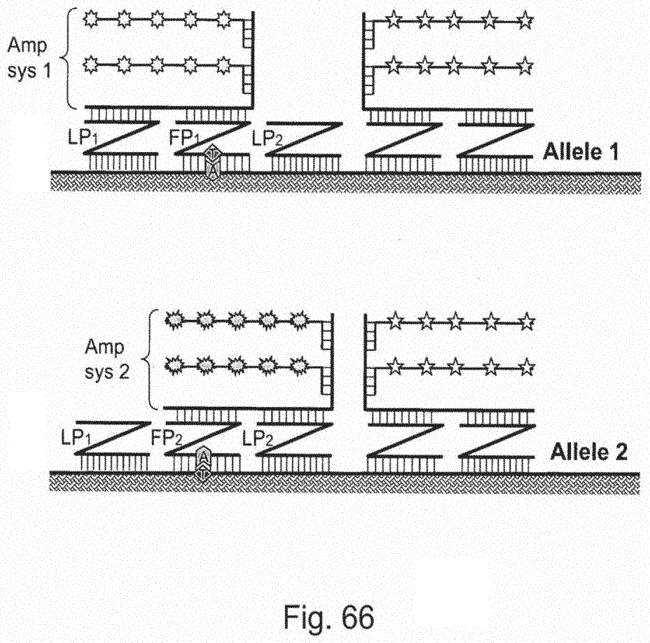

Two subsets of label extenders were designed to bind to a human GAPD nucleic acid target and to a preamplifier, as schematically illustrated in FIG. 60. Two label extenders bind each copy of the preamplifier. As shown in Panel A, in one subset of label extenders, the two label extenders in each pair bind the preamplifier through the same end (the 5′ end, in this example) and bind the target nucleic acid through the other end (double Z configuration). As shown in Panel B, in the other subset of label extenders, the two label extenders in each pair bind the preamplifier through opposite ends: the 5′ end of one label extender hybridizes to the preamplifier and the 3′ end to the target, while the 3′ end of the other label extender hybridizes to the preamplifier and the 5′ end to the target (cruciform configuration). Sequence L-2 (complementary to the preamplifier) is 14 nucleotides in length for each label extender, and comparable sequences L-2 and L-1 were used for the corresponding label extenders in both configurations. Sequences of the label extenders are presented in Tables 2 and 3. The sequence of the preamplifier is 5′ AGGCATAGGACCCGTGTCT tttttttttt AGGCATAGGACCCGTGTCT 11111 ATGCTTTGACTCAG AAAACGGTAACTTC 3′ (SEQ ID NO:1); the underlined sequences are complementary to sequences in the label extenders.

TABLE 2
Label extenders for the cruciform configuration. In each
label extender, sequence L-2 (complementary to a
sequence in the preamplifier) is underlined.

| | | | |
|---|---|---|---|
| GAPD127 | ccagtggactccacgacgtacTTTTTgaagttaccgtttt | CP1 tail | SEQ ID NO: 2 |
| GAPD128 | ctgagtcaaagcatTTTTTttctccatggtggtgaagacg | CP2 head | SEQ ID NO: 3 |
| GAPD129 | tcttgaggctgttgtcatacttctTTTTTgaagttaccgtttt | CP1 tail | SEQ ID NO: 4 |
| GADP130 | ctgagtcaaagcatTTTTTgcaggaggcattgctgatga | CP2 head | SEQ ID NO: 5 |
| GAPD131 | cagtagaggcagggatgatgttcTTTTTgaagttaccgtttt | CP1 tail | SEQ ID NO: 6 |
| GAPD132 | ctgagtcaaagcatTTTTTcacagccttggcagcgc | CP2 head | SEQ ID NO: 7 |

TABLE 3
Label extenders for the double Z configuration. In each
label extender, sequence L-2 (complementary to a
sequence in the preamplifier) is underlined.

| | | | |
|---|---|---|---|
| GAPD217 | ccagtggactccacgacgtacTTTTTgaagttaccgtttt | CP1 tail | SEQ ID NO: 8 |
| GAPD218 | ttctccatggtggtgaagacgTTTTTctgagtcaaagcat | CP2 tail | SEQ ID NO: 9 |
| GAPD219 | tcttgaggctgttgtcatacttctTTTTTgaagttaccgtttt | CP1 tail | SEQ ID NO: 10 |
| GAPD220 | gcaggaggcattgctgatgaTTTTTctgagtcaaagcat | CP2 tail | SEQ ID NO: 11 |
| GAPD221 | cagtagaggcagggatgatgttcTTTTTgaagttaccgtttt | CP1 tail | SEQ ID NO: 12 |
| GAPD222 | cacagccttggcagcgcTTTTTctgagtcaaagcat | CP2 tail | SEQ ID NO: 13 |

The double Z and cruciform label extender configurations were assessed in single plex QuantiGene™ bDNA assays using essentially standard QuantiGene™ assay conditions. QuantiGene™ kits are commercially available from Panomics, Inc. (on the world wide web at ((www.)panomics.com). Assays were performed basically as described in the supplier's instructions, with incubation at 53° C. on day one and 46° C. on day two, lx GAPD probe set, 10 amole/well of GAPD in vitro transcribed RNA, preamplifier concentration of 100 fmol/well with incubation for one hour at 46° C., amplification multimer (1.0 amp, Bayer) at 100 fmol/well with incubation for one hour at 46° C., followed by label probe at 100 fmol/well (1:1000 dilution) for one hour at 46° C., then substrate for 30 minutes at 46° C. In this experiment, the only difference between the two assays is whether the cruciform configuration label extender set or the double Z configuration label extender set is used.

The results are illustrated in FIG. 60 Panel C, which shows background-subtracted luminescence (Relative Light Units, signal minus background) measured for the cruciform configuration and the double Z configuration label extenders. The signal for the assay using the double Z configuration label extenders (DT LE1-LE2) is almost 2.5 fold higher than that for the assay using the cruciform configuration label extenders (CF LE1-LE2). For comparison, assays in which only one label extender from each pair was included in the assay gave similar signals regardless of whether the single label extender binding to the preamplifier was from the cruciform (CF LE1-) or the double Z (DT LE1-) subset.

The stronger signal observed using the double Z configuration label extenders demonstrates that this design enables more efficient capture of the preamplifier than does the cruciform design.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1-256. (canceled)

257. A composition comprising:
(a) a target nucleic acid;
(b) at least one set of two or more capture probes hybridized to the target nucleic acid;
(c) at least one set of two or more preamplifiers hybridized to the two or more capture probes;
(d) at least one set of two or more linker capture probes hybridized to the two or more preamplifiers;
(e) at least one amplifier hybridized to the two or more linker capture probes; and
(f) at least one label molecule hybridized to a section of the at least one amplifier;
wherein each capture probe comprises a T section complementary to a region of the target nucleic acid and an L section complementary to a region of one of the two or more preamplifiers, wherein each T section is complementary to a non-overlapping region of the target nucleic acid and each L section is complementary to a non-overlapping region of one of the two or more preamplifiers; and
wherein the at least one set of two or more linker capture probes cannot bind stably to either the two or more preamplifiers or the at least one amplifier alone.

258. The composition of claim 257, wherein each of the two or more linker capture probes hybridizes to different preamplifiers.

259. The composition of claim 258, wherein each of the two or more linker capture probes comprises a region complementary to a region of one of the two or more preamplifiers.

260. The composition of claim 257, wherein both of the two or more linker capture probes hybridize to the same amplifier.

261. The composition of claim 260, wherein each of the two or more linker capture probes comprises a region complementary to non-overlapping regions of the same amplifier.

262. The composition of claim 257, further comprising at least a second set of two or more linker capture probes hybridized to the two or more preamplifiers.

263. The composition of claim 262, wherein each of the two or more linker capture probes in the second set hybridizes to different preamplifiers.

264. The composition of claim 263, wherein each of the two or more linker capture probes in the second set comprises a region complementary to a region of one of the two or more preamplifiers.

265. The composition of claim 263, wherein each of the two or more linker capture probes in the second set comprises a region complementary to non-overlapping regions of the same amplifier.

266. The composition of claim 257, wherein the at least one set of two or more linker capture probes is integrated into the at least one amplifier.

267. The composition of claim 257, wherein the T section of at least one of the two or more capture probes is 3′ of its L section.

268. The composition of claim 257, wherein the T section of at least one of the two or more capture probes is 5′ of its L section.

269. The composition of claim 257, wherein the target nucleic acid is RNA.

270. The composition of claim 257, further comprising a cell comprising or suspected of comprising the target nucleic acid.

271. The composition of claim 257, wherein the T sections of the two or more capture probes are at least 20 nucleotides in length.

272. The composition of claim 257, wherein the L sections of the two or more capture probes are at least 13 nucleotides in length.

273. The composition of claim 257, wherein each T section of the two or more capture probes comprises a nucleotide sequence having a melting temperature that is above the melting temperature of its corresponding L section.

274. A kit comprising:
(a) at least one set of two or more capture probes designed to hybridize to a target nucleic acid;
(b) at least one set of two or more preamplifiers designed to hybridize to the two or more capture probes;
(c) at least one set of two or more linker capture probes designed to hybridize to the two or more preamplifiers;
(e) at least one amplifier designed to hybridize to the two or more linker capture probes; and
(f) at least one label molecule designed to hybridize to a section of the at least one amplifier;
wherein each capture probe comprises a T section complementary to a region of the target nucleic acid and an L section complementary to a region of one of the two or more preamplifiers, wherein each T section is complementary to a non-overlapping region of the target nucleic acid and each L section is complementary to a non-overlapping region of one of the two or more preamplifiers; and
wherein the at least one set of two or more linker capture probes cannot bind stably to either the two or more preamplifiers or the at least one amplifier alone.

275. A method of in situ detection of a target nucleic acid in a sample of cells, the method comprising:
(a) contacting the sample, wherein the cells of the sample are fixed and permeabilized, with at least one set of two or more capture probes designed to hybridize to the target nucleic acid;
(b) contacting the sample with at least one set of two or more preamplifiers designed to hybridize to the two or more capture probes; at least one set of two or more linker capture probes designed to hybridize to the two or more preamplifiers; at least one amplifier designed to hybridize to the two or more linker capture probes; and a plurality of label molecules designed to hybridize to a section of the at least one amplifier;
wherein each capture probe comprises a T section complementary to a region of the target nucleic acid and an L section complementary to a region of one of the two or more preamplifiers, wherein each T section is complementary to a non-overlapping region of the target nucleic acid and each L section is complementary to a non-overlapping region of one of the two or more preamplifiers; and wherein the at least one set of two or more linker capture probes cannot bind stably to either the two or more preamplifiers or the at least one amplifier alone;
(c) removing unbound probes, preamplifiers, amplifiers, and label molecules from the sample; and
(d) detecting in situ signal generated from the plurality of label molecules, thereby detecting the target nucleic acid.

276. The method of claim 275, wherein step (a) is performed at a hybridization temperature that is higher than the melting temperature of each of the T sections of the two or more capture probes.


SEQUENCE LISTING (13 sequences)

SEQ ID NO: 1 - DNA, 81 bp, Artificial Sequence
  synthetic oligonucleotide probe
aggcataggacccgtgtctttttttttttaggcataggacccgtgtcttttttatgctttgactcagaaaacggtaacttc

SEQ ID NO: 2 - DNA, 40 bp, Artificial Sequence
  synthetic oligonucleotide probe
ccagtggactccacgacgtactttttgaagttaccgtttt

SEQ ID NO: 3 - DNA, 40 bp, Artificial Sequence
  synthetic oligonucleotide probe
ctgagtcaaagcattttttttctccatggtggtgaagacg

SEQ ID NO: 4 - DNA, 43 bp, Artificial Sequence
  synthetic oligonucleotide probe
tcttgaggctgttgtcatacttcttttttgaagttaccgtttt

SEQ ID NO: 5 - DNA, 39 bp, Artificial Sequence
  synthetic oligonucleotide probe
ctgagtcaaagcattttttgcaggaggcattgctgatga

SEQ ID NO: 6 - DNA, 42 bp, Artificial Sequence
  synthetic oligonucleotide probe
cagtagaggcagggatgatgttctttttgaagttaccgtttt

SEQ ID NO: 7 - DNA, 36 bp, Artificial Sequence
  synthetic oligonucleotide probe
ctgagtcaaagcattttttcacagccttggcagcgc

SEQ ID NO: 8 - DNA, 40 bp, Artificial Sequence; identical to SEQ ID NO: 2
  synthetic oligonucleotide probe

SEQ ID NO: 9 - DNA, 40 bp, Artificial Sequence
  synthetic oligonucleotide probe
ttctccatggtggtgaagacgtttttctgagtcaaagcat

SEQ ID NO: 10 - DNA, 43 bp, Artificial Sequence; identical to SEQ ID NO: 4
  synthetic oligonucleotide probe

SEQ ID NO: 11 - DNA, 39 bp, Artificial Sequence
  synthetic oligonucleotide probe
gcaggaggcattgctgatgatttttctgagtcaaagcat

SEQ ID NO: 12 - DNA, 42 bp, Artificial Sequence; identical to SEQ ID NO: 6
  synthetic oligonucleotide probe

SEQ ID NO: 13 - DNA, 36 bp, Artificial Sequence
  synthetic oligonucleotide probe
cacagccttggcagcgctttttctgagtcaaagcat